(19) United States
(12) Patent Application Publication
(10) Pub. No.: US 2024/0417420 A1
(43) Pub. Date: Dec. 19, 2024

(54) POLAR CD33 LIGANDS SUITABLE FOR INCORPORATION INTO CARRIERS

(71) Applicant: GLYCOMIMETICS, INC., Rockville, MD (US)

(72) Inventors: John L. MAGNANI, Gaithersburg, MD (US); John M. PETERSON, Slate Hill, NY (US); lndranath GHOSH, Olney, MD (US); Jason NOGUEIRA, Germantown, MD (US); Miti RAHMAN, Rockville, MD (US)

(73) Assignee: GLYCOMIMETICS, INC., Rockville, MD (US)

(21) Appl. No.: 18/698,081

(22) PCT Filed: Sep. 30, 2022
(86) PCT No.: PCT/US2022/077375

§ 371 Date: Apr. 3, 2024

Related U.S. Application Data
(60) Provisional application No. 63/262,068, filed on Oct. 4, 2021.

(51) Int. Cl. C07H 15/26; A61K 47/54; A61K 47/69; C07H 1/00; C07H 17/02

(52) U.S. Cl. CPC C07H 15/26; A61K 47/549; A61K 47/6911; A61K 47/6929; C07H 1/00; C07H 17/02

(57) ABSTRACT

CD33 ligands which are useful for the synthesis of CD33 ligand-bearing carriers, wherein said CD33 ligand-bearing carriers are directly or indirectly linked to or associated with at least one anti-cancer agent, are described herein. Uses of said CD33 ligand-bearing carriers for treating and/or preventing a disease, disorder, or condition such as acute myeloid leukemia (AML) are also described.

This application claims the benefit under 35 U.S.C. § 119(e) to U.S. Provisional Application No. 63/262,068 filed Oct. 4, 2021, which application is incorporated by reference herein in its entirety.

CD33 ligands which are useful for the synthesis of CD33 ligand-bearing carriers, wherein said CD33 ligand-bearing carriers are directly or indirectly linked to or associated with at least one anti-cancer agent, and uses of said CD33 ligand-bearing carriers for treating and/or preventing a disease, disorder, or condition such as acute myeloid leukemia (AML) are described herein.

Acute myeloma leukemia (AML) is one of the most common types of leukemia among adults, and the most common acute leukemia affecting adults. AML is a cancer of myeloid stem cells, characterized by the rapid growth of abnormal cells that build up in the bone marrow and blood and interfere with normal blood cells. Symptoms may include fatigue, shortness of breath, easy bruising and bleeding, and increased risk of infection. It can progress rapidly and is typically fatal within weeks or months if left untreated. The 5-year survival rate for AML is 27.4%. It accounts for roughly 1.8% of cancer deaths in the United States.

First-line treatment of AML consists primarily of chemotherapy with an anthracycline/cytarabine combination, and is divided into two phases: induction and post-remission (or consolidation) therapy. The goal of induction therapy is to achieve a complete remission by reducing the number of leukemic cells to an undetectable level: the goal of consolidation therapy is to eliminate any residual undetectable disease and achieve a cure. The specific genetic mutations present within the cancer cells may guide therapy, as well as determine how long that person is likely to survive.

Despite advances in our understanding of the pathogenesis of AML, the short- and long-term outcomes for AML patients have remained unchanged over three decades. (Roboz et al., (2012) Curr. Opin. Oncol., 24: 711-719.) The median age at diagnosis is 66 years, with cure rates of less than 10% and median survival of less than 1 year. (Burnett et al., (2010), J. Clin. Oncol., 28: 586-595.) Although 70-80% of patients younger than 60 years old achieve complete remission, most eventually relapse, and overall survival is only 40-50% at 5 years. (Fernandez et al., (2009) N. Engl. J. Med., 361: 1249-1259; Mandelli et al., (2009) J. Clin. Oncol., 27: 5397-5403; Ravandi et al., (2006) Clin. Can. Res., 12(2): 340-344.) Relapse is thought to occur due to leukemic stem cells that escape initial induction therapy and drive reoccurrence of AML. (Dean et al., (2005) Nat. Rev. Cancer, 5(4): 275-294; Guan et al., (2003) Blood, 101(8): 3142; and Konopleva et al., (2002) Br. J. Haeratol. 118(2): 521-534.) Chemoresistance, the ability of cancer cells to evade or to cope with the presence of therapeutics, is also a key challenge for therapeutic success.

Siglecs comprise a family of receptors that are differentially expressed on leukocytes and other immune cells. Each siglec contains an N-terminal ‘V-set’ Ig domain that binds sialic acid containing ligands, followed by a variable number (1-16) of ‘C2-set’ Ig domains that extend the ligand binding site away from the membrane surface. Each siglec also exhibits distinct and varied specificity for sialoside sequences on glycoprotein and glycolipid glycans that are expressed on the same cell (cis) or on adjacent cells (trans). There are currently 14 known siglecs in humans, four of which are highly conserved in all mammalian species and the rest are classified as Siglec-3 (CD33) related siglecs, which comprise a rapidly evolving sub-family. (Padler-Karavani et al., The FASEB Journal, 23(3), 2017, pp. 1280-1293.)

The restricted expression of several siglecs to one or a few cell types makes them attractive targets for cell-directed therapies. (O'Reilly and Paulson, Trends Pharmacol Sci., 30(5), 2009, pp. 240-248.) As siglecs are endocytic receptors, attaching suitable siglec ligands to carriers of therapeutic agents can facilitate efficient targeting of cells expressing the siglec receptors, enabling the therapeutic agents to be efficiently carried into the cells.

CD33 was identified as a marker of myeloid leukemias and is overexpressed in AML cells. Thus, therapeutics incorporating CD33 ligands may be useful for cell-directed drug delivery to treat AML. Accordingly, there is a need in the art for CD33 ligands, and for the development of methods employing such compounds as a way to target diseases, disorders, and/or conditions associated with expression of CD33, such as AML. The present disclosure may fulfill one or more of these needs and/or may provide other advantages.

Disclosed are compounds of Formula (I):

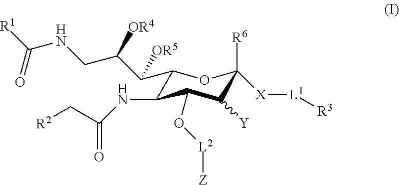

and pharmaceutically acceptable salts thereof, wherein R¹, R², R³, R⁴, R⁵, R⁶, L¹, L², X, Y, and Z are as defined herein.

In some embodiments, a process for making CD33 ligand-bearing carriers of Formula (II) is provided, wherein the process comprises reacting or associating a compound of Formula (I) with a compound of Formula (III):

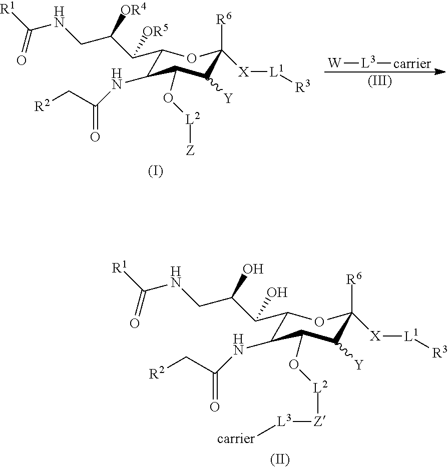

wherein R¹, R², R³, R⁴, R⁵, R⁶, L¹, L², L³, X, Y, Z, Z′, W, and carrier are as defined herein.

In some embodiments, a method for treatment and/or prevention of AML is disclosed, the method comprising administering to a subject in need thereof at least one compound of Formula (II) or a composition comprising same, wherein said at least one compound of Formula (II) delivers an effective amount of at least one anti-cancer agent.

In some embodiments, a method for treatment and/or prevention of AML is disclosed, the method comprising administering to a subject in need thereof an effective amount of at least one compound of Formula (II) or a composition comprising same, wherein at least one anti-cancer agent is directly or indirectly linked to or associated with said at least one compound of Formula (II).

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the prophetic synthesis of compound 7.

FIG. 2 is a diagram illustrating the prophetic synthesis of compound 12.

FIG. 3 is a diagram illustrating the prophetic synthesis of compound 142.

FIG. 4 is a diagram illustrating the prophetic synthesis of compound 146.

FIG. 5 is a diagram illustrating the prophetic synthesis of compound 160.

FIG. 6 is a diagram illustrating the prophetic syntheses of compound 170.

FIG. 7 is a diagram illustrating the prophetic synthesis of compound 177.

FIG. 8 is a diagram illustrating the prophetic synthesis of compound 185.

FIG. 9 is a diagram illustrating the prophetic synthesis of compound 191.

FIG. 10 is a diagram illustrating the prophetic synthesis of compounds 194a and 194b.

FIG. 11 is a diagram illustrating the prophetic synthesis of compound 201.

FIG. 12 is a diagram illustrating the prophetic synthesis of compound 211.

FIG. 13 is a diagram illustrating the prophetic synthesis of compound 217.

FIG. 14 is a diagram illustrating exemplary conjugation chemistry useful for the synthesis of compounds of Formula (II).

Disclosed herein are CD33 ligands, which are useful for the synthesis of CD33 ligand-bearing carriers. The CD33 ligand-bearing carriers may be useful for treating and/or preventing at least one disease, disorder, or condition, including AML.

In some embodiments, disclosed are compounds of Formula (I):

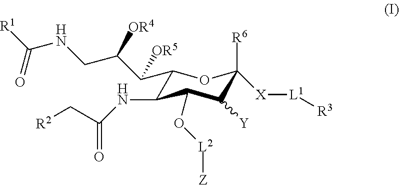

and pharmaceutically acceptable salts thereof, wherein
R¹ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from halo, C₁₋₈ alkyl, C₂₋₈ alkenyl, C₂₋₈ alkynyl, C₁₋₈ haloalkyl, C₂₋₈ haloalkenyl, C₁₋₈ haloalkynyl, C₁₋₈ hydroxyalkyl, C₂₋₈ hydroxyalkenyl, C₂₋₈ hydroxyalkynyl, C₆₋₁₈ aryl, C₁₋₃ heteroaryl, —OT¹, —ST¹, —C(═O)OT¹, —C(═O)NT¹ T², —NT¹ T², —NT¹ C(═O)T², —NT¹ SO₂T², —S(═O)T¹, —SO₂T¹ and —OSO₃T¹ groups, wherein T¹ and T², which may be identical or different, are independently chosen from H, C₁₋₈ alkyl, C₁₋₄ haloalkyl, and hydroxy protecting groups, or T¹ and T² join together along with the heteroatom to which they are attached to form an optionally substituted, saturated or unsaturated, 3-10 membered ring;
R² is chosen from C₆₋₈ aryl and C₁₋₃ heteroaryl groups, wherein the C₆₋₈ aryl and C₁₋₁₃ heteroaryl groups are optionally substituted with one or more groups independently chosen from halo, C₁₋₈ alkyl, C₂₋₈ alkenyl, C₂₋₄ alkynyl, C₁₋₈ haloalkyl, C₂₋₈ haloalkenyl, C₂₋₈ haloalkynyl, C₆₋₁₈ aryl, C₁₋₁₃ heteroaryl, -J¹-OT³, -J¹-ST³, -J¹-C(═O)OT³, -J¹-C(═O)NT³T⁴, -J¹-NT³T⁴, -J¹-NT³C(═O)T⁴, -J¹-NT³SO₂T⁴, -J¹-S(═O)T³, -J¹-SO₂T³-J¹-S(═O)₂OT³, and -J¹-P(═O)(OT³)(OT⁴) groups, wherein J¹ is chosen from a bond, divalent C₁₋₁₀ alkyl, and divalent C₁₋₁₀ haloalkyl groups and wherein T³ and T⁴, which may be identical or different, are independently chosen from H, C₁₋₄ alkyl, and C₁₋₈ haloalkyl groups, or T³ and T⁴ join together along with the heteroatom to which they are attached to form an optionally substituted, saturated or unsaturated, 3-10 membered ring;
R³ is chosen from -J²-OT¹, -J²-C(═O)OT⁵, -J²-C(═O)NT⁵T⁶, -J²-S(═O)₂OT⁵, -J²-OS(═O)₂OT, -J²-P(═O)(OT⁵)(OT⁶), C₆₋₄₈ aryloxy, C₇₋₁₉ arylalkoxy, C₆₋₈ aryl, and C₁₋₁₀ heteroaryl groups, wherein J² is chosen from a bond, divalent C₁₋₁₀ alkyl, and divalent C₁₋₁₀ haloalkyl groups, wherein T¹ and T, which may be identical or different, are independently chosen from H and C₁₋₈ alkyl groups, or T⁵ and T⁶ join together along with the heteroatoms to which they are attached to form an optionally substituted, saturated or unsaturated, 3-10 membered ring, and wherein the C₆₋₁₈ aryloxy, C₇₋₁₉ arylalkoxy, C₆₋₈ aryl, and C₁₋₃ heteroaryl groups are optionally substituted with one or more groups independently chosen from R¹, halo, C₁₋₈ alkyl, C₁₋₈ haloalkyl, -OT¹, —C(═O)OT⁷, and —C(═O)NT⁷T⁸ groups, wherein R¹ is independently chosen from C₆₋₁₈ aryl groups which are optionally substituted with one or more groups independently chosen from halo, C₁₋₈ alkyl, C₆₋₁₈ aryl, -OT⁹, —C(═O)OT⁹, and —C(═O)NT⁹T¹⁰ groups, wherein T⁷, T⁸, T¹, and T¹ o, which may be identical or different, are independently chosen from H and C₁₋₈ alkyl groups, or T¹ and T⁸ join together along with the nitrogen atom to which they are attached to form an optionally substituted, saturated or unsaturated, 3-10 membered ring and/or T⁹ and T¹⁰ join together along with the nitrogen atom to which they are attached to form an optionally substituted, saturated or unsaturated, 3-10 membered ring;
R⁴ and R⁵, which may be identical or different, are independently chosen from H and hydroxy protecting groups, or R⁴ and R⁵ join together along with the oxygen atoms to which they are attached to form an optionally substituted, saturated or unsaturated, 3-10 membered ring;
R⁶ is chosen from —C(═O)OR⁸, and —C(═O)NT¹¹T¹², wherein R⁸ is chosen from H and carboxy protecting groups and wherein T¹¹ and T¹², which may be identical or different, are independently chosen from H and C₁₋₈ alkyl groups, or T¹ and T¹ join together along with the nitrogen atom to which they are attached to form an optionally substituted, saturated or unsaturated, 3-10 membered ring;
L¹ and L², which may be identical or different, are independently chosen from linker groups; X is chosen from —O—, —S—, —CH₂—, and —N(T¹³)—, wherein T¹³ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and —C(═O)T¹ groups, wherein T¹⁴ is chosen from H, halo, C₁₋₈ alkyl, C₆₋₁₈ aryl, and C₁₋₃ heteroaryl groups;
Y is chosen from H, halo, and —OT¹⁴ groups, wherein T¹⁴ is chosen from H and C₁₋₈ alkyl groups; and
Z is chosen from lipids, nucleophiles, electrophiles, and groups capable of undergoing a cycloaddition reaction.

R¹ is chosen from C₆₋₉ aryl and C₁₋₃ heteroaryl groups, wherein the C₆₋₁₈ aryl and C₁₋₃ heteroaryl groups are optionally substituted with one or more groups independently chosen from —OT¹, —ST¹, —C(═O)OT¹, —C(═O)NT¹ T², —NT¹ T², —NT¹ C(═O)T², —NT¹ SO₂T², —S(═O)T¹, —SO₂T¹ and —OSO₃T¹ groups.

In some embodiments, R¹ is chosen from C₆₋₁₈ i aryl and C₁₋₃ heteroaryl groups, wherein the C₆₋₁₈ aryl and C₁₋₁₃ heteroaryl groups are optionally substituted with one or more groups independently chosen from —OT¹, —ST¹, and —NT¹ T² groups.

In some embodiments, R¹ is chosen from C₆₋₁₈ aryl and C₁₋₃ heteroaryl groups, wherein the C₆₋₁₈ aryl and C₁₋₁₃; heteroaryl groups are optionally substituted with one or more groups independently chosen from —OH, —SH, and —NH₂.

In some embodiments, R¹ is chosen from C₆₋₁₈ aryl and C₁₋₁₃ heteroaryl groups, wherein the C₆₋₈ aryl and C₁₋₁₃ heteroaryl groups are optionally substituted with one or more groups independently chosen from —C(═O)OT¹ and —C(═O)NT¹ T² groups.

In some embodiments, R¹ is chosen from C₆₋₁₈ aryl and C₁₋₁₃ heteroaryl groups, wherein the C₆₋₁₈ aryl and C₁₋₃ heteroaryl groups are optionally substituted with one or more groups independently chosen from —C(═O)OH and —C(═O)NH₂.

In some embodiments, R¹ is chosen from C₆₋₈ aryl and C₁₋₃ heteroaryl groups, wherein the C₆₋₁₈ aryl and C₁₋₃ heteroaryl groups are optionally substituted with one or more groups independently chosen from —NT¹ C(═O)T² and —NT¹ SO₂T² groups.

In some embodiments, R¹ is chosen from C₆₋₁₈ aryl and C₁₋₃ heteroaryl groups, wherein the C₆₋₁₈ aryl and C₁₋₃ heteroaryl groups are optionally substituted with one or more groups independently chosen from —S(═O)T¹, —SO₂T¹ and —OSO₃T¹ groups.

In some embodiments, R¹ is chosen from C₆₋₁₈ i aryl and C₁₋₃ heteroaryl groups, wherein the C₆₋₁₈ aryl and C₁₋₁₃ heteroaryl groups are optionally substituted with one or more groups independently chosen from —SO₂H and —OSO₃H.

In some embodiments, R¹ is chosen from C₆₋₂ aryl and C₁₋₂ heteroaryl groups, wherein the C₆₋₂ aryl and C₁₋₂ heteroaryl groups are optionally substituted with one or more groups independently chosen from halo, C₁₋₈ alkyl, C₁₋₈ haloalkyl, C₁₋₈ hydroxyalkyl, C₆₋₂ aryl, C₁₋₁₂ heteroaryl, —OT¹, —C(═O)OT¹, and —C(═O)NT¹ T² groups, wherein T¹ and T², which may be identical or different, are independently chosen from H, C₁₋₈ alkyl, and C₁₋₈ haloalkyl groups, or T¹ and T² join together along with the heteroatom to which they are attached to form an optionally substituted, saturated or unsaturated, 3-10 membered ring.

In some embodiments, R¹ is chosen from C₆₋₂ aryl and C₁₋₁₂ heteroaryl groups, wherein the C₆₋₁₂ aryl and C₁₋₂ heteroaryl groups are optionally substituted with one or more groups independently chosen from halo, C₁₋₈ alkyl, C₁₋₈ haloalkyl, C₁₋₈ hydroxyalkyl, —OT¹ and —C(═O)OT¹ groups, wherein T¹ is chosen from H, C₁₋₈ alkyl, and C₁₋₈ haloalkyl groups.

In some embodiments, R¹ is chosen from C₆₋₁₀ aryl and C₁₋₅ heteroaryl groups, wherein the C₆₋₁₀ aryl and C₁₋₅ heteroaryl groups are optionally substituted with one or more groups independently chosen from halo, C₁₋₈ alkyl, C₁i. haloalkyl, C₁₋₄ hydroxyalkyl, —OT¹ and —C(═O)OT¹ groups, wherein T¹ is chosen from H, C₁₋₈ alkyl, and C₁₋₈ haloalkyl groups.

In some embodiments, R¹ is chosen from C₆₋₁₀ aryl and C₁₋₅ heteroaryl groups, wherein the C₆₋₁₀ aryl and C₁₋₅ heteroaryl groups are optionally substituted with one or more groups independently chosen from halo, C₁₋₄ alkyl, C₁₋₈ hydroxyalkyl, and —OT¹, wherein T¹ is chosen from H and C₁₋₈ alkyl groups.

In some embodiments, R¹ is chosen from C₆₋₁₀ aryl and C₁₋₅ heteroaryl groups, wherein the C₆₋₁₀ aryl and C₁₋₈ heteroaryl groups are optionally substituted with one or more groups independently chosen from halo, C₁₋₄ alkyl, C₁₋₄ hydroxyalkyl, and —OT¹ groups, wherein T¹ is chosen from hydroxy protecting groups. In some embodiments, the hydroxy protecting group is chosen from formyl, acetyl, chloroacetyl, o-nitrophenylacetyl, o-nitrophenoxy-acetyl, trifluoroacetyl, acetoacetyl, 4-chlorobutyryl, isobutyryl, o-nitrocinnamoyl, picolinoyl, acylisothiocyanate, aminocaproyl, benzoyl, benzyl, β-methoxyethoxymethyl, dimethoxytrityl, methoxymethyl, methoxytrityl [(4-methoxyphenyl)diphenylmethyl], p-methoxybenzyl, methylthiomethyl, pivaloyl, tetrahydropyranyl, tetrahydrofuranyl, trityl, and silyl groups. In some embodiments, the hydroxy protecting group is a benzoyl group.

In some embodiments, R¹ is chosen from C₆₋₁₀ aryl and C₁₋₅ heteroaryl groups, wherein the C₆₋₁₀ aryl and C₁₋₈ heteroaryl groups are optionally substituted with one or more groups independently chosen from halo, C₁₋₈ alkyl, C₁₋₈ hydroxyalkyl, and —OH groups.

In some embodiments, R¹ is chosen from C₆₋₁₀ aryl groups optionally substituted with one or more groups independently chosen from halo, C₁₋₈ alkyl, C₁₋₈ hydroxyalkyl, and —OH groups.

In some embodiments, R¹ is phenyl substituted with one or more groups independently chosen from halo, C₁₋₈ alkyl, C₁₋₈ hydroxyalkyl, and —OH groups.

In some embodiments, R¹ is phenyl substituted with one or more groups independently chosen from halo, C₁₋₄ alkyl, and —OH groups.

In some embodiments, R¹ is phenyl substituted with one or more groups independently chosen from fluoro, methyl, and —OH.

In some embodiments, R¹ is

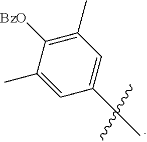

In some embodiments, R¹ is

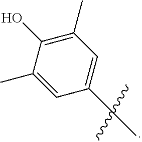 [SMILES: *c1cc(C)c(O)c(C)c1]

In some embodiments, R² is chosen from C₆₋₁₈ aryl and C₁₋₃ heteroaryl groups, wherein the C₆₋₁₈ aryl and C₁₋₃ heteroaryl groups are optionally substituted with one or more groups independently chosen from halo, C₁₋₈ alkyl, C₂₋₈ alkenyl, C₂₋₈ alkynyl, C₁₋₈ haloalkyl, C₂₋₈ haloalkenyl, C₂₋₄ haloalkynyl, C₆₋₁₈ aryl, and C₁₋₁₃ heteroaryl groups.

In some embodiments, R² is chosen from C₆₋₁₈ aryl and C₁₋₃ heteroaryl groups, wherein the C₆₋₁₈ aryl and C₁₋₁₃ heteroaryl groups are optionally substituted with one or more groups independently chosen from -JP-OT³, -J¹-ST³, -J¹-C(═O)OT³, -J¹-C(═O)NT³T⁴, -J¹-NT³T⁴, -J¹-NT³C(═O)T⁴, -J¹-NT³SO₂T⁴, -J¹-S(═O)T³, -J¹-SO₂T³ and -J¹-S(═O)₂OT³, and -J¹-P(═O)(OT³)(OT⁴) groups.

In some embodiments, J¹ is a bond. In some embodiments, J¹ is chosen from divalent C₁₋₁₀ alkyl groups. In some embodiments, J¹ is chosen from divalent C₁₋₈ alkyl groups. In some embodiments, J¹ is chosen from divalent C₁₋₆ alkyl groups. In some embodiments, J¹ is chosen from divalent C₁₋₄ alkyl groups. In some embodiments, J¹ is chosen from divalent C₁₋₃ alkyl groups. In some embodiments, J¹ is chosen from divalent C₁₋₂ alkyl groups. In some embodiments, J¹ is a divalent C₁ alkyl group, i.e., divalent methylene.

In some embodiments, J¹ is chosen from —CH₂—, —(CH₂)₂—, —(CH₂)₂—, —(CH₂)₄—, —(CH₂)₅—, —(CH₂)₆—, —(CH₂)₇—, —(CH₂)₈—, —(CH₂)₉—, and —(CH₂)₁₀₋₈ In some embodiments, J¹ is —CH₂—. In some embodiments, J¹ is —(CH₂)₂₋₈ In some embodiments, J¹ is —(CH₂)₃₋₈ In some embodiments, J¹ is —(CH₂)₄₋₈ In some embodiments, J¹ is —(CH₂)₅₋₈ In some embodiments, J¹ is —(CH₂)₆₋₈ In some embodiments, J¹ is —(CH₂)₇₋₈ In some embodiments, J¹ is —(CH₂)₅—. In some embodiments, J¹ is —(CH₂)₉₋₈ In some embodiments, J¹ is —(CH₂)₁₀₋₈

In some embodiments, J¹ is chosen from

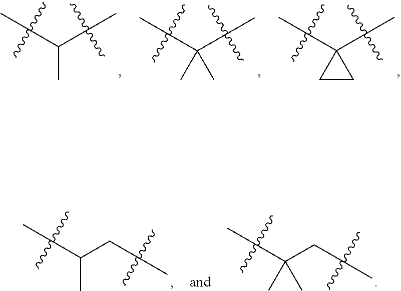

In some embodiments, J¹ is chosen from divalent C₁₋₁₀ haloalkyl groups. In some embodiments, J¹ is chosen from divalent C₁₋₈ haloalkyl groups. In some embodiments, J¹ is chosen from divalent C₁₋₆ haloalkyl groups. In some embodiments, J¹ is chosen from divalent C₁₋₄ haloalkyl groups. In some embodiments, J¹ is chosen from divalent C₁₋₃ haloalkyl groups. In some embodiments, J¹ is chosen from divalent C₁₋₂ haloalkyl groups. In some embodiments, J¹ is chosen from divalent C₁₋₁₀ haloalkyl groups.

In some embodiments, R² is chosen from C₆₋₁₈ aryl and C₁₋₃ heteroaryl groups, wherein the C₆₋₁₈ aryl and C₁₋₁₃ heteroaryl groups are optionally substituted with one or more groups independently chosen from -J¹-OT³, -J¹-ST³, and -J¹-NT³T groups.

In some embodiments, R² is chosen from C₆₋₁₈ aryl and C₁₋₃ heteroaryl groups, wherein the C₆₋₁₈ aryl and C₁₋₁₃ heteroaryl groups are optionally substituted with one or more groups independently chosen from -J¹-OH, -J¹-SH, and -J¹-NH₂ groups.

In some embodiments, R² is chosen from C₆₋₁₈ aryl and C₁₋₃ heteroaryl groups, wherein the C₆₋₈ aryl and C₁₋₃ heteroaryl groups are optionally substituted with one or more groups independently chosen from -J-OT³, -J¹-ST³, and -J¹-NT³T⁴ groups, wherein J¹ is a bond.

In some embodiments, R² is chosen from C₆₋₁₈ aryl and C₁₋₃ heteroaryl groups, wherein the C₆₋₈ aryl and C₁₋₃ heteroaryl groups are optionally substituted with one or more groups independently chosen from -J¹-OH, -J¹-SH, and -J¹-NH₂ groups, wherein J¹ is a bond.

In some embodiments, R² is chosen from C₆₋₁₈ aryl and C₁₋₁₃ heteroaryl groups, wherein the C₆₋₈ aryl and C₁₋₃ heteroaryl groups are optionally substituted with one or more groups independently chosen from -J¹-OT³, -J¹-ST³, and -J¹-NT³T⁴ groups, wherein J¹ is chosen from divalent C₁₋₁₃ alkyl groups.

In some embodiments, R² is chosen from C₆₋₈ aryl and C₁₋₁₃ heteroaryl groups, wherein the C₆₋₈ aryl and C₁₋₃ heteroaryl groups are optionally substituted with one or more groups independently chosen from -J¹-OH, -J¹-SH, and -J¹-NH₂ groups, wherein J¹ is chosen from divalent C₁₋₁₀ alkyl groups.

In some embodiments, R² is chosen from C₆₋₈ aryl and C₁₋₁₃ heteroaryl groups, wherein the C₆₋₈ aryl and C₁₋₃ heteroaryl groups are optionally substituted with one or more groups independently chosen from -J¹-C(═O)OT³ and -J¹-C(═O)NT³T⁴ groups.

In some embodiments, R² is chosen from C₆₋₈ aryl and C₁₋₃ heteroaryl groups, wherein the C₆₋₁₈ aryl and C₁₋₁₃ heteroaryl groups are optionally substituted with one or more groups independently chosen from -J¹-C(═O)OH and -J¹-C(═O)NH₂ groups.

In some embodiments, R² is chosen from C₆₋₈ aryl and C₁₋₃ heteroaryl groups, wherein the C₆₋₁₈ aryl and C₁₋₃ heteroaryl groups are optionally substituted with one or more groups independently chosen from -J¹-C(═O)OH groups.

In some embodiments, R² is chosen from C₆₋₁₈ i aryl and C₁₋₃ heteroaryl groups, wherein the C₆₋₁₈ aryl and C₁₋₃ heteroaryl groups are optionally substituted with one or more groups independently chosen from -J¹-C(═O)OT³ and -J¹-C(═O)NT³T⁴ groups, wherein J¹ is a bond.

In some embodiments, R² is chosen from C₆₋₁₈ i aryl and C₁₋₁₃ heteroaryl groups, wherein the C₆₋₁₈ aryl and C₁₋₁₃ heteroaryl groups are optionally substituted with one or more groups independently chosen from -J¹-C(═O)OH and -J¹-C(═O)NH₂ groups, wherein J¹ is a bond.

In some embodiments, R² is chosen from C₆₋₁₈ i aryl and C₁₋₁₃ heteroaryl groups, wherein the C₆₋₁₈ aryl and C₁₋₁₃ heteroaryl groups are optionally substituted with one or more groups independently chosen from -J¹-C(═O)OH groups, wherein J¹ is a bond.

In some embodiments, R² is chosen from C₆₋₁₈ aryl and C₁₋₃ heteroaryl groups, wherein the C₆₋₈ aryl and C₁₋₁₃ heteroaryl groups are optionally substituted with one or more groups independently chosen from -J¹-C(═O)OT; and -J¹-C(═O)NT³T⁴ groups, wherein J¹ is chosen from divalent C₁₋₁₀ alkyl groups.

In some embodiments, R² is chosen from C₆₋₁₈ aryl and C₁₋₃ heteroaryl groups, wherein the C₆₋₁₈ aryl and C₁₋₃ heteroaryl groups are optionally substituted with one or more groups independently chosen from -J¹-C(═O)OH and -J¹-C(═O)NH₂ groups, wherein J¹ is chosen from divalent C₁₋₁₀ alkyl groups.

In some embodiments, R² is chosen from C₆₋₁₈ aryl and C₁₋₁₃ heteroaryl groups, wherein the C₆₋₈ aryl and C₁₋₁₃ heteroaryl groups are optionally substituted with one or more groups independently chosen from -J¹-C(═O)OH groups, wherein J¹ is chosen from divalent C₁₋₁₀ alkyl groups.

In some embodiments, R² is chosen from C₆₋₁₈ aryl and C₁₋₃ heteroaryl groups, wherein the C₆₋₈ aryl and C₁₋₁₃ heteroaryl groups are optionally substituted with one or more groups independently chosen from -J¹-NT³C(═O)T⁴ and -J¹-NT³SO₂T⁴ groups.

In some embodiments, R² is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from -J¹—NT³C(═O)T⁴ and -J¹-NT³SO₂T⁴ groups, wherein J¹ is a bond.

In some embodiments, R² is chosen from C₆₋₁₈ aryl and C₁₋₁₃ heteroaryl groups, wherein the C₆₋₈ aryl and C₁₋₃ heteroaryl groups are optionally substituted with one or more groups independently chosen from -J¹—NT³C(═O)T⁴ and -J¹-NT³SO₂T⁴ groups, wherein J¹ is chosen from divalent C₁₋₁₀ alkyl groups.

In some embodiments, R² is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from -J¹-S(═O)T³, -J¹-SO₂T³, -J¹-S(═O)₂OT³, and -J¹-P(═O)(OT³)(OT⁴) groups.

In some embodiments, R² is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from -J¹-SO₂H, -J¹-S(═O)₂OH, and -J¹-P(═O)(OH)₂ groups.

In some embodiments, R² is chosen from C₆₋₈ aryl and C₁₋₁₃ heteroaryl groups, wherein the C₆₋₁₈ aryl and C₁₋₁₃ heteroaryl groups are optionally substituted with one or more groups independently chosen from -J¹-S(═O)T³, -J¹-SO₂T³, -J¹-S(═O)₂OT³, and -J¹-P(═O)(OT³)(OT⁴) groups, wherein J¹ is a bond.

In some embodiments, R² is chosen from C₆₋₈ aryl and C₁₋₁₃ heteroaryl groups, wherein the C₆₋₁₈ aryl and C₁₋₁₃ heteroaryl groups are optionally substituted with one or more groups independently chosen from -J¹-SO₂H, -J¹-S(═O)₂OH, and -J¹-P(═O)(OH)₂ groups, wherein J¹ is a bond.

In some embodiments, R² is chosen from C₆₋₈ aryl and C₁₋₁₃ heteroaryl groups, wherein the C₆₋₁₈ aryl and C₁₋₁₃ heteroaryl groups are optionally substituted with one or more groups independently chosen from -J¹-S(═O)T³, -J¹-SO₂T³, -J¹-S(═O)₂OT³, and -J¹-P(═O)(OT³)(OT⁴) groups, wherein J¹ is chosen from divalent C₁₋₁₀ alkyl groups.

In some embodiments, R² is chosen from C₆₋₈ aryl and C₁₋₁₃ heteroaryl groups, wherein the C₆₋₁₈ aryl and C₁₋₁₃ heteroaryl groups are optionally substituted with one or more groups independently chosen from -J¹-SO₂H, -J¹-S(═O)₂OH, and -J¹-P(═O)(OH)₂ groups, wherein J¹ is chosen from divalent C₁₋₁₀ alkyl groups.

In some embodiments, R² is chosen from C₁₋₃ heteroaryl groups optionally substituted with one or more groups independently chosen from halo, C₁₋₄ alkyl, C₁₋₈ haloalkyl, C₁₋₈ hydroxyalkyl, C₆₋₁₈ aryl, C₁₋₃ heteroaryl, —OT³, and —C(═O)OT³ groups.

In some embodiments, R² is chosen from C₁₋₃ heteroaryl groups optionally substituted with one or more groups independently chosen from halo, C₁₋₈ alkyl, C₁₋₈ haloalkyl, C₁₋₈ hydroxyalkyl, and —OT³ groups.

In some embodiments, R² is chosen from C₂₋₆ heteroaryl groups optionally substituted with one or more groups independently chosen from halo, C₁₋₈ alkyl, C₁₋₈ haloalkyl, and —OT³ groups.

In some embodiments, R² is chosen from triazole groups optionally substituted with one or more groups independently chosen from halo, C₁₋₈ alkyl, C₁₋₈ haloalkyl, and —OT³ groups.

In some embodiments, R² is chosen from triazole groups substituted with one or more groups independently chosen from C₁₋₈ alkyl groups.

In some embodiments, R² is chosen from

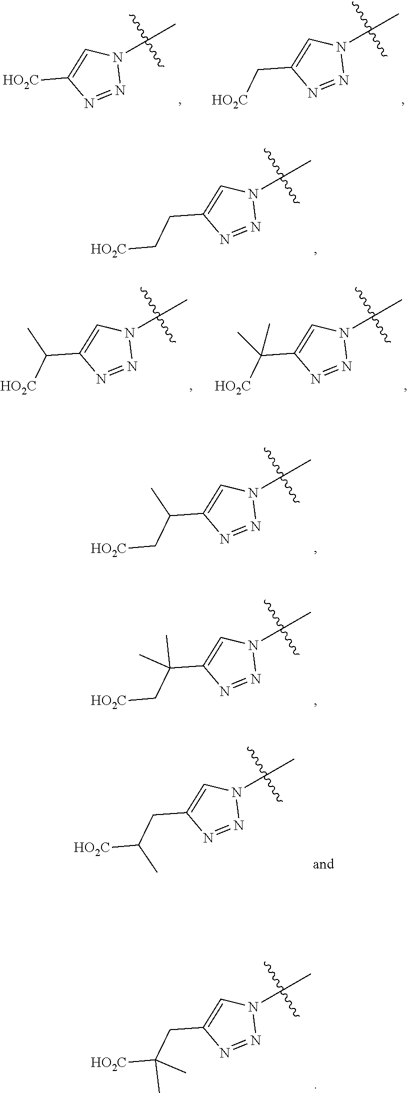

In some embodiments, R² is chosen from

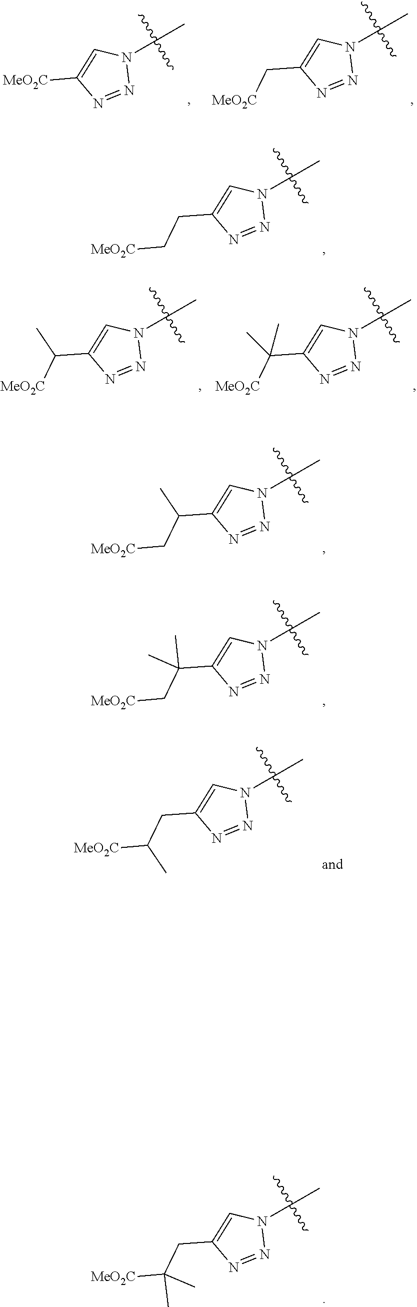

In some embodiments, R² is chosen from

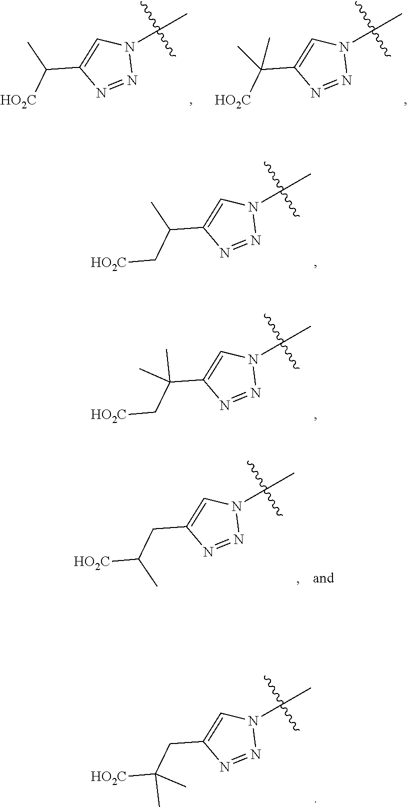 [SMILES: *n1cc(C(C)C(=O)O)nn1 ; *n1cc(C(C)(C)C(=O)O)nn1 ; *n1cc(C(C)CC(=O)O)nn1 ; *n1cc(C(C)(C)CC(=O)O)nn1 ; *n1cc(CC(C)C(=O)O)nn1 ; *n1cc(CC(C)(C)C(=O)O)nn1]

In some embodiments, R² is

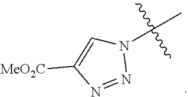 [SMILES: *n1cc(C(=O)OC)nn1]

In some embodiments, R² is

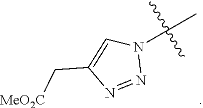 [SMILES: *n1cc(CC(=O)OC)nn1]

In some embodiments, R² is

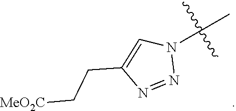 [SMILES: *n1cc(CCC(=O)OC)nn1]

In some embodiments, R² is

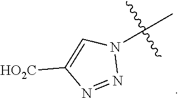 [SMILES: *n1cc(C(=O)O)nn1]

In some embodiments, R² is

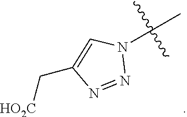 [SMILES: *n1cc(CC(=O)O)nn1]

In some embodiments, R² is

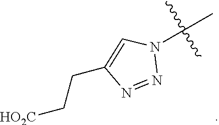 [SMILES: *n1cc(CCC(=O)O)nn1]

In some embodiments, R² is

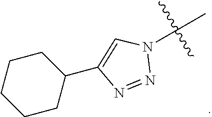 [SMILES: *n1cc(C2CCCCC2)nn1]

In some embodiments, R³ is chosen from -J²-OT⁵, -J²-C(═O)OT⁵, -J²-C(═O)NT⁵T⁶, -J²-S(═O)₂OT⁵, -J²-OS(═O)₂OT⁵, and -J²-P(═O)(OT⁵)(OT⁶) groups, wherein J² is chosen from a bond, divalent C₁-10 alkyl, and divalent C₁₋₁₀ haloalkyl groups, wherein T⁵ and 16, which may be identical or different, are independently chosen from H and C₁₋₈ alkyl groups, or T¹ and T⁶ join together along with the heteroatoms to which they are attached to form an optionally substituted, saturated or unsaturated, 3-10 membered ring.

In some embodiments, J² is a bond. In some embodiments, J² is chosen from divalent C₁₋₁₀ alkyl groups. In some embodiments, J² is chosen from divalent C₁₋₈ alkyl groups. In some embodiments, J² is chosen from divalent C₁₋₆ alkyl groups. In some embodiments, J² is chosen from divalent C₁₋₄ alkyl groups. In some embodiments, J² is chosen from divalent C₁₋₃ alkyl groups. In some embodiments, J² is chosen from divalent C₁₋₂ alkyl groups. In some embodiments, J² is a divalent C₁ alkyl group, i.e., divalent methylene.

In some embodiments, J² is chosen from —CH₂—, —(CH₂)₂—, —(CH₂)₃—, —(CH₂)ₙ-, —(CH₂)₅—, —(CH₂)₆—, —(CH₂)₇—, —(CH₂)₅—, —(CH₂)₉—, and —(CH₂)₁₀₋₈ In some embodiments, J² is —CH₂—. In some embodiments, J² is —(CH₂)₂₋₈ In some embodiments, J² is —(CH₂)₃₋₈ In some embodiments, J² is —(CH₂)₄₋₈ In some embodiments, J² is —(CH₂)₅—. In some embodiments, J² is —(CH₂)₆₋₈ In some embodiments, J² is —(CH₂)₇₋₈ In some embodiments, J² is —(CH₂)₅—. In some embodiments, J² is —(CH₂)₉₋₈ In some embodiments, J² is —(CH₂)₁₀₋₈

In some embodiments, J² is chosen from divalent C₁₋₁₀ haloalkyl groups. In some embodiments, J² is chosen from divalent C₁₋₈ haloalkyl groups. In some embodiments, J² is chosen from divalent C₁₋₆ haloalkyl groups. In some embodiments, J² is chosen from divalent C₁₋₄ haloalkyl groups. In some embodiments, J² is chosen from divalent C₁₋₃ haloalkyl groups. In some embodiments, J² is chosen from divalent C₁₋₂ haloalkyl groups. In some embodiments, J² is chosen from divalent C₁₋₁₀ haloalkyl groups.

In some embodiments, R³ is chosen from -J²-OT groups. In some embodiments, R³ is chosen from -J²-OT⁵ groups, wherein J² is a bond. In some embodiments, R³ is chosen from -J²-OH groups, wherein J² is a bond. In some embodiments, R³ is chosen from -J²-OT groups, wherein J² is chosen from divalent C₁₋₁₀ alkyl groups. In some embodiments, R³ is chosen from -J²-OH groups, wherein J² is chosen from divalent C₁₋₁₀ alkyl groups.

In some embodiments, R³ is chosen from -J²-C(═O)OT³ and -J²-C(═O)NT⁵T⁶ groups. In some embodiments, R³ is chosen from -J²-C(═O)OT⁵ and -J²-C(═O)NT⁵T groups, wherein J² is a bond. In some embodiments, R³ is chosen from -J²-C(═O)OH and -J²-C(═O)NH₂ groups, wherein J² is a bond. In some embodiments, R³ is chosen from -J²-C(═O)OT⁵ and -J²-C(═O)NT⁵T^{h} groups, wherein J² is chosen from divalent C₁₋₁₀ alkyl groups. In some embodiments, R³ is chosen from -J²-C(═O)OH and -J²-C(═O)NH₂ groups, wherein J² is chosen from divalent C₁₋₁₀ alkyl groups.

In some embodiments, R³ is chosen from -J²-S(═O)₂OT⁵, -J²-OS(═O)₂OT, and -J²-P(═O)(OT)(OT⁶) groups. In some embodiments, R³ is chosen from -J²-S(═O)₂OT⁵, -J²-OS(═O)₂OT, and -J²-P(═O)(OT⁵)(OT⁶) groups, wherein J² is a bond. In some embodiments, R³ is chosen from -J²-S(═O)₂OH, -J²-OS(═O)₂OH, and -J²-P(═O)(OH)₂ groups, wherein J² is a bond. In some embodiments, R³ is chosen from -J²-S(═O)₂OT⁵, -J²-OS(═O)₂OT³, and -J²-P(═O)(OT⁵)(OT⁶) groups, wherein J² is chosen from divalent C₁₋₁₀ alkyl groups. In some embodiments, R³ is chosen from -J²-S(═O)₂OH, -J²-OS(═O)₂OH, and -J²-P(═O)(OH)₂ groups, wherein J² is chosen from divalent C₁₋₁₀ alkyl groups.

In some embodiments, R³ is chosen from C₇₋₁₉ arylalkoxy, C₆₋₈ aryl, and C₁₋₁₃ heteroaryl groups, wherein the C₇₋₁₉ arylalkoxy, C₆₋₁₈ aryl, and C₁₋₃ heteroaryl groups are optionally substituted with one or more groups independently chosen from R¹, C₁₋₈ alkyl, and C₁₋₈ haloalkyl groups, wherein R¹ is independently chosen from C₆₋₁₈ aryl groups which are optionally substituted with one or more groups independently chosen from halo and C₁₋₈ aryl groups.

In some embodiments, R³ is chosen from C₁₋₁₃ heteroaryl groups optionally substituted with one or more groups independently chosen from R¹.

In some embodiments, R³ is chosen from C₂₋₆ heteroaryl groups optionally substituted with one or more groups independently chosen from R⁷.

In some embodiments, R³ is chosen from triazole groups substituted with one or more groups independently chosen from R⁷.

In some embodiments, R³ is chosen from triazole groups substituted with one or more phenyl which is optionally substituted with one or more groups independently chosen from halo and C₆₋₁₈ aryl groups.

In some embodiments, R³ is chosen from C₇₋₁₉ arylalkoxy groups optionally substituted with one or more groups independently chosen from R⁷, C₁₋₈ alkyl, and C₁₋₈ haloalkyl groups.

In some embodiments, R³ is —OBn optionally substituted with one or more groups independently chosen from R⁷, C₁₋₈ alkyl, and C₁₋₈ haloalkyl groups.

In some embodiments, R³ is chosen from —OH, —CO₂H, —SO₃H, —OSO₃H, and —PO₃H₂.

In some embodiments, R³ is —OH.

In some embodiments, R³ is —CO₂H.

In some embodiments, R³ is —SO₃H.

In some embodiments, R³ is —OSO₃H.

In some embodiments, R³ is —PO₃H₂.

In some embodiments, R³ is

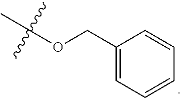 [SMILES: *OCc1ccccc1]

In some embodiments, R³ is chosen from

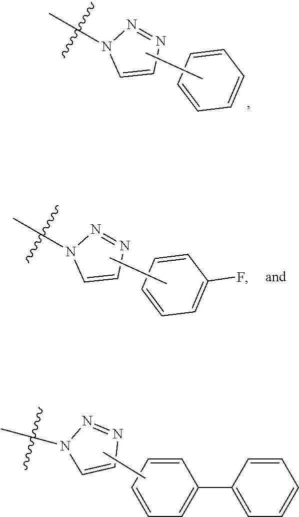

In some embodiments, R³ is chosen from

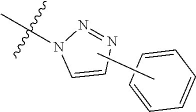

groups.

In some embodiments, R³ is chosen from

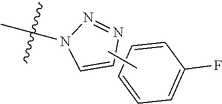

groups.

In some embodiments, R³ is chosen from

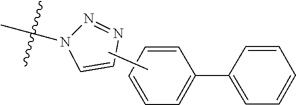

groups.

In some embodiments, R³ is chosen from

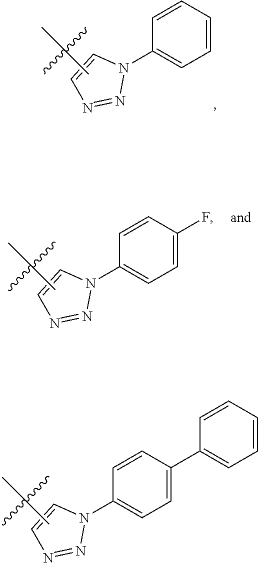

groups.

In some embodiments, R³ is chosen from

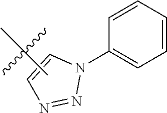

groups.

In some embodiments, R³ is chosen from

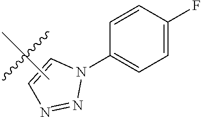

groups.

In some embodiments, R³ is chosen from

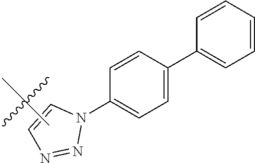

groups.

In some embodiments, R³ is chosen from

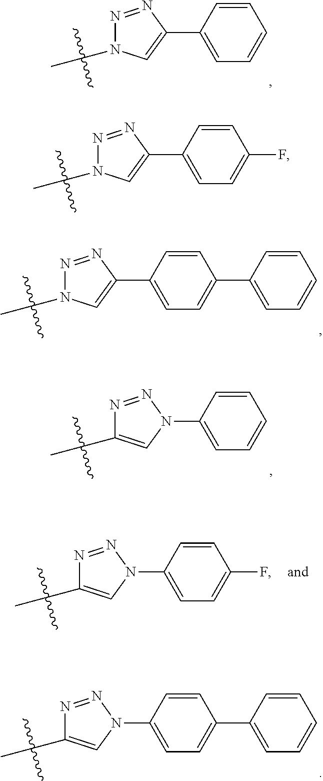 [SMILES: *n1cc(-c2ccccc2)nn1 ; *n1cc(-c2ccc(F)cc2)nn1 ; *n1cc(-c2ccc(-c3ccccc3)cc2)nn1 ; *c1cn(-c2ccccc2)nn1 ; *c1cn(-c2ccc(F)cc2)nn1 ; *c1cn(-c2ccc(-c3ccccc3)cc2)nn1]

In some embodiments, R³ is

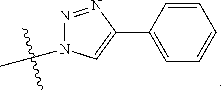 [SMILES: *n1cc(-c2ccccc2)nn1]

In some embodiments, R³ is

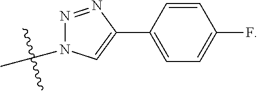 [SMILES: *n1cc(-c2ccc(F)cc2)nn1]

In some embodiments, R³ is

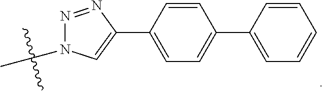 [SMILES: *n1cc(-c2ccc(-c3ccccc3)cc2)nn1]

In some embodiments, R³ is

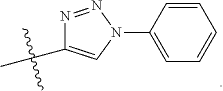 [SMILES: *c1cn(-c2ccccc2)nn1]

In some embodiments, R³ is

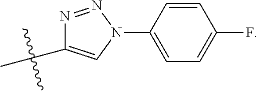 [SMILES: *c1cn(-c2ccc(F)cc2)nn1]

In some embodiments, R³ is

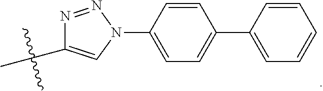 [SMILES: *c1cn(-c2ccc(-c3ccccc3)cc2)nn1]

In some embodiments, at least one of R⁴ and R⁵ is H. In some embodiments, R⁴ and R⁵ are H. In some embodiments, at least one of R⁴ and R⁵ is chosen from hydroxy protecting groups. In some embodiments, R⁴ and R⁵, which may be identical or different, are independently chosen from hydroxy protecting groups. In some embodiments, the hydroxy protecting groups are independently chosen from formyl, acetyl, chloroacetyl, o-nitrophenylacetyl, o-nitrophenoxy-acetyl, trifluoroacetyl, acetoacetyl, 4-chlorobutyryl, isobutyryl, o-nitrocinnamoyl, picolinoyl, acylisothiocyanate, aminocaproyl, benzoyl, benzyl, p-methoxyethoxymethyl, dimethoxytrityl, methoxymethyl, methoxytrityl [(4-methoxyphenyl)diphenylmethyl], p-methoxybenzyl, methylthiomethyl, pivaloyl, tetrahydropyranyl, tetrahydrofuranyl, trityl, and silyl groups.

In some embodiments, R⁴ and R⁵ join together along with the oxygen atoms to which they are attached to form an optionally substituted, saturated or unsaturated, 5-12 membered ring. In some embodiments, R⁴ and R⁵ join together along with the oxygen atoms to which they are attached to form a ketal. In some embodiments, R⁴ and R⁵ join together along with the oxygen atoms to which they are attached to form an acetal. In some embodiments, R⁴ and R⁵ join together along with the oxygen atoms to which they are attached to form an acetonide. In some embodiments, R⁴ and R⁵ join together along with the oxygen atoms to which they are attached to form a carbonate. In some embodiments, R⁴ and R⁵ join together along with the oxygen atoms to which they are attached to form a benzaldehyde acetal.

In some embodiments, R⁶ is chosen from —C(═O)OR⁸ groups. In some embodiments, R⁶ is chosen from —C(═O)NT¹T¹² groups. In some embodiments, R⁶ is chosen from —C(═O)OR⁸ groups wherein R⁸ is chosen from carboxy protecting groups. In some embodiments, the carboxy protecting groups are chosen from methyl, benzyl, tert-butyl, and silyl groups. In some embodiments, R⁶ is —C(═O)OH. In some embodiments, R¹ is —C(═O)OCH₃.

In some embodiments, R⁴ and R join together along with the oxygen atoms to which they are attached to form an acetonide and R⁶ is methyl.

In some embodiments, R⁴ and R⁵ are H and R⁶ is methyl. In some embodiments, R⁴, R, and R⁶ are H.

In some embodiments, L¹ is chosen from divalent C₁₋₂₄ alkyl groups. In some embodiments, L¹ is chosen from divalent C₁₋₂₀ alkyl groups. In some embodiments, L¹ is chosen from divalent C₁₋₁₈ alkyl groups. In some embodiments, L¹ is chosen from divalent C₁₋₆ alkyl groups. In some embodiments, L¹ is chosen from divalent C₁₋₁₄ alkyl groups. In some embodiments, L¹ is chosen from divalent C₁₋₂ alkyl groups. In some embodiments, L¹ is chosen from divalent C₁₋₁₈ alkyl groups. In some embodiments, L¹ is chosen from divalent C₁₋₈ alkyl groups. In some embodiments, L¹ is chosen from divalent C₁₋₆ alkyl groups. In some embodiments, L¹ is chosen from divalent C₁₋₄ alkyl groups. In some embodiments, L¹ is chosen from divalent C₁₋₂ alkyl groups.

In some embodiments, L¹ is chosen from —(CH₂)ₘ—, —(CH₂)ₘO(CH₂)ₙ-, and —(CH₂)ₘS(CH₂)ₙ— groups, wherein m and n, which may be the same or different, are independently chosen from integers ranging from 1 to 24.

In some embodiments, L¹ is chosen from —(CH₂)ₘ—, —(CH₂)ₘO(CH₂)˜—, and —(CH₂)ₘS(CH₂)ₙ- groups, wherein m and n, which may be the same or different, are independently chosen from integers ranging from 2 to 20.

In some embodiments, L¹ is chosen from —(CH₂)ₘ—, —(CH₂)ₘO(CH₂)ₙ—, and 4CH₂)ₙS(CH₂)ₙ— groups, wherein m and n, which may be the same or different, are independently chosen from integers ranging from 2 to 10.

In some embodiments, L¹ is chosen from —(CH₂)ₙ—, —(CH₂)ₘO(CH₂)ₙ—, and —(CH₂)ₘS(CH₂)ₙ— groups, wherein m and n, which may be the same or different, are independently chosen from integers ranging from 2 to 4.

In some embodiments, L¹ is chosen from —(CH₂)ₘ- groups, wherein m is chosen from integers ranging from 2 to 4.

In some embodiments, L¹ is —(CH₂)₂₋₈

In some embodiments, L¹ is —(CH₂)₃₋₈

In some embodiments, L¹ is —(CH₂)ₙ₋₈

In some embodiments, L¹ is chosen from

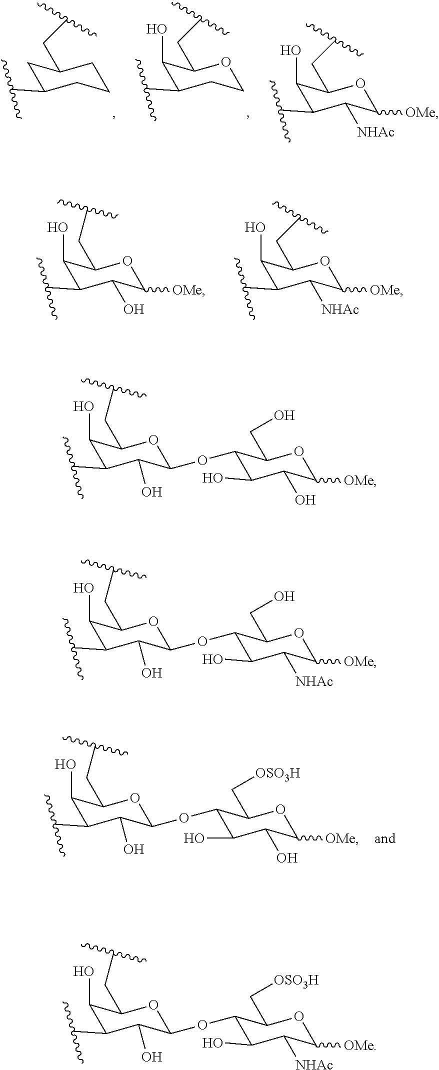

In some embodiments, L² is chosen from -Q(CH₂)ₘCH₂V—, -QCH₂(OCH₂CH₂)ₘOCH₂V—, -QCH₂CH₂(OCH₂CH₂)ₘOCH₂V—, -QCH₂(OCH₂CH₂)ₘOCH₂CH₂V—, and -QCH₂CH₂(OCH₂CH₂)ₘOCH₂CH₂V-groups, wherein Q is chosen from a bond, —CH₂C(═O)NH—, and —CH₂CH₂CH₂SCH₂CH₂NH(C═O)—, wherein V is chosen from a bond,

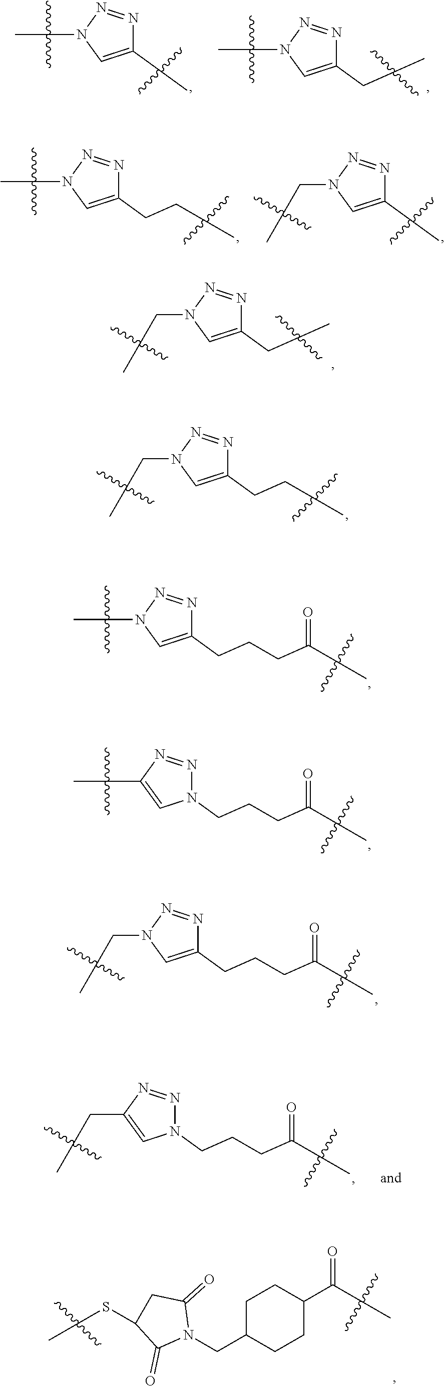

and wherein m is chosen from integers ranging from 0 to 46.

In some embodiments, L² is chosen from -Q(CH₂)ₘCH₂V— groups, wherein m is chosen from integers ranging from 0 to 46. In some embodiments, L² is chosen from -Q(CH₂)ₘCH₂V— groups, wherein m is chosen from integers ranging from 0 to 36. In some embodiments, L² is chosen from -Q(CH₂)ₘCH₂V— groups, wherein m is chosen from integers ranging from 0 to 24. In some embodiments, L² is chosen from -Q(CH₂)ₘCH₂V— groups, wherein m is chosen from integers ranging from 0 to 18. In some embodiments, L² is chosen from -Q(CH₂)ₘCH₂V— groups, wherein m is chosen from integers ranging from 0 to 12.

In some embodiments, L² is chosen from -Q(CH₂)ₘCH₂V— groups, wherein Q is a bond, and wherein m is chosen from integers ranging from 0 to 46. In some embodiments, L² is chosen from -Q(CH₂)ₘCH₂V— groups, wherein Q is a bond, and wherein m is chosen from integers ranging from 0 to 36. In some embodiments, L² is chosen from -Q(CH₂)ₘCH₂V-groups, wherein Q is a bond, and wherein m is chosen from integers ranging from 0 to 24. In some embodiments, L² is chosen from -Q(CH₂)ₘCH₂V— groups, wherein Q is a bond, and wherein m is chosen from integers ranging from 0 to 18. In some embodiments, L² is chosen from -Q(CH₂)ₘCH₂V— groups, wherein Q is a bond, and wherein m is chosen from integers ranging from 0 to 12.

In some embodiments, L² is chosen from -Q(CH₂)ₘCH₂V— groups, wherein V is a bond, and wherein m is chosen from integers ranging from 0 to 46. In some embodiments, L² is chosen from -Q(CH₂)ₘCH₂V— groups, wherein V is a bond, and wherein m is chosen from integers ranging from 0 to 36. In some embodiments, L² is chosen from -Q(CH₂)ₘCH₂V-groups, wherein V is a bond, and wherein m is chosen from integers ranging from 0 to 24. In some embodiments, L² is chosen from -Q(CH₂)ₘCH₂V— groups, wherein V is a bond, and wherein m is chosen from integers ranging from 0 to 18. In some embodiments, L² is chosen from -Q(CH₂)ₘCH₂V— groups, wherein V is a bond, and wherein m is chosen from integers ranging from 0 to 12.

In some embodiments, L² is chosen from -Q(CH₂)ₘCH₂V— groups, wherein Q is —CH₂C(═O)NH—, and wherein m is chosen from integers ranging from 0 to 46. In some embodiments, L² is chosen from -Q(CH₂)ₙCH₂V— groups, wherein Q is —CH₂C(═O)NH—, and wherein m is chosen from integers ranging from 0 to 36. In some embodiments, L² is chosen from -Q(CH₂)ₘCH₂V— groups, wherein Q is —CH₂C(═O)NH—, and wherein m is chosen from integers ranging from 0 to 24. In some embodiments, L² is chosen from -Q(CH₂)ₘCH₂V-groups, wherein Q is —CH₂C(═O)NH—, and wherein m is chosen from integers ranging from 0 to 18. In some embodiments, L² is chosen from -Q(CH₂)ₘCH₂V— groups, wherein Q is —CH₂C(═O)NH—, and wherein m is chosen from integers ranging from 0 to 12.

In some embodiments, L² is chosen from -Q(CH₂)ₘCH₂V— groups, wherein Q is —CH₂C(═O)NH—, wherein V is a bond, and wherein m is chosen from integers ranging from 0 to 46. In some embodiments, L² is chosen from -Q(CH₂)ₘCH₂V— groups, wherein Q is —CH₂C(═O)NH—, wherein V is a bond, and wherein m is chosen from integers ranging from 0 to 36. In some embodiments, L² is chosen from -Q(CH₂)ₘCH₂V— groups, wherein Q is —CH₂C(═O)NH—, wherein V is a bond, and wherein m is chosen from integers ranging from 0 to 24. In some embodiments, L² is chosen from -Q(CH₂)ₘCH₂V— groups, wherein Q is —CH₂C(═O)NH—, wherein V is a bond, and wherein m is chosen from integers ranging from 0 to 18. In some embodiments, L² is chosen from -Q(CH₂)ₙCH₂V— groups, wherein Q is —CH₂C(═O)NH—, wherein V is a bond, and wherein m is chosen from integers ranging from 0 to 12.

In some embodiments, L² is chosen from -Q(CH₂)ₘCH₂V— groups, wherein Q is —CH₂CH₂CH₂SCH₂CH₂NH(C=O)—, and wherein m is chosen from integers ranging from 0 to 46. In some embodiments, L² is chosen from -Q(CH₂)ₘCH₂V— groups, wherein Q is —CH₂CH₂CH₂SCH₂CH₂NH(C═O)—, and wherein m is chosen from integers ranging from 0 to 36. In some embodiments, L² is chosen from -Q(CH₂)ₘCH₂V— groups, wherein Q is —CH₂CH₂CH₂SCH₂CH₂NH(C═O)—, and wherein m is chosen from integers ranging from 0 to 24. In some embodiments, L² is chosen from -Q(CH₂)ₘCH₂V— groups, wherein Q is —CH₂CH₂CH₂SCH₂CH₂NH(C═O)—, and wherein m is chosen from integers ranging from 0 to 18. In some embodiments, L² is chosen from -Q(CH₂)·CH₂V— groups, wherein Q is —CH₂CH₂CH₂SCH₂CH₂NH(C═O)—, and wherein m is chosen from integers ranging from 0 to 12.

In some embodiments, L² is chosen from -Q(CH₂)ₘCH₂V— groups, wherein Q is —CH₂CH₂CH₂SCH₂CH₂NH(C═O)—, wherein V is a bond, and wherein m is chosen from integers ranging from 0 to 46. In some embodiments, L² is chosen from -Q(CH₂)ₘCH₂V— groups, wherein Q is —CH₂CH₂CH₂SCH₂CH₂NH(C═O)—, wherein V is a bond, and wherein m is chosen from integers ranging from 0 to 36. In some embodiments, L² is chosen from -Q(CH₂)ₘCH₂V-groups, wherein Q is —CH₂CH₂CH₂SCH₂CH₂NH(C═O)—, wherein V is a bond, and wherein m is chosen from integers ranging from 0 to 24. In some embodiments, L² is chosen from -Q(CH₂)ₘCH₂V— groups, wherein Q is —CH₂CH₂CH₂SCH₂CH₂NH(C═O)—, wherein V is a bond, and wherein m is chosen from integers ranging from 0 to 18. In some embodiments, L² is chosen from -Q(CH₂)ₘCH₂V— groups, wherein Q is —CH₂CH₂CH₂SCH₂CH₂NH(C═O)—, wherein V is a bond, and wherein m is chosen from integers ranging from 0 to 12.

In some embodiments, L² is chosen from -QCH₂(OCH₂CH₂)ₘOCH₂V— groups, wherein m is chosen from integers ranging from 0 to 46. In some embodiments, L² is chosen from -QCH₂(OCH₂CH₂)ₘOCH₂V— groups, wherein m is chosen from integers ranging from 0 to 36. In some embodiments, L² is chosen from -QCH₂(OCH₂CH₂)ₘOCH₂V— groups, wherein m is chosen from integers ranging from 0 to 24. In some embodiments, L² is chosen from -QCH₂(OCH₂CH₂)ₘOCH₂V— groups, wherein m is chosen from integers ranging from 0 to 18. In some embodiments, L² is chosen from -QCH₂(OCH₂CH₂)ₘOCH₂V— groups, wherein m is chosen from integers ranging from 0 to 12.

In some embodiments, L is chosen from -QCH₂(OCH₂CH₂)ₘOCH₂V— groups, wherein Q is a bond, and wherein m is chosen from integers ranging from 0 to 46. In some embodiments, L² is chosen from -QCH₂(OCH₂CH₂)ₘOCH₂V— groups, wherein Q is a bond, and wherein m is chosen from integers ranging from 0 to 36. In some embodiments, L² is chosen from QCH₂(OCH₂CH₂)ₘOCH₂V— groups, wherein Q is a bond, and wherein m is chosen from integers ranging from 0 to 24. In some embodiments, L² is chosen from -QCH₂(OCH₂CH₂)ₘOCH₂V— groups, wherein Q is a bond, and wherein m is chosen from integers ranging from 0 to 18. In some embodiments, L² is chosen from -QCH₂(OCH₂CH₂)ₘOCH₂V— groups, wherein Q is a bond, and wherein m is chosen from integers ranging from 0 to 12.

In some embodiments, L² is chosen from -QCH₂(OCH₂CH₂)ₘOCH₂V— groups, wherein V is a bond, and wherein m is chosen from integers ranging from 0 to 46. In some embodiments, L² is chosen from -QCH₂(OCH₂CH₂)ₘOCH₂V— groups, wherein V is a bond, and wherein m is chosen from integers ranging from 0 to 36. In some embodiments, L² is chosen from -QCH₂(OCH₂CH₂)ₘOCH₂V— groups, wherein V is a bond, and wherein m is chosen from integers ranging from 0 to 24. In some embodiments, L² is chosen from -QCH₂(OCH₂CH₂)ₘOCH₂V— groups, wherein V is a bond, and wherein m is chosen from integers ranging from 0 to 18. In some embodiments, L² is chosen from QCH₂(OCH₂CH₂)ₘOCH₂V— groups, wherein V is a bond, and wherein m is chosen from integers ranging from 0 to 12.

In some embodiments, L² is chosen from -QCH₂(OCH₂CH₂)ₘOCH₂V— groups, wherein Q is —CH₂C(═O)NH—, and wherein m is chosen from integers ranging from 0 to 46. In some embodiments, L² is chosen from -QCH₂(OCH₂CH₂)ₘOCH₂V— groups, wherein Q is —CH₂C(═O)NH—, and wherein m is chosen from integers ranging from 0 to 36. In some embodiments, L² is chosen from -QCH₂(OCH₂CH₂)ₘOCH₂V— groups, wherein Q is —CH₂C(═O)NH—, and wherein m is chosen from integers ranging from 0 to 24. In some embodiments, L² is chosen from -QCH₂(OCH₂CH₂)ₘOCH₂V— groups, wherein Q is —CH₂C(═O)NH—, and wherein m is chosen from integers ranging from 0 to 18. In some embodiments, L² is chosen from -QCH₂(OCH₂CH₂)ₘOCH₂V— groups, wherein Q is —CH₂C(═O)NH—, and wherein m is chosen from integers ranging from 0 to 12.

In some embodiments, L² is chosen from -QCH₂(OCH₂CH₂)ₘOCH₂V— groups, wherein Q is —CH₂C(═O)NH—, V is a bond, and wherein m is chosen from integers ranging from 0 to 46. In some embodiments, L² is chosen from -QCH₂(OCH₂CH₂)ₘOCH₂V— groups, wherein Q is —CH₂C(═O)NH—, V is a bond, and wherein m is chosen from integers ranging from 0 to 36. In some embodiments, L¹ is chosen from -QCH₂(OCH₂CH₂)ₘOCH₂V— groups, wherein Q is —CH₂C(═O)NH—, V is a bond, and wherein m is chosen from integers ranging from 0 to 24. In some embodiments, L² is chosen from -QCH₂(OCH₂CH₂)ₘOCH₂V— groups, wherein Q is —CH₂C(═O)NH—, V is a bond, and wherein m is chosen from integers ranging from 0 to 18. In some embodiments, L² is chosen from -QCH₂(OCH₂CH₂)ₘOCH₂V— groups, wherein Q is —CH₂C(═O)NH—, V is a bond, and wherein m is chosen from integers ranging from 0 to 12.

In some embodiments, L² is chosen from -QCH₂(OCH₂CH₂)ₘOCH₂V— groups, wherein Q is —CH₂CH₂CH₂SCH₂CH₂NH(C═O)—, and wherein m is chosen from integers ranging from 0 to 46. In some embodiments, L² is chosen from -QCH₂(OCH₂CH₂)ₘOCH₂V-groups, wherein Q is —CH₂CH₂CH₂SCH₂CH₂NH(C═O)—, and wherein m is chosen from integers ranging from 0 to 36. In some embodiments, L² is chosen from -QCH₂(OCH₂CH₂)ₘOCH₂V— groups, wherein Q is —CH₂CH₂CH₂SCH₂CH₂NH(C═O)—, and wherein m is chosen from integers ranging from 0 to 24. In some embodiments, L² is chosen from -QCH₂(OCH₂CH₂)ₘOCH₂V— groups, wherein Q is —CH₂CH₂CH₂SCH₂CH₂NH(C═O)—, and wherein m is chosen from integers ranging from 0 to 18. In some embodiments, L² is chosen from -QCH₂(OCH₂CH₂)ₘOCH₂V— groups, wherein Q is —CH₂CH₂CH₂SCH₂CH₂NH(C═O)—, and wherein m is chosen from integers ranging from 0 to 12.

In some embodiments, L² is chosen from -QCH₂(OCH₂CH₂)ₘOCH₂V— groups, wherein Q is —CH₂CH₂CH₂SCH₂CH₂NH(C═O)—, wherein V is a bond, and wherein m is chosen from integers ranging from 0 to 46. In some embodiments, L² is chosen from -QCH₂(OCH₂CH₂)ₘOCH₂V— groups, wherein Q is —CH₂CH₂CH₂SCH₂CH₂NH(C═O)—, wherein V is a bond, and wherein m is chosen from integers ranging from 0 to 36. In some embodiments, L² is chosen from -QCH₂(OCH₂CH₂)ₘOCH₂V— groups, wherein Q is —CH₂CH₂CH₂SCH₂CH₂NH(C═O)—, wherein V is a bond, and wherein m is chosen from integers ranging from 0 to 24. In some embodiments, L² is chosen from -QCH₂(OCH₂CH₂)ₘOCH₂V-groups, wherein Q is —CH₂CH₂CH₂SCH₂CH₂NH(C═O)—, wherein V is a bond, and wherein m is chosen from integers ranging from 0 to 18. In some embodiments, L² is chosen from -QCH₂(OCH₂CH₂)ₘOCH₂V— groups, wherein Q is —CH₂CH₂CH₂SCH₂CH₂NH(C═O)—, wherein V is a bond, and wherein m is chosen from integers ranging from 0 to 12.

In some embodiments, L² is chosen from -QCH₂CH₂(OCH₂CH₂)ₘOCH₂V— groups, wherein m is chosen from integers ranging from 0 to 46. In some embodiments, L² is chosen from -QCH₂CH₂(OCH₂CH₂)ₘOCH₂V— groups, wherein m is chosen from integers ranging from 0 to 36. In some embodiments, L² is chosen from -QCH₂CH₂(OCH₂CH₂)OCH₂V— groups, wherein m is chosen from integers ranging from 0 to 24. In some embodiments, L² is chosen from -QCH₂CH₂(OCH₂CH₂)ₘOCH₂V— groups, wherein m is chosen from integers ranging from 0 to 18. In some embodiments, L² is chosen from -QCH₂CH₂(OCH₂CH₂)ₘOCH₂V— groups, wherein m is chosen from integers ranging from 0 to 12.

In some embodiments, L² is chosen from -QCH₂CH₂(OCH₂CH₂)ₘOCH₂V— groups, wherein Q is a bond, and wherein m is chosen from integers ranging from 0 to 46. In some embodiments, L² is chosen from -QCH₂CH₂(OCH₂CH₂)ₘOCH₂V— groups, wherein Q is a bond, and wherein m is chosen from integers ranging from 0 to 36. In some embodiments, L² is chosen from -QCH₂CH₂(OCH₂CH₂)ₘOCH₂V— groups, wherein Q is a bond, and wherein m is chosen from integers ranging from 0 to 24. In some embodiments, L² is chosen from -QCH₂CH₂(OCH₂CH₂)ₘOCH₂V— groups, wherein Q is a bond, and wherein m is chosen from integers ranging from 0 to 18. In some embodiments, L² is chosen from -QCH₂CH₂(OCH₂CH₂)ₘOCH₂V— groups, wherein Q is a bond, and wherein m is chosen from integers ranging from 0 to 12.

In some embodiments, L² is chosen from -QCH₂CH₂(OCH₂CH₂)ₘOCH₂V— groups, wherein V is a bond, and wherein m is chosen from integers ranging from 0 to 46. In some embodiments, L² is chosen from -QCH₂CH₂(OCH₂CH₂)ₘOCH₂V— groups, wherein V is a bond, and wherein m is chosen from integers ranging from 0 to 36. In some embodiments, L² is chosen from -QCH₂CH₂(OCH₂CH₂)ₘOCH₂V— groups, wherein V is a bond, and wherein m is chosen from integers ranging from 0 to 24. In some embodiments, L² is chosen from -QCH₂CH₂(OCH₂CH₂)·OCH₂V— groups, wherein V is a bond, and wherein m is chosen from integers ranging from 0 to 18. In some embodiments, L² is chosen from -QCH₂CH₂(OCH₂CH₂)ₘOCH₂V— groups, wherein V is a bond, and wherein m is chosen from integers ranging from 0 to 12.

In some embodiments, L² is chosen from -QCH₂CH₂(OCH₂CH₂)ₘOCH₂V— groups, wherein Q is —CH₂C(═O)NH—, and wherein m is chosen from integers ranging from 0 to 46. In some embodiments, L² is chosen from -QCH₂CH₂(OCH₂CH₂)ₘOCH₂V— groups, wherein Q is —CH₂C(═O)NH—, and wherein m is chosen from integers ranging from 0 to 36. In some embodiments, L² is chosen from -QCH₂CH₂(OCH₂CH₂)ₘOCH₂V— groups, wherein Q is —CH₂C(═O)NH—, and wherein m is chosen from integers ranging from 0 to 24. In some embodiments, L² is chosen from -QCH₂CH₂(OCH₂CH₂)ₘOCH₂V— groups, wherein Q is —CH₂C(═O)NH—, and wherein m is chosen from integers ranging from 0 to 18. In some embodiments, L² is chosen from -QCH₂CH₂(OCH₂CH₂)ₘOCH₂V— groups, wherein Q is —CH₂C(═O)NH—, and wherein m is chosen from integers ranging from 0 to 12.

In some embodiments, L² is chosen from -QCH₂CH₂(OCH₂CH₂)ₘOCH₂V— groups, wherein Q is —CH₂C(═O)NH—, V is a bond, and wherein m is chosen from integers ranging from 0 to 46. In some embodiments, L² is chosen from -QCH₂CH₂(OCH₂CH₂)ₘOCH₂V— groups, wherein Q is —CH₂C(═O)NH—, V is a bond, and wherein m is chosen from integers ranging from 0 to 36. In some embodiments, L² is chosen from -QCH₂CH₂(OCH₂CH₂)OCH₂V— groups, wherein Q is —CH₂C(═O)NH—, V is a bond, and wherein m is chosen from integers ranging from 0 to 24. In some embodiments, L² is chosen from -QCH₂CH₂(OCH₂CH₂)ₙOCH₂V— groups, wherein Q is —CH₂C(═O)NH—, V is a bond, and wherein m is chosen from integers ranging from 0 to 18. In some embodiments, L² is chosen from -QCH₂CH₂(OCH₂CH₂)ₘOCH₂V— groups, wherein Q is —CH₂C(═O)NH—, V is a bond, and wherein m is chosen from integers ranging from 0 to 12.

In some embodiments, L² is chosen from -QCH₂CH₂(OCH₂CH₂)ₘOCH₂V— groups, wherein Q is —CH₂CH₂CH₂SCH₂CH₂NH(C═O)—, and wherein m is chosen from integers ranging from 0 to 46. In some embodiments, L is chosen from -QCH₂CH₂(OCH₂CH₂)ₘOCH₂V— groups, wherein Q is —CH₂CH₂CH₂SCH₂CH₂NH(C═O)—, and wherein m is chosen from integers ranging from 0 to 36. In some embodiments, L² is chosen from -QCH₂CH₂(OCH₂CH₂)ₘOCH₂V— groups, wherein Q is —CH₂CH₂CH₂SCH₂CH₂NH(C═O)—, and wherein m is chosen from integers ranging from 0 to 24. In some embodiments, L² is chosen from -QCH₂CH₂(OCH₂CH₂)·OCH₂V— groups, wherein Q is —CH₂CH₂CH₂SCH₂CH₂NH(C═O)—, and wherein m is chosen from integers ranging from 0 to 18. In some embodiments, L² is chosen from -Q CH₂CH₂(OCH₂CH₂)ₘOCH₂V— groups, wherein Q is —CH₂CH₂CH₂SCH₂CH₂NH(C═O)—, and wherein m is chosen from integers ranging from 0 to 12.

In some embodiments, L² is chosen from -QCH₂CH₂(OCH₂CH₂)ₘOCH₂V— groups, wherein Q is —CH₂CH₂CH₂SCH₂CH₂NH(C═O)—, wherein V is a bond, and wherein m is chosen from integers ranging from 0 to 46. In some embodiments, L² is chosen from -QCH₂CH₂(OCH₂CH₂)ₘOCH₂V— groups, wherein Q is —CH₂CH₂CH₂SCH₂CH₂NH(C═O)—, wherein V is a bond, and wherein m is chosen from integers ranging from 0 to 36. In some embodiments, L² is chosen from -QCH₂CH₂(OCH₂CH₂)ₘOCH₂V— groups, wherein Q is —CH₂CH₂CH₂SCH₂CH₂NH(C═O)—, wherein V is a bond, and wherein m is chosen from integers ranging from 0 to 24. In some embodiments, L² is chosen from -QCH₂CH₂(OCH₂CH₂)ₘOCH₂V— groups, wherein Q is —CH₂CH₂CH₂SCH₂CH₂NH(C═O)—, wherein V is a bond, and wherein m is chosen from integers ranging from 0 to 18. In some embodiments, L² is chosen from -QCH₂CH₂(OCH₂CH₂)ₘOCH₂V— groups, wherein Q is —CH₂CH₂CH₂SCH₂CH₂NH(C═O)—, wherein V is a bond, and wherein m is chosen from integers ranging from 0 to 12.

In some embodiments, L² is chosen from -QCH₂(OCH₂CH₂)ₘOCH₂CH₂V— groups, wherein m is chosen from integers ranging from 0 to 46. In some embodiments, L² is chosen from -QCH₂(OCH₂CH₂)ₘOCH₂CH₂V— groups, wherein m is chosen from integers ranging from 0 to 36. In some embodiments, L² is chosen from -QCH₂(OCH₂CH₂)ₘOCH₂CH₂V— groups, wherein m is chosen from integers ranging from 0 to 24. In some embodiments, L² is chosen from -QCH₂(OCH₂CH₂)ₘOCH₂CH₂V— groups, wherein m is chosen from integers ranging from 0 to 18. In some embodiments, L² is chosen from -QCH₂(OCH₂CH₂)ₘOCH₂CH₂V— groups, wherein m is chosen from integers ranging from 0 to 12.

In some embodiments, L² is chosen from -QCH₂(OCH₂CH₂)ₘOCH₂CH₂V— groups, wherein Q is a bond, and wherein m is chosen from integers ranging from 0 to 46. In some embodiments, L² is chosen from -QCH₂(OCH₂CH₂)ₘOCH₂CH₂V— groups, wherein Q is a bond, and wherein m is chosen from integers ranging from 0 to 36. In some embodiments, L² is chosen from -QCH₂(OCH₂CH₂)ₘOCH₂CH₂V— groups, wherein Q is a bond, and wherein m is chosen from integers ranging from 0 to 24. In some embodiments, L² is chosen from -QCH₂(OCH₂CH₂)ₘOCH₂CH₂V— groups, wherein Q is a bond, and wherein m is chosen from integers ranging from 0 to 18. In some embodiments, L² is chosen from QCH₂(OCH₂CH₂)ₘOCH₂CH₂V— groups, wherein Q is a bond, and wherein m is chosen from integers ranging from 0 to 12.

In some embodiments, L² is chosen from -QCH₂(OCH₂CH₂)ₘOCH₂CH₂V— groups, wherein V is a bond, and wherein m is chosen from integers ranging from 0 to 46. In some embodiments, L² is chosen from -QCH₂(OCH₂CH₂)ₘOCH₂CH₂V— groups, wherein V is a bond, and wherein m is chosen from integers ranging from 0 to 36. In some embodiments, L² is chosen from -QCH₂(OCH₂CH₂)ₘOCH₂CH₂V— groups, wherein V is a bond, and wherein m is chosen from integers ranging from 0 to 24. In some embodiments, L² is chosen from -QCH₂(OCH₂CH₂)ₘOCH₂CH₂V— groups, wherein V is a bond, and wherein m is chosen from integers ranging from 0 to 18. In some embodiments, L² is chosen from -QCH₂(OCH₂CH₂)ₘOCH₂CH₂V— groups, wherein V is a bond, and wherein m is chosen from integers ranging from 0 to 12.

In some embodiments, L² is chosen from -QCH₂(OCH₂CH₂)ₘOCH₂CH₂V— groups, wherein Q is —CH₂C(═O)NH—, and wherein m is chosen from integers ranging from 0 to 46. In some embodiments, L² is chosen from -QCH₂(OCH₂CH₂)ₘOCH₂CH₂V— groups, wherein Q is —CH₂C(═O)NH—, and wherein m is chosen from integers ranging from 0 to 36. In some embodiments, L¹ is chosen from -QCH₂(OCH₂CH₂)ₘOCH₂CH₂V— groups, wherein Q is —CH₂C(═O)NH—, and wherein m is chosen from integers ranging from 0 to 24. In some embodiments, L² is chosen from -QCH₂(OCH₂CH₂)ₘOCH₂CH₂V— groups, wherein Q is —CH₂C(═O)NH—, and wherein m is chosen from integers ranging from 0 to 18. In some embodiments, L² is chosen from -QCH₂(OCH₂CH₂)ₘOCH₂CH₂V— groups, wherein Q is —CH₂C(═O)NH—, and wherein m is chosen from integers ranging from 0 to 12.

In some embodiments, L² is chosen from -QCH₂(OCH₂CH₂)ₘOCH₂CH₂V— groups, wherein Q is —CH₂C(═O)NH—, wherein V is a bond, and wherein m is chosen from integers ranging from 0 to 46. In some embodiments, L² is chosen from QCH₂(OCH₂CH₂)ₘOCH₂CH₂V— groups, wherein Q is —CH₂C(═O)NH—, wherein V is a bond, and wherein m is chosen from integers ranging from 0 to 36. In some embodiments, L² is chosen from -QCH₂(OCH₂CH₂)ₘOCH₂CH₂V— groups, wherein Q is —CH₂C(═O)NH—, wherein V is a bond, and wherein m is chosen from integers ranging from 0 to 24. In some embodiments, L² is chosen from -QCH₂(OCH₂CH₂)ₘOCH₂CH₂V— groups, wherein Q is —CH₂C(═O)NH—, wherein V is a bond, and wherein m is chosen from integers ranging from 0 to 18. In some embodiments, L² is chosen from -QCH₂(OCH₂CH₂)ₘOCH₂CH₂V— groups, wherein Q is —CH₂C(═O)NH—, wherein V is a bond, and wherein m is chosen from integers ranging from 0 to 12.

In some embodiments, L is chosen from -QCH₂(OCH₂CH₂)ₘOCH₂CH₂V— groups, wherein Q is —CH₂CH₂CH₂SCH₂CH₂NH(C═O)—, and wherein m is chosen from integers ranging from 0 to 46. In some embodiments, L² is chosen from -QCH₂(OCH₂CH₂)ₘOCH₂CH₂V— groups, wherein Q is —CH₂CH₂CH₂SCH₂CH₂NH(C═O)—, and wherein m is chosen from integers ranging from 0 to 36. In some embodiments, L² is chosen from -QCH₂(OCH₂CH₂)ₘOCH₂CH₂V— groups, wherein Q is —CH₂CH₂CH₂SCH₂CH₂NH(C═O)—, and wherein m is chosen from integers ranging from 0 to 24. In some embodiments, L² is chosen from -QCH₂(OCH₂CH₂)ₘOCH₂CH₂V— groups, wherein Q is —CH₂CH₂CH₂SCH₂CH₂NH(C=O)—, and wherein m is chosen from integers ranging from 0 to 18. In some embodiments, L² is chosen from -QCH₂(OCH₂CH₂)ₘOCH₂CH₂V— groups, wherein Q is —CH₂CH₂CH₂SCH₂CH₂NH(C═O)—, and wherein m is chosen from integers ranging from 0 to 12.

In some embodiments, L² is chosen from -QCH₂(OCH₂CH₂)ₘOCH₂CH₂V— groups, wherein Q is —CH₂CH₂CH₂SCH₂CH₂NH(C═O)—, wherein V is a bond, and wherein m is chosen from integers ranging from 0 to 46. In some embodiments, L² is chosen from QCH₂(OCH₂CH₂)ₘOCH₂CH₂V— groups, wherein Q is —CH₂CH₂CH₂SCH₂CH₂NH(C═O)—, wherein V is a bond, and wherein m is chosen from integers ranging from 0 to 36. In some embodiments, L¹ is chosen from -QCH₂(OCH₂CH₂)ₘOCH₂CH₂V— groups, wherein Q is —CH₂CH₂CH₂SCH₂CH₂NH(C═O)—, wherein V is a bond, and wherein m is chosen from integers ranging from 0 to 24. In some embodiments, L² is chosen from -QCH₂(OCH₂CH₂)ₘOCH₂CH₂V— groups, wherein Q is —CH₂CH₂CH₂SCH₂CH₂NH(C═O)—, wherein V is a bond, and wherein m is chosen from integers ranging from 0 to 18. In some embodiments, L² is chosen from -QCH₂(OCH₂CH₂)ₘOCH₂CH₂V— groups, wherein Q is —CH₂CH₂CH₂SCH₂CH₂NH(C═O)—, wherein V is a bond, and wherein m is chosen from integers ranging from 0 to 12.

In some embodiments, L² is chosen from -QCH₂CH₂(OCH₂CH₂)ₘOCH₂CH₂V-groups, wherein m is chosen from integers ranging from 0 to 46. In some embodiments, L² is chosen from -QCH₂CH₂(OCH₂CH₂)ₘOCH₂CH₂V— groups, wherein m is chosen from integers ranging from 0 to 36. In some embodiments, L² is chosen from -QCH₂CH₂(OCH₂CH₂)ₘOCH₂CH₂V— groups, wherein m is chosen from integers ranging from 0 to 24. In some embodiments, L² is chosen from -QCH₂CH₂(OCH₂CH₂)ₘOCH₂CH₂V— groups, wherein m is chosen from integers ranging from 0 to 18. In some embodiments, L² is chosen from -QCH₂CH₂(OCH₂CH₂)ₘOCH₂CH₂V— groups, wherein m is chosen from integers ranging from 0 to 12.

In some embodiments, L² is chosen from -QCH₂CH₂(OCH₂CH₂)ₘOCH₂CH₂V-groups, wherein Q is a bond, and wherein m is chosen from integers ranging from 0 to 46. In some embodiments, L² is chosen from -QCH₂CH₂(OCH₂CH₂)ₘOCH₂CH₂V— groups, wherein Q is a bond, and wherein m is chosen from integers ranging from 0 to 36. In some embodiments, L² is chosen from -QCH₂CH₂(OCH₂CH₂)ₘOCH₂CH₂V— groups, wherein Q is a bond, and wherein m is chosen from integers ranging from 0 to 24. In some embodiments, L² is chosen from -QCH₂CH₂(OCH₂CH₂)ₘOCH₂CH₂V— groups, wherein Q is a bond, and wherein m is chosen from integers ranging from 0 to 18. In some embodiments, L² is chosen from -QCH₂CH₂(OCH₂CH₂)ₘOCH₂CH₂V— groups, wherein Q is a bond, and wherein m is chosen from integers ranging from 0 to 12.

In some embodiments, L is chosen from -QCH₂CH₂(OCH₂CH₂)ₘOCH₂CH₂V-groups, wherein V is a bond, and wherein m is chosen from integers ranging from 0 to 46. In some embodiments, L² is chosen from -QCH₂CH₂(OCH₂CH₂)ₘOCH₂CH₂V— groups, wherein V is a bond, and wherein m is chosen from integers ranging from 0 to 36. In some embodiments, L² is chosen from -QCH₂CH₂(OCH₂CH₂)ₘOCH₂CH₂V— groups, wherein V is a bond, and wherein m is chosen from integers ranging from 0 to 24. In some embodiments, L² is chosen from -QCH₂CH₂(OCH₂CH₂)ₘOCH₂CH₂V— groups, wherein V is a bond, and wherein m is chosen from integers ranging from 0 to 18. In some embodiments, L² is chosen from QCH₂CH₂(OCH₂CH₂)ₘOCH₂CH₂V— groups, wherein V is a bond, and wherein m is chosen from integers ranging from 0 to 12.

In some embodiments, L² is chosen from -QCH₂CH₂(OCH₂CH₂)MOCH₂CH₂V-groups, wherein Q is —CH₂C(═O)NH—, and wherein m is chosen from integers ranging from 0 to 46. In some embodiments, L² is chosen from -QCH₂CH₂(OCH₂CH₂)ₙOCH2CH₂V— groups, wherein Q is —CH₂C(═O)NH—, and wherein m is chosen from integers ranging from 0 to 36. In some embodiments, L² is chosen from -QCH₂CH₂(OCH₂CH₂)ₘOCH₂CH₂V— groups, wherein Q is —CH₂C(═O)NH—, and wherein m is chosen from integers ranging from 0 to 24. In some embodiments, L² is chosen from -QCH₂CH₂(OCH₂CH₂)ₘOCH₂CH₂V— groups, wherein Q is —CH₂C(═O)NH—, and wherein m is chosen from integers ranging from 0 to 18. In some embodiments, L² is chosen from -QCH₂CH₂(OCH₂CH₂)ₘOCH₂CH₂V— groups, wherein Q is —CH₂C(═O)NH—, and wherein m is chosen from integers ranging from 0 to 12.

In some embodiments, L² is chosen from -QCH₂CH₂(OCH₂CH₂)ₘOCH₂CH₂V-groups, wherein Q is —CH₂C(═O)NH—, wherein V is a bond, and wherein m is chosen from integers ranging from 0 to 46. In some embodiments, L² is chosen from -QCH₂CH₂(OCH₂CH₂)ₘOCH₂CH₂V— groups, wherein Q is —CH₂C(═O)NH—, wherein V is a bond, and wherein m is chosen from integers ranging from 0 to 36. In some embodiments, L² is chosen from -QCH₂CH₂(OCH₂CH₂)ₘOCH₂CH₂V— groups, wherein Q is-CH₂C(═O)NH—, wherein V is a bond, and wherein m is chosen from integers ranging from 0 to 24. In some embodiments, L² is chosen from -QCH₂CH₂(OCH₂CH₂)ₘOCH₂CH₂V— groups, wherein Q is —CH₂C(═O)NH—, wherein V is a bond, and wherein m is chosen from integers ranging from 0 to 18. In some embodiments, L² is chosen from -QCH₂CH₂(OCH₂CH₂)MOCH₂CH₂V— groups, wherein Q is —CH₂C(═O)NH—, wherein V is a bond, and wherein m is chosen from integers ranging from 0 to 12.

In some embodiments, L² is chosen from -QCH₂CH₂(OCH₂CH₂)ₘOCH₂CH₂V-groups, wherein Q is —CH₂CH₂CH₂SCH₂CH₂NH(C═O)—, and wherein m is chosen from integers ranging from 0 to 46. In some embodiments, L² is chosen from -QCH₂CH₂(OCH₂CH₂)ₘOCH₂CH₂V— groups, wherein Q is —CH₂CH₂CH₂SCH₂CH₂NH(C═O)—, and wherein m is chosen from integers ranging from 0 to 36. In some embodiments, L² is chosen from -QCH₂CH₂(OCH₂CH₂)ₘOCH₂CH₂V— groups, wherein Q is —CH₂CH₂CH₂SCH₂CH₂NH(C═O)—, and wherein m is chosen from integers ranging from 0 to 24. In some embodiments, L² is chosen from -QCH₂CH₂(OCH₂CH₂)·OCH₂CH₂V— groups, wherein Q is —CH₂CH₂CH₂SCH₂CH₂NH(C═O)—, and wherein m is chosen from integers ranging from 0 to 18. In some embodiments, L² is chosen from -QCH₂CH₂(OCH₂CH₂)ₘOCH₂CH₂V— groups, wherein Q is —CH₂CH₂CH₂SCH₂CH₂NH(C=O)—, and wherein m is chosen from integers ranging from 0 to 12.

In some embodiments, L² is chosen from -QCH₂CH₂(OCH₂CH₂)ₘOCH₂CH₂V-groups, wherein Q is —CH₂CH₂CH₂SCH₂CH₂NH(C═O)—, wherein V is a bond, and wherein m is chosen from integers ranging from 0 to 46. In some embodiments, L² is chosen from QCH₂CH₂(OCH₂CH₂)ₘOCH₂CH₂V— groups, wherein Q is —CH₂CH₂CH₂SCH₂CH₂NH(C=O)—, wherein V is a bond, and wherein m is chosen from integers ranging from 0 to 36. In some embodiments, L² is chosen from -QCH₂CH₂(OCH₂CH₂)ₘOCH₂CH₂V— groups, wherein Q is —CH₂CH₂CH₂SCH₂CH₂NH(C═O)—, wherein V is a bond, and wherein m is chosen from integers ranging from 0 to 24. In some embodiments, L² is chosen from QCH₂CH₂(OCH₂CH₂)ₘOCH₂CH₂V— groups, wherein Q is —CH₂CH₂CH₂SCH₂CH₂NH(C═O)—, wherein V is a bond, and wherein m is chosen from integers ranging from 0 to 18. In some embodiments, L² is chosen from -QCH₂CH₂(OCH₂CH₂)ₘOCH₂CH₂V— groups, wherein Q is —CH₂CH₂CH₂SCH₂CH₂NH(C═O)—, wherein V is a bond, and wherein m is chosen from integers ranging from 0 to 12.

In some embodiments, L² is chosen from -Q(CH₂)₂₄CH₂V— groups. In some embodiments, L² is chosen from -Q(CH₂)₂OCH₂V— groups. In some embodiments, L² is chosen from -Q(CH₂)₁₆CH₂V— groups. In some embodiments, L² is chosen from -Q(CH₂)₁₂CH₂V-groups. In some embodiments, L² is chosen from -Q(CH₂)₈CH₂V— groups. In some embodiments, L² is chosen from -Q(CH₂)₂CH₂V— groups. In some embodiments, L² is chosen from -Q(CH₂)₆CH₂V— groups. In some embodiments, L² is chosen from -Q(CH₂)₅CH₂V-groups. In some embodiments, L² is chosen from -Q(CH₂)₄CH₂V— groups. In some embodiments, L¹ is chosen from -Q(CH₂)₃CH₂V— groups. In some embodiments, L² is chosen from -Q(CH₂)₂CH₂V— groups. In some embodiments, L² is chosen from -QCH₂CH₂V— groups. In some embodiments, L² is chosen from -QCH₂V— groups.

In some embodiments, L² is chosen from -Q(CH₂)₂₄CH₂V— groups, wherein V is a bond. In some embodiments, L² is chosen from -Q(CH₂)₂OCH₂V— groups, wherein V is a bond. In some embodiments, L² is chosen from -Q(CH₂)₁₆CH₂V— groups, wherein V is a bond. In some embodiments, L² is chosen from -Q(CH₂)₁₂CH₂V— groups, wherein V is a bond. In some embodiments, L² is chosen from -Q(CH₂)₈CH₂V— groups, wherein V is a bond. In some embodiments, L² is chosen from -Q(CH₂)₇CH₂V— groups, wherein V is a bond. In some embodiments, L² is chosen from -Q(CH₂)₆CH₂V— groups, wherein V is a bond. In some embodiments, L² is chosen from -Q(CH₂)₈CH₂V— groups, wherein V is a bond. In some embodiments, L¹ is chosen from -Q(CH₂)₄CH₂V— groups, wherein V is a bond. In some embodiments, L² is chosen from -Q(CH₂)₃CH₂V— groups, wherein V is a bond. In some embodiments, L² is chosen from -Q(CH₂)₂CH₂V— groups, wherein V is a bond. In some embodiments, L² is chosen from -QCH₂CH₂V— groups, wherein V is a bond. In some embodiments, L² is chosen from -QCH₂V— groups, wherein V is a bond.

In some embodiments, L² is chosen from -QCH₂(OCH₂CH₂)₂₄OCH₂V— groups. In some embodiments, L² is chosen from -QCH₂(OCH₂CH₂)₂OOCH₂V— groups. In some embodiments, L² is chosen from -QCH₂(OCH₂CH₂)₁₆OCH₂V— groups. In some embodiments, L² is chosen from -QCH₂(OCH₂CH₂)₁₂OCH₂V— groups. In some embodiments, L² is chosen from -QCH₂(OCH₂CH₂)₅OCH₂V— groups. In some embodiments, L is chosen from -QCH₂(OCH₂CH₂)₇OCH₂V— groups. In some embodiments, L² is chosen from -QCH₂(OCH₂CH₂)₆OCH₂V— groups. In some embodiments, L² is chosen from -QCH₂(OCH₂CH₂)₅OCH₂V— groups. In some embodiments, L² is chosen from -QCH₂(OCH₂CH₂)₄OCH₂V— groups. In some embodiments, L² is chosen from -QCH₂(OCH₂CH₂)₃OCH₂V— groups. In some embodiments, L² is chosen from -QCH₂(OCH₂CH₂)₂OCH₂V— groups. In some embodiments, L² is chosen from -QCH₂OCH₂CH₂OCH₂V— groups. In some embodiments, L² is chosen from -QCH₂OCH₂V-groups.

In some embodiments, L² is chosen from -QCH₂(OCH₂CH₂)₂₄OCH₂V— groups, wherein V is a bond. In some embodiments, L² is chosen from -QCH₂(OCH₂CH₂)₂OOCH₂V-groups, wherein V is a bond. In some embodiments, L² is chosen from -QCH₂(OCH₂CH₂)₁₆OCH₂V— groups, wherein V is a bond. In some embodiments, L² is chosen from -QCH₂(OCH₂CH₂)₁₂OCH₂V— groups, wherein V is a bond. In some embodiments, L² is chosen from -QCH₂(OCH₂CH₂)₅OCH₂V— groups, wherein V is a bond. In some embodiments, L² is chosen from -QCH₂(OCH₂CH₂)₇OCH₂V— groups, wherein V is a bond. In some embodiments, L² is chosen from -QCH₂(OCH₂CH₂)₆OCH₂V— groups, wherein V is a bond. In some embodiments, L² is chosen from -QCH₂(OCH₂CH₂)₅OCH₂V— groups, wherein V is a bond. In some embodiments, L² is chosen from -QCH₂(OCH₂CH₂)₄OCH₂V— groups, wherein V is a bond. In some embodiments, L² is chosen from -QCH₂(OCH₂CH₂)₃₀CH₂V— groups, wherein V is a bond. In some embodiments, L² is chosen from -QCH₂(OCH₂CH₂)₂OCH₂V-groups, wherein V is a bond. In some embodiments, L² is chosen from -QCH₂OCH₂CH₂OCH₂V— groups, wherein V is a bond. In some embodiments, L² is chosen from -QCH₂OCH₂V— groups, wherein V is a bond.

In some embodiments, L² is chosen from -QCH₂CH₂(OCH₂CH₂)₂₄OCH₂V— groups. In some embodiments, L² is chosen from -QCH₂CH₂(OCH₂CH₂)₂OOCH₂V— groups. In some embodiments, L¹ is chosen from -QCH₂CH₂(OCH₂CH₂)₁₆OCH₂V— groups. In some embodiments, L² is chosen from -QCH₂(OCH₂CH₂)₁₂OCH₂V— groups. In some embodiments, L² is chosen from -QCH₂CH₂(OCH₂CH₂)₅OCH₂V— groups. In some embodiments, L² is chosen from -QCH₂CH₂(OCH₂CH₂)₇OCH₂V— groups. In some embodiments, L² is chosen from -QCH₂CH₂(OCH₂CH₂)₆OCH₂V— groups. In some embodiments, L² is chosen from -QCH₂CH₂(OCH₂CH₂)₅OCH₂V— groups. In some embodiments, L² is chosen from -QCH₂CH₂(OCH₂CH₂)₄OCH₂V— groups. In some embodiments, L² is chosen from -QCH₂CH₂(OCH₂CH₂)₃OCH₂V— groups. In some embodiments, L² is chosen from -QCH₂CH₂(OCH₂CH₂)₂OCH₂V— groups. In some embodiments, L² is chosen from -QCH₂CH₂OCH₂CH₂OCH₂V— groups. In some embodiments, L² is chosen from -QCH₂CH₂OCH₂V— groups.

In some embodiments, L² is chosen from -QCH₂CH₂(OCH₂CH₂)₂₄OCH₂V— groups, wherein V is a bond. In some embodiments, L² is chosen from -QCH₂CH₂(OCH₂CH₂)₂OOCH₂V— groups, wherein V is a bond. In some embodiments, L² is chosen from -QCH₂CH₂(OCH₂CH₂)₁₆OCH₂V— groups, wherein V is a bond. In some embodiments, L² is chosen from -QCH₂(OCH₂CH₂)₁₂OCH₂V— groups, wherein V is a bond. In some embodiments, L² is chosen from -QCH₂CH₂(OCH₂CH₂)₅OCH₂V— groups, wherein V is a bond. In some embodiments, L² is chosen from -QCH₂CH₂(OCH₂CH₂)₂OCH₂V— groups, wherein V is a bond. In some embodiments, L² is chosen from -QCH₂CH₂(OCH₂CH₂)₆OCH₂V— groups, wherein V is a bond. In some embodiments, L¹ is chosen from -QCH₂CH₂(OCH₂CH₂)₅OCH₂V— groups, wherein V is a bond. In some embodiments, L² is chosen from -QCH₂CH₂(OCH₂CH₂)₄OCH₂V— groups, wherein V is a bond. In some embodiments, L² is chosen from -QCH₂CH₂(OCH₂CH₂)₂OCH₂V— groups, wherein V is a bond. In some embodiments, L² is chosen from -QCH₂CH₂(OCH₂CH₂)₂OCH₂V— groups, wherein V is a bond. In some embodiments, L² is chosen from -QCH₂CH₂OCH₂CH₂OCH₂V-groups, wherein V is a bond. In some embodiments, L² is chosen from -QCH₂CH₂OCH₂V-groups, wherein V is a bond.

In some embodiments, L² is chosen from -QCH₂(OCH₂CH₂)₂₄OCH₂CH₂V— groups. In some embodiments, L¹ is chosen from -QCH₂(OCH₂CH₂)₂OOCH₂CH₂V— groups. In some embodiments, L² is chosen from -QCH₂(OCH₂CH₂)₁₆OCH₂CH₂V— groups. In some embodiments, L² is chosen from -QCH₂(OCH₂CH₂)₁₂OCH₂CH₂V— groups. In some embodiments, L¹ is chosen from -QCH₂(OCH₂CH₂)₅OCH₂CH₂V— groups. In some embodiments, L² is chosen from -QCH₂(OCH₂CH₂)₇OCH₂CH₂V— groups. In some embodiments, L² is chosen from -QCH₂(OCH₂CH₂)₆OCH₂CH₂V— groups. In some embodiments, L² is chosen from -QCH₂(OCH₂CH₂)₅OCH₂CH₂V— groups. In some embodiments, L² is chosen from -QCH₂(OCH₂CH₂)₄OCH₂CH₂V— groups. In some embodiments, L¹ is chosen from -QCH₂(OCH₂CH₂)₃OCH₂CH₂V— groups. In some embodiments, L² is chosen from -QCH₂(OCH₂CH₂)₂OCH₂CH₂V— groups. In some embodiments, L² is chosen from -QCH₂OCH₂CH₂OCH₂CH₂V— groups. In some embodiments, L² is chosen from -QCH₂OCH₂CH₂V— groups.

In some embodiments, L² is chosen from -QCH₂(OCH₂CH₂)₂₄OCH₂CH₂V— groups, wherein V is a bond. In some embodiments, L² is chosen from -QCH₂(OCH₂CH₂)₂OOCH₂CH₂V— groups, wherein V is a bond. In some embodiments, L² is chosen from -QCH₂(OCH₂CH₂)₁₆OCH₂CH₂V— groups, wherein V is a bond. In some embodiments, L² is chosen from -QCH₂(OCH₂CH₂)₁₂OCH₂CH₂V— groups, wherein V is a bond. In some embodiments, L² is chosen from -QCH₂(OCH₂CH₂)₅OCH₂CH₂V— groups, wherein V is a bond. In some embodiments, L² is chosen from -QCH₂(OCH₂CH₂)₇OCH₂CH₂V— groups, wherein V is a bond. In some embodiments, L² is chosen from -QCH₂(OCH₂CH₂)₆OCH₂CH₂V— groups, wherein V is a bond. In some embodiments, L² is chosen from -QCH₂(OCH₂CH₂)₅OCH₂CH₂V— groups, wherein V is a bond. In some embodiments, L² is chosen from -QCH₂(OCH₂CH₂)₄OCH₂CH₂V— groups, wherein V is a bond. In some embodiments, L² is chosen from -QCH₂(OCH₂CH₂)₃OCH₂CH₂V— groups, wherein V is a bond. In some embodiments, L² is chosen from -QCH₂(OCH₂CH₂)₂OCH₂CH₂V— groups, wherein V is a bond. In some embodiments, L² is chosen from -QCH₂OCH₂CH₂OCH₂CH₂V-groups, wherein V is a bond. In some embodiments, L² is chosen from -QCH₂OCH₂CH₂V-groups, wherein V is a bond.

In some embodiments, L² is chosen from -QCH₂CH₂(OCH₂CH₂)₂₄OCH₂CH₂V-groups. In some embodiments, L² is chosen from -QCH₂CH₂(OCH₂CH₂)₂OOCH₂CH₂V-groups. In some embodiments, L² is chosen from -QCH₂CH₂(OCH₂CH₂)₁₆OCH₂CH₂V-groups. In some embodiments, L² is chosen from -QCH₂CH₂(OCH₂CH₂)₁₂OCH₂CH₂V-groups. In some embodiments, L² is chosen from -QCH₂CH₂(OCH₂CH₂)xOCH₂CH₂V— groups. In some embodiments, L² is chosen from -QCH₂CH₂(OCH₂CH₂)₇OCH₂CH₂V— groups. In some embodiments, L¹ is chosen from -QCH₂CH₂(OCH₂CH₂)₆OCH₂CH₂V— groups. In some embodiments, L² is chosen from -QCH₂CH₂(OCH₂CH₂)₅OCH₂CH₂V— groups. In some embodiments, L² is chosen from -QCH₂CH₂(OCH₂CH₂)₄₀CH₂CH₂V— groups. In some embodiments, L² is chosen from -QCH₂CH₂(OCH₂CH₂)₃OCH₂CH₂V— groups. In some embodiments, L² is chosen from -QCH₂CH₂(OCH₂CH₂)₂OCH₂CH₂V— groups. In some embodiments, L¹ is chosen from -QCH₂CH₂OCH₂CH₂OCH₂CH₂V— groups. In some embodiments, L² is chosen from -QCH₂CH₂OCH₂CH₂V— groups.

In some embodiments, L² is chosen from -QCH₂CH₂(OCH₂CH₂)₂₄OCH₂CH₂V-groups, wherein V is a bond. In some embodiments, L² is chosen from -QCH₂CH₂(OCH₂CH₂)₂OOCH₂CH₂V— groups, wherein V is a bond. In some embodiments, L² is chosen from -QCH₂CH₂(OCH₂CH₂)₁₆OCH₂CH₂V— groups, wherein V is a bond. In some embodiments, L² is chosen from -QCH₂CH₂(OCH₂CH₂)₁₂OCH₂CH₂V— groups, wherein V is a bond. In some embodiments, L² is chosen from -QCH₂CH₂(OCH₂CH₂)₅OCH₂CH₂V— groups, wherein V is a bond. In some embodiments, L² is chosen from -QCH₂CH₂(OCH₂CH₂)₂OCH₂CH₂V— groups, wherein V is a bond. In some embodiments, L² is chosen from -QCH₂CH₂(OCH₂CH₂)₆OCH₂CH₂V— groups, wherein V is a bond. In some embodiments, L² is chosen from -QCH₂CH₂(OCH₂CH₂)₅OCH₂CH₂V— groups, wherein V is a bond. In some embodiments, L² is chosen from -QCH₂CH₂(OCH₂CH₂)₄OCH₂CH₂V— groups, wherein V is a bond. In some embodiments, L² is chosen from -QCH₂CH₂(OCH₂CH₂)₂OCH₂CH₂V— groups, wherein V is a bond. In some embodiments, L² is chosen from -QCH₂CH₂(OCH₂CH₂)₂OCH₂CH₂V— groups, wherein V is a bond. In some embodiments, L² is chosen from -QCH₂CH₂OCH₂CH₂OCH₂CH₂V— groups, wherein V is a bond. In some embodiments, L² is chosen from -QCH₂CH₂OCH₂CH₂V— groups, wherein V is a bond.

In some embodiments, Q is a bond. In some embodiments, Q is —CH₂C(═O)NH—. In some embodiments, Q is —CH₂CH₂CH₂SCH₂CH₂NH(C═O)—.

In some embodiments, V is a bond. In some embodiments, V is chosen from

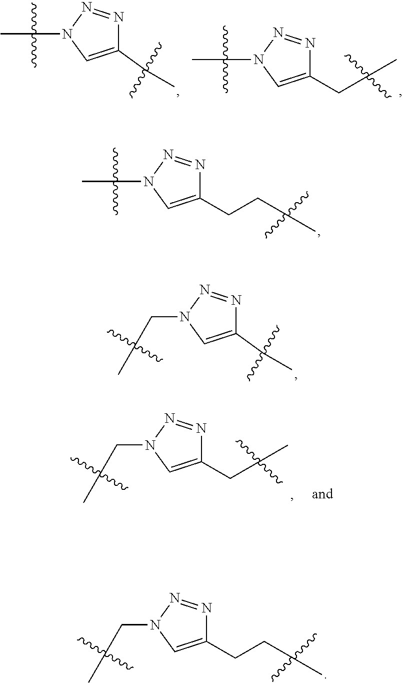 [SMILES: *c1cn(*)nn1 ; *Cc1cn(*)nn1 ; *CCc1cn(*)nn1 ; *Cn1cc(*)nn1 ; *Cc1cn(C*)nn1 ; *CCc1cn(C*)nn1]

In some embodiments, V is chosen from

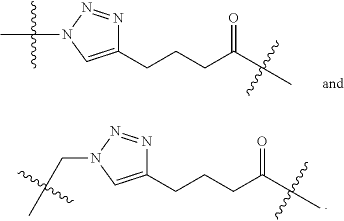 [SMILES: *C(=O)CCCc1cn(*)nn1 ; *Cn1cc(CCCC(*)=O)nn1]

In some embodiments, V is chosen from

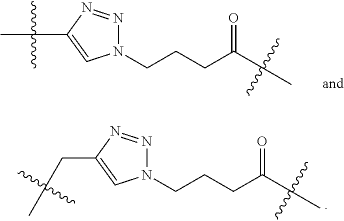 [SMILES: *C(=O)CCCn1cc(*)nn1 ; *Cc1cn(CCCC(*)=O)nn1]

In some embodiments, V is

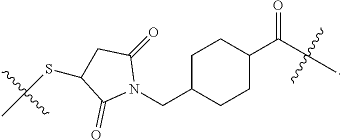 [SMILES: *SC1CC(=O)N(CC2CCC(C(*)=O)CC2)C1=O]

In some embodiments, X is —O—.

In some embodiments, X is —S—.

In some embodiments, X is —CH₂₋₈

In some embodiments, Y is H.

In some embodiments, Y is chosen from halo groups.

In some embodiments, Y is fluoro.

In some embodiments, Y is —OH.

In some embodiments, Y is —OMe.

In some embodiments, Z is chosen from lipids. In some embodiments, Z is chosen from glycerol-based lipids. In some embodiments, Z is chosen from phospholipids. In some embodiments, Z is chosen from symmetric phospholipids. In some embodiments, Z is chosen from asymmetric phospholipids. In some embodiments, Z is chosen from sphingolipids. In some embodiments, Z is chosen from ceramides.

In some embodiments, Z is chosen from phospholipids that have 16 to 30 carbon atoms. In some embodiments, Z is chosen from phospholipids that have 16, 20, 22, 24, 26, 28, or 30 carbon atoms. In some embodiments, Z is chosen from phospholipids that have 16 carbon atoms. In some embodiments, Z is chosen from phospholipids that have 18 carbon atoms. In some embodiments, Z is chosen from phospholipids that have 20 carbon atoms. In some embodiments, Z is chosen from phospholipids that have 22 carbon atoms. In some embodiments, Z is chosen from phospholipids that have 24 carbon atoms. In some embodiments, Z is chosen from phospholipids that have 26 carbon atoms. In some embodiments, Z is chosen from phospholipids that have 28 carbon atoms. In some embodiments, Z is chosen from phospholipids that have 30 carbon atoms.

In some embodiments, Z is chosen from saturated phospholipids. In some embodiments, Z is chosen from unsaturated phospholipids. In some embodiments, Z is chosen from phosphatidylcholine, phosphatidyethanolamine, phosphatidic acid, and phosphatidyl inositol. In some embodiments, Z is chosen from phospholipid derivatives of 6-cis-octadecenoic, 9-cis-octadecenoic, 9-trans-octadecenoic, 9-cis-12-octadecadienoic, 9-cis-12-cis-15-cis-octadecatrienoic, 11-cis-eicosenoic, 5, 8, 11, 14 (all-cis) eicosatetraenoic, 13-cis-docosenoic, 4, 7, 10, 13, 16, 19 (all-cis) docosahexaenoic, and/or 15-cis-tetracosenoic acids. In some embodiments, Z is chosen from phospholipid derivatives of stearoyl, oleoyl, linoleoyl, arachidonoyl, and/or docosahexaenoyl acids.

In some embodiments, Z is chosen from nucleophiles. In some embodiments, the nucleophiles are chosen from amines, mercaptans, and alcohols. In some embodiments, Z is chosen from amine groups.

In some embodiments, Z is chosen from electrophiles. In some embodiments, the electrophiles are chosen from epoxides, aziridines, episulfides, sulfates, sulfonates, carbonates, lactones, lactams, halides, acid halides, and esters. In some embodiments, the electrophiles are chosen from epoxides. In some embodiments, the electrophiles are chosen from aziridines. In some embodiments, the electrophiles are chosen from episulfides. In some embodiments, the electrophiles are chosen from sulfates. In some embodiments, the electrophiles are chosen from sulfonates. In some embodiments, the electrophiles are chosen from carbonates. In some embodiments, the electrophiles are chosen from lactones. In some embodiments, the electrophiles are chosen from lactams. In some embodiments, the electrophiles are chosen from halides. In some embodiments, the electrophiles are chosen from acid halides. In some embodiments, the electrophiles are chosen from esters. In some embodiments, the esters are chosen from activated esters.

In some embodiments, Z is chosen from esters. In some embodiments, the esters are chosen from anhydrides, carboxylic acid-succinimides, carboxylic acid phosphoesters, and carboxylic acid imidazolides. In some embodiments, Z is chosen from esters formed with t-butanol, p-nitrophenol, 2,4-dinitrophenol, trichlorophenol, 1-hydroxy-1H-benzotriazole, 1-hydroxy-6-chloro-1H-benzotriazole, and N-hydroxysuccinimide.

In some embodiments, Z is chosen from —C(═O)T¹⁴ groups, wherein T¹⁶ is chosen from H, —OH, —O^{t}Bu, C₂₋₈ alkenyl, C₁₋₈ haloalkyl, C₁₋₈ alkoxy, halo, C₆₋₁₈ aryloxy, C₁₋₃ heteroaryloxy, and C₂₋₁₂ heterocyclyloxy groups and wherein the C₁₋₈ alkoxy, C₆₋₈ aryloxy, C₁₋₃ heteroaryloxy, and C₂₋₁₂ heterocyclyloxy groups are optionally substituted with at least one halo group.

In some embodiments, Z is chosen from groups capable of undergoing a cycloaddition reaction. In some embodiments, the groups capable of undergoing a cycloaddition reaction are chosen from alkenes, alkynes, dienes, dienophiles, 1,3-dipoles, and 1,3-dipolarophiles.

In some embodiments, the dienophiles are chosen from —C(═O)T⁷ groups, wherein T¹⁴ is chosen from alkene and alkyne groups, wherein said alkene and alkyne groups are optionally substituted with C₁₋₈ alkoxy, —C(═O)H, —C(═O)OT¹⁸, and —C(═O)NT¹⁸T¹⁹ groups, wherein T¹⁸ and T¹⁹, which may be identical or different, are independently chosen from H and C₁₋₈ alkyl groups, or T¹⁸ and T¹⁹ join together along with the nitrogen to which they are attached to form an optionally substituted, saturated or unsaturated, 3-10 membered ring.

In some embodiments, the 1,3-dipoles are chosen from nitrile ylides, nitrile imines, nitrile oxides, diazolalkanes, azides, azomethine ylides, azomethine imines, nitrones, carbonyl ylides, carbonyl imines, and carbonyl oxides.

In some embodiments, Z is chosen from alkene and alkyne groups. In some embodiments, Z is chosen from alkene groups. In some embodiments, Z is chosen from alkyne groups. In some embodiments, Z is —CH═CH₂. In some embodiments, Z is —C—CH.

In some embodiments, Z is chosen from dienes and dienophiles. In some embodiments, Z is chosen from dienes. In some embodiments, Z is chosen from dienophiles.

In some embodiments, Z is chosen from 1,3-dipoles and 1,3-dipolarophiles. In some embodiments, Z is chosen from 1,3-dipoles. In some embodiments, Z is chosen from 1,3-dipolarophiles.

In some embodiments, Z is chosen from —N₃, —NH₂, —SH, —OH, —Cl, —Br, —I, —CH═CH₂, —C≡CH,

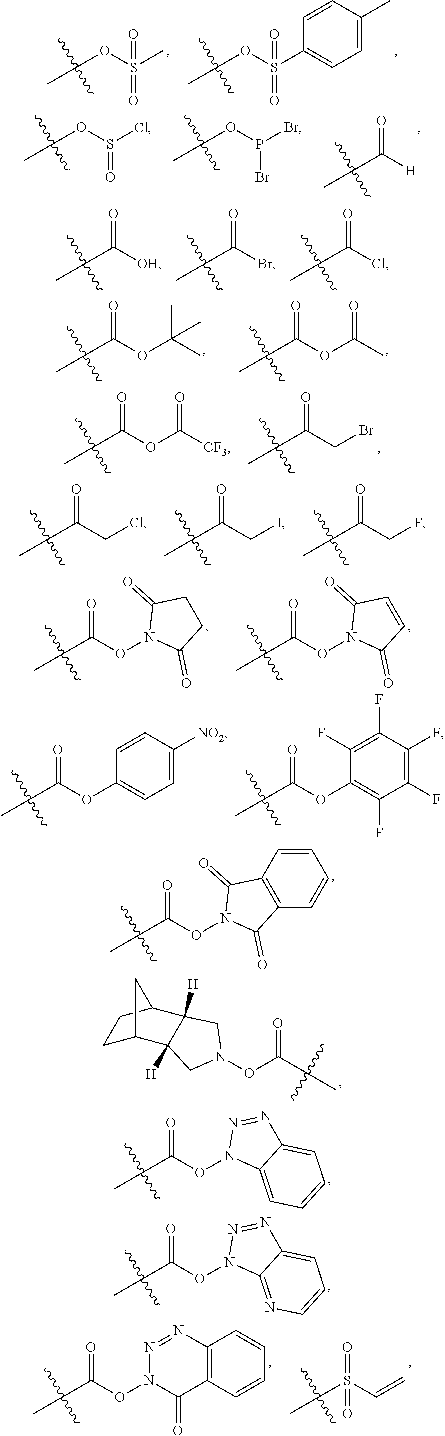

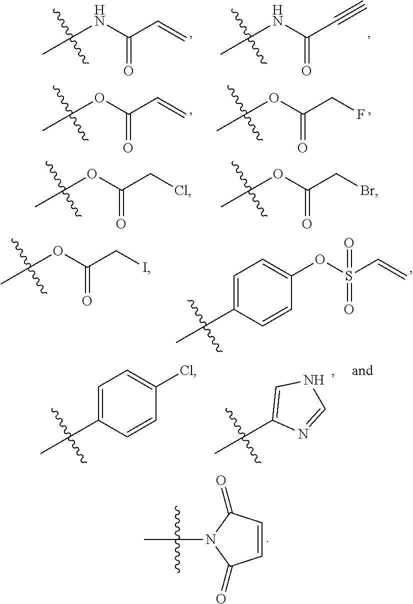

In some embodiments, Z is —N₃.

In some embodiments, Z is chosen from —NH₂, —SH, and —OH. In some embodiments, Z is —NH₂. In some embodiments, Z is —SH. In some embodiments, Z is —OH.

In some embodiments, Z is —C(═O)H. In some embodiments, Z is —C(═O)OH. In some embodiments, Z is —C(═O)Cl. In some embodiments, Z is —C(═O)O^{t}Bu.

In some embodiments, the compound of Formula (I) is chosen from:

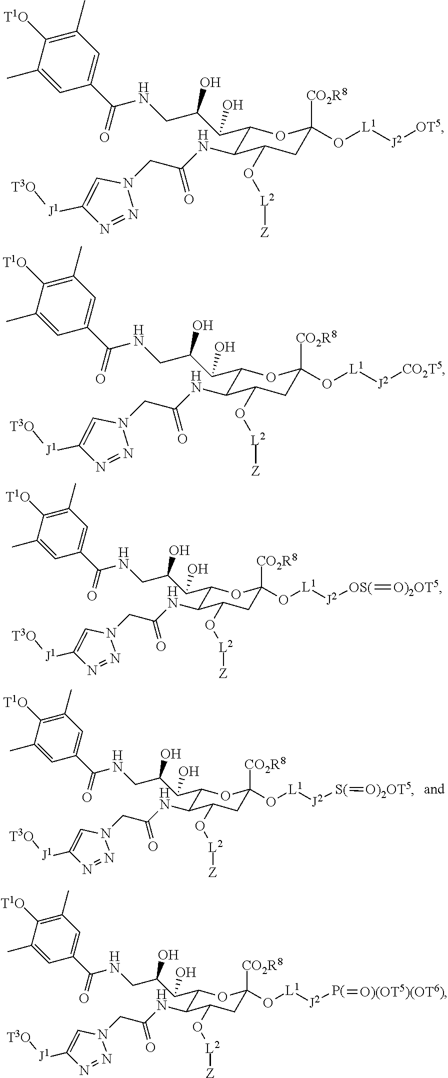

and pharmaceutically acceptable salts of any of the foregoing.

In some embodiments, the compound of Formula (I) is chosen from:

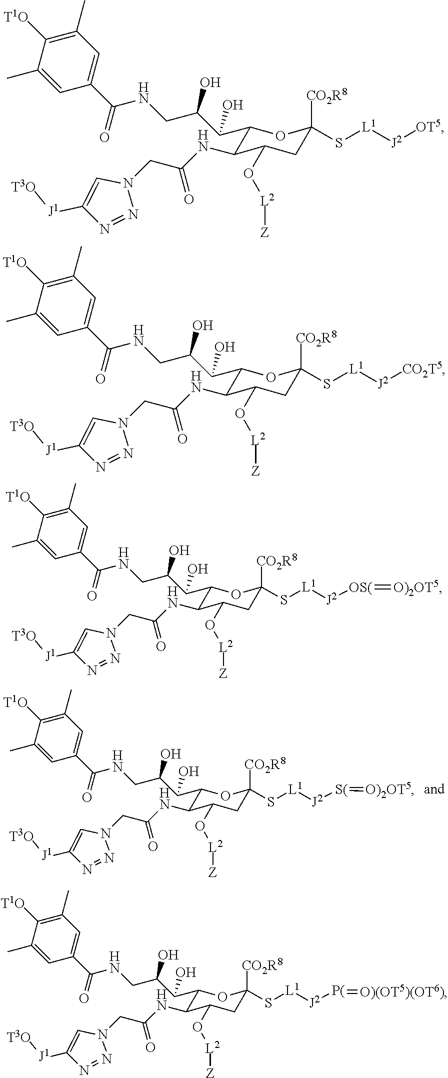

and pharmaceutically acceptable salts of any of the foregoing.

In some embodiments, the compound of Formula (I) is chosen from:

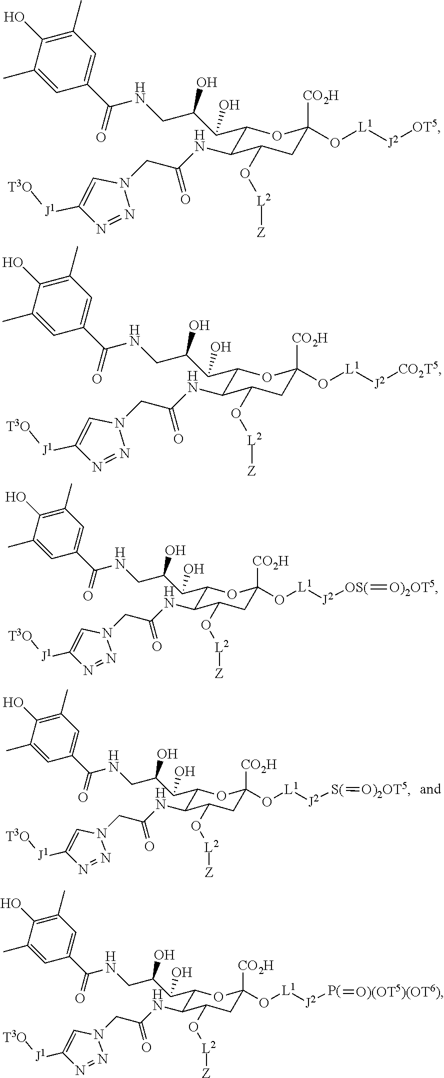

and pharmaceutically acceptable salts of any of the foregoing.

In some embodiments, the compound of Formula (I) is chosen from:

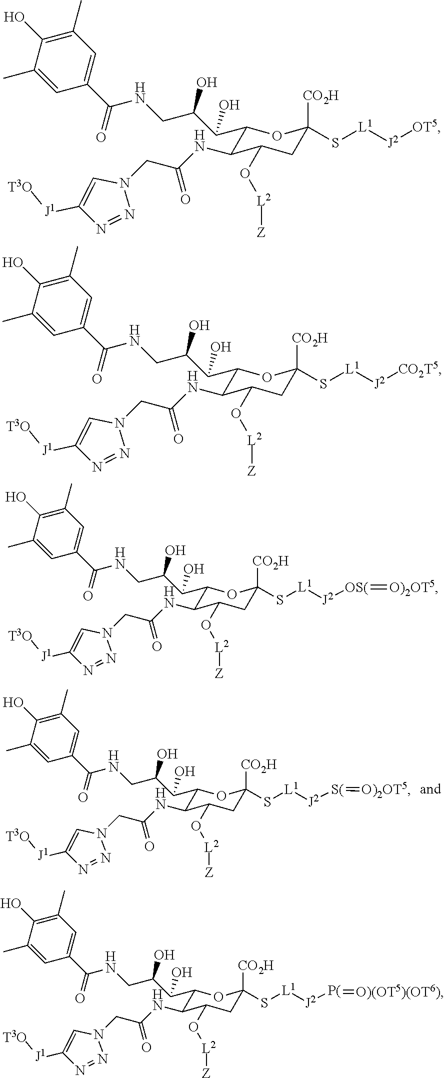

and pharmaceutically acceptable salts of any of the foregoing.

In some embodiments, the compound of Formula (I) is chosen from:

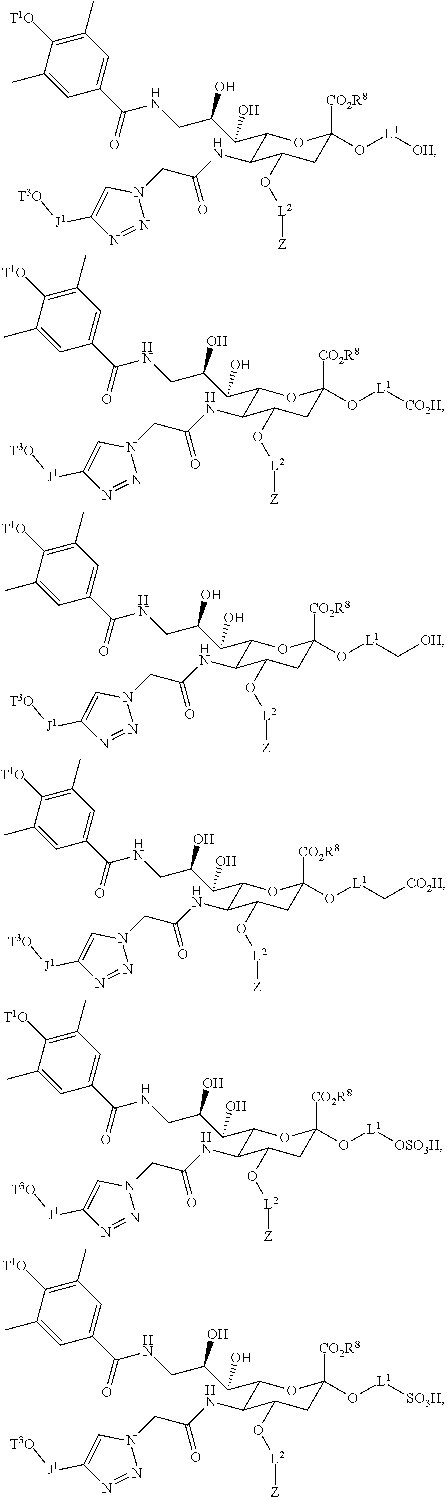

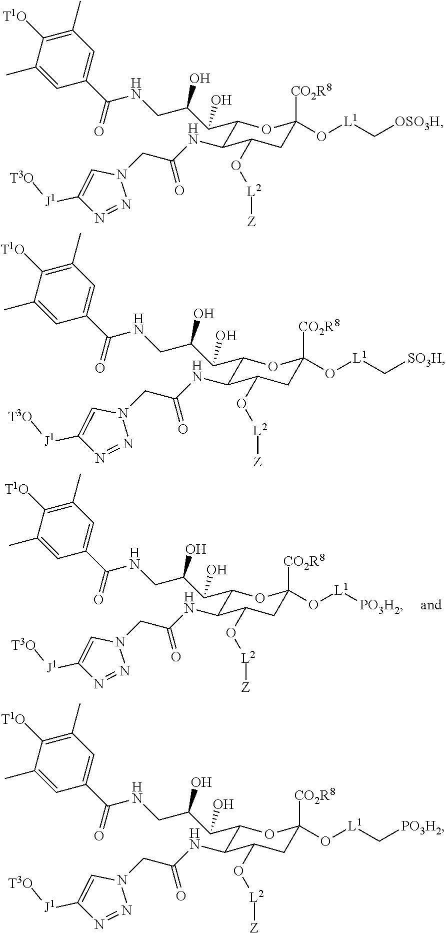

and pharmaceutically acceptable salts of any of the foregoing.

In some embodiments, the compound of Formula (I) is chosen from:

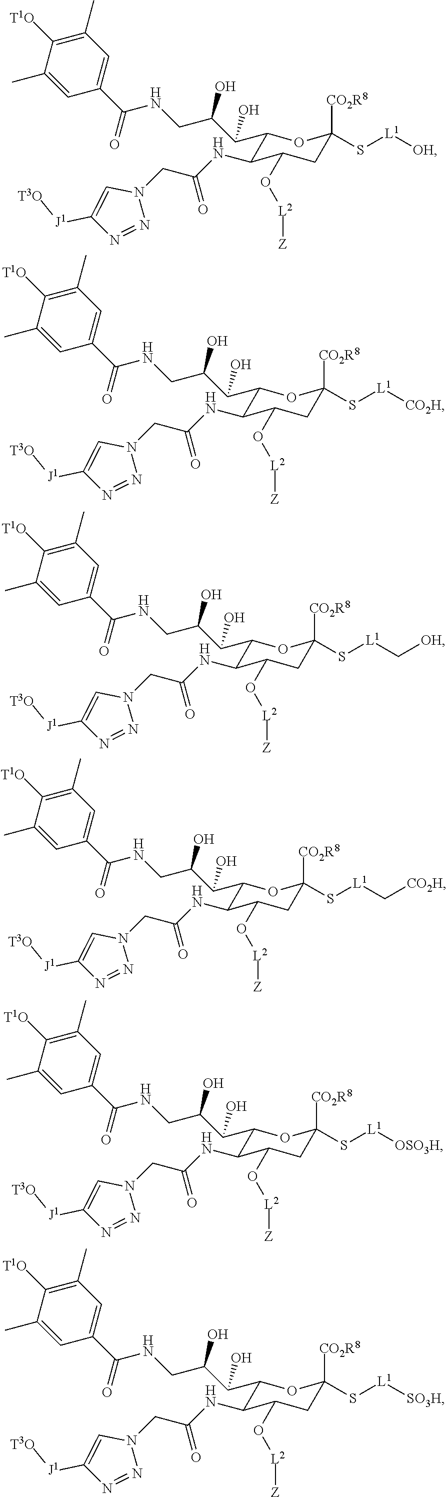

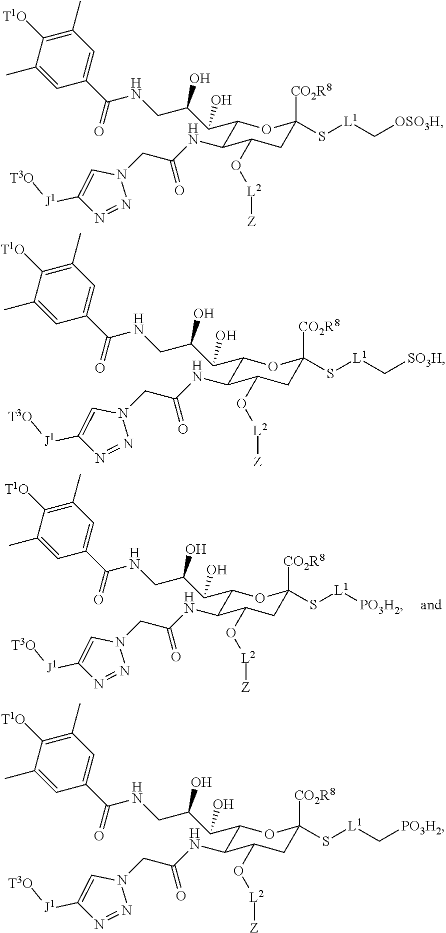

and pharmaceutically acceptable salts of any of the foregoing.

In some embodiments, the compound of Formula (I) is chosen from:

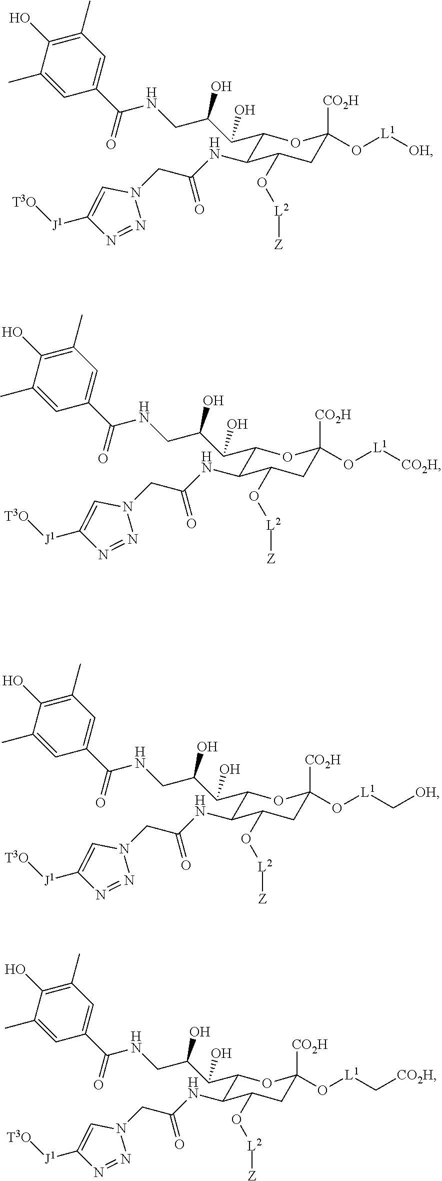

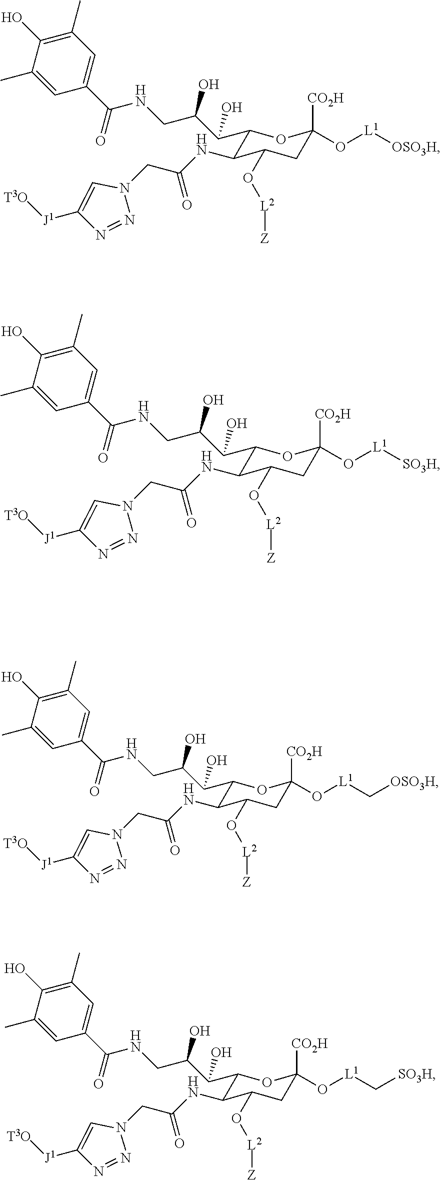

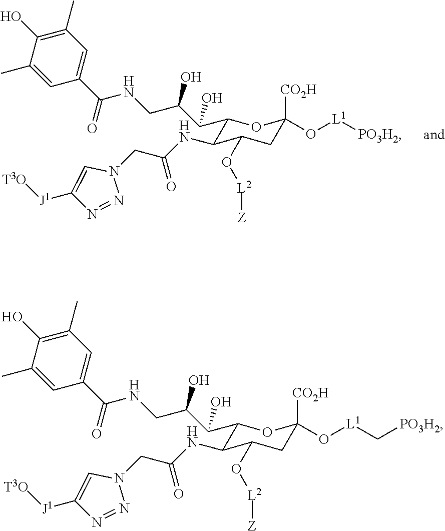

and pharmaceutically acceptable salts of any of the foregoing.

In some embodiments, the compound of Formula (I) is chosen from:

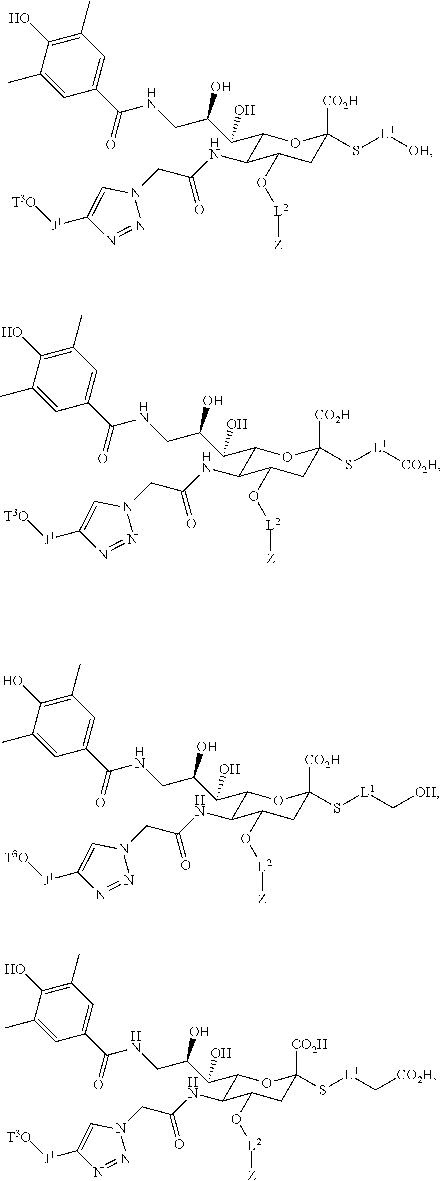

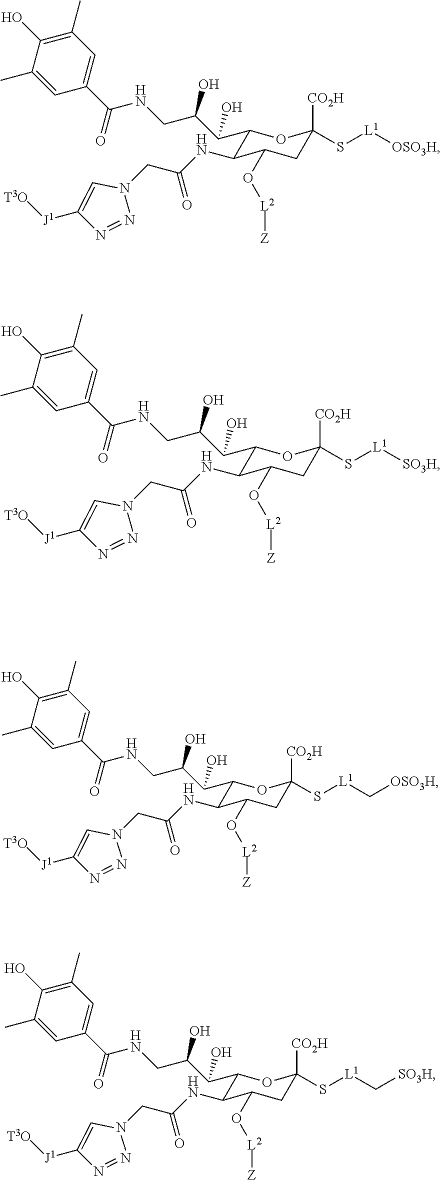

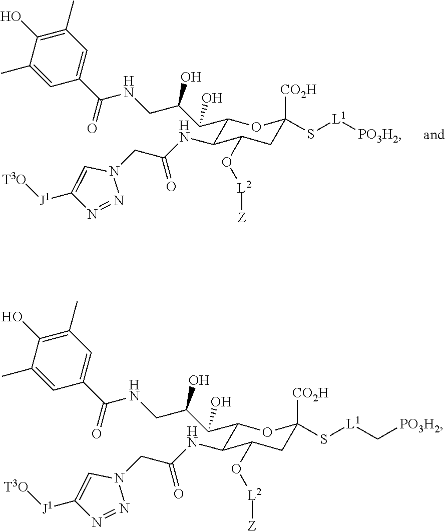

and pharmaceutically acceptable salts of any of the foregoing.

In some embodiments, the compound of Formula (I) is chosen from:

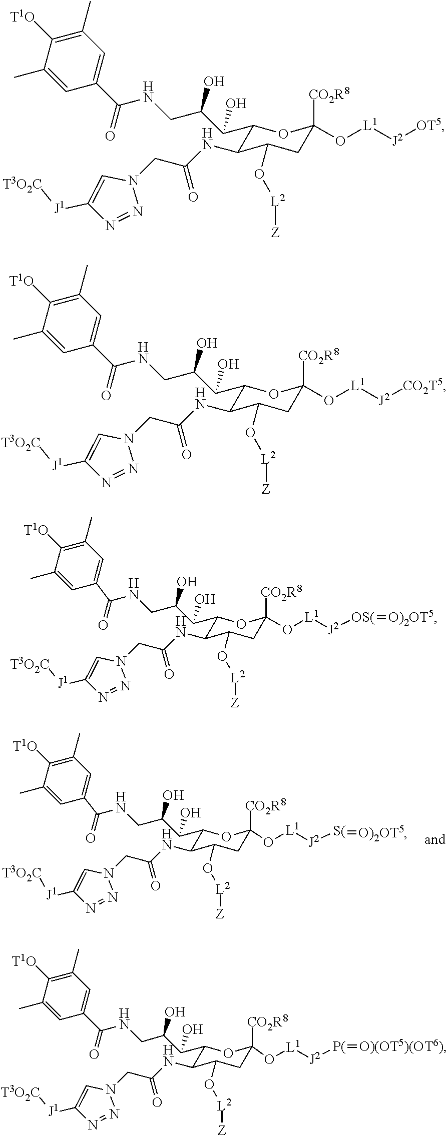

and pharmaceutically acceptable salts of any of the foregoing.

In some embodiments, the compound of Formula (I) is chosen from:

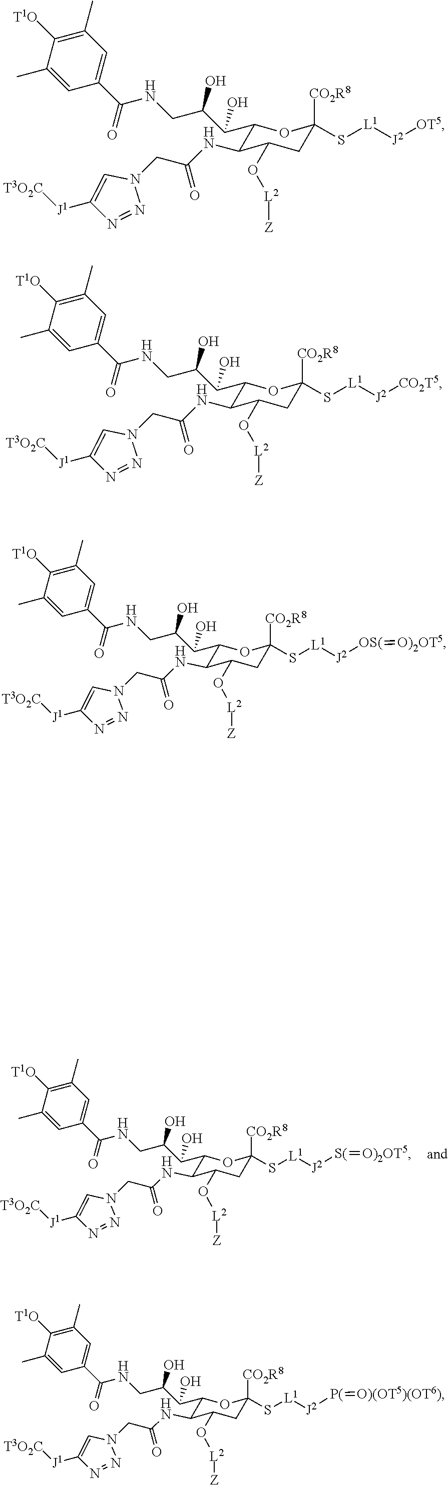

and pharmaceutically acceptable salts of any of the foregoing.

In some embodiments, the compound of Formula (I) is chosen from:

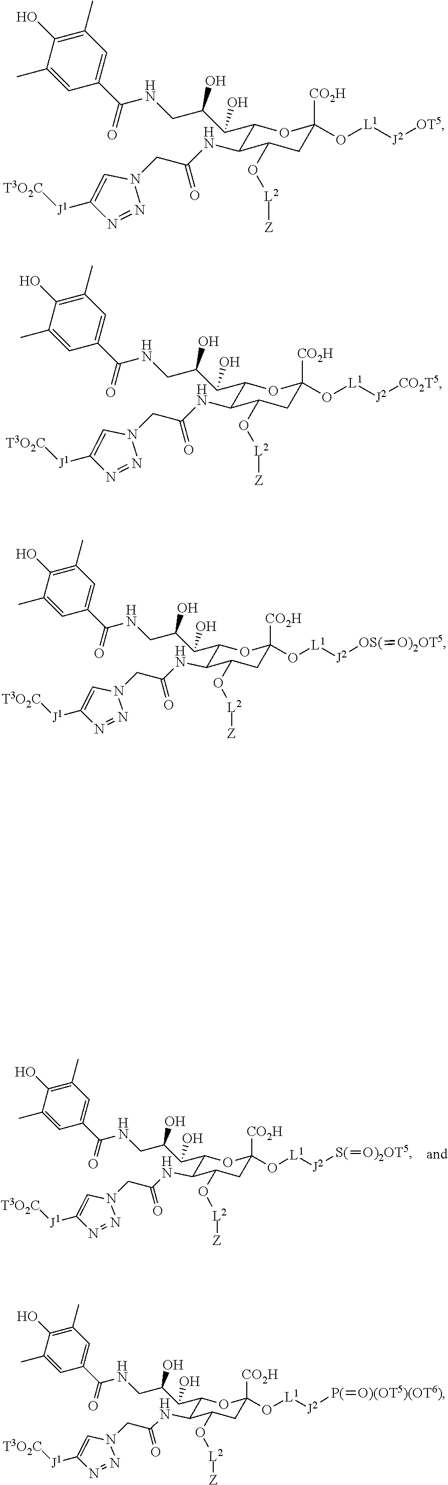

and pharmaceutically acceptable salts of any of the foregoing.

In some embodiments, the compound of Formula (I) is chosen from:

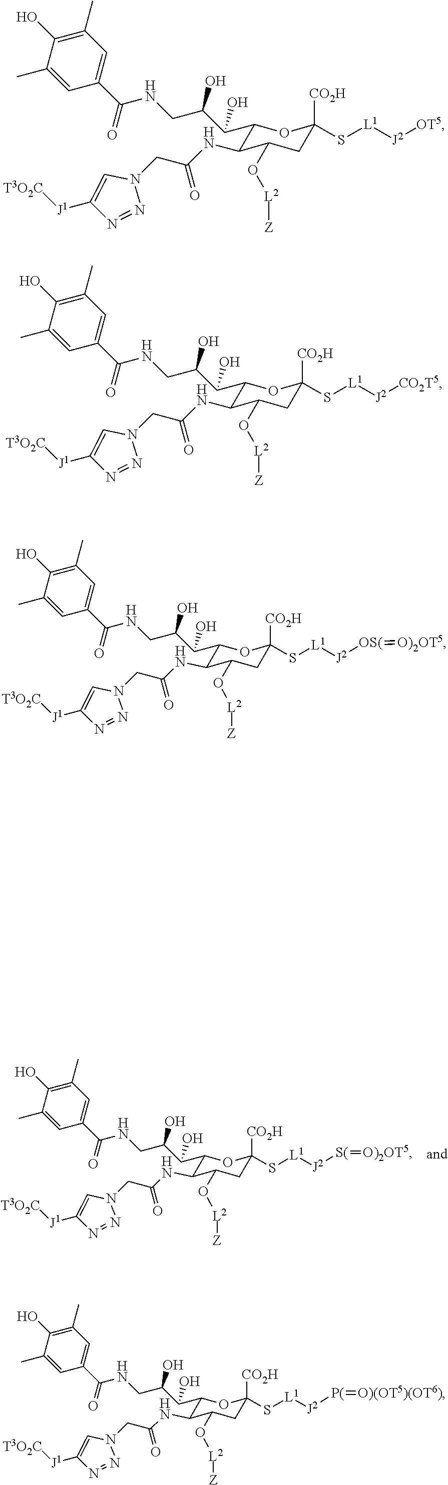

and pharmaceutically acceptable salts of any of the foregoing.

In some embodiments, the compound of Formula (I) is chosen from:

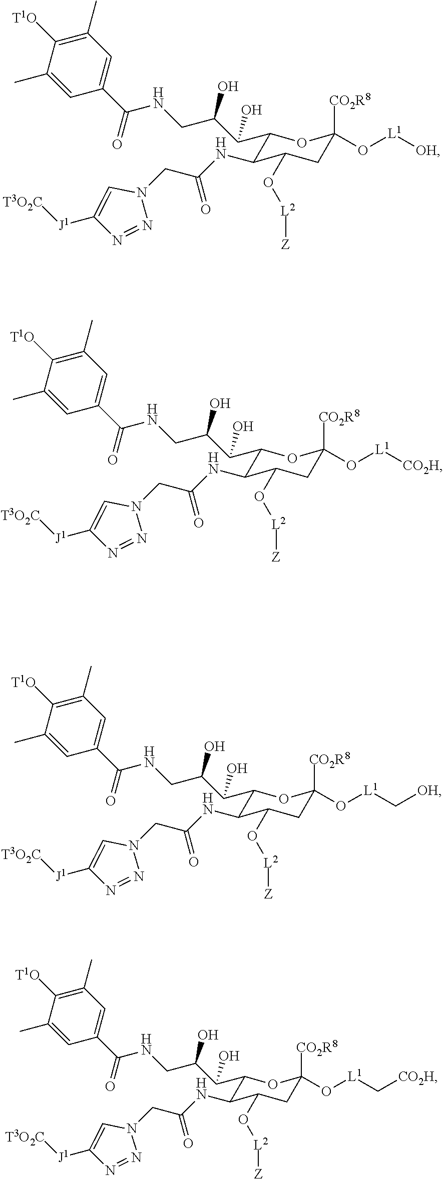

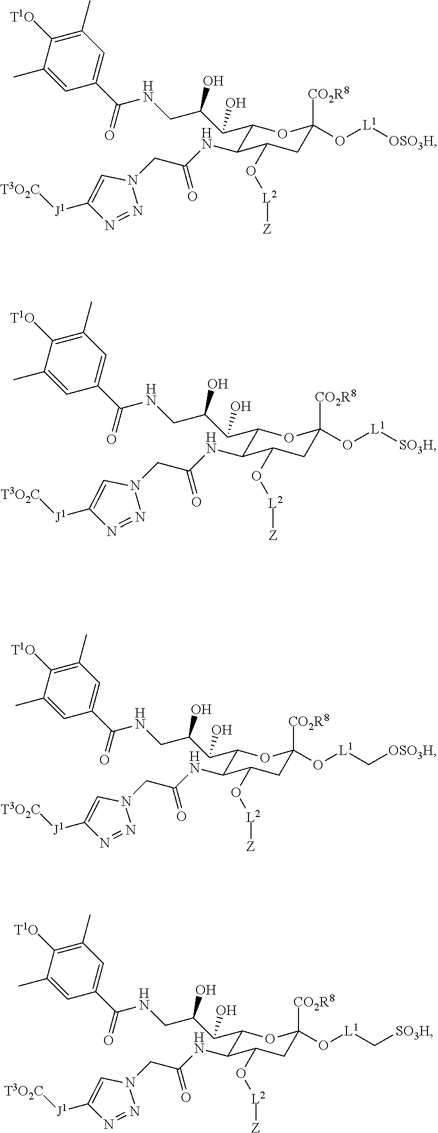

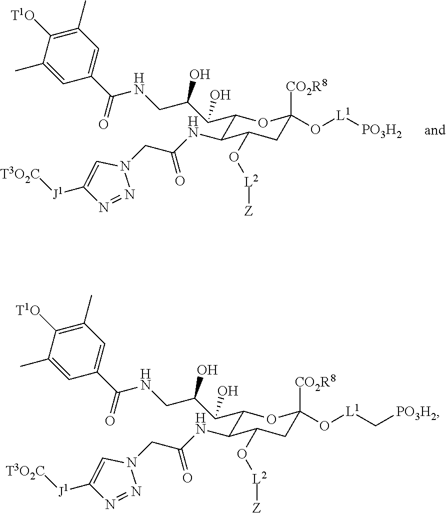

and pharmaceutically acceptable salts of any of the foregoing.

In some embodiments, the compound of Formula (I) is chosen from:

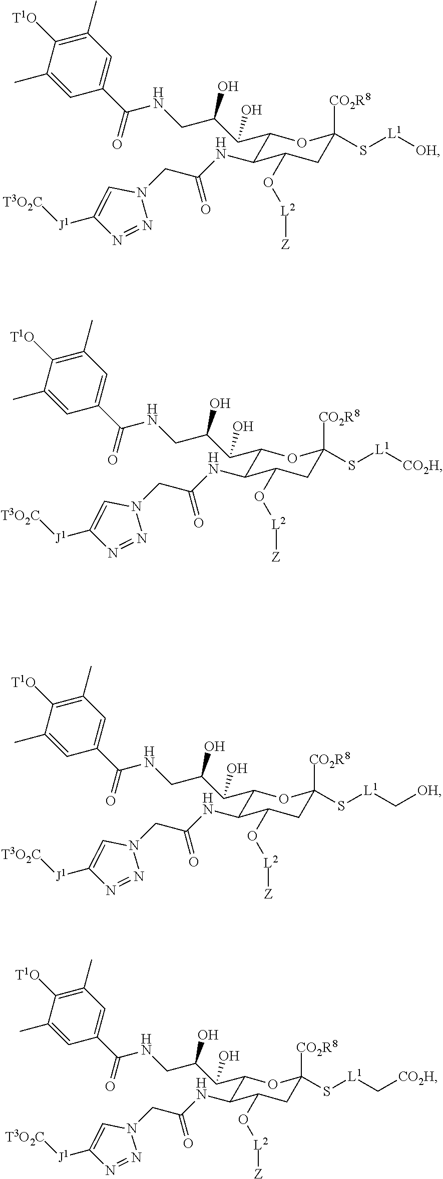

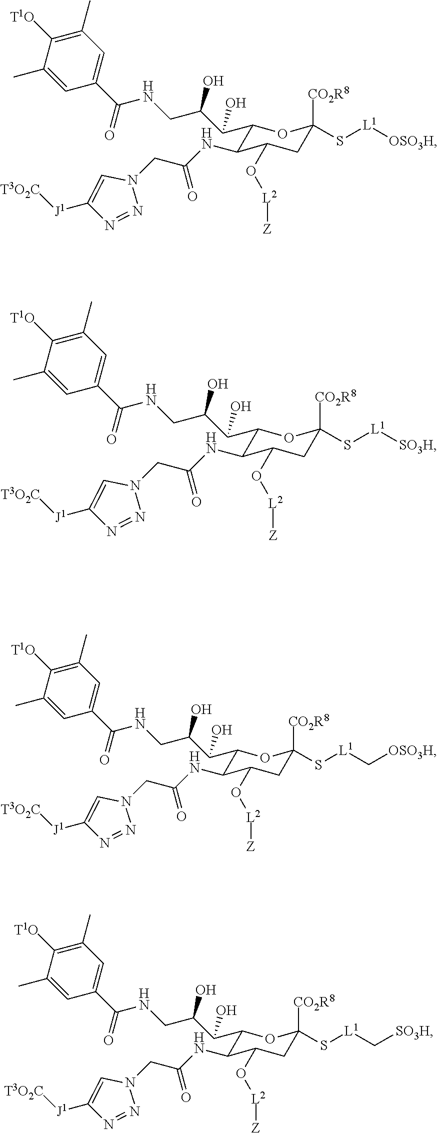

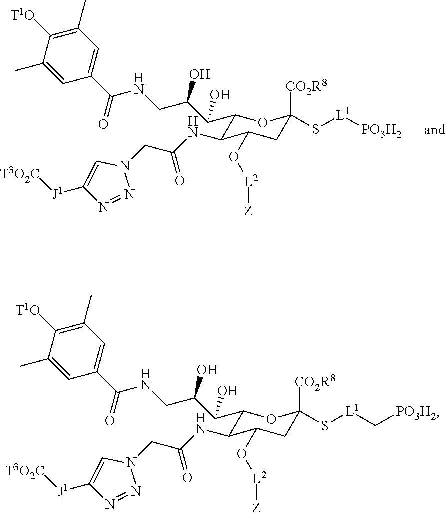

and pharmaceutically acceptable salts of any of the foregoing.

In some embodiments, the compound of Formula (I) is chosen from:

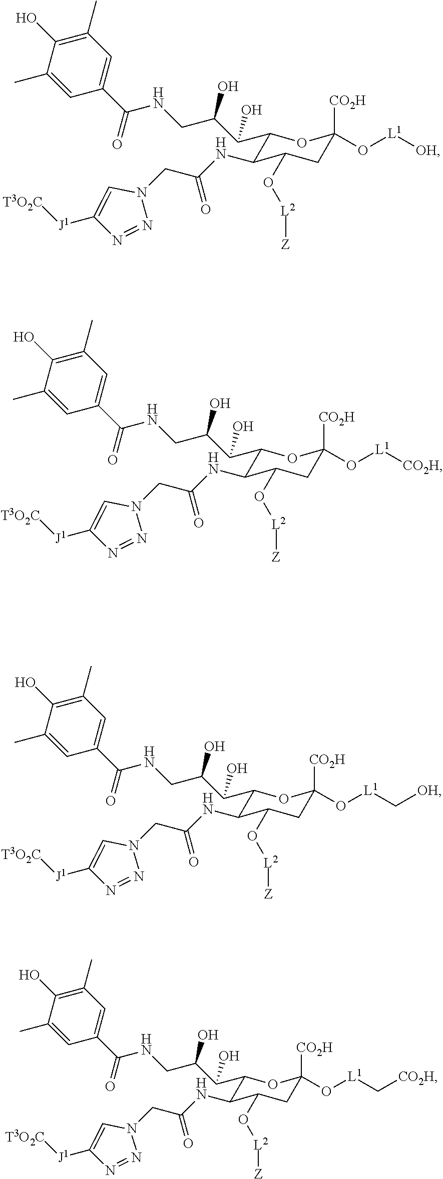

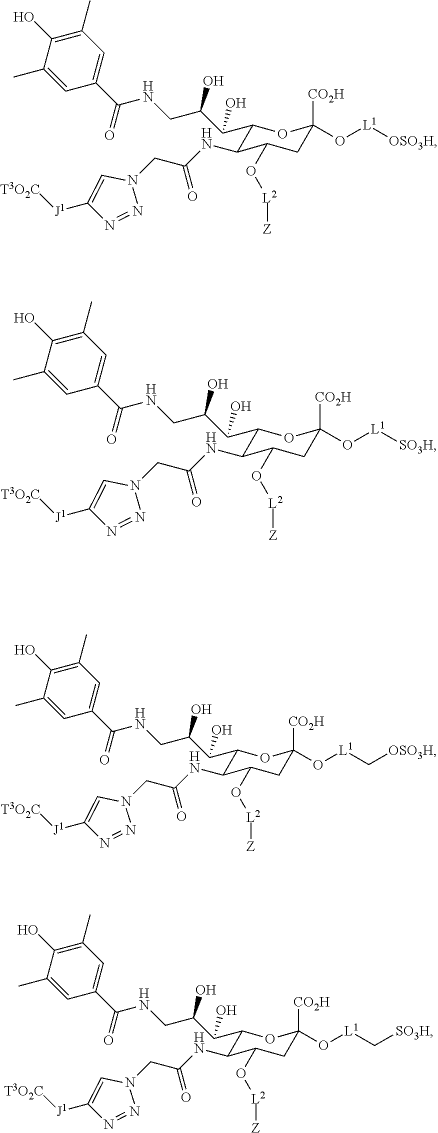

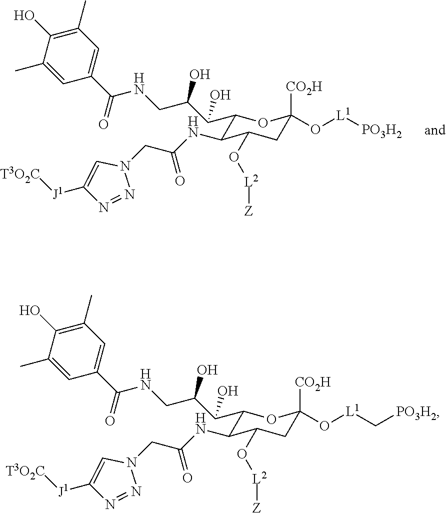

and pharmaceutically acceptable salts of any of the foregoing.

In some embodiments, the compound of Formula (I) is chosen from:

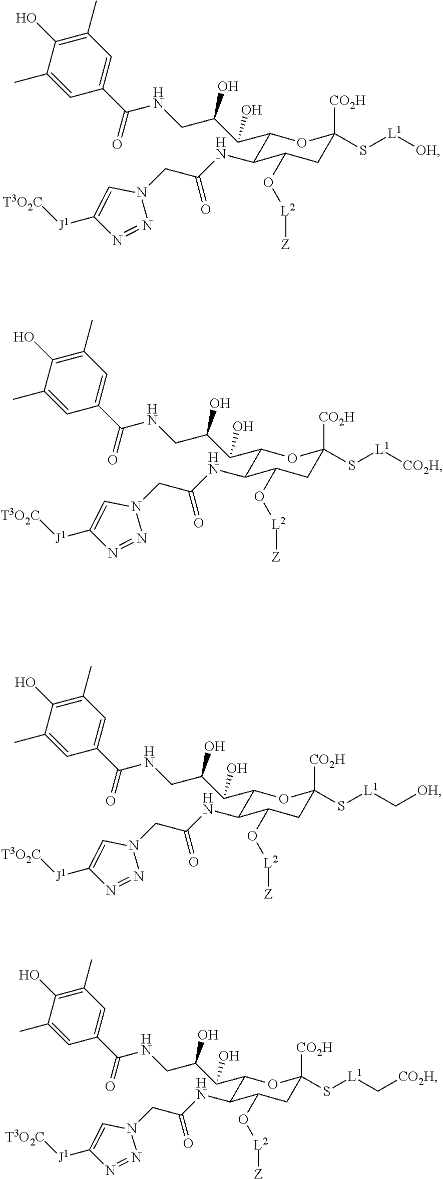

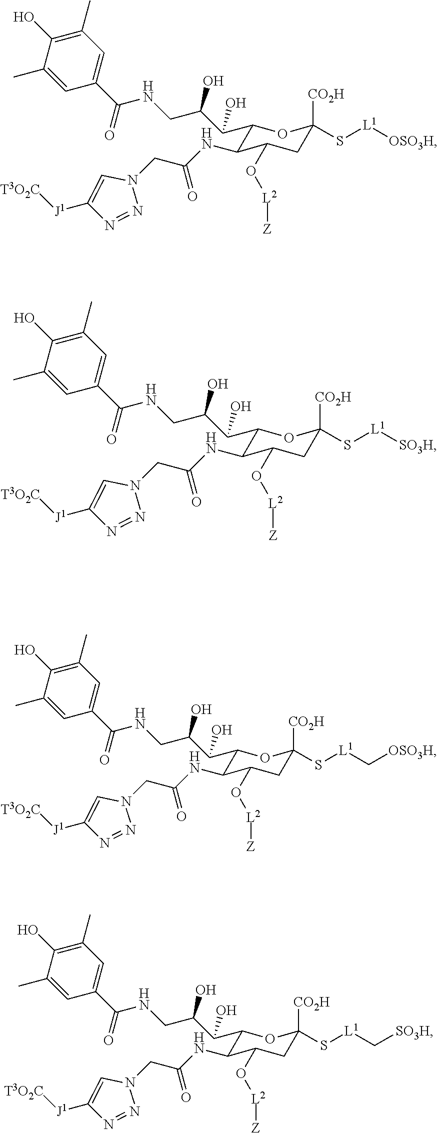

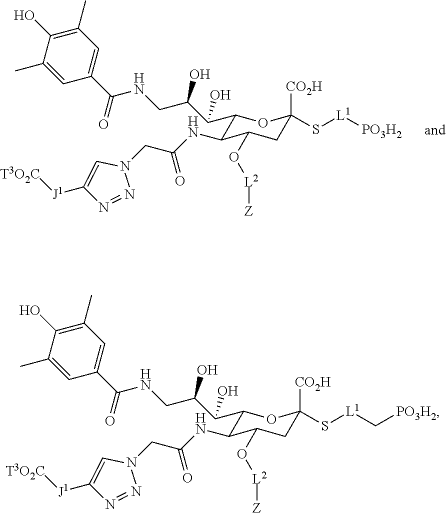

and pharmaceutically acceptable salts of any of the foregoing.

In some embodiments, the compound of Formula (I) is chosen from:

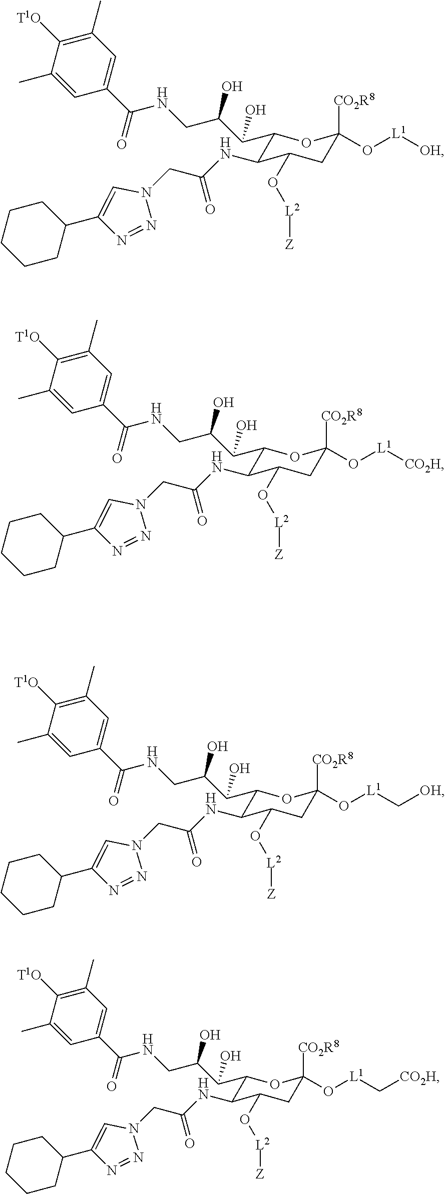

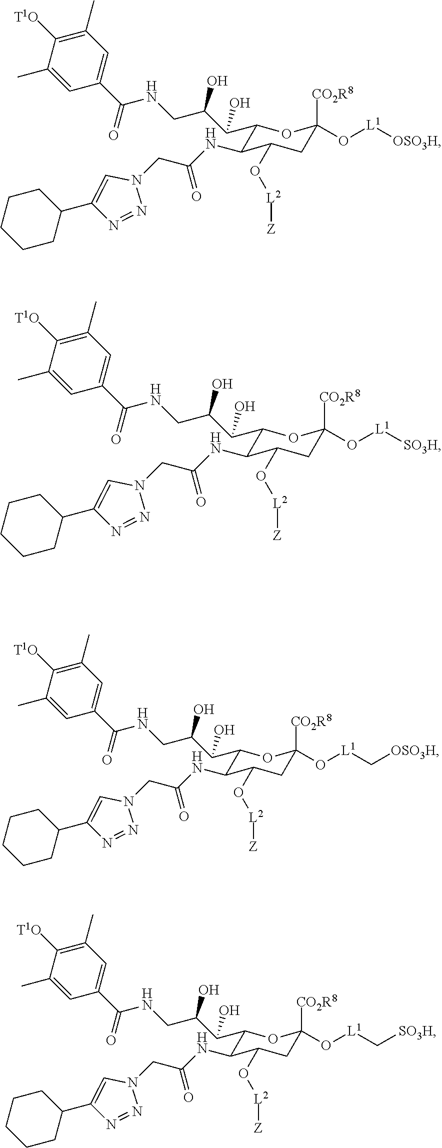

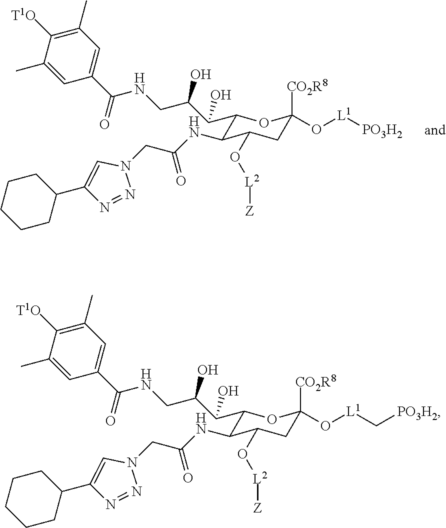

and pharmaceutically acceptable salts of any of the foregoing.

In some embodiments, the compound of Formula (I) is chosen from:

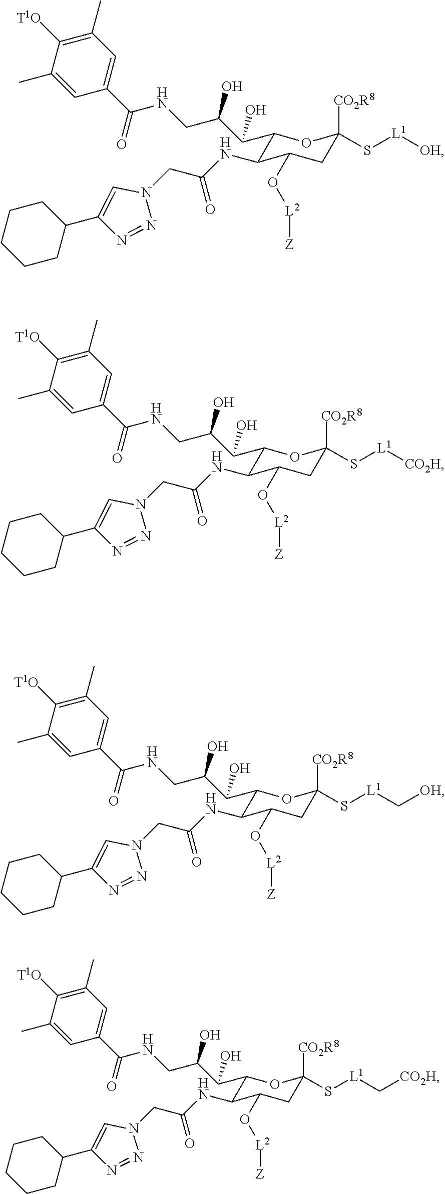

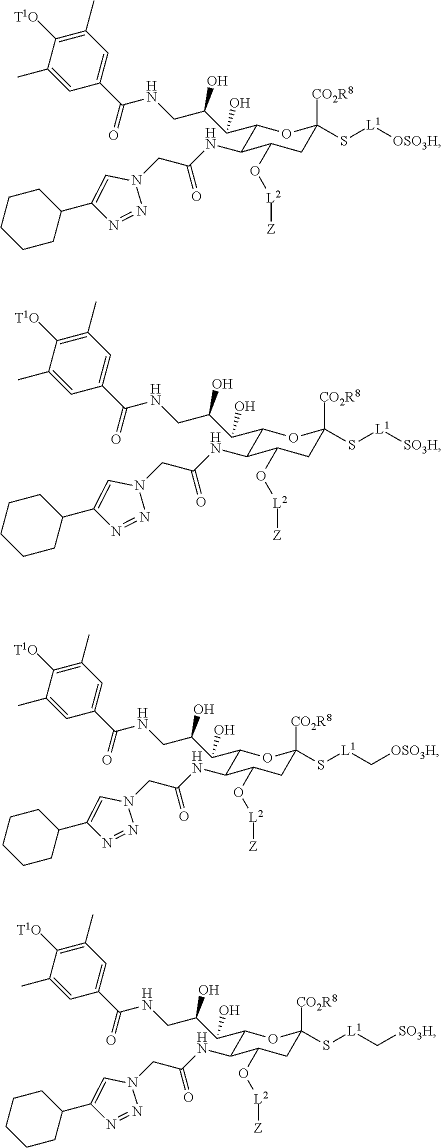

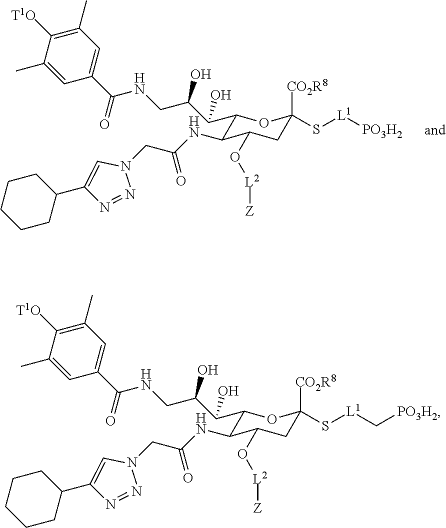

and pharmaceutically acceptable salts of any of the foregoing.

In some embodiments, the compound of Formula (I) is chosen from:

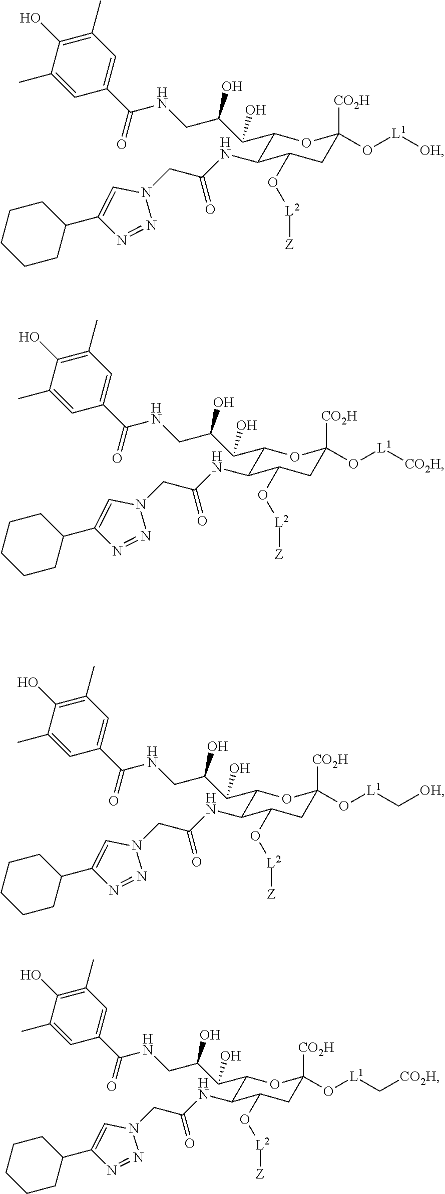

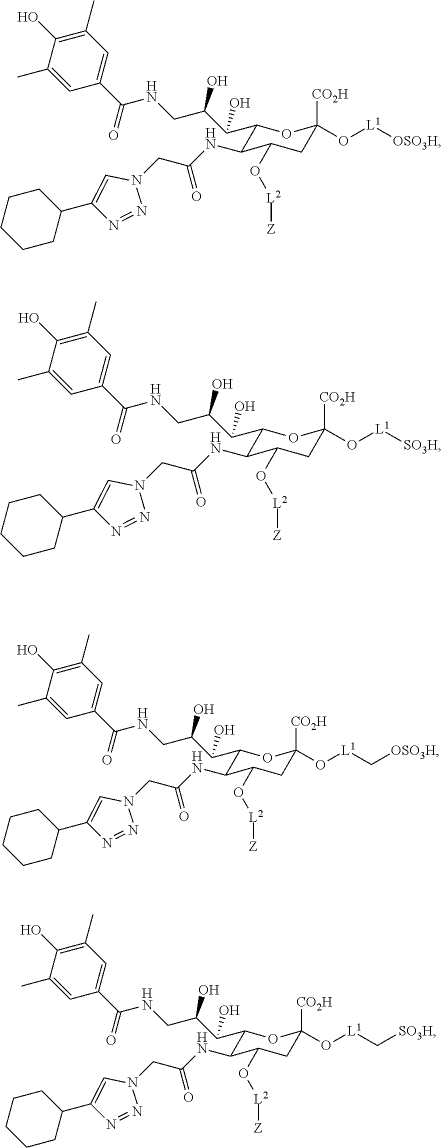

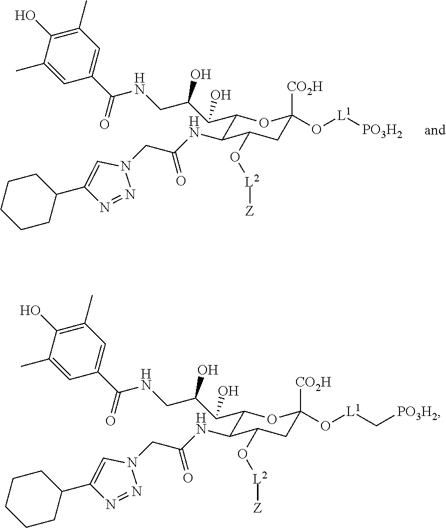

and pharmaceutically acceptable salts of any of the foregoing.

In some embodiments, the compound of Formula (I) is chosen from:

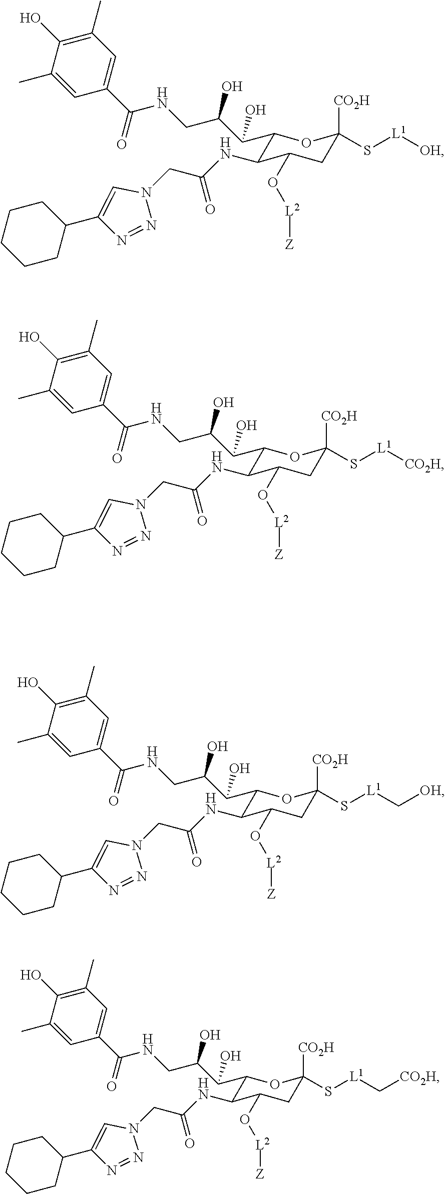

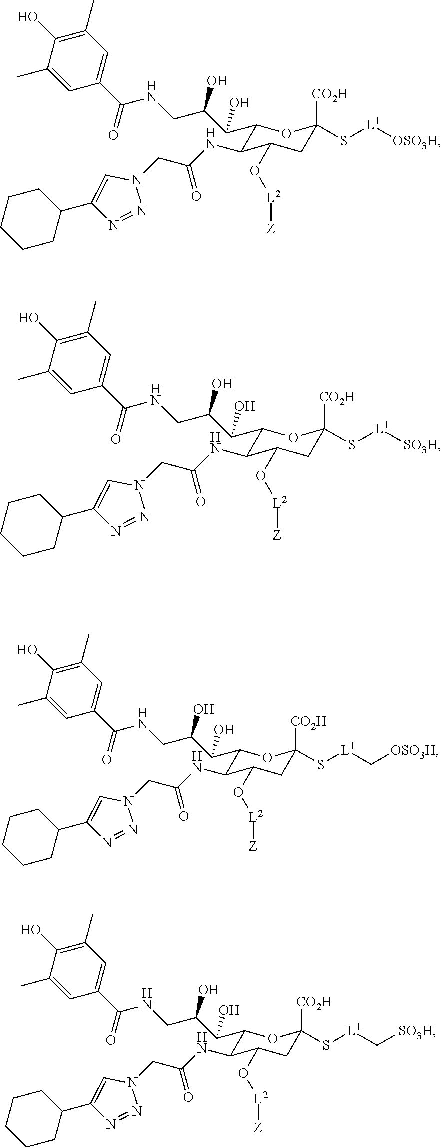

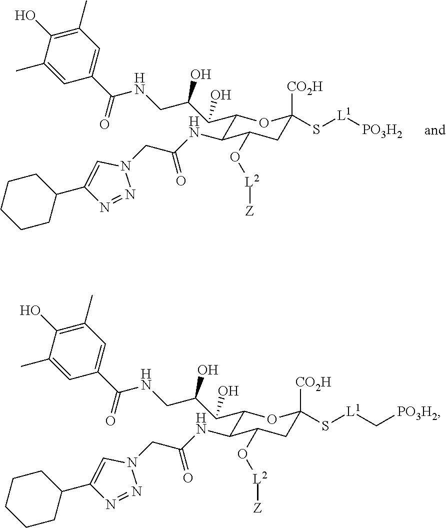

and pharmaceutically acceptable salts of any of the foregoing.
In some embodiments, the compound of Formula (I) is chosen from:

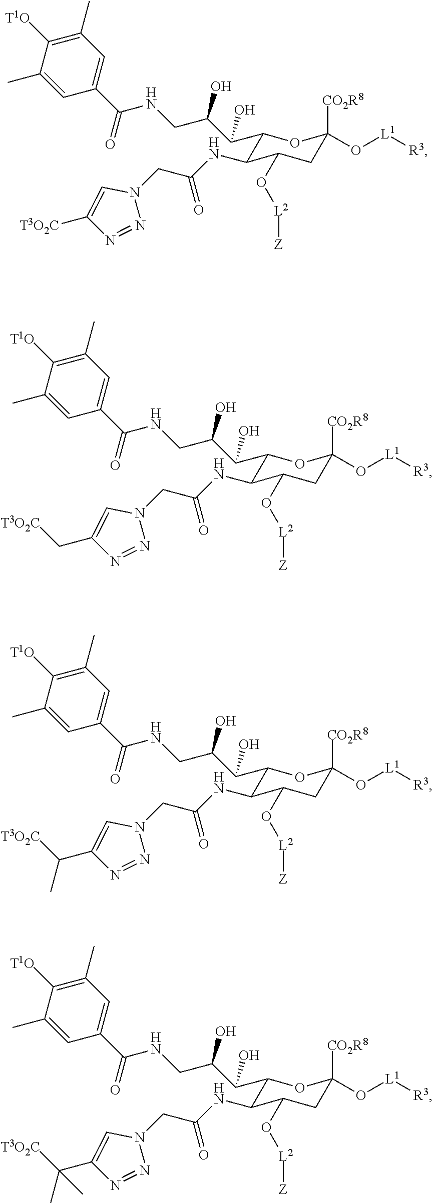

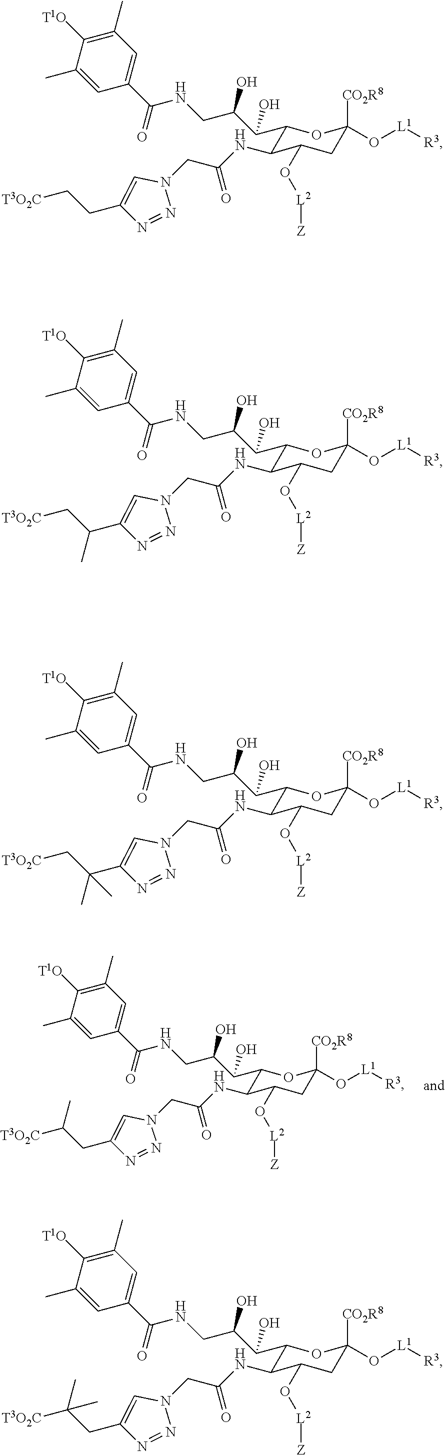

and pharmaceutically acceptable salts of any of the foregoing.

In some embodiments, the compound of Formula (I) is chosen from:

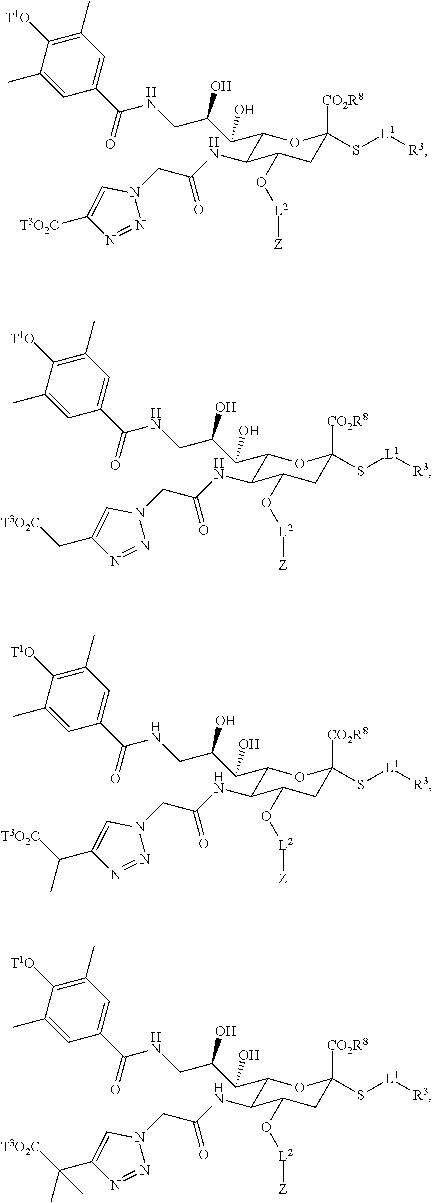

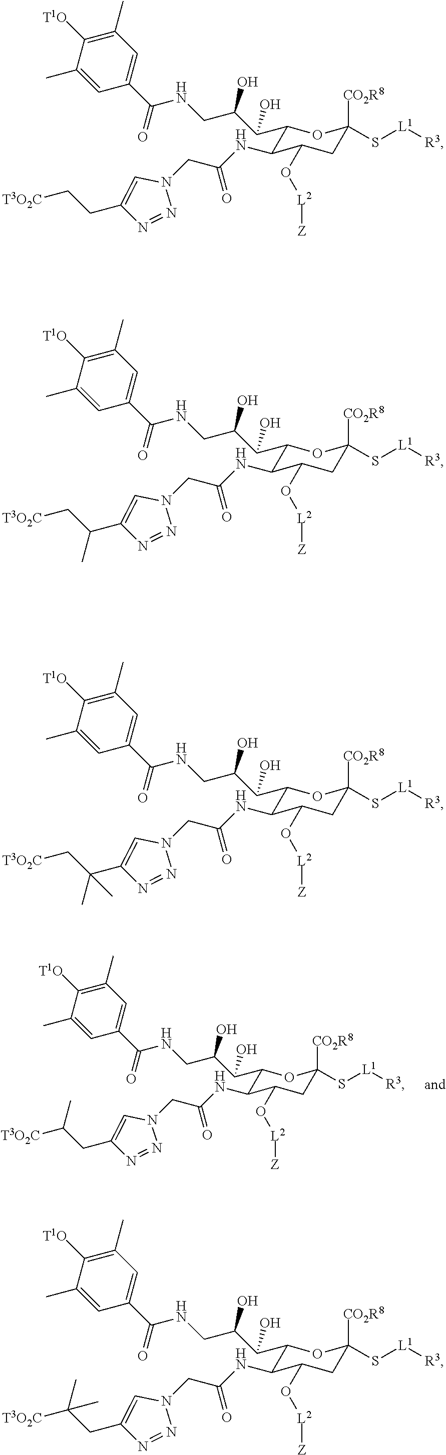

and pharmaceutically acceptable salts of any of the foregoing.

In some embodiments, the compound of Formula (I) is chosen from:

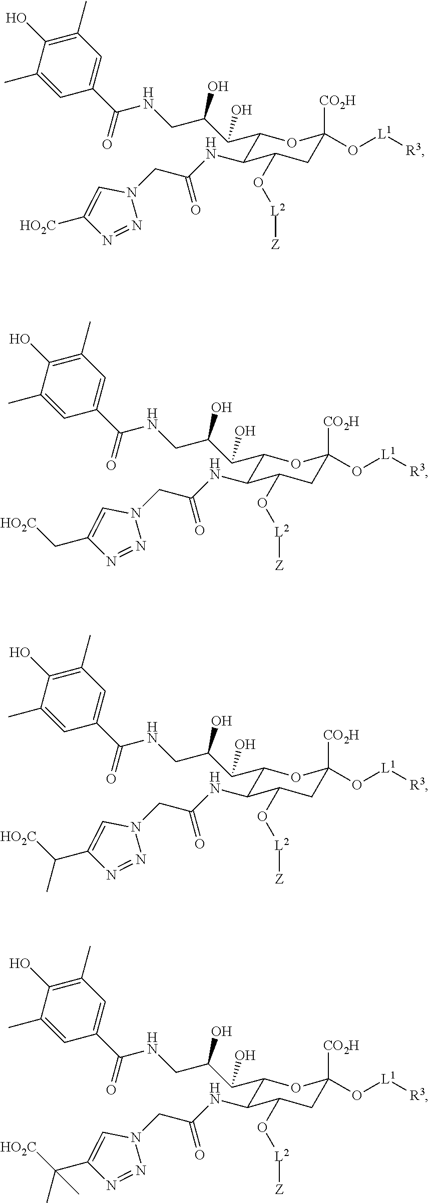

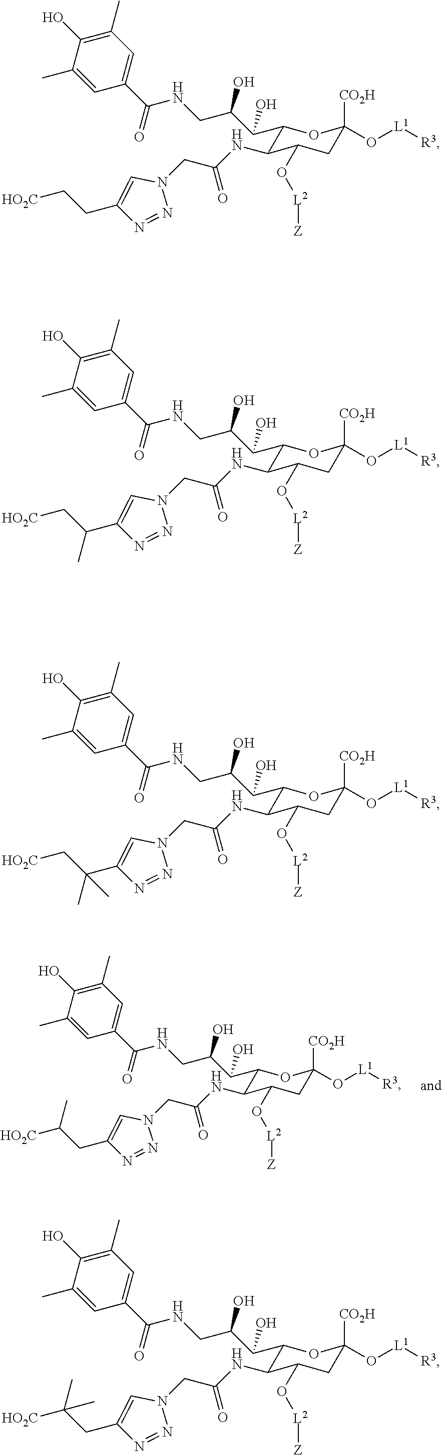

and pharmaceutically acceptable salts of any of the foregoing.

In some embodiments, the compound of Formula (I) is chosen from:

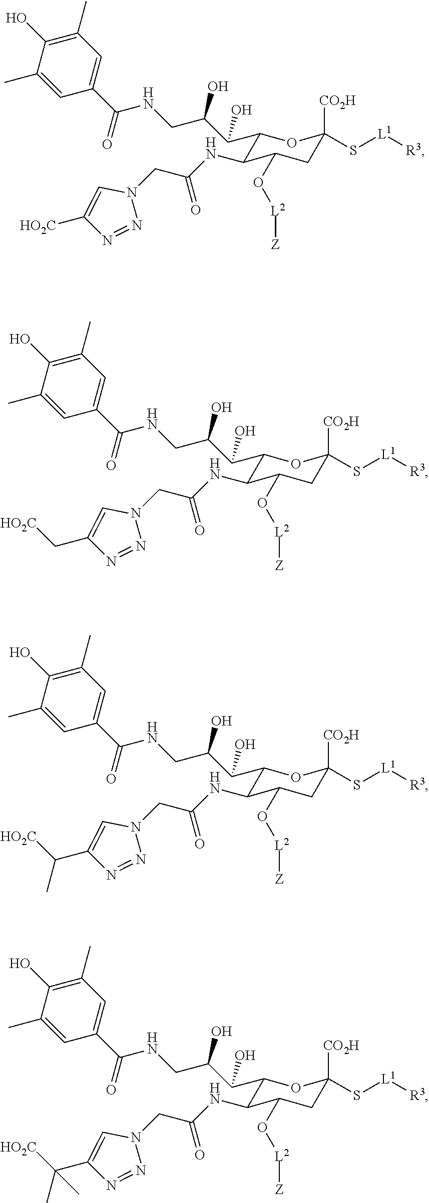

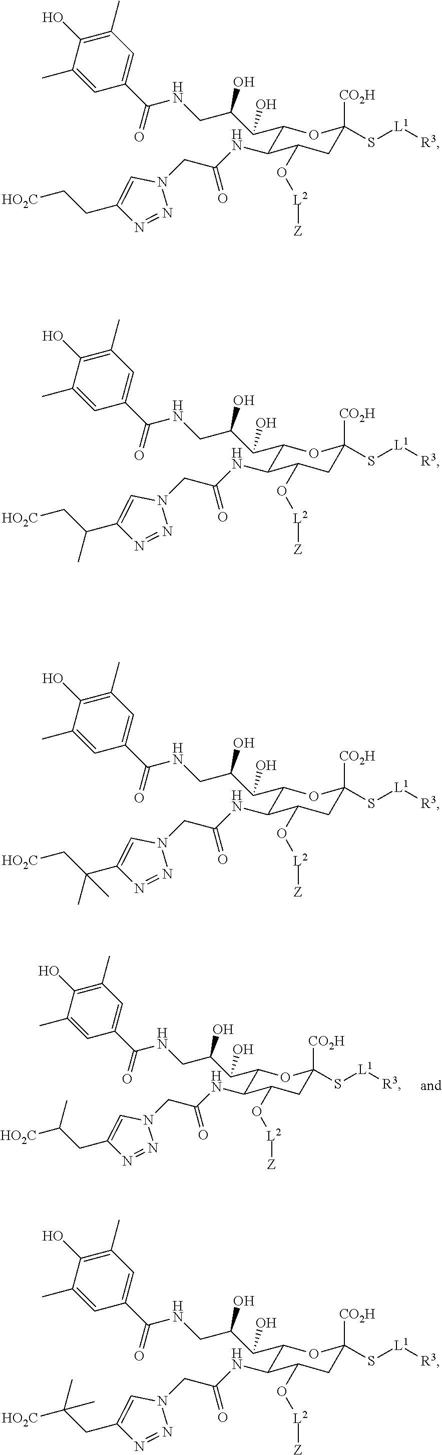

and pharmaceutically acceptable salts of any of the foregoing.

In some embodiments, the compound of Formula (I) is chosen from:

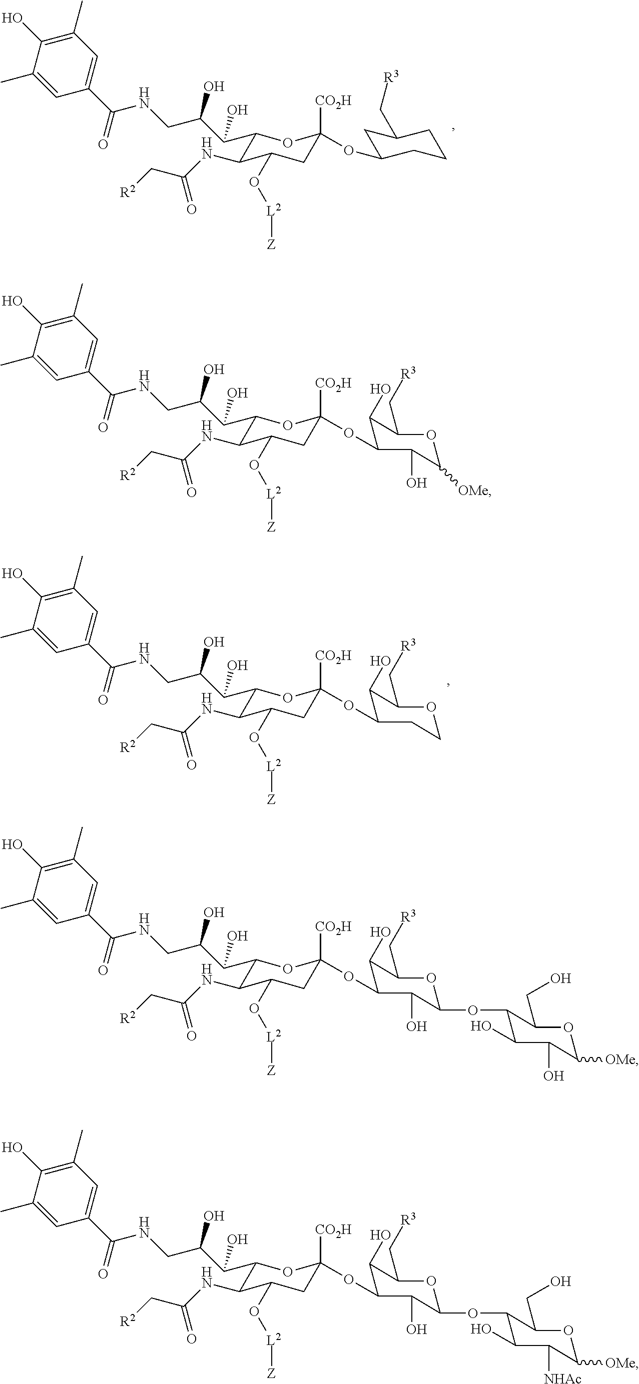

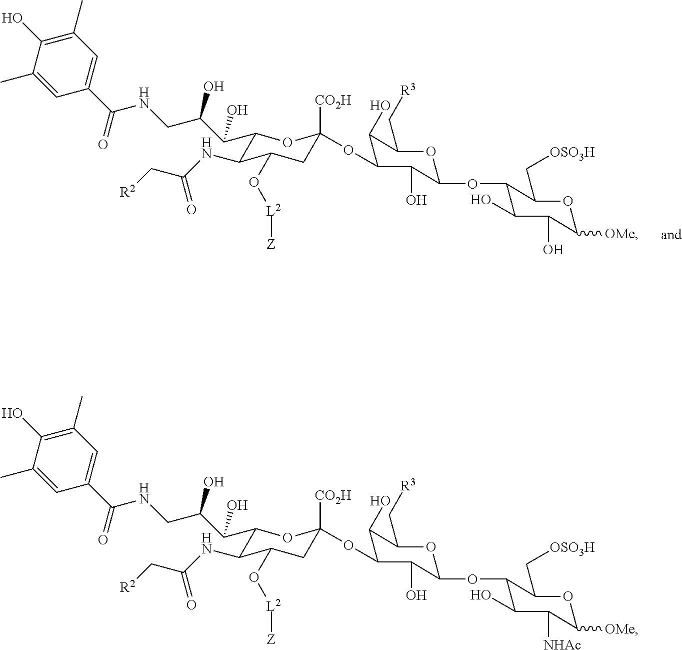

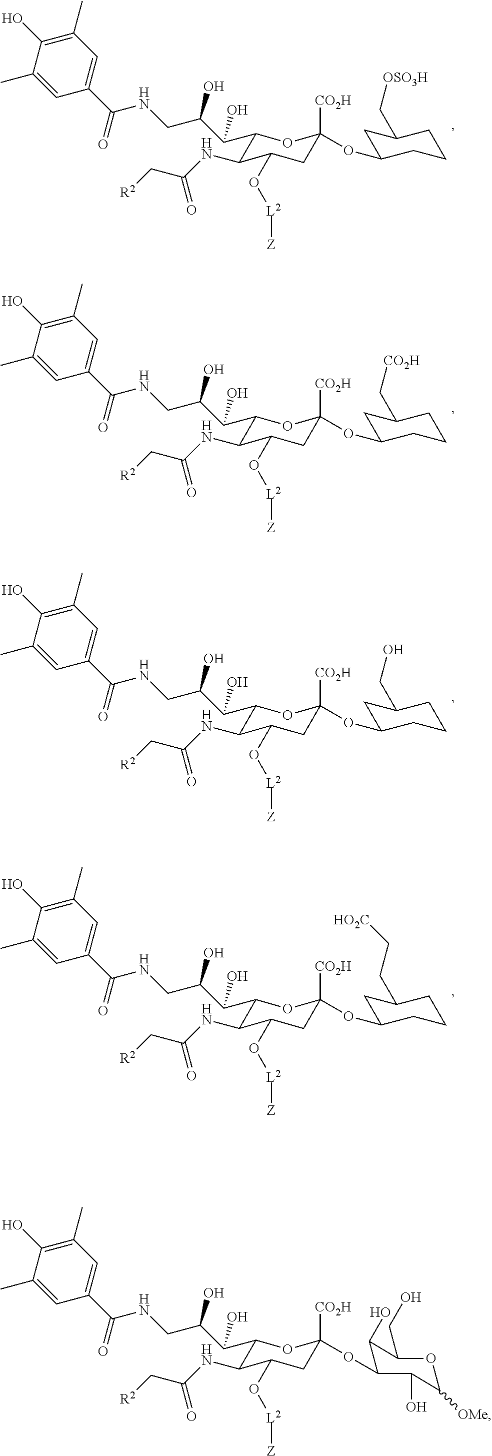

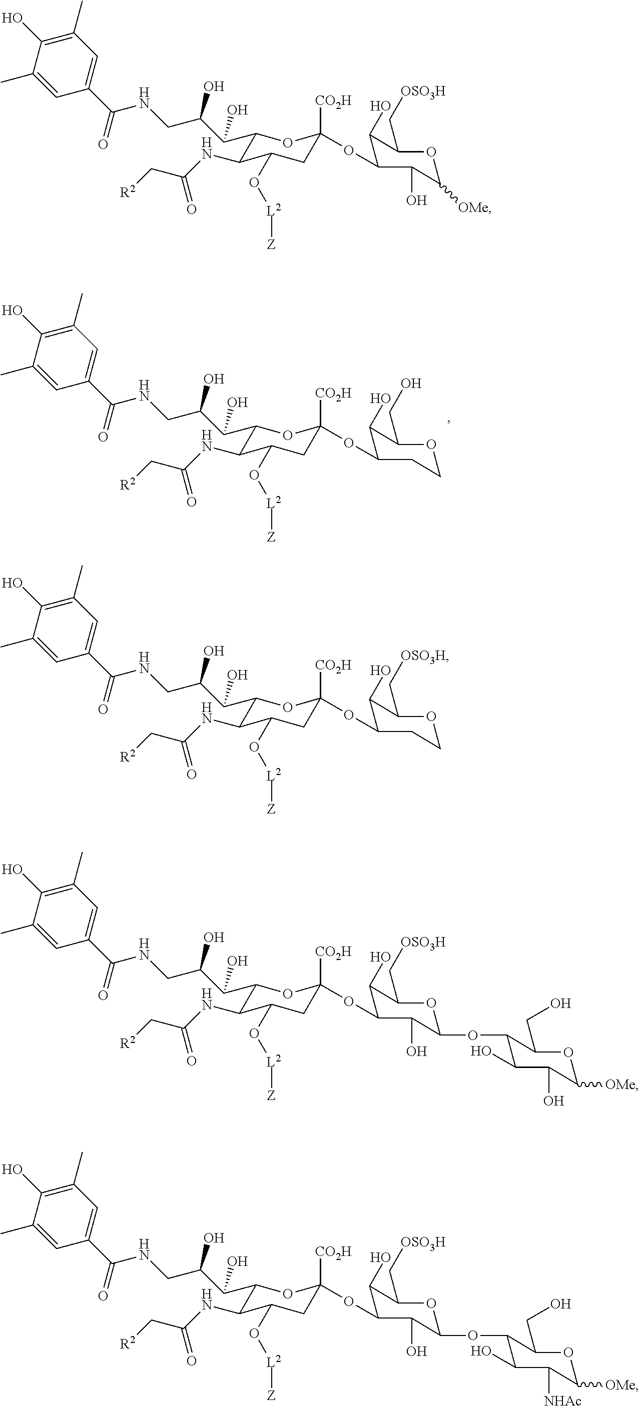

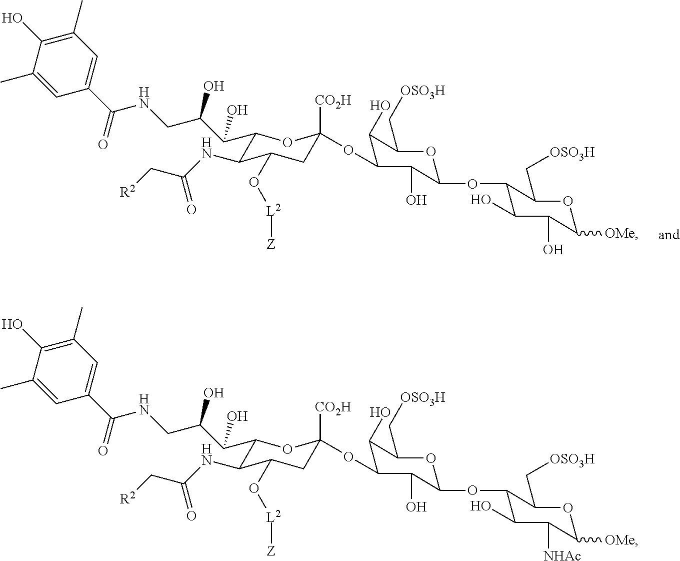

and pharmaceutically acceptable salts of any of the foregoing.

In some embodiments, the compound of Formula (I) is chosen from:

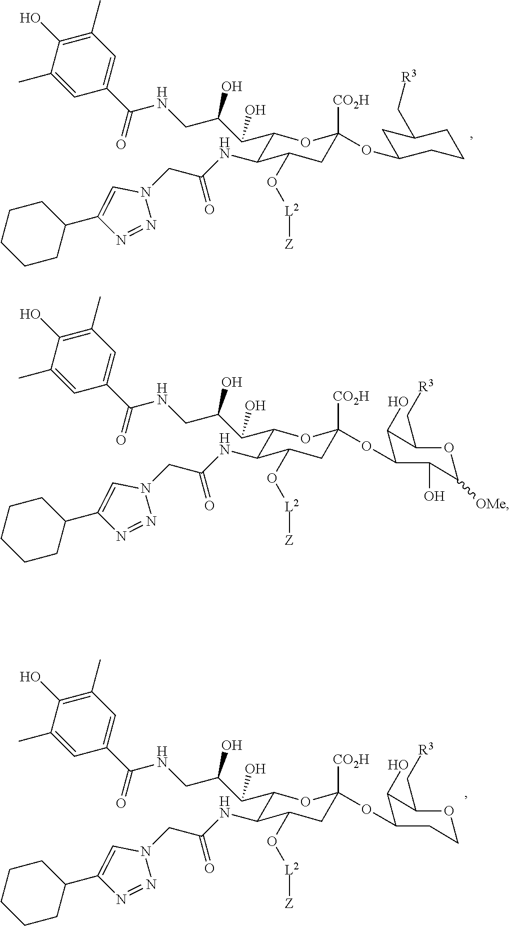

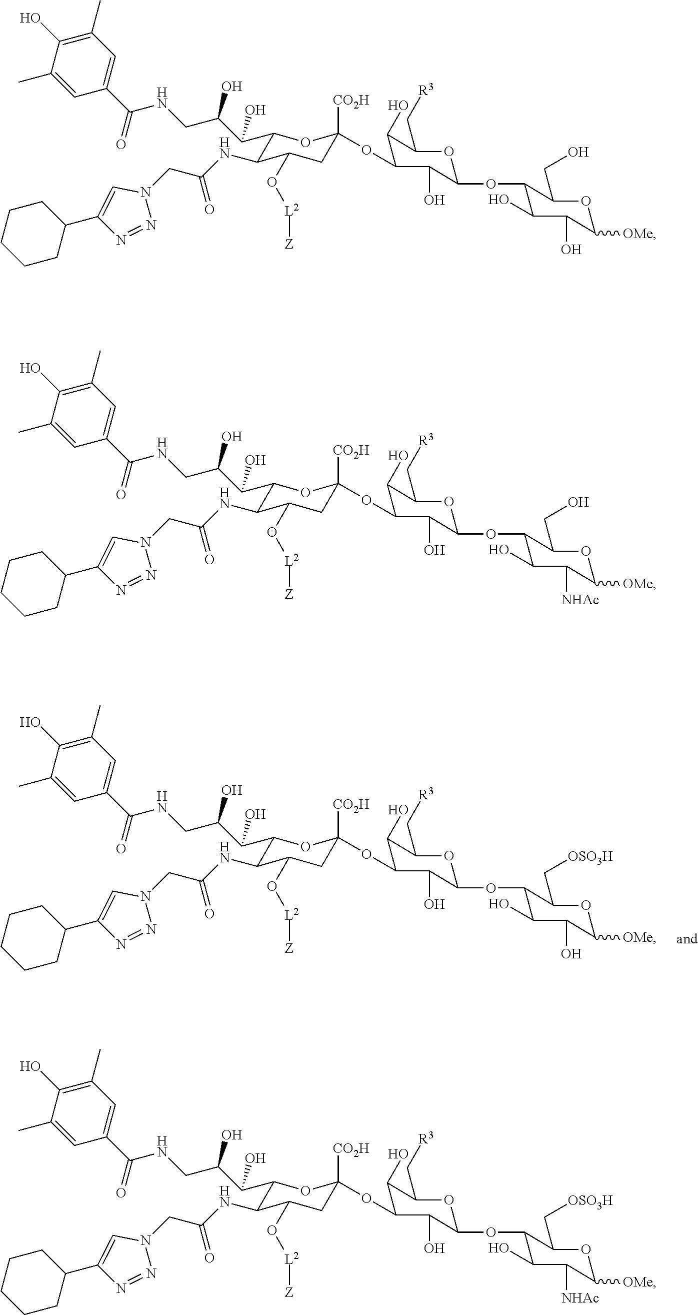

and pharmaceutically acceptable salts of any of the foregoing.

In some embodiments, the compound of Formula (I) is chosen from:

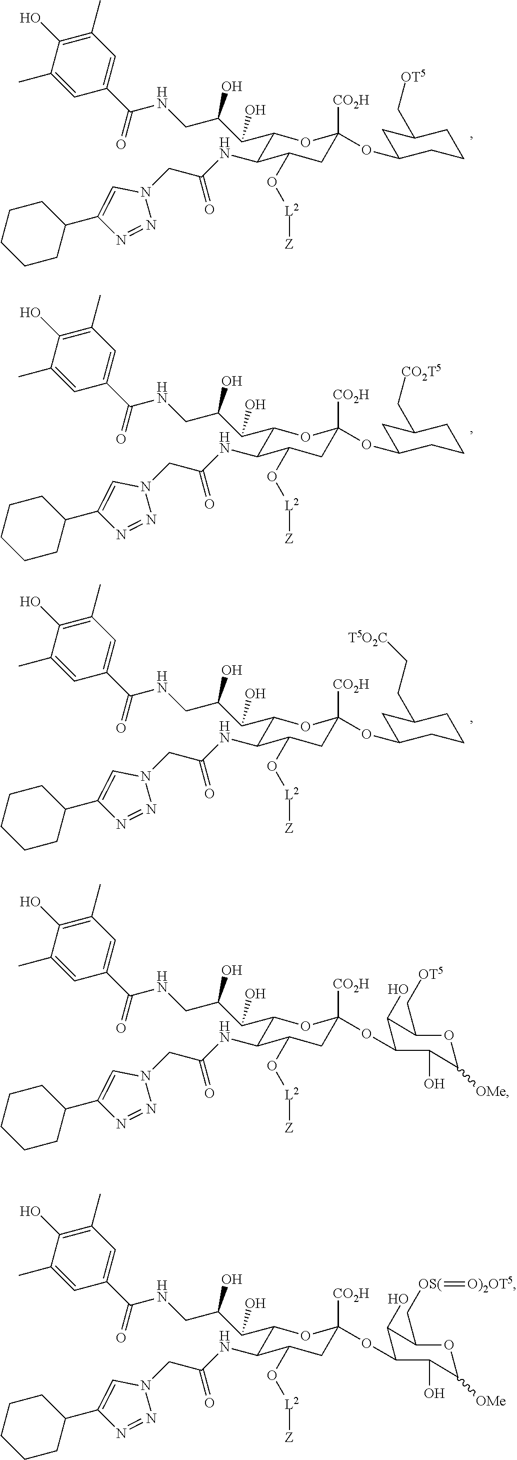

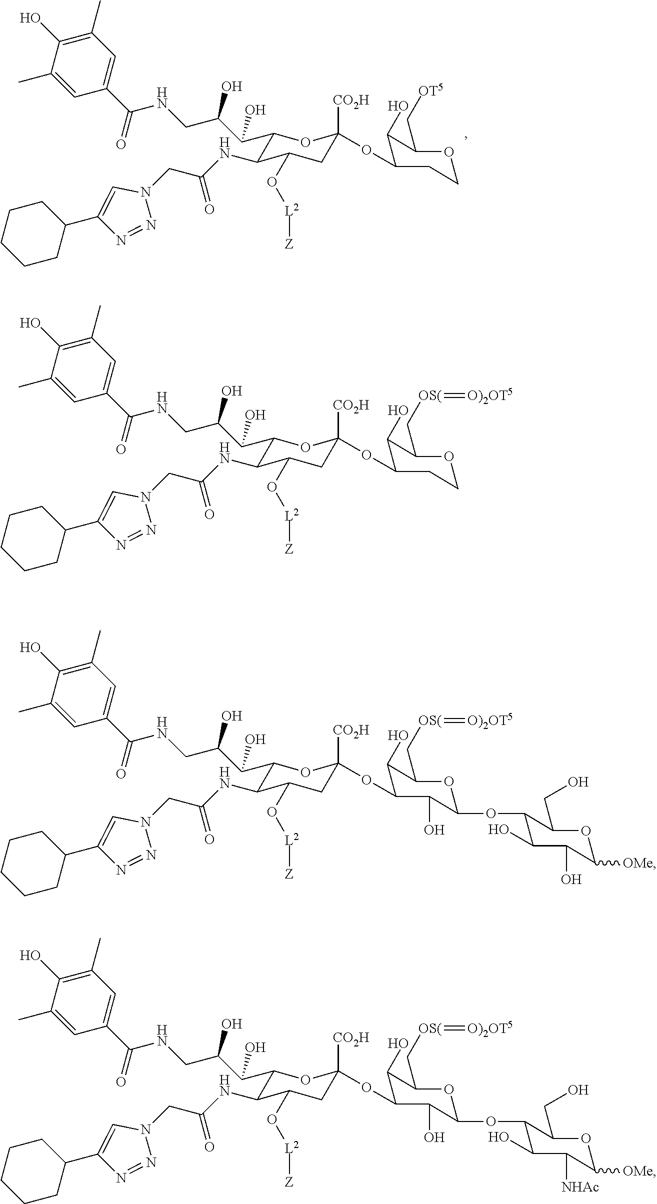

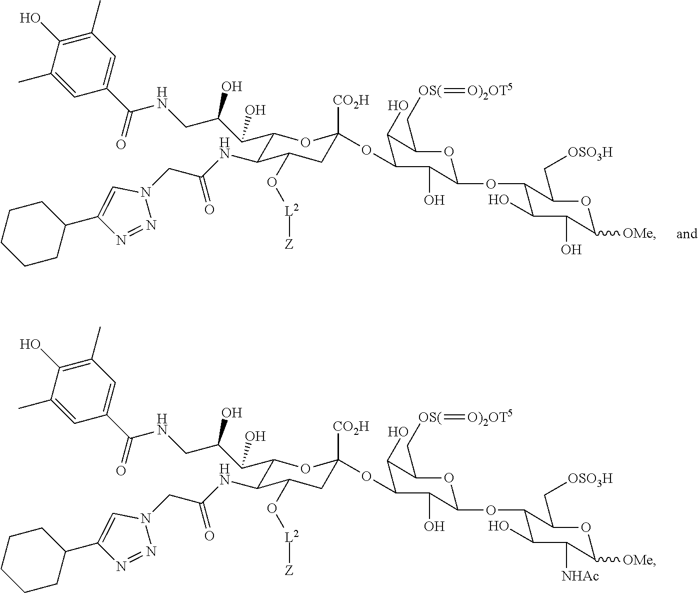

and pharmaceutically acceptable salts of any of the foregoing.

In some embodiments, the compound of Formula (I) is chosen from:

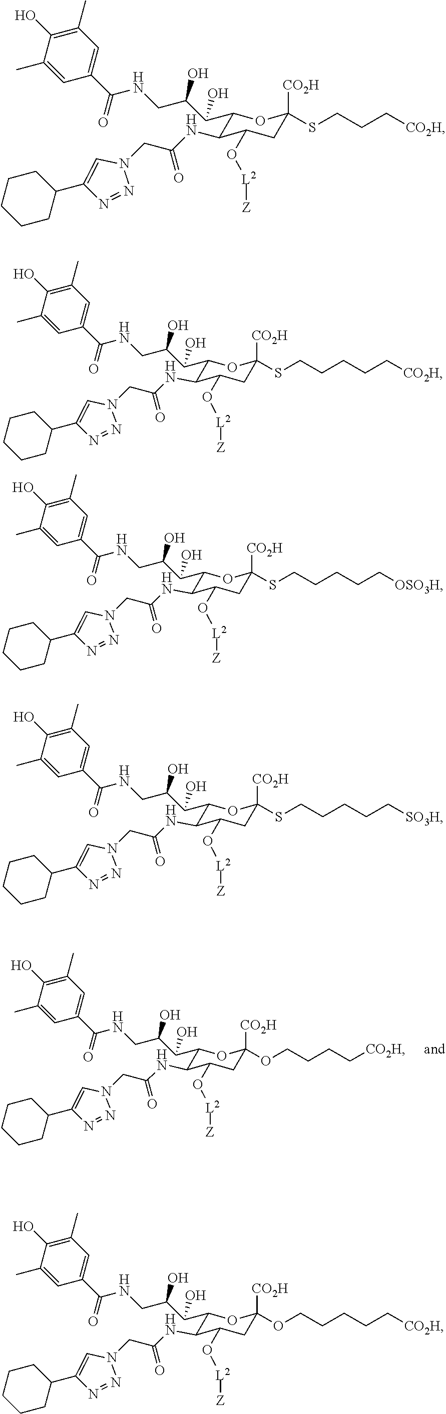

and pharmaceutically acceptable salts of any of the foregoing.

In some embodiments, the compound of Formula (I) is chosen from:

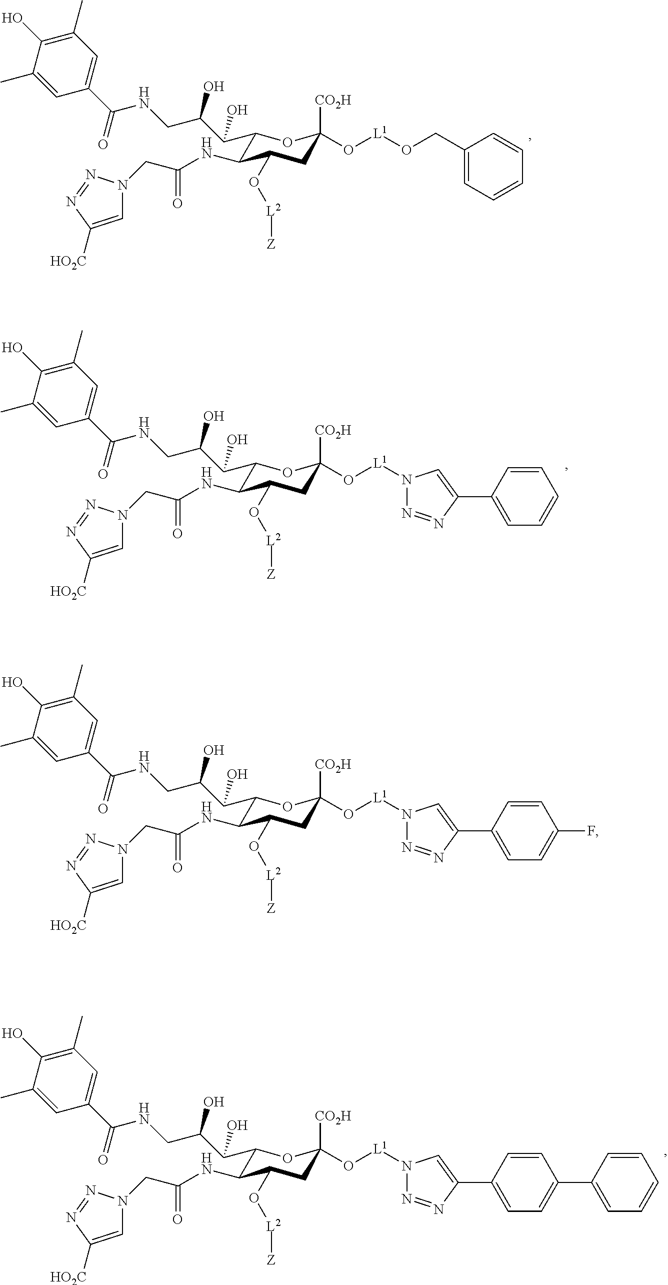

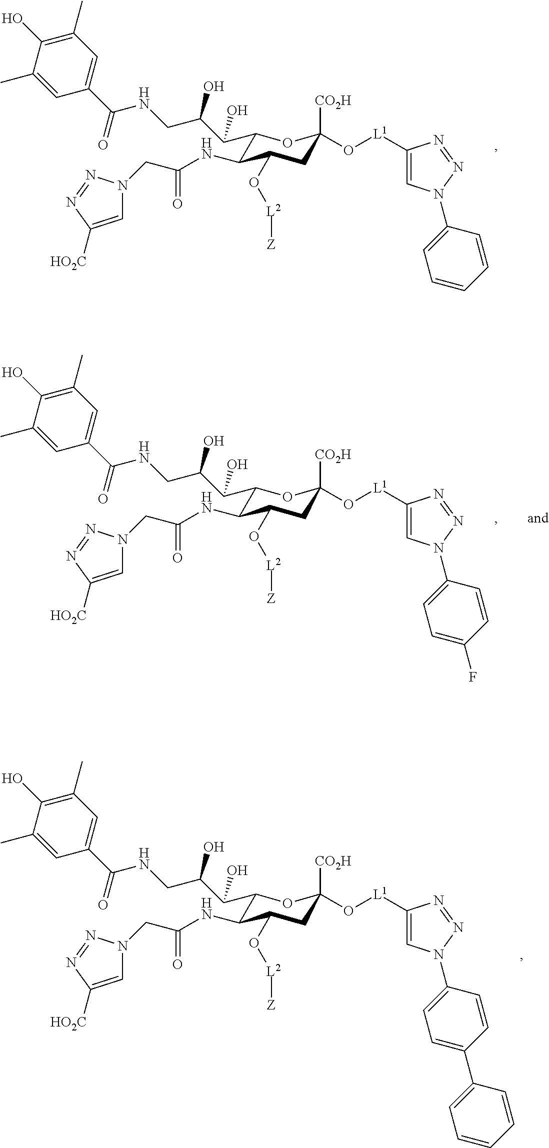

and pharmaceutically acceptable salts of any of the foregoing.

In some embodiments, the compound of Formula (I) is chosen from:

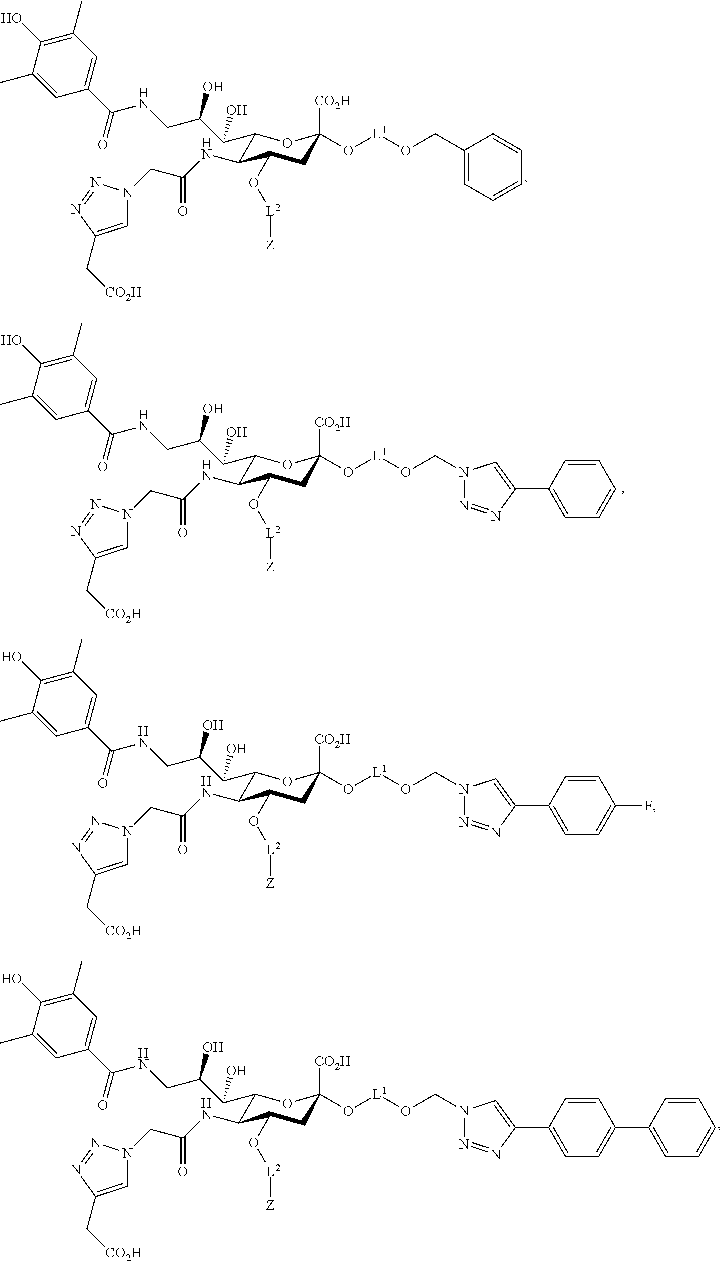

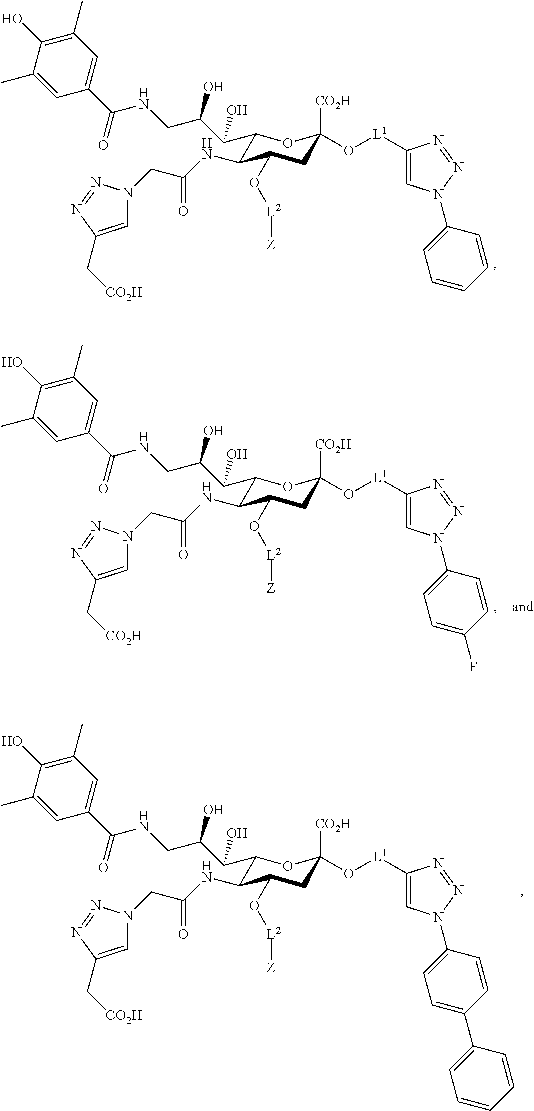

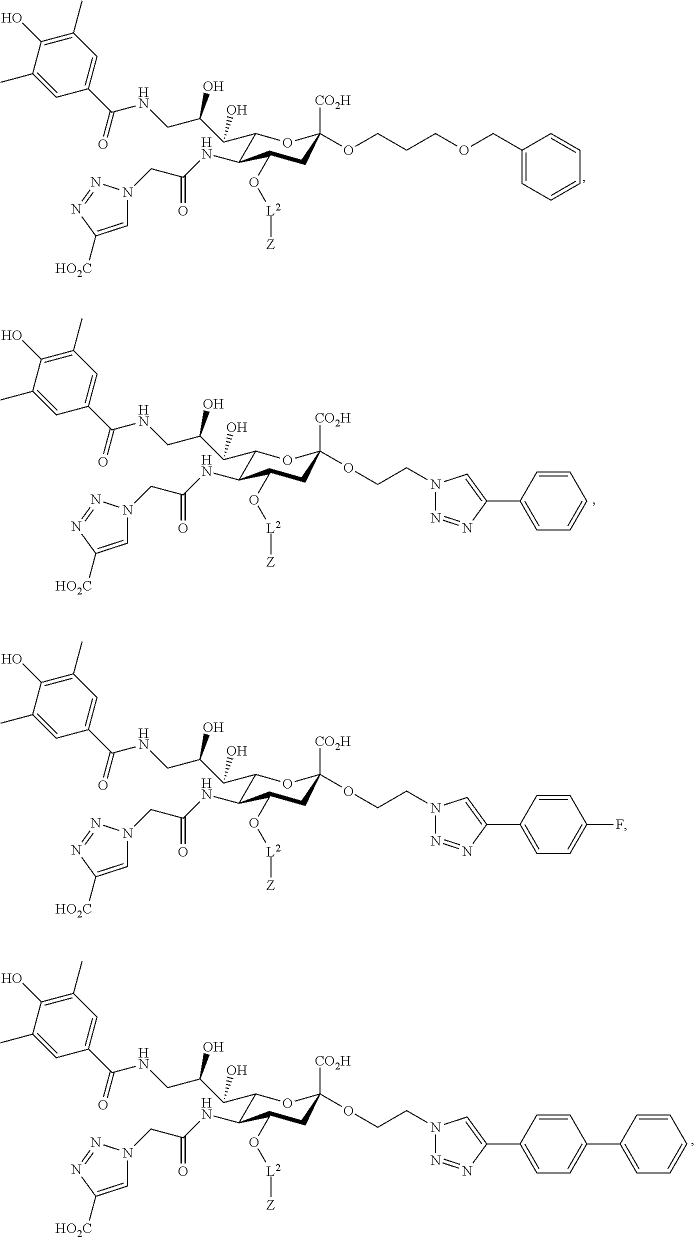

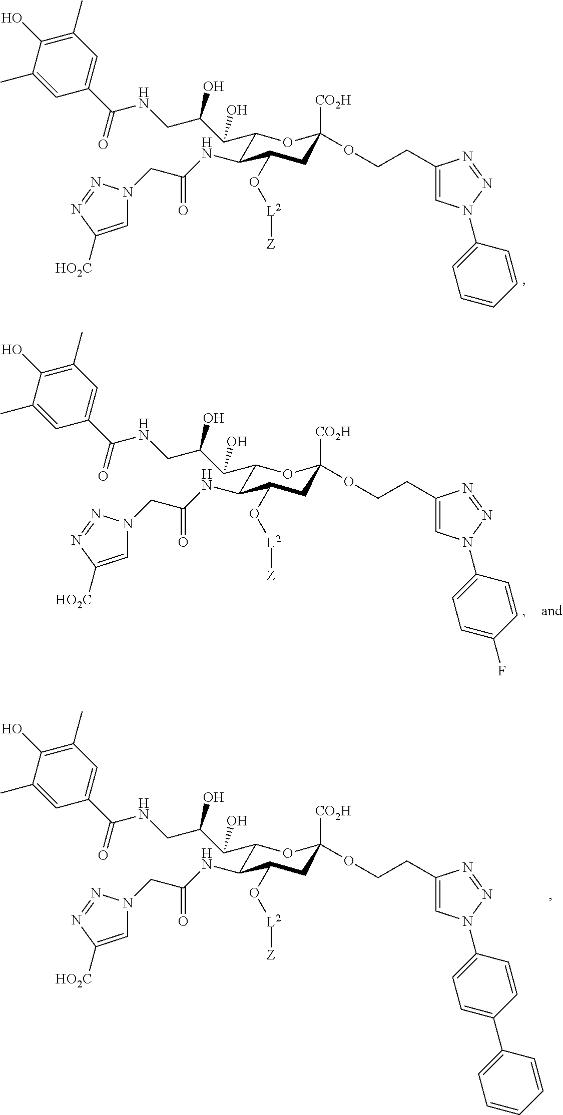

and pharmaceutically acceptable salts of any of the foregoing.

In some embodiments, the compound of Formula (I) is chosen from:

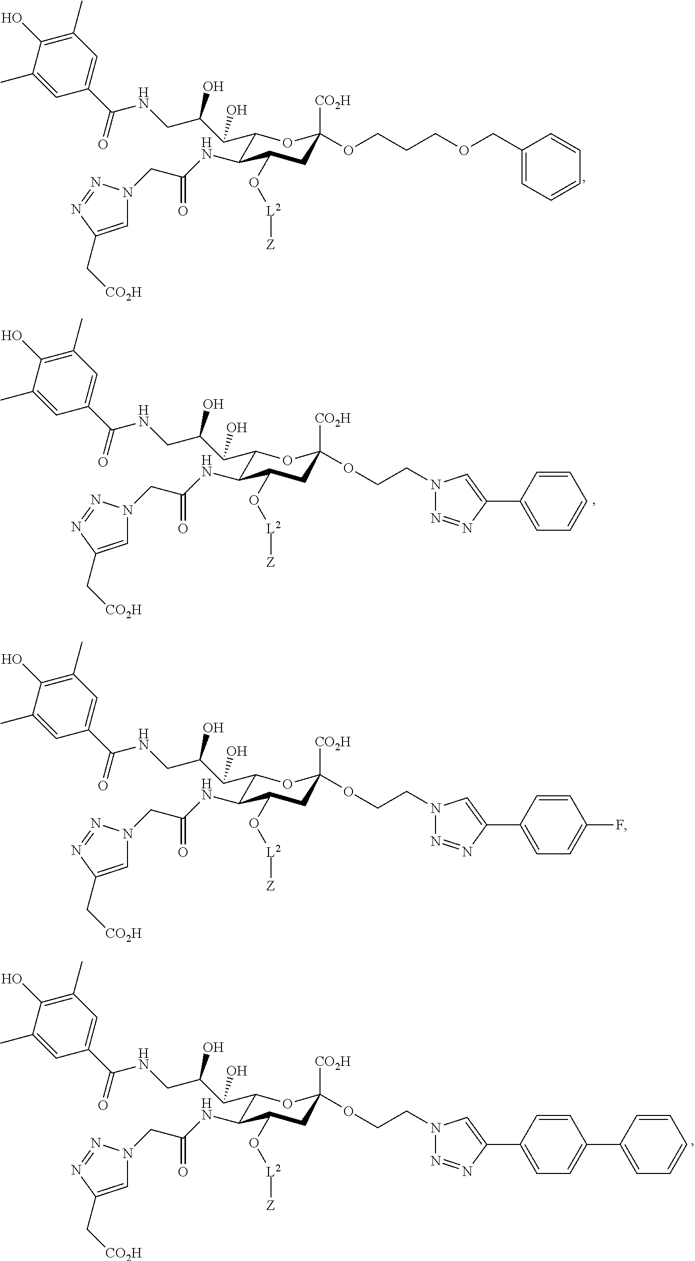

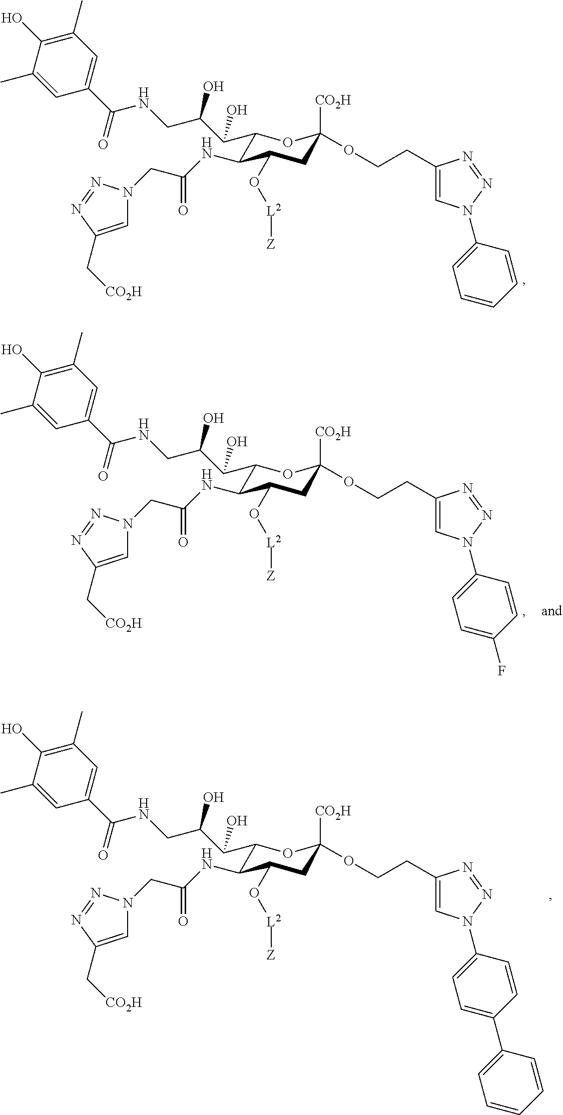

and pharmaceutically acceptable salts of any of the foregoing.

In some embodiments, the compound of Formula (I) is chosen from:

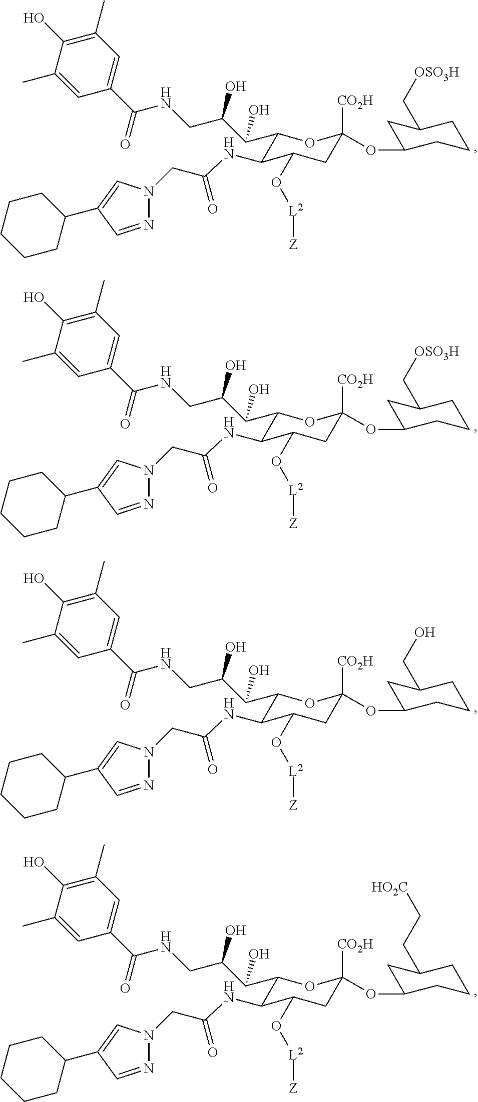

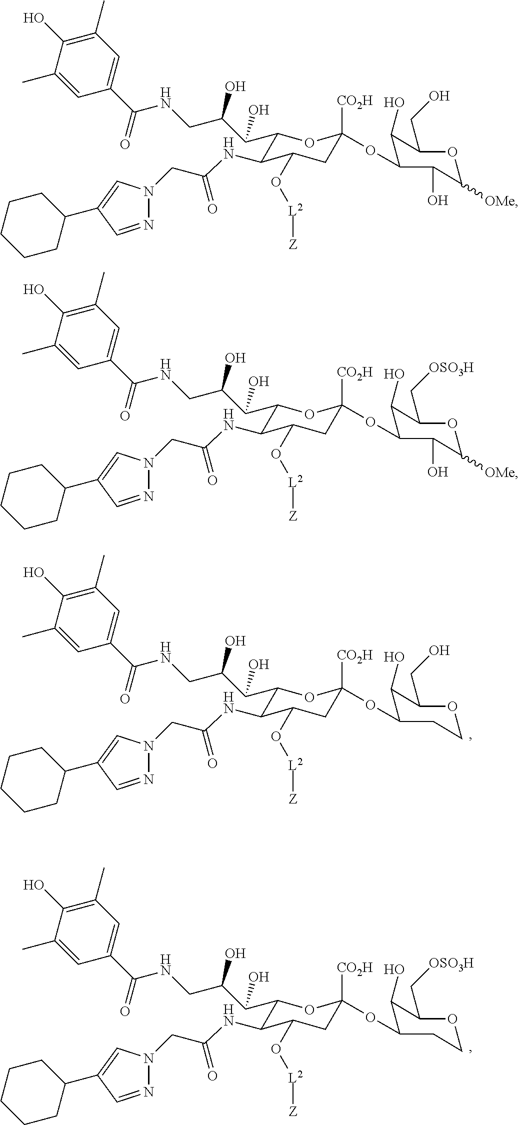

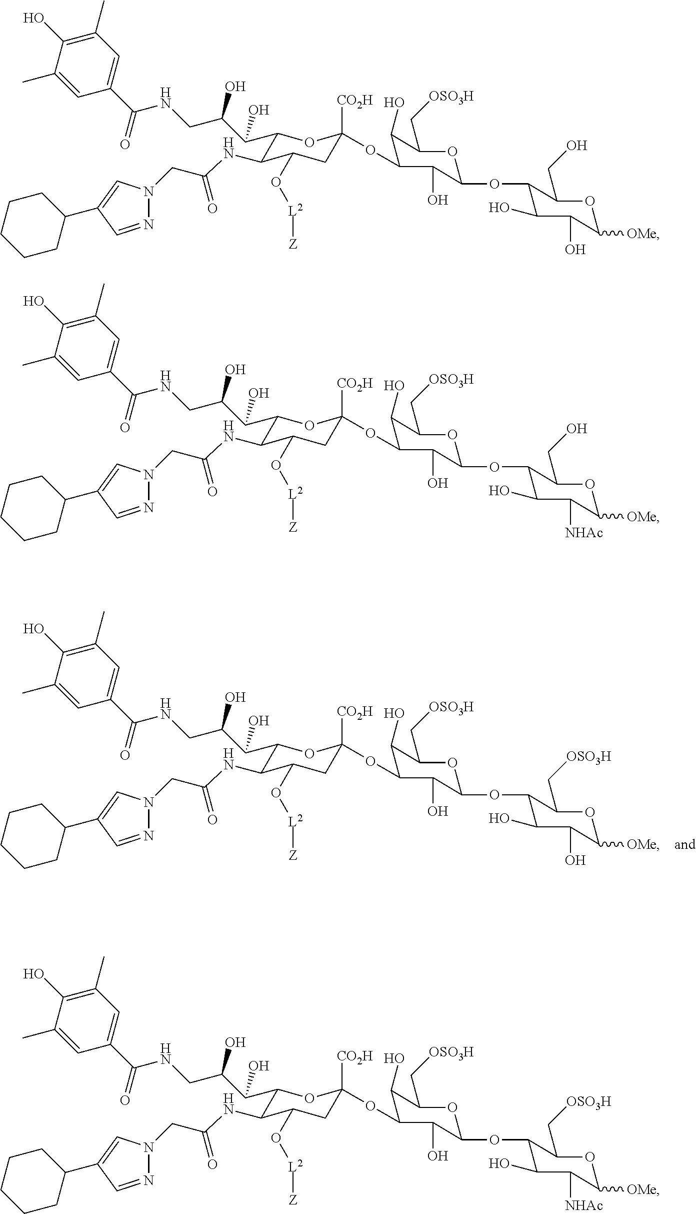

and pharmaceutically acceptable salts of any of the foregoing.

In some embodiments, the compound of Formula (I) is chosen from:

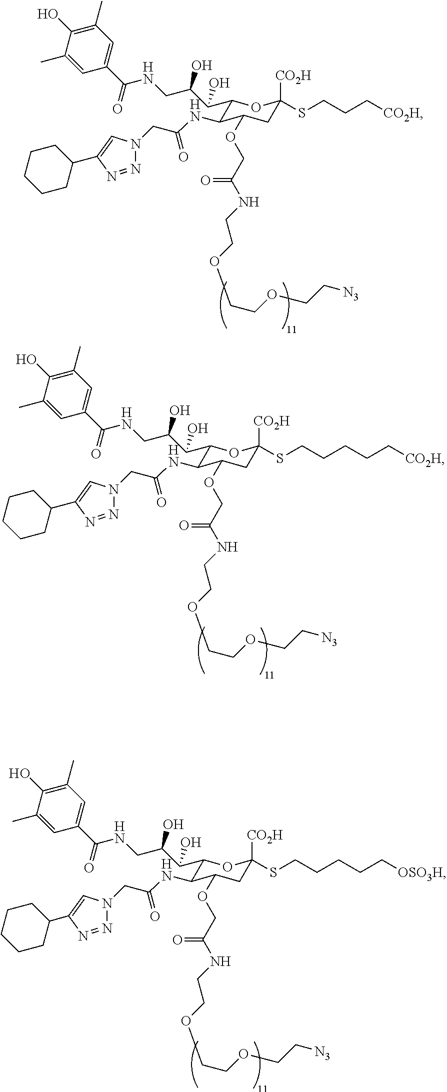

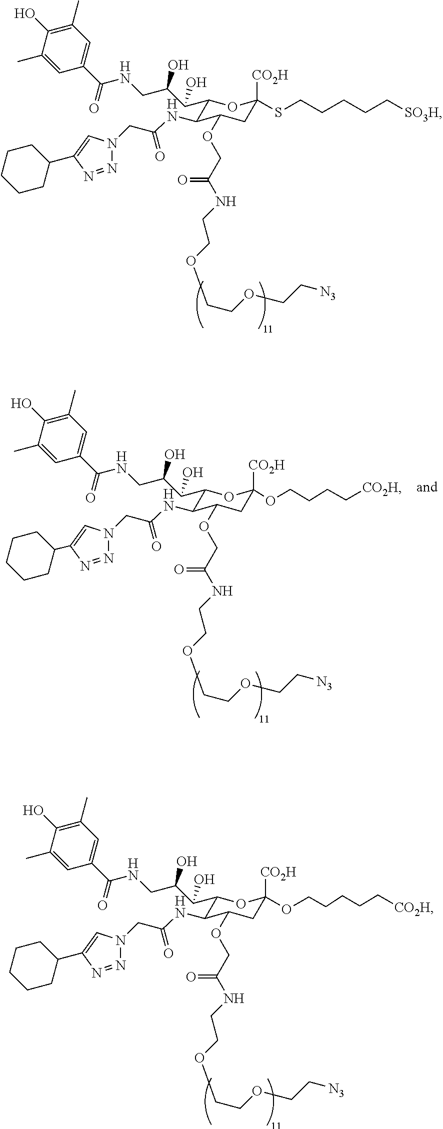

and pharmaceutically acceptable salts of any of the foregoing.

In some embodiments, the compound of Formula (I) is chosen from:

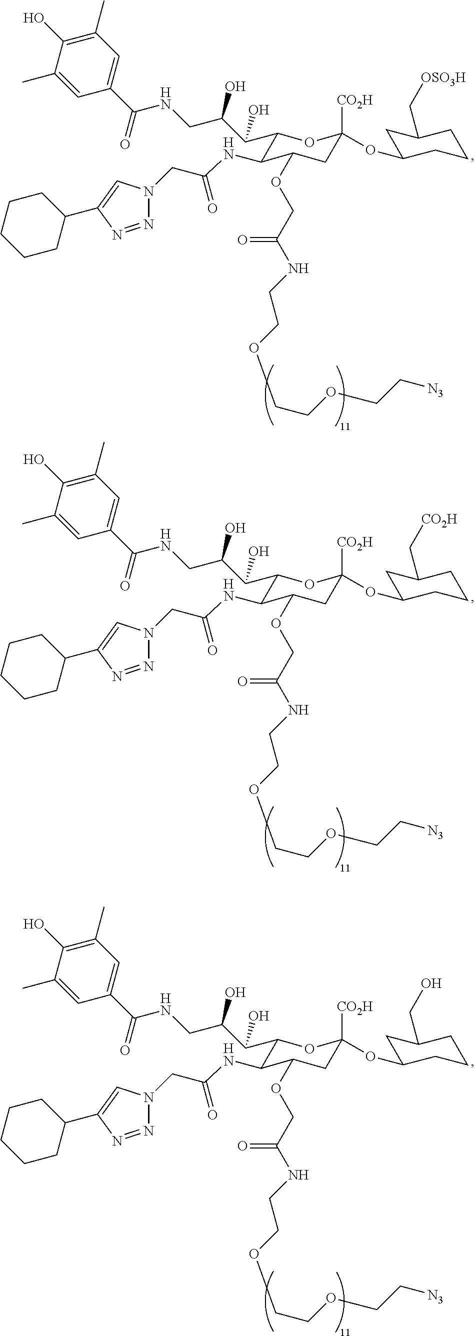

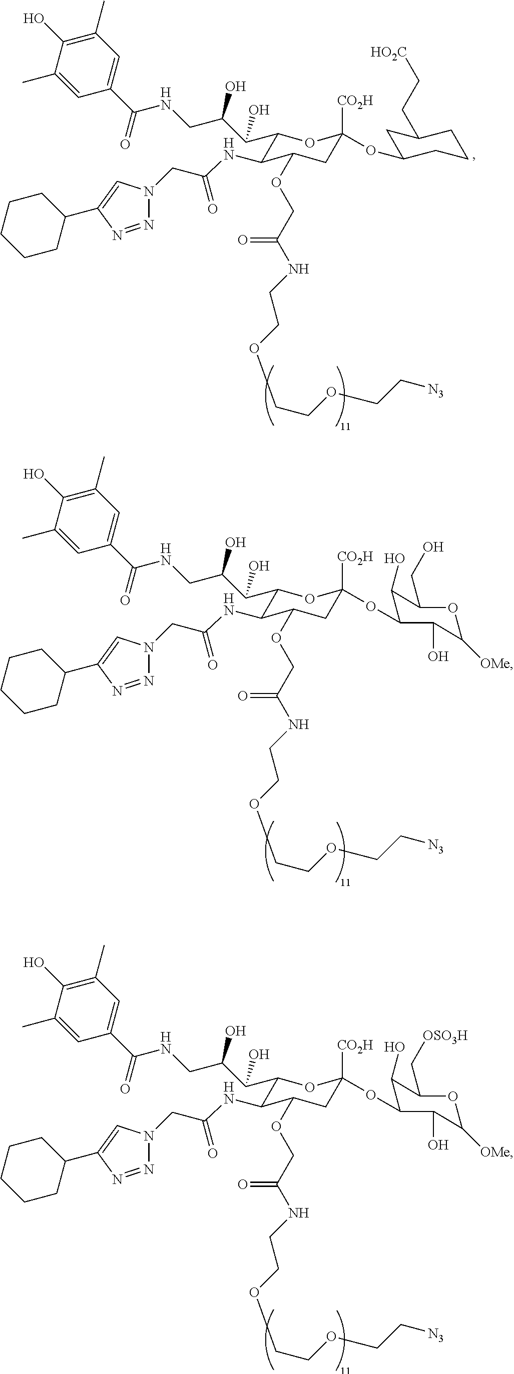

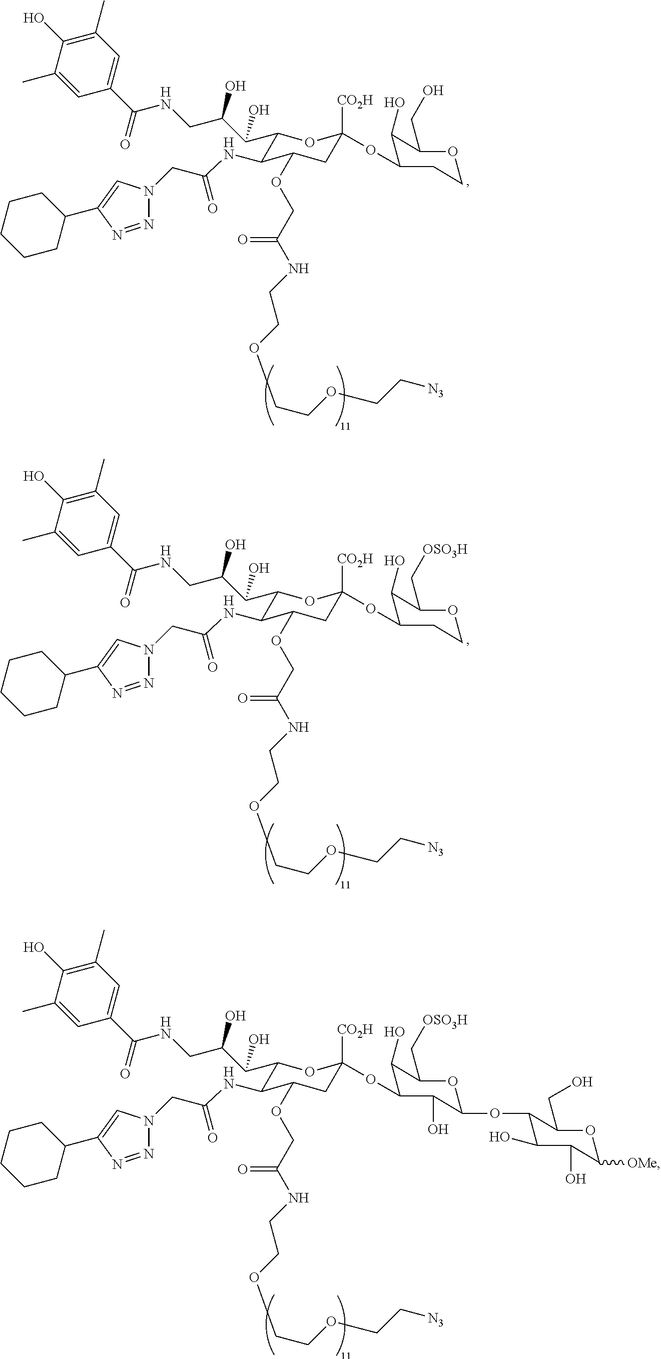

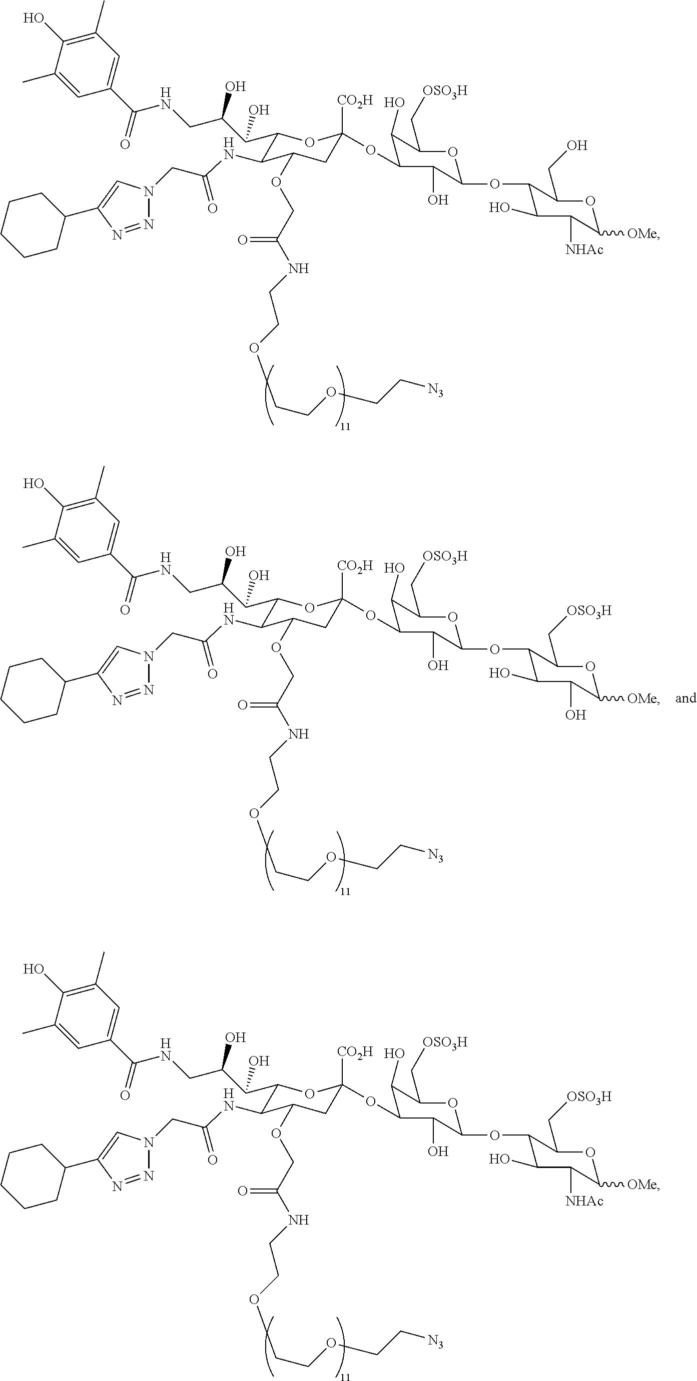

an pamaeuialyacepaleslt oay fhefoeoig

In some embodiments, the compound of Formula (I) is chosen from:

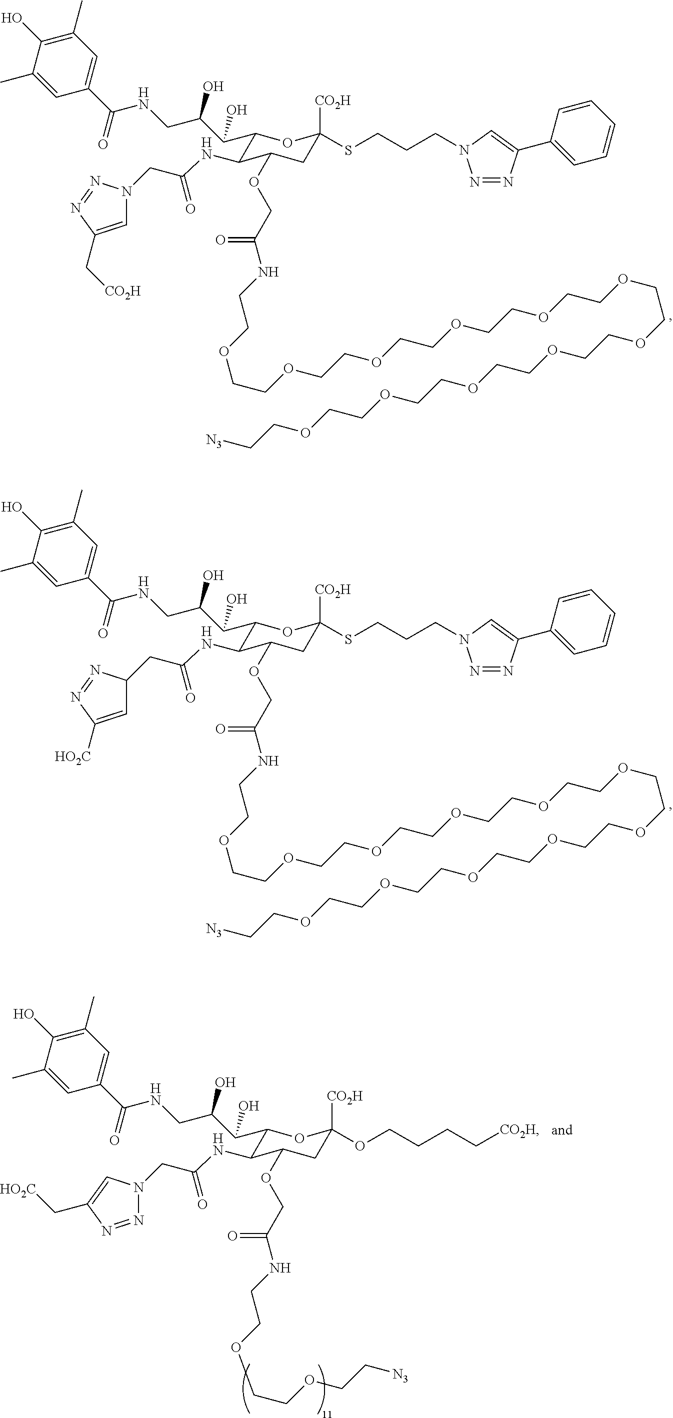

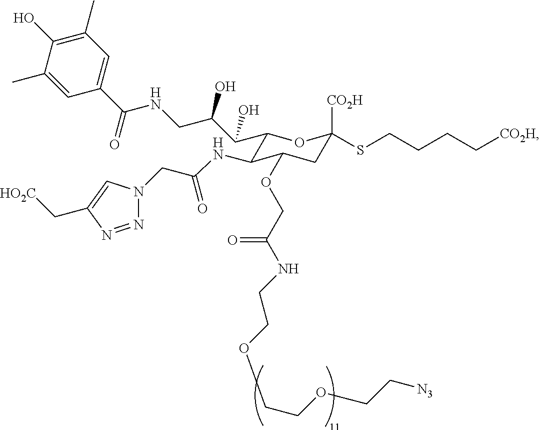

and pharmaceutically acceptable salts of any of the foregoing.

In some embodiments, a process for making CD33 ligand-bearing carriers of Formula (II) is provided, wherein the process comprises reacting or associating a compound of Formula (I) with a compound of Formula (III):

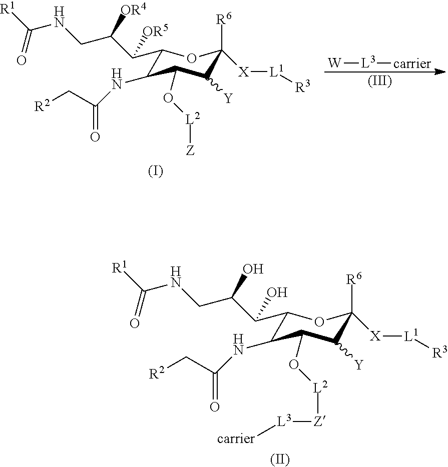

wherein:
R¹, R², R³, R⁴, R⁵, R⁶, L¹, L², X, Y, and Z are as defined above;
L³ is chosen from a bond and linker groups;
W is chosen from lipids, nucleophiles, electrophiles, and groups capable of undergoing a cycloaddition reaction; and
Z′ is a moiety generated by the reaction or association of the Z group of the compound of Formula (I) with the W group of the compound of Formula (III).

In some embodiments, L³ is a bond. In some embodiments, L³ is chosen from linker groups.

In some embodiments, W is chosen from lipids. In some embodiments, W is chosen from glycerol-based lipids. In some embodiments, W is chosen from phospholipids. In some embodiments, W is chosen from symmetric phospholipids. In some embodiments, W is chosen from asymmetric phospholipids. In some embodiments, W is chosen from sphingolipids. In some embodiments, W is chosen from ceramides.

In some embodiments, W is chosen from phospholipids that have 16 to 30 carbon atoms. In some embodiments, W is chosen from phospholipids that have 16, 20, 22, 24, 26, 28, or 30 carbon atoms. In some embodiments, W is chosen from phospholipids that have 16 carbon atoms. In some embodiments, W is chosen from phospholipids that have 18 carbon atoms. In some embodiments, W is chosen from phospholipids that have 20 carbon atoms. In some embodiments, W is chosen from phospholipids that have 22 carbon atoms. In some embodiments, W is chosen from phospholipids that have 24 carbon atoms. In some embodiments, W is chosen from phospholipids that have 26 carbon atoms. In some embodiments, W is chosen from phospholipids that have 28 carbon atoms. In some embodiments, W is chosen from phospholipids that have 30 carbon atoms.

In some embodiments, W is chosen from saturated phospholipids. In some embodiments, W is chosen from unsaturated phospholipids. In some embodiments, W is chosen from phosphatidylcholine, phosphatidyethanolamine, phosphatidic acid, and phosphatidyl inositol. In some embodiments, W is chosen from phospholipid derivatives of 6-cis-octadecenoic, 9-cis-octadecenoic, 9-trans-octadecenoic, 9-cis-12-octadecadienoic, 9-cis-12-cis-15-cis-octadecatrienoic, 11-cis-eicosenoic, 5, 8, 11, 14 (all-cis) eicosatetraenoic, 13-cis-docosenoic, 4, 7, 10, 13, 16, 19 (all-cis) docosahexaenoic, and/or 15-cis-tetracosenoic acids. In some embodiments, W is chosen from phospholipid derivatives of stearoyl, oleoyl, linoleoyl, arachidonoyl, and/or docosahexaenoyl acids.

In some embodiments, W is chosen from nucleophiles. In some embodiments, the nucleophiles are chosen from amines, mercaptans, and alcohols. In some embodiments, W is chosen from amine groups.

In some embodiments, W is chosen from electrophiles. In some embodiments, the electrophiles are chosen from epoxides, aziridines, episulfides, sulfates, sulfonates, carbonates, lactones, lactams, halides, acid halides, and esters. In some embodiments, the electrophiles are chosen from epoxides. In some embodiments, the electrophiles are chosen from aziridines. In some embodiments, the electrophiles are chosen from episulfides. In some embodiments, the electrophiles are chosen from sulfates. In some embodiments, the electrophiles are chosen from sulfonates. In some embodiments, the electrophiles are chosen from carbonates. In some embodiments, the electrophiles are chosen from lactones. In some embodiments, the electrophiles are chosen from lactams. In some embodiments, the electrophiles are chosen from halides. In some embodiments, the electrophiles are chosen from acid halides. In some embodiments, the electrophiles are chosen from esters. In some embodiments, the esters are chosen from activated esters.

In some embodiments, W is chosen from esters. In some embodiments, the esters are chosen from anhydrides, carboxylic acid-succinimides, carboxylic acid phosphoesters, and carboxylic acid imidazolides. In some embodiments, W is chosen from esters formed with t-butanol, p-nitrophenol, 2,4-dinitrophenol, trichlorophenol, 1-hydroxy-1H-benzotriazole, 1-hydroxy-6-chloro-1H-benzotriazole, and N-hydroxysuccinimide.

In some embodiments, W is chosen from —C(═O)T²⁰ groups, wherein T²⁰ is chosen from H, —OH, —O^{t}Bu, C₂₋₈ alkenyl, C₁₋₈ haloalkyl, C₁₋₈ alkoxy, halo, C₆₋₁₈ aryloxy, C₁₋₁₃ heteroaryloxy, and C₂₋₁₂ heterocyclyloxy groups and wherein the C₁₋₈ alkoxy, C₆₋₁₈ aryloxy, C₁₋₃ heteroaryloxy, and C₂₋₁₂ heterocyclyloxy groups are optionally substituted with at least one halo group.

In some embodiments, W is chosen from groups capable of undergoing a cycloaddition reaction. In some embodiments, the groups capable of undergoing a cycloaddition reaction are chosen from alkenes, alkynes, dienes, dienophiles, 1,3-dipoles, and 1,3-dipolarophiles.

In some embodiments, the dienophiles are chosen from —C(═O)T²¹ groups, wherein T²¹ is chosen from alkene and alkyne groups, wherein said alkene and alkyne groups are optionally substituted with C₁₋₈ alkoxy, —C(═O)H, —C(═O)OT²², and —C(═O)NT²²T²³ groups, wherein T²² and T²³ which may be identical or different, are independently chosen from H and C₁₋₈. alkyl groups, or T²² and T²³ join together along with the nitrogen to which they are attached to form an optionally substituted, saturated or unsaturated, 3-10 membered ring.

In some embodiments, the 1,3-dipoles are chosen from nitrile ylides, nitrile imines, nitrile oxides, diazolalkanes, azides, azomethine ylides, azomethine imines, nitrones, carbonyl ylides, carbonyl imines, and carbonyl oxides.

In some embodiments, W is chosen from alkene and alkyne groups. In some embodiments, W is chosen from alkene groups. In some embodiments, W is chosen from alkyne groups. In some embodiments, W is —CH═CH₂. In some embodiments, W is —C≡CH.

In some embodiments, W is chosen from dienes and dienophiles. In some embodiments, W is chosen from dienes. In some embodiments, W is chosen from dienophiles.

In some embodiments, W is chosen from 1,3-dipoles and 1,3-dipolarophiles. In some embodiments, W is chosen from 1,3-dipoles. In some embodiments, W is chosen from 1,3-dipolarophiles.

In some embodiments, W is chosen from —N₃, —NH₂, —SH, —OH, —Cl, —Br, —I, —CH═CH₂, —C≡CH,

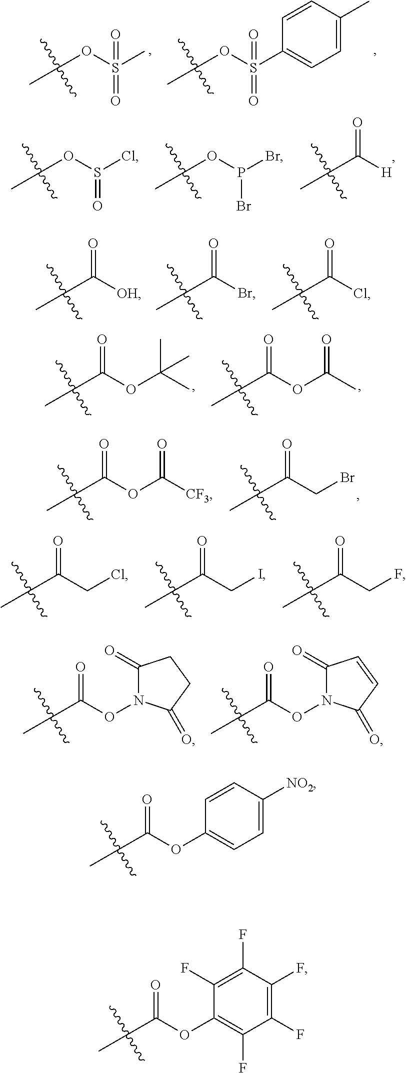

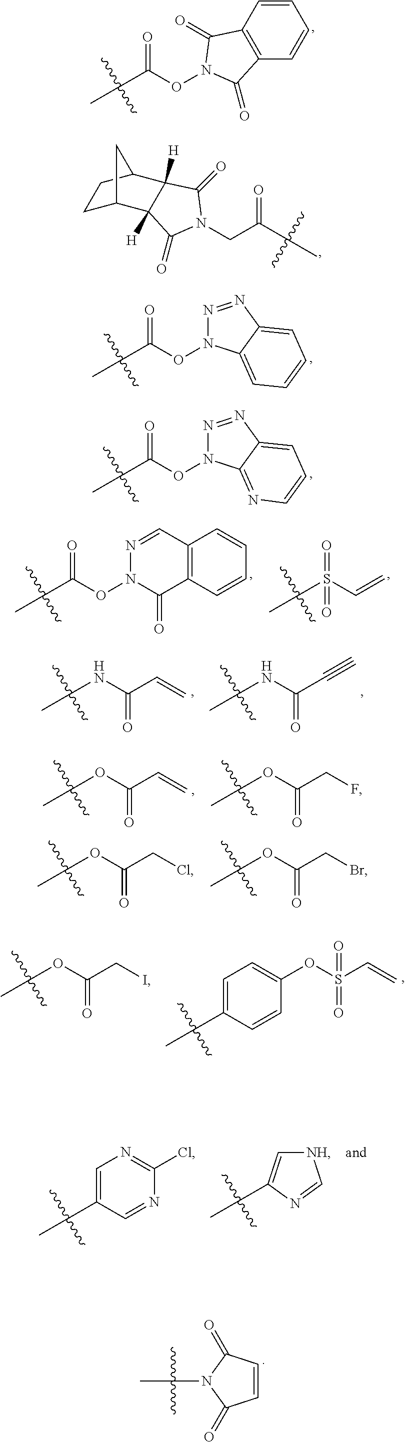

In some embodiments, W is —N.

In some embodiments, W is chosen from —NH₂, —SH, and —OH. In some embodiments, W is —NH₂. In some embodiments, W is —SH. In some embodiments, W is —OH.

In some embodiments, W is —C(═O)H. In some embodiments, W is —C(═O)OH. In some embodiments, W is —C(═O)C₁. In some embodiments, W is —C(═O)O^{t}Bu.

In some embodiments, a process for making CD33 ligand-bearing carriers of Formula (II) is provided, wherein the process comprises reacting or associating a compound of Formula (I) with a compound of Formula (III):

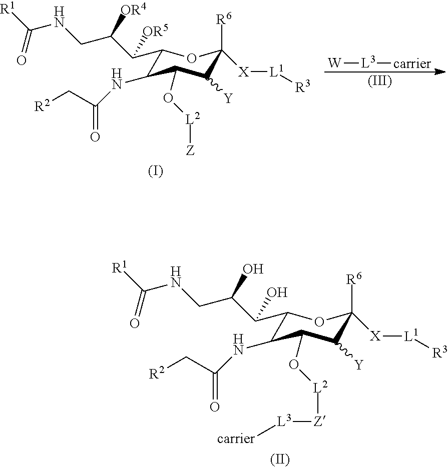

wherein;
R¹, R², R³, R⁴, R⁵, R⁶, L¹, L², L³, X, and Y are as defined above; and

| Z | W | Z′ |
|---|---|---|
| Lipid | lipid | lipid-lipid non-covalent association |
| —N₃ | HC═C— | 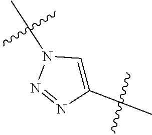 [SMILES: *c1cn(*)nn1] |
| | HC≡C— | 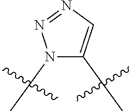 [SMILES: *c1cnnn1*] |
| —N₃ | 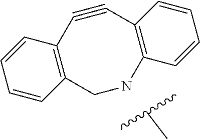 | 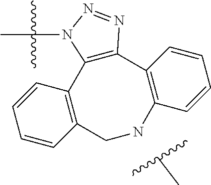 |
| —N₃ | 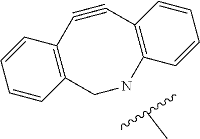 | 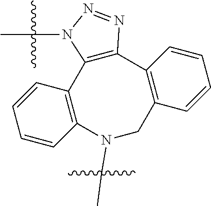 [SMILES: *N1Cc2ccccc2-c2nnn(*)c2-c2ccccc21] |
| —C≡CH | N₃— | 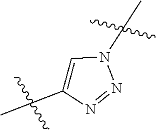 [SMILES: *c1cn(*)nn1] |
| —C≡CH | | 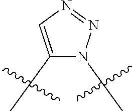 [SMILES: *c1cnnn1*] |
| —NH₂ | HO(O═)C— | —NH(O═)C— |
| | Cl(O═)C— | |
| | 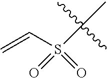 [SMILES: *S(=O)(=O)C=C] | 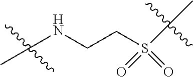 [SMILES: *NCCS(*)(=O)=O] |
| | 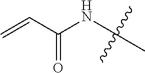 [SMILES: *NC(=O)C=C] | 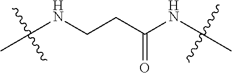 [SMILES: *NCCC(=O)N*] |
| | 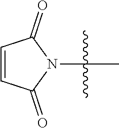 [SMILES: *N1C(=O)C=CC1=O] | 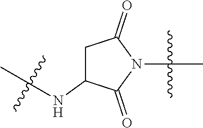 [SMILES: *NC1CC(=O)N(*)C1=O] |
| | BrH₂C— | —NHCH₂— |
| | 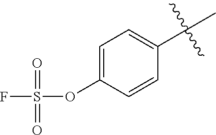 [SMILES: *c1ccc(OS(=O)(=O)F)cc1] | 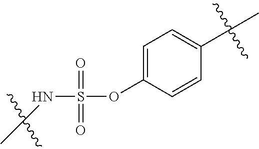 [SMILES: *NS(=O)(=O)Oc1ccc(*)cc1] |
| | 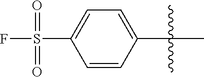 [SMILES: *c1ccc(S(=O)(=O)F)cc1] | 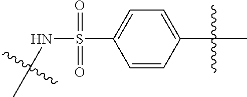 [SMILES: *NS(=O)(=O)c1ccc(*)cc1] |
| | 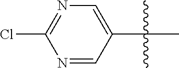 [SMILES: *c1cnc(Cl)nc1] | 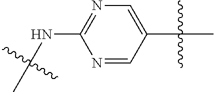 [SMILES: *Nc1ncc(*)cn1] |
| | 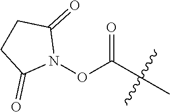 [SMILES: *C(=O)ON1C(=O)CCC1=O] | 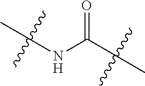 [SMILES: *NC(*)=O] |
| —SH | 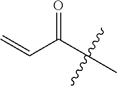 [SMILES: *C(=O)C=C] | 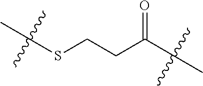 [SMILES: *SCCC(*)=O] |
| | 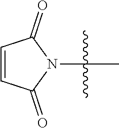 [SMILES: *N1C(=O)C=CC1=O] | 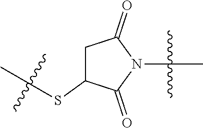 [SMILES: *SC1CC(=O)N(*)C1=O] |
| | 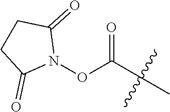 [SMILES: *C(=O)ON1C(=O)CCC1=O] | 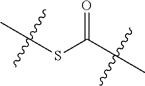 [SMILES: *SC(*)=O] |
| | 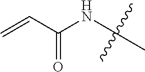 [SMILES: *NC(=O)C=C] | 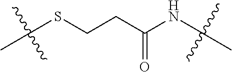 [SMILES: *NC(=O)CCS*] |
| | 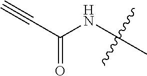 [SMILES: *NC(=O)C#C] | 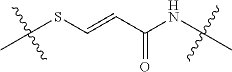 [SMILES: *NC(=O)/C=C/S*] |
| | 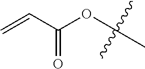 [SMILES: *OC(=O)C=C] | 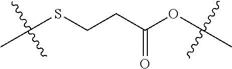 [SMILES: *OC(=O)CCS*] |
| | 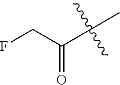 [SMILES: *C(=O)CF] | 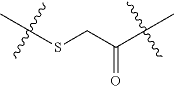 [SMILES: *SCC(*)=O] |
| | 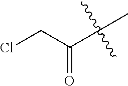 [SMILES: *C(=O)CCl] | |
| | 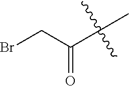 [SMILES: *C(=O)CBr] | |
| | 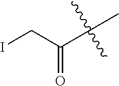 [SMILES: *C(=O)CI] | |
| | 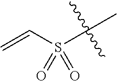 [SMILES: *S(=O)(=O)C=C] | 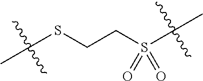 [SMILES: *SCCS(*)(=O)=O] |
| | BrH₂C— | —SH₂C— |
| | 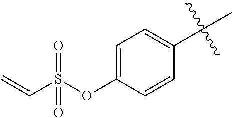 [SMILES: *c1ccc(OS(=O)(=O)C=C)cc1] | 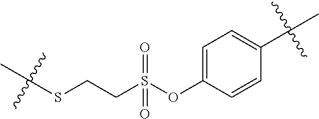 [SMILES: *SCCS(=O)(=O)Oc1ccc(*)cc1] |
| —OH | 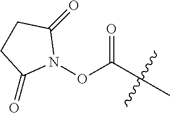 [SMILES: *C(=O)ON1C(=O)CCC1=O] | 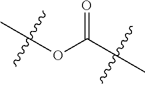 [SMILES: *OC(*)=O] |
| | 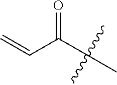 [SMILES: *C(=O)C=C] | 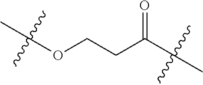 [SMILES: *OCCC(*)=O] |
| | 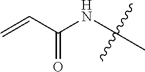 [SMILES: *NC(=O)C=C] | 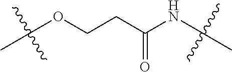 [SMILES: *NC(=O)CCO*] |
| | 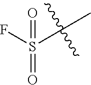 [SMILES: *S(=O)(=O)F] | 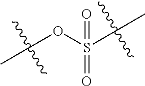 [SMILES: *OS(*)(=O)=O] |
| | 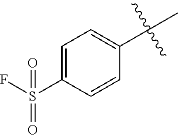 [SMILES: *c1ccc(S(=O)(=O)F)cc1] | 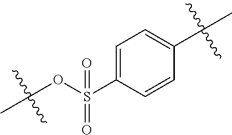 [SMILES: *OS(=O)(=O)c1ccc(*)cc1] |
| | 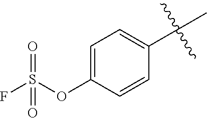 [SMILES: *c1ccc(OS(=O)(=O)F)cc1] | 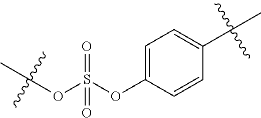 [SMILES: *OS(=O)(=O)Oc1ccc(*)cc1] |
| 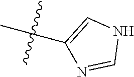 [SMILES: *c1c[nH]cn1] | BrH₂C— | 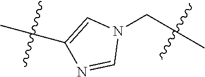 [SMILES: *Cn1cnc(*)c1] |
| | 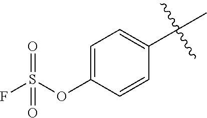 [SMILES: *c1ccc(OS(=O)(=O)F)cc1] | 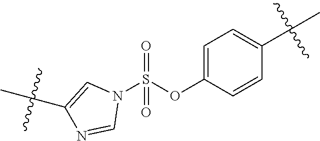 [SMILES: *c1ccc(OS(=O)(=O)n2cnc(*)c2)cc1] |
| —C(═O)OH | H₂N— | —C(═O)HN— |
| —C(═O)Cl | | —C(═O)HN— |
| 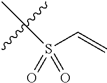 [SMILES: *S(=O)(=O)C=C] | H₂N— | 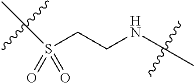 [SMILES: *NCCS(*)(=O)=O] |
| | HS— | 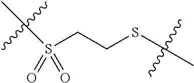 [SMILES: *SCCS(*)(=O)=O] |
| 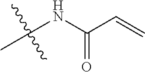 [SMILES: *NC(=O)C=C] | H₂N— | 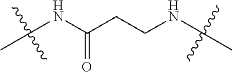 [SMILES: *NCCC(=O)N*] |
| | HS— | 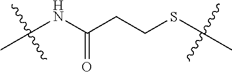 [SMILES: *NC(=O)CCS*] |
| | HO— | 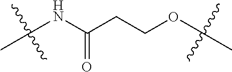 [SMILES: *NC(=O)CCO*] |
| 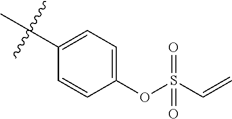 [SMILES: *c1ccc(OS(=O)(=O)C=C)cc1] | H₂N— | 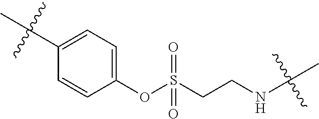 [SMILES: *NCCS(=O)(=O)Oc1ccc(*)cc1] |
| | HS— | 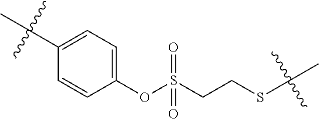 [SMILES: *SCCS(=O)(=O)Oc1ccc(*)cc1] |
| | HO— | 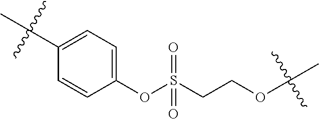 [SMILES: *OCCS(=O)(=O)Oc1ccc(*)cc1] |
| 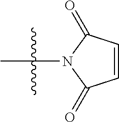 [SMILES: *N1C(=O)C=CC1=O] | H₂N— | 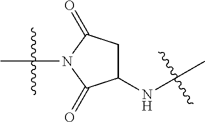 [SMILES: *NC1CC(=O)N(*)C1=O] |
| | HS— | 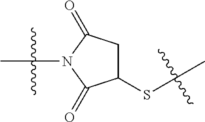 [SMILES: *SC1CC(=O)N(*)C1=O] |
| —CH₂Br | H₂N— | —CH₂NH— |
| | HS— | —CH₂S— |
| | 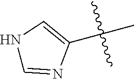 [SMILES: *c1c[nH]cn1] | 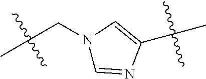 [SMILES: *Cn1cnc(*)c1] |
| 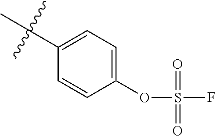 [SMILES: *c1ccc(OS(=O)(=O)F)cc1] | H₂N— | 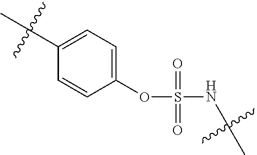 [SMILES: *NS(=O)(=O)Oc1ccc(*)cc1] |
| | HO— | 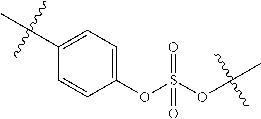 [SMILES: *OS(=O)(=O)Oc1ccc(*)cc1] |
| | 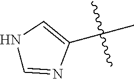 [SMILES: *c1c[nH]cn1] | 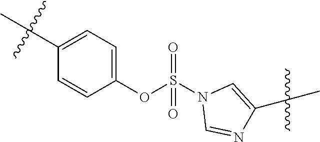 [SMILES: *c1ccc(OS(=O)(=O)n2cnc(*)c2)cc1] |
| 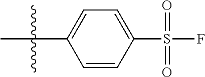 [SMILES: *c1ccc(S(=O)(=O)F)cc1] | H₂N— | 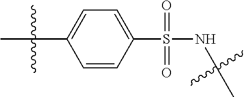 [SMILES: *NS(=O)(=O)c1ccc(*)cc1] |
| | HO— | 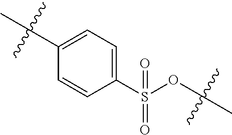 [SMILES: *OS(=O)(=O)c1ccc(*)cc1] |
| 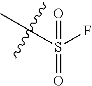 [SMILES: *S(=O)(=O)F] | HO— | 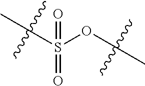 [SMILES: *OS(*)(=O)=O] |
| 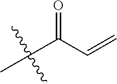 [SMILES: *C(=O)C=C] | HO— | 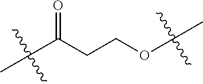 [SMILES: *OCCC(*)=O] |
| | HS— | 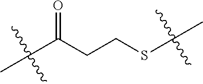 [SMILES: *SCCC(*)=O] |
| 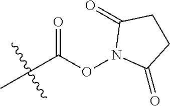 [SMILES: *C(=O)ON1C(=O)CCC1=O] | H₂N— | 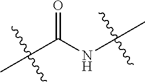 [SMILES: *NC(*)=O] |
| | HS— | 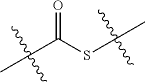 [SMILES: *SC(*)=O] |
| | HO— | 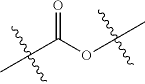 [SMILES: *OC(*)=O] |
| 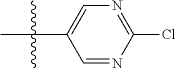 [SMILES: *c1cnc(Cl)nc1] | H₂N— | 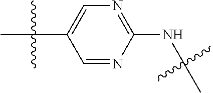 [SMILES: *Nc1ncc(*)cn1] |
| 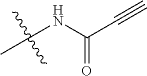 [SMILES: *NC(=O)C#C] | HS— | 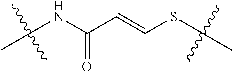 [SMILES: *NC(=O)/C=C/S*] |
| 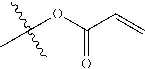 [SMILES: *OC(=O)C=C] | | 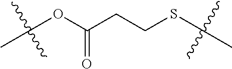 [SMILES: *OC(=O)CCS*] |
| 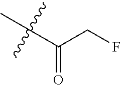 [SMILES: *C(=O)CF] | | 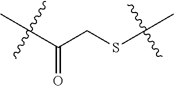 [SMILES: *SCC(*)=O] |
| 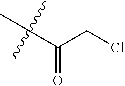 [SMILES: *C(=O)CCl] | | 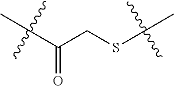 [SMILES: *SCC(*)=O] |
| 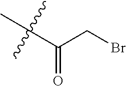 [SMILES: *C(=O)CBr] | | 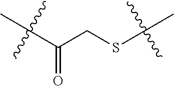 [SMILES: *SCC(*)=O] |
| 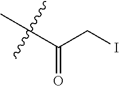 [SMILES: *C(=O)CI] | | 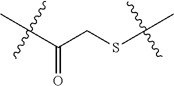 [SMILES: *SCC(*)=O] |

In some embodiments, the carriers are chosen from particles, nanoparticles, liposomes, beads, proteins, polysaccharides, lipids, and combinations thereof. In some embodiments, the carriers are chosen from lipid-based, protein-based, nucleic acid based, and carbohydrate-based carriers. In some embodiments, the carriers are chosen from carriers composed of polymer and/or non-polymer molecules. In some embodiments, the carriers are chosen from macromolecular carriers. In some embodiments, the carriers comprise crosslinking chains of molecules. In some embodiments, the crosslinking chains of molecules are chosen from nucleic acids. In some embodiments, the carriers are chosen from polyamino-based carriers.

Liposomes bearing Siglec ligands can be prepared as described herein and other sources such as PCT Publication No. WO2012/018377 and WO2007/056525 and U.S. Patent Appl. Publication No. US20190151444, which are incorporated by reference herein in their entireties.

In some embodiments, the carriers are chosen from lipid-based nanoparticles, polymeric nanoparticles, metallic nanoparticles, surfactant-based emulsion, dendrimers, and nanoparticles.

In some embodiments, the carriers are formed by self-assembly. In some embodiments, self-assembly occurs in vitro. In some embodiments, self-assembly occurs in vivo. In some embodiments, the carriers are formed using amphiphilic biomaterials which orient themselves with respect to one another to form carriers of predictable dimension, constituents, and placement of constituents.

In some embodiments, the carriers are chosen from carriers having a mean geometric diameter that is less than 500 nm. In some embodiments, the carriers are chosen from carriers having a mean geometric diameter that is greater than 50 nm but less than 500 nm.

In some embodiments, the carriers are nanoparticles.

In embodiments, the Z group of Formula (I) is a lipid and the carrier is chosen from liposomes.

In some embodiments, the compound of Formula (II) is chosen from:

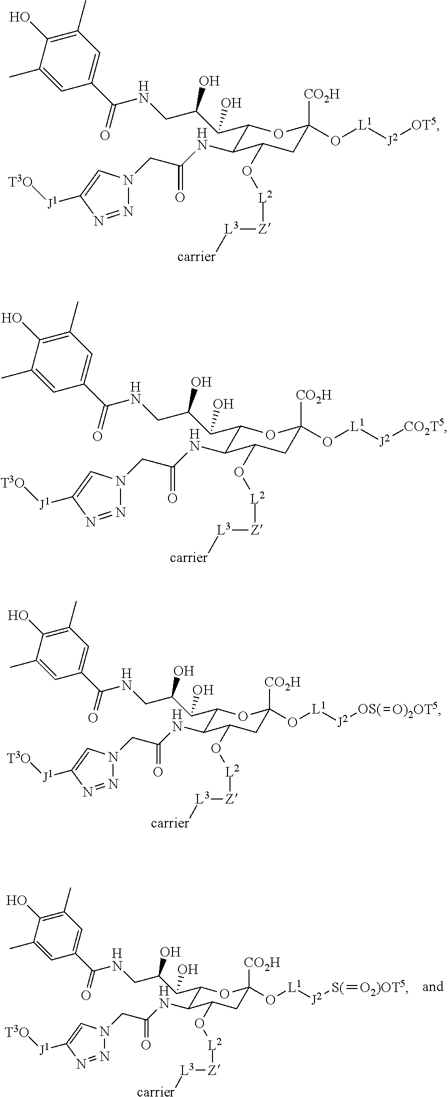

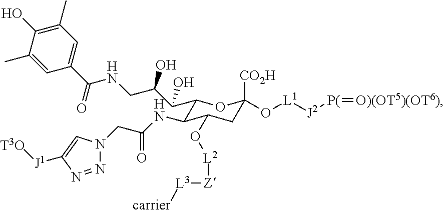

and pharmaceutically acceptable salts of any of the foregoing.

In some embodiments, the compound of Formula (II) is chosen from:

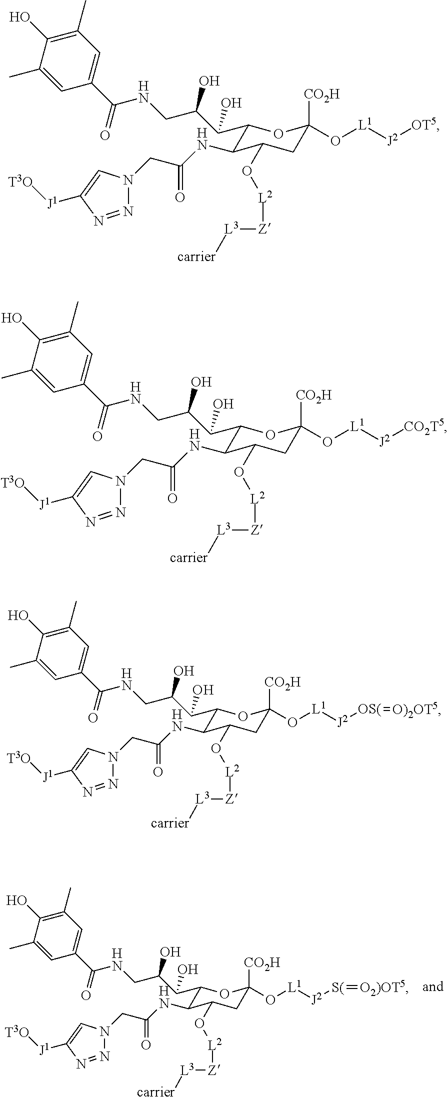

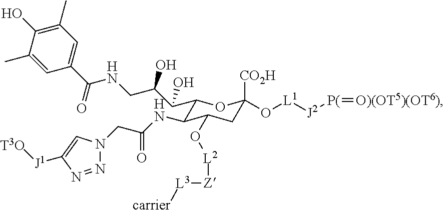

and pharmaceutically acceptable salts of any of the foregoing.

In some embodiments, the compound of Formula (II) is chosen from:

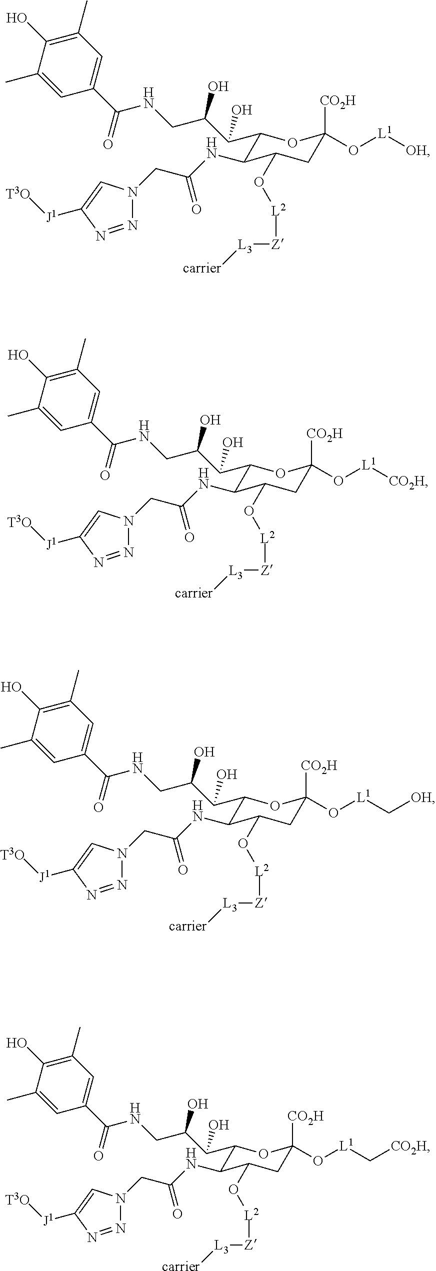

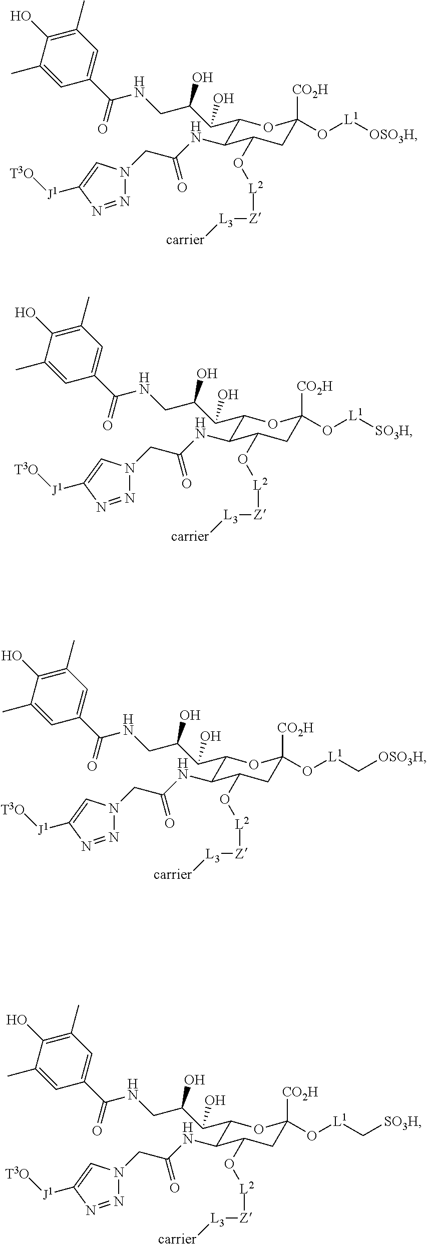

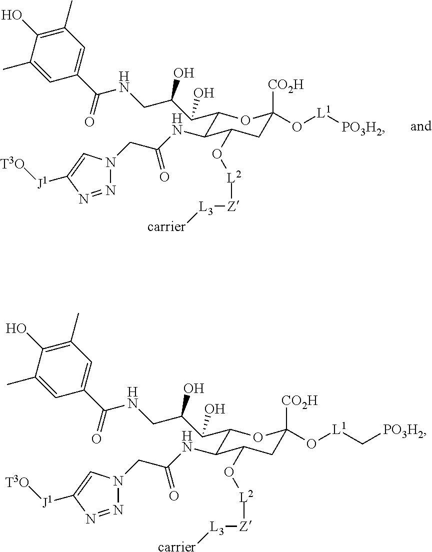

and pharmaceutically acceptable salts of any of the foregoing.

In some embodiments, the compound of Formula (II) is chosen from:

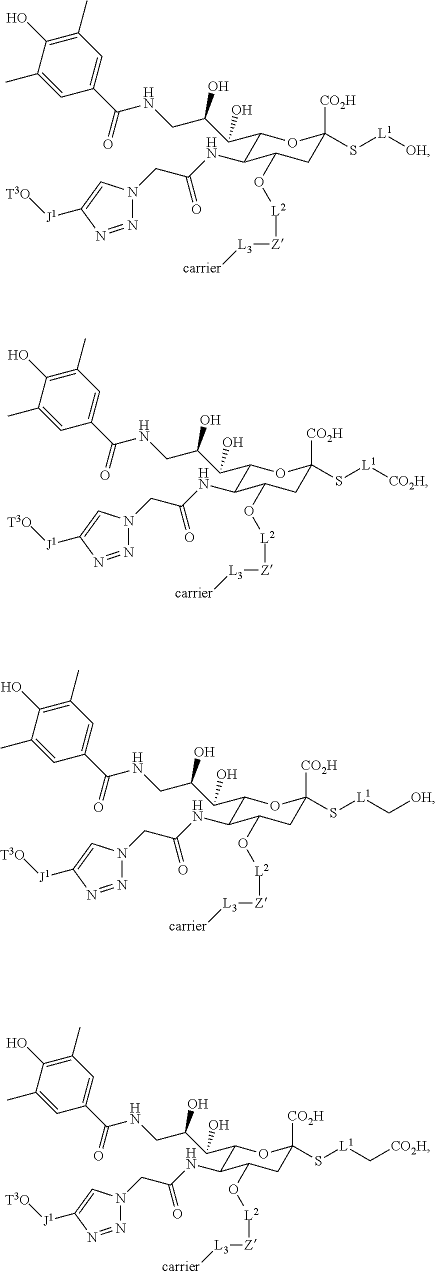

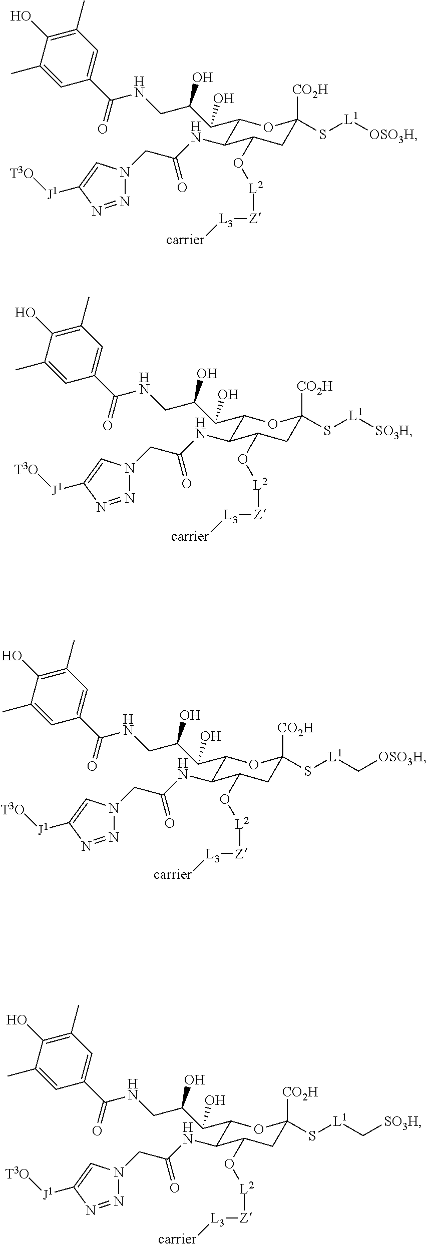

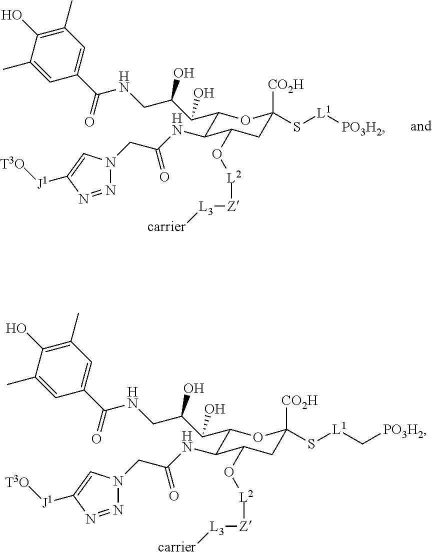

and pharmaceutically acceptable salts of any of the foregoing.

In some embodiments, the compound of Formula (II) is chosen from:

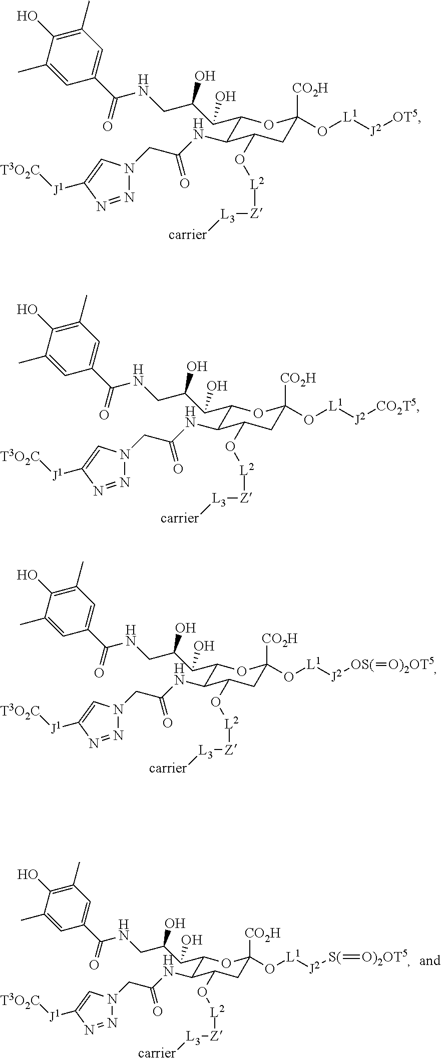

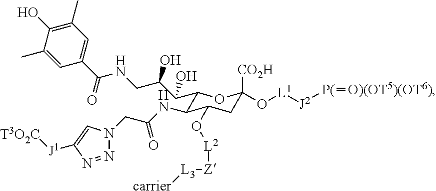

and pharmaceutically acceptable salts of any of the foregoing.

In some embodiments, the compound of Formula (II) is chosen from:

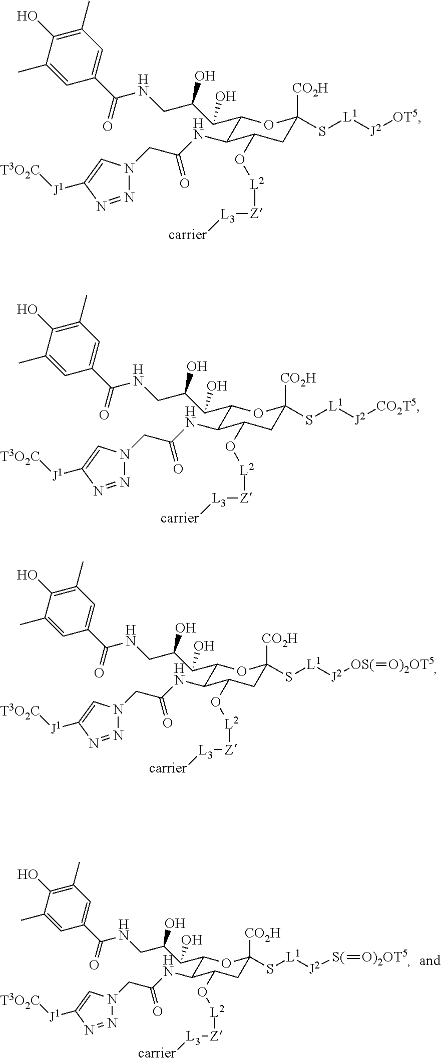

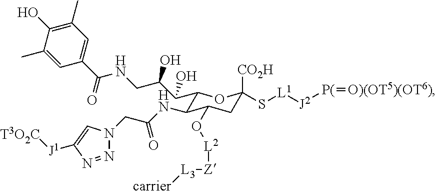

and pharmaceutically acceptable salts of any of the foregoing.

In some embodiments, the compound of Formula (II) is chosen from:

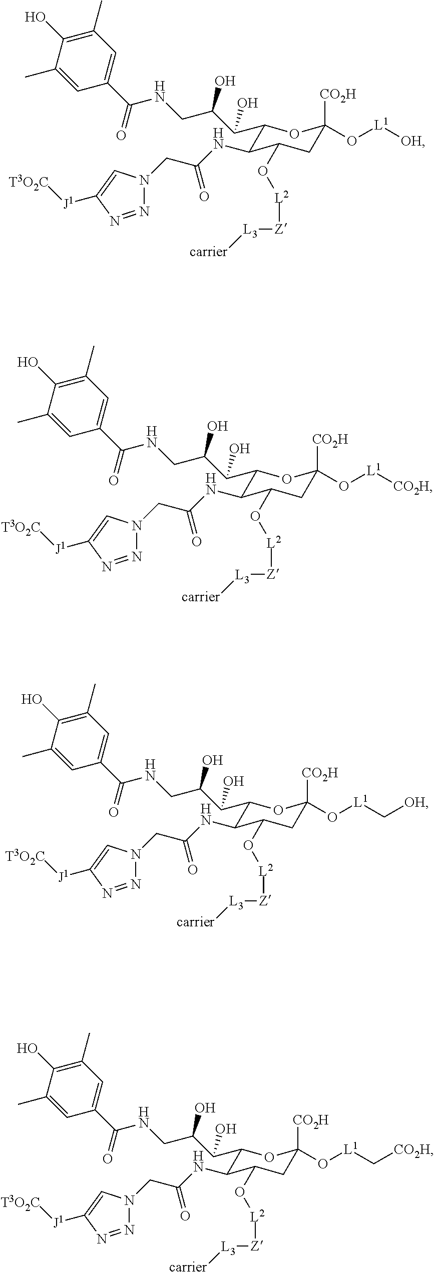

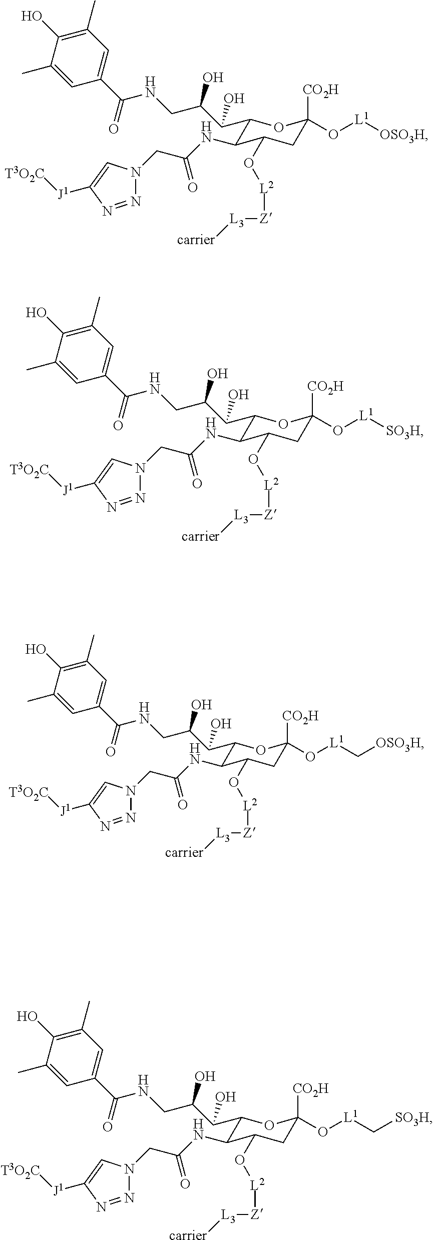

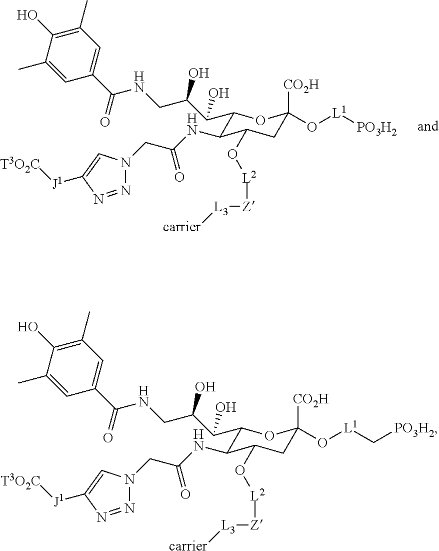

and pharmaceutically acceptable salts of any of the foregoing.

In some embodiments, the compound of Formula (II) is chosen from:

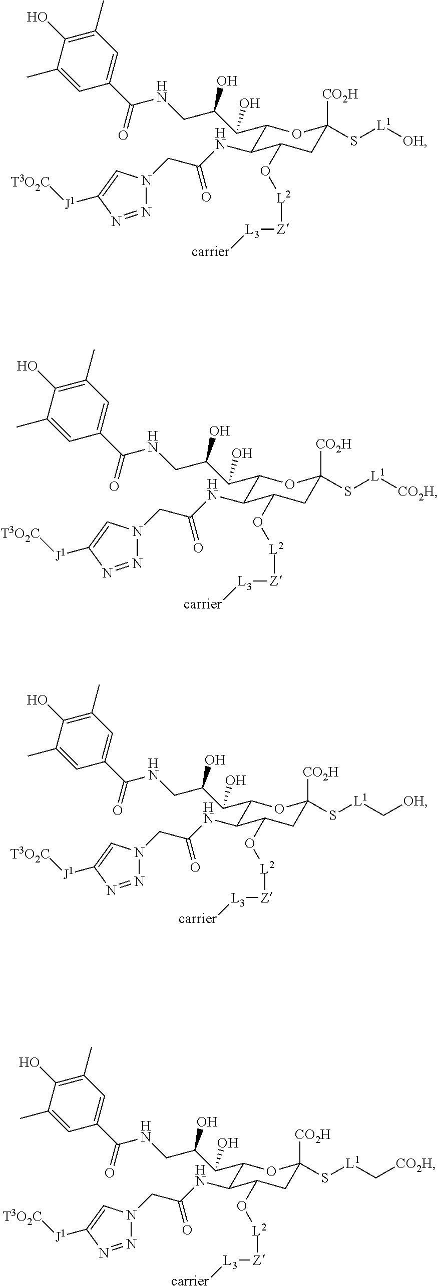

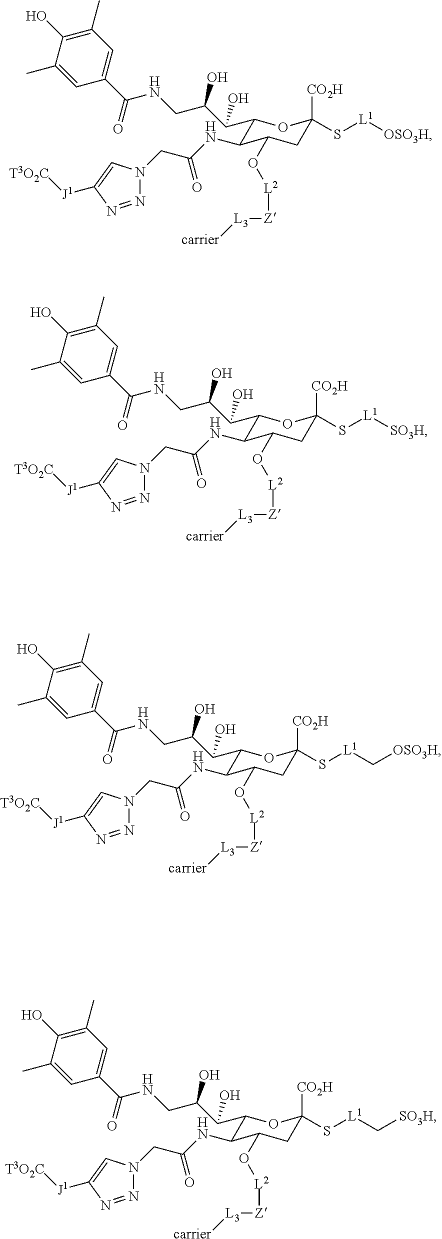

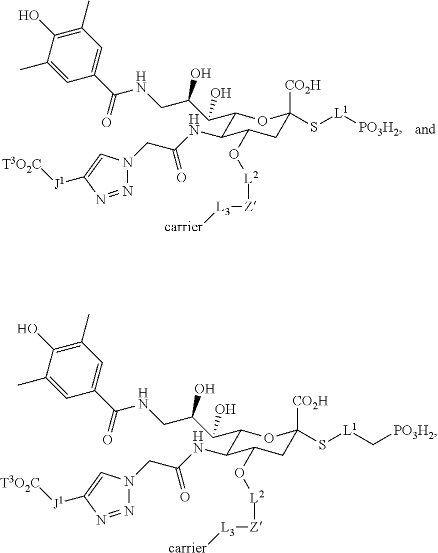

and pharmaceutically acceptable salts of any of the foregoing.

In some embodiments, the compound of Formula (II) is chosen from:

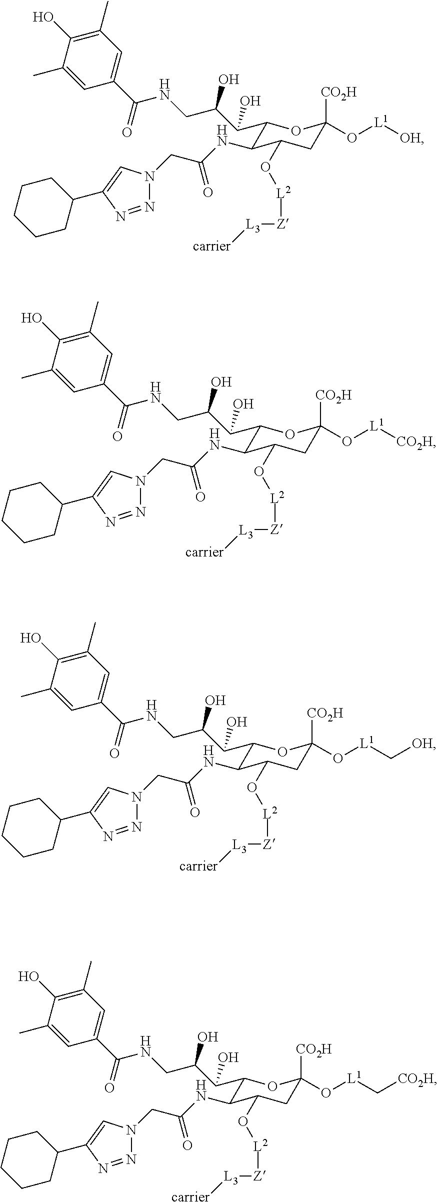

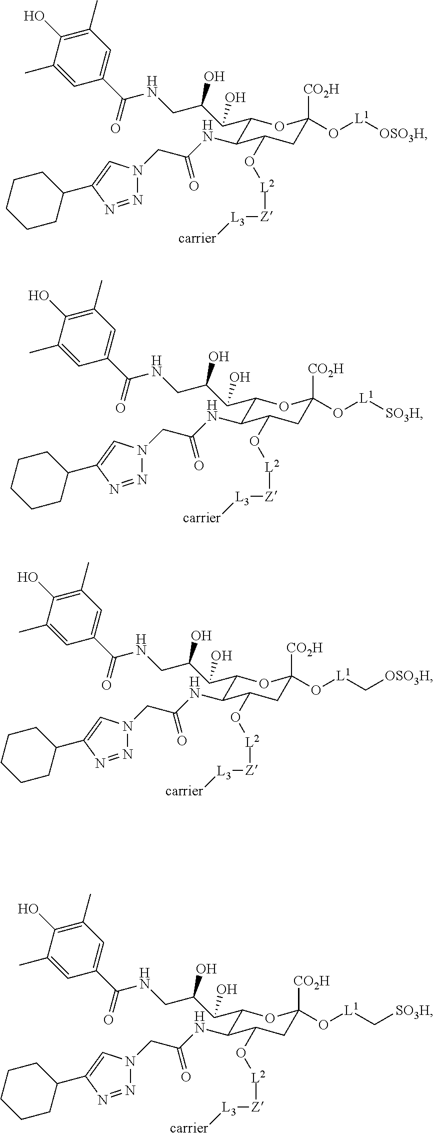

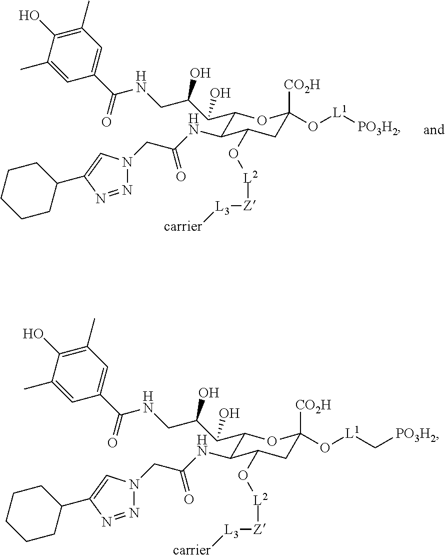

and pharmaceutically acceptable salts of any of the foregoing.

In some embodiments, the compound of Formula (II) is chosen from:

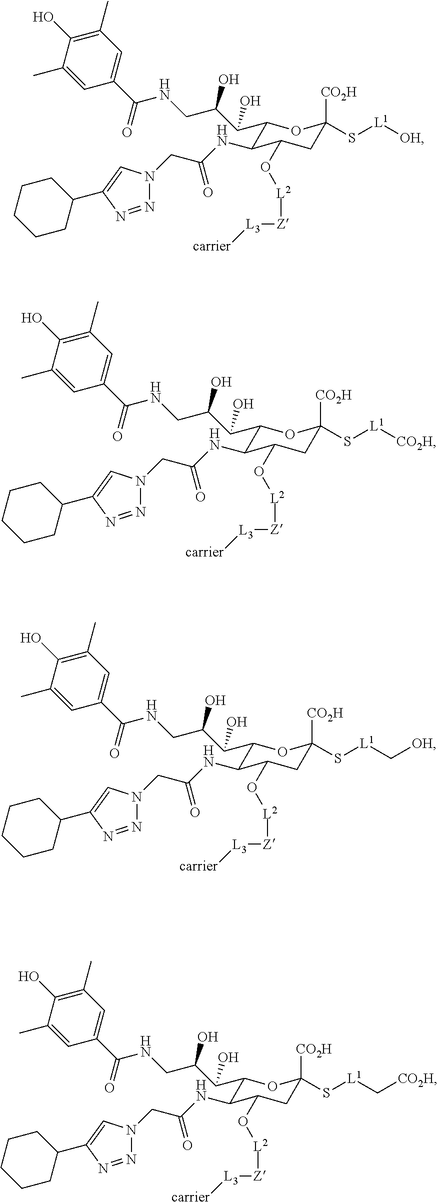

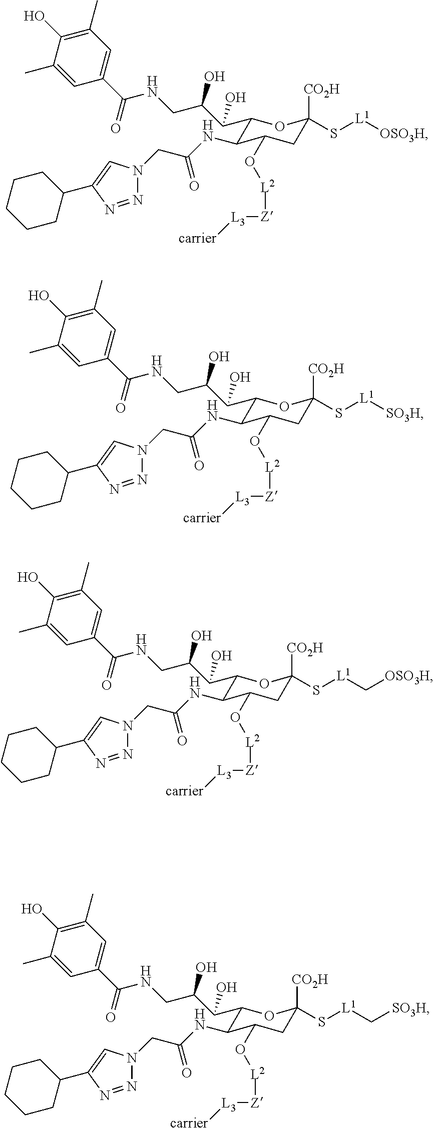

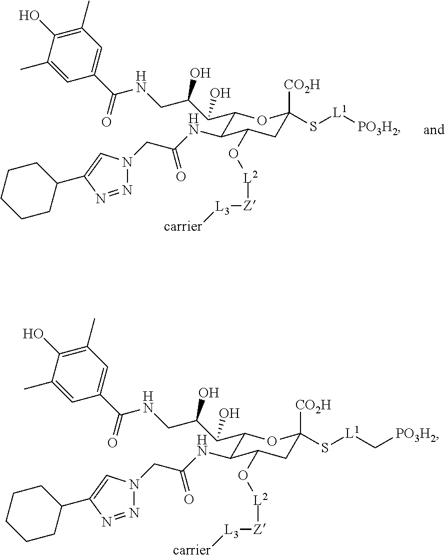

and pharmaceutically acceptable salts of any of the foregoing.

In some embodiments, the compound of Formula (II) is chosen from:

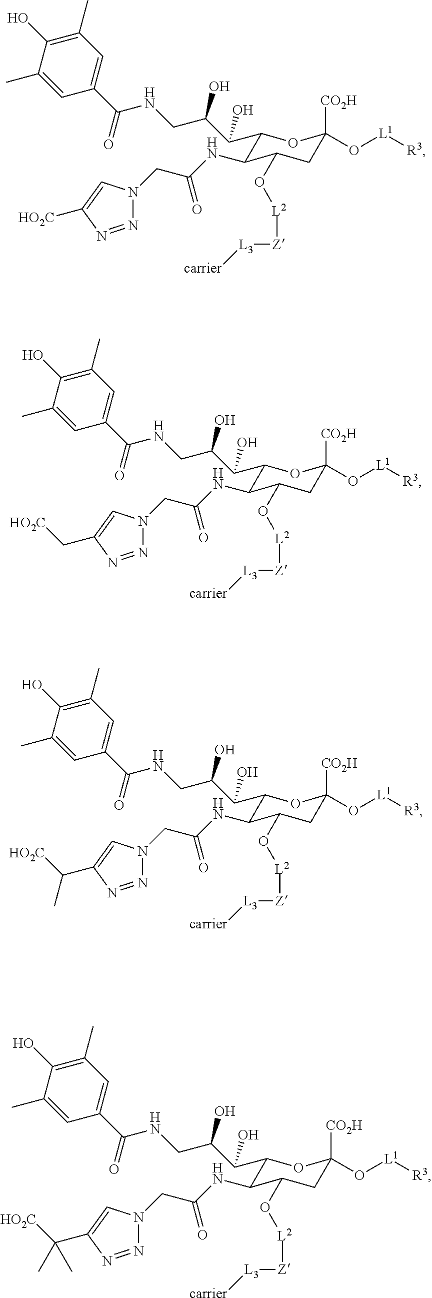

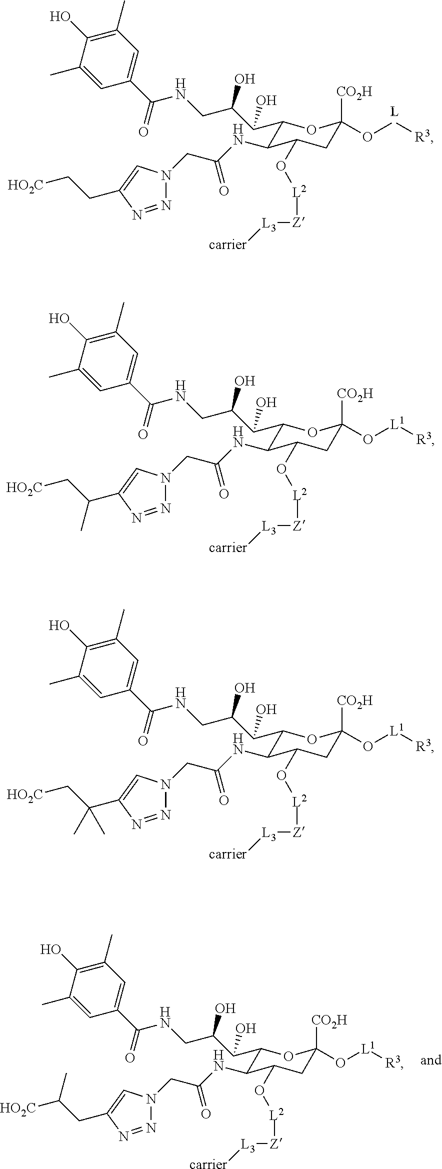

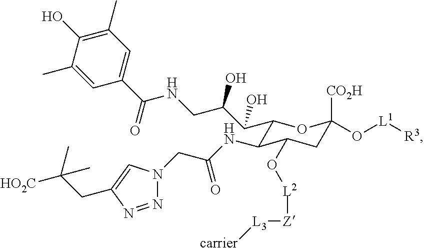

and pharmaceutically acceptable salts of any of the foregoing.

In some embodiments, the compound of Formula (II) is chosen from:

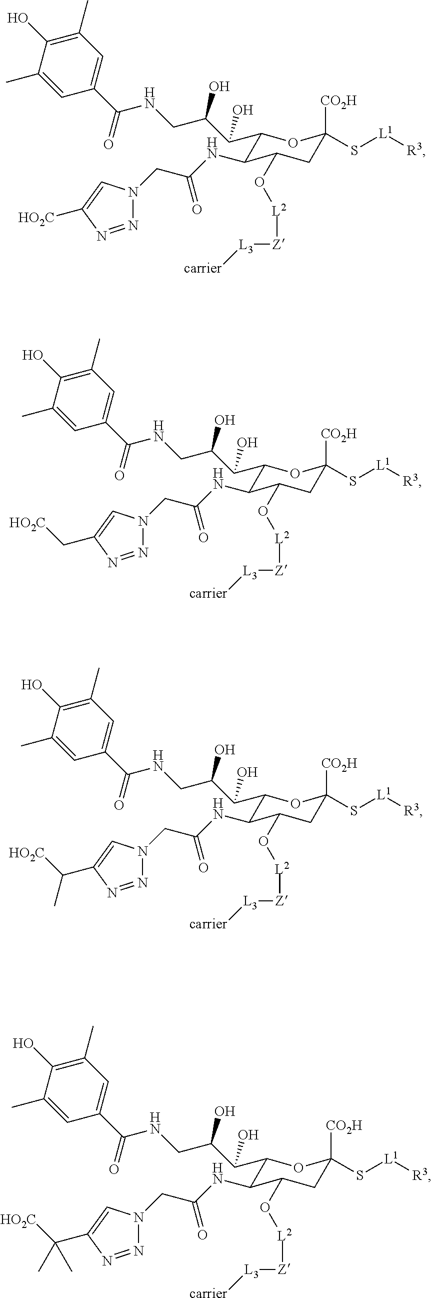

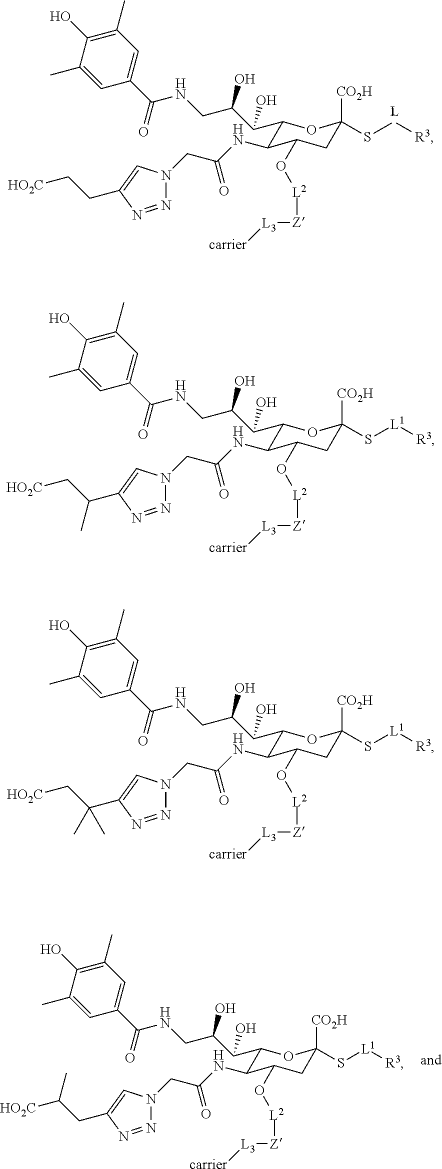

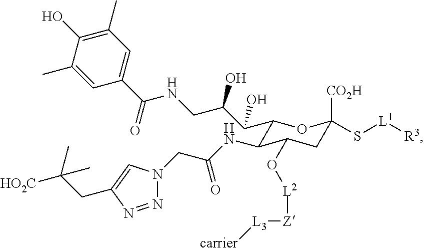

and pharmaceutically acceptable salts of any of the foregoing.

In some embodiments the compound of Formula II is chosen from:

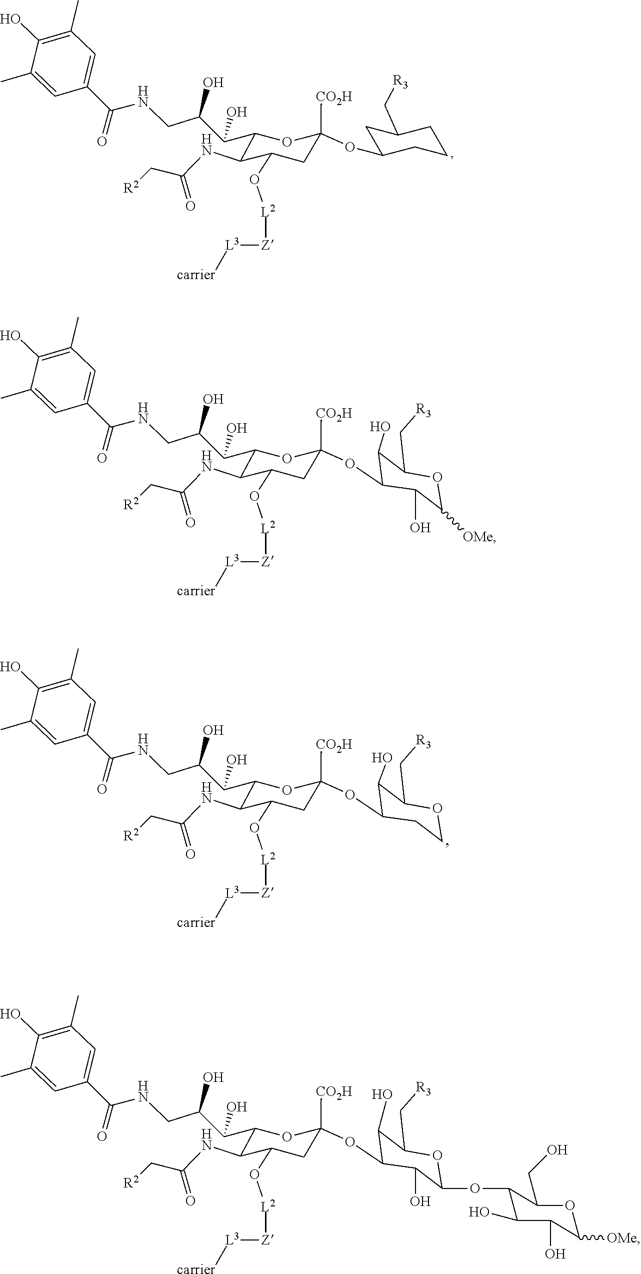

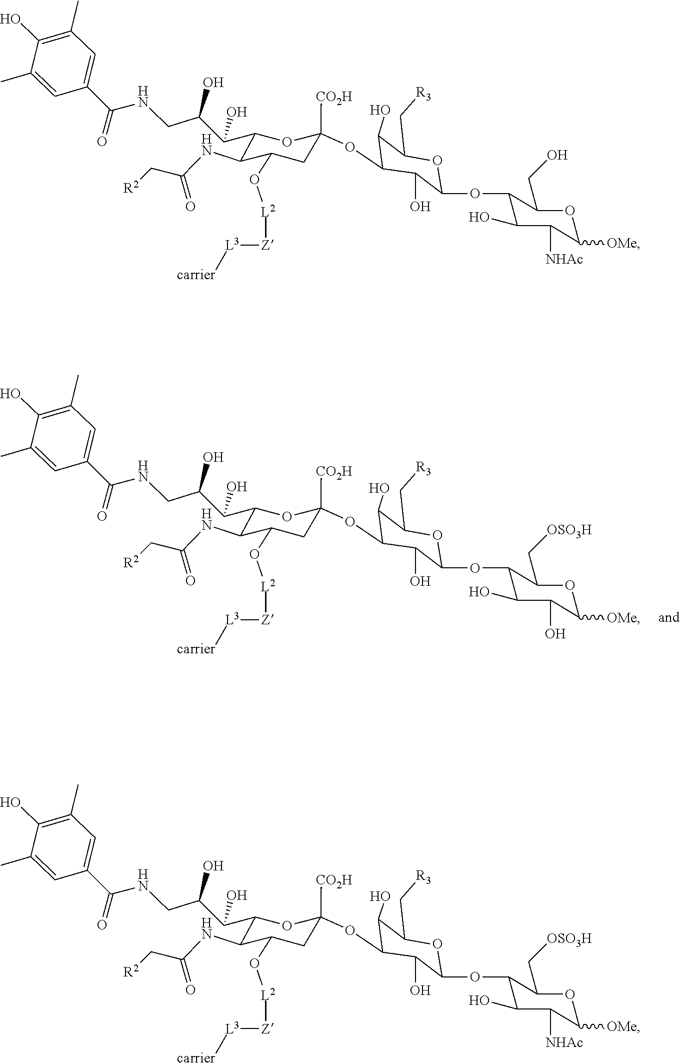

and pharmaceutically acceptable salts of any of the foregoing.

In some embodiments, the compound of Formula (HI) is chosen from:

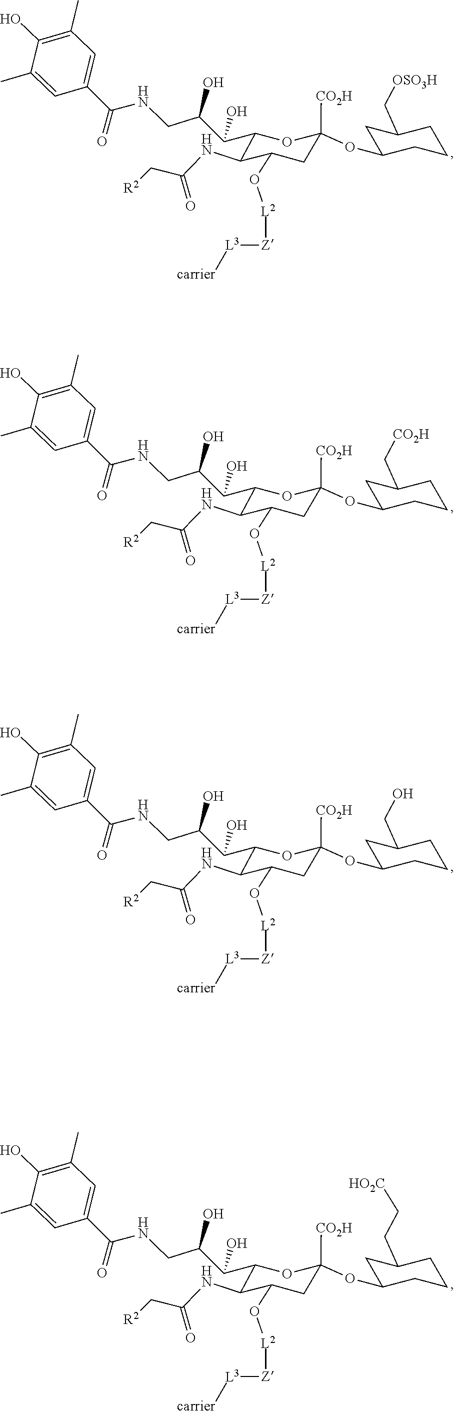

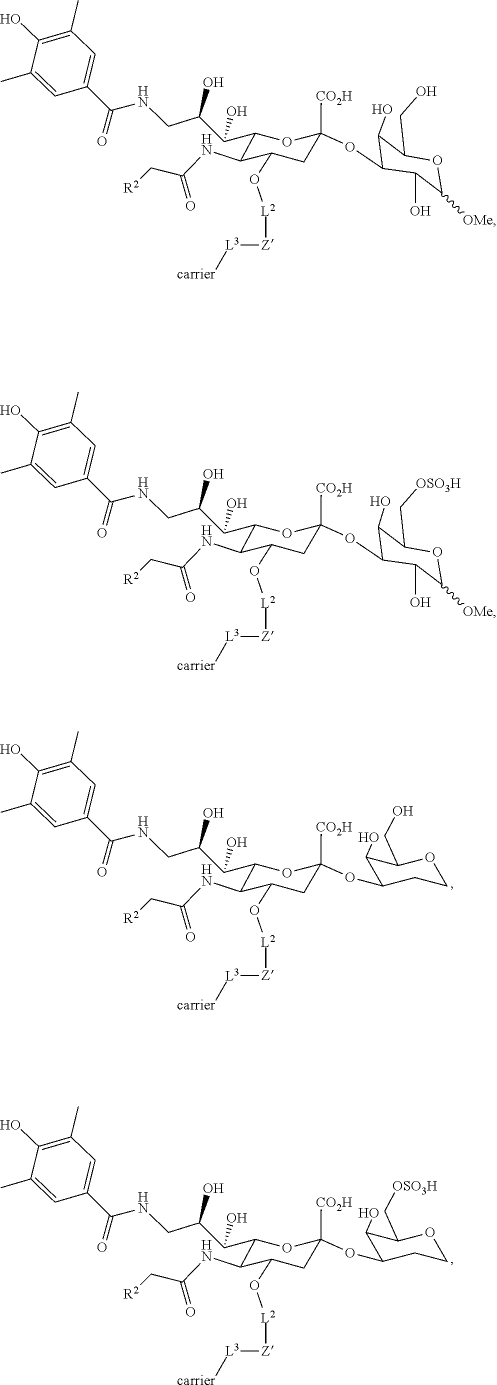

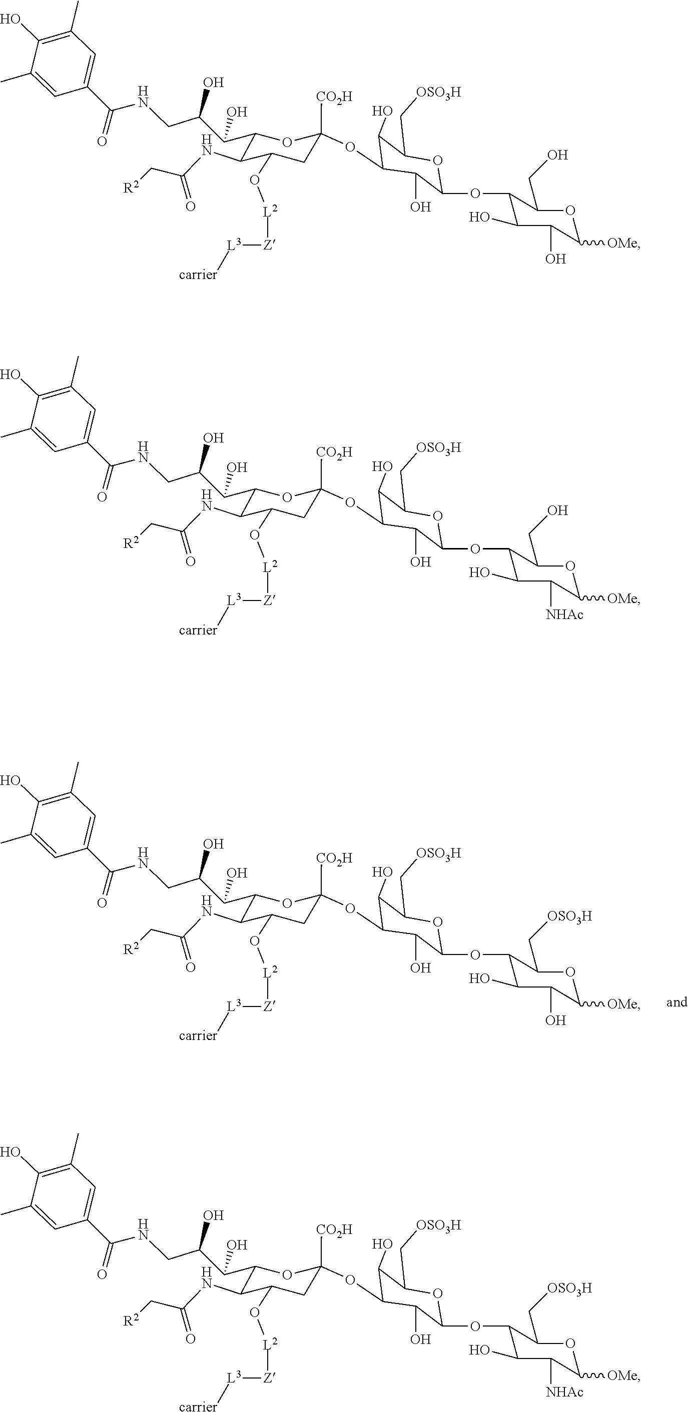

and pharmaceutically acceptable salts of any of the foregoing.

In some embodiments, the compound of Formula (HI) is chosen from:

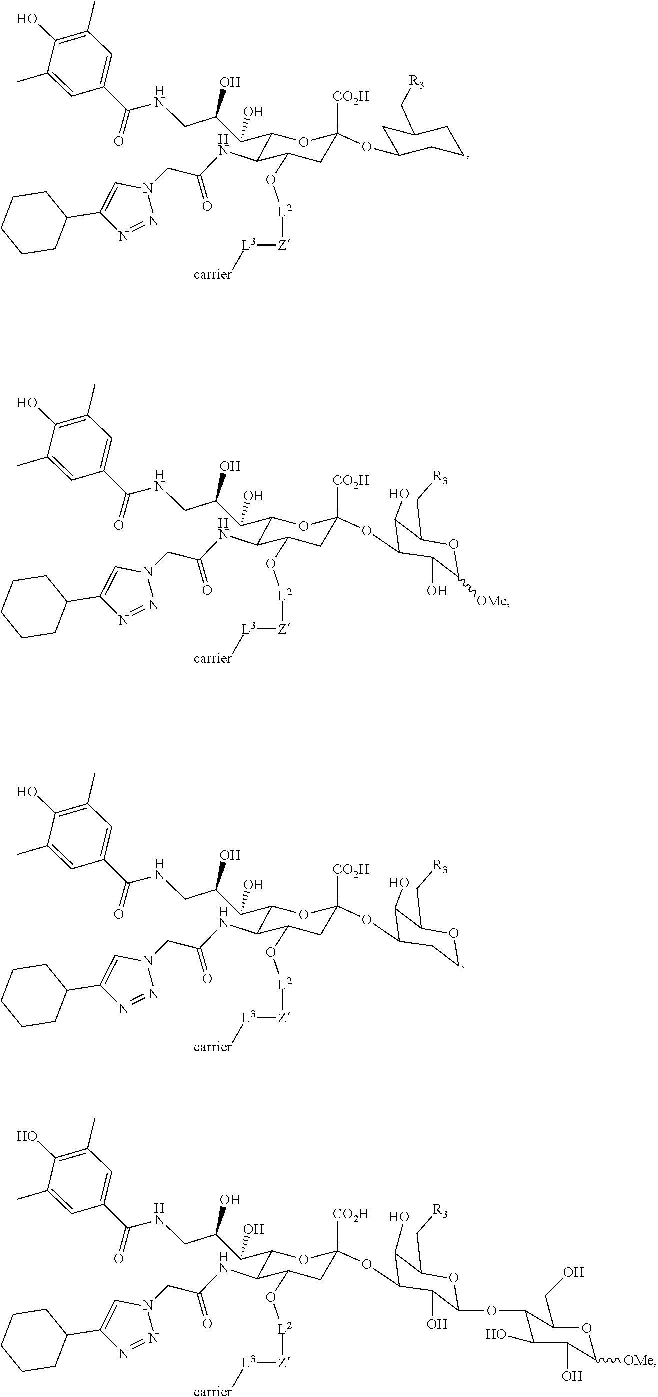

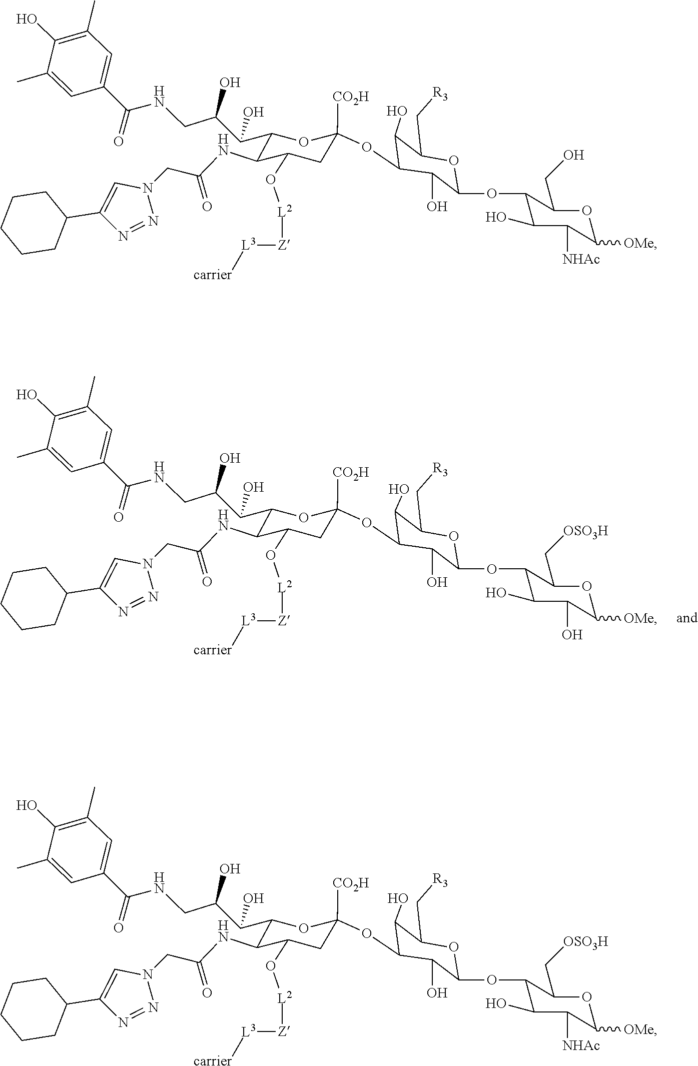

and pharmaceutically acceptable salts of any of the foregoing.

In some embodiments, the compound of Formula (II) is chosen from:

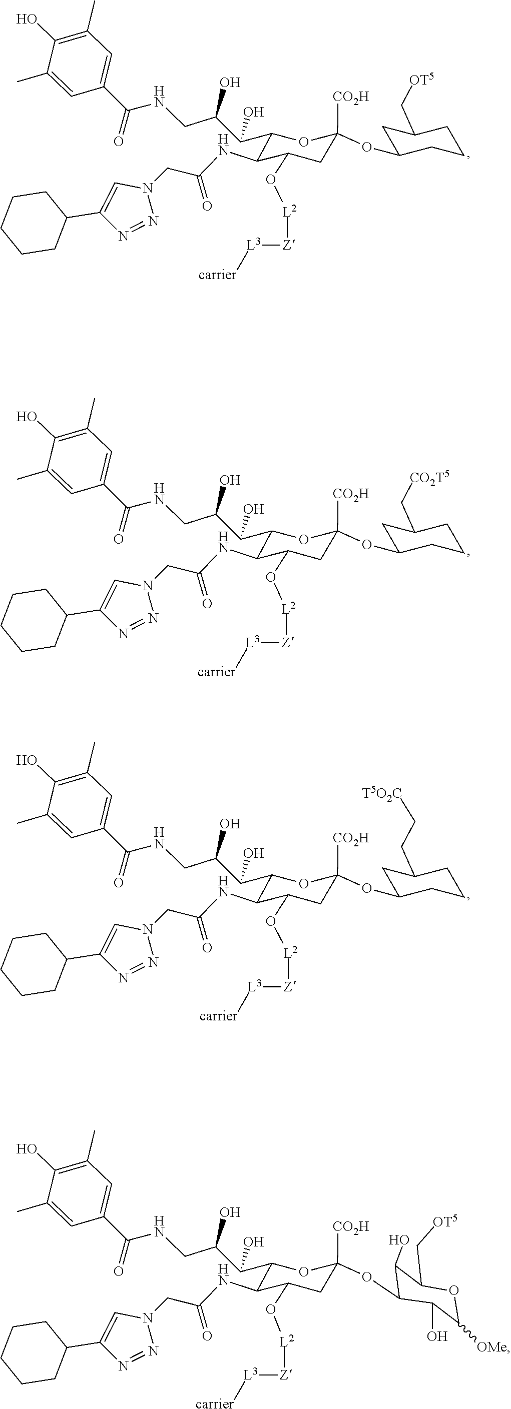

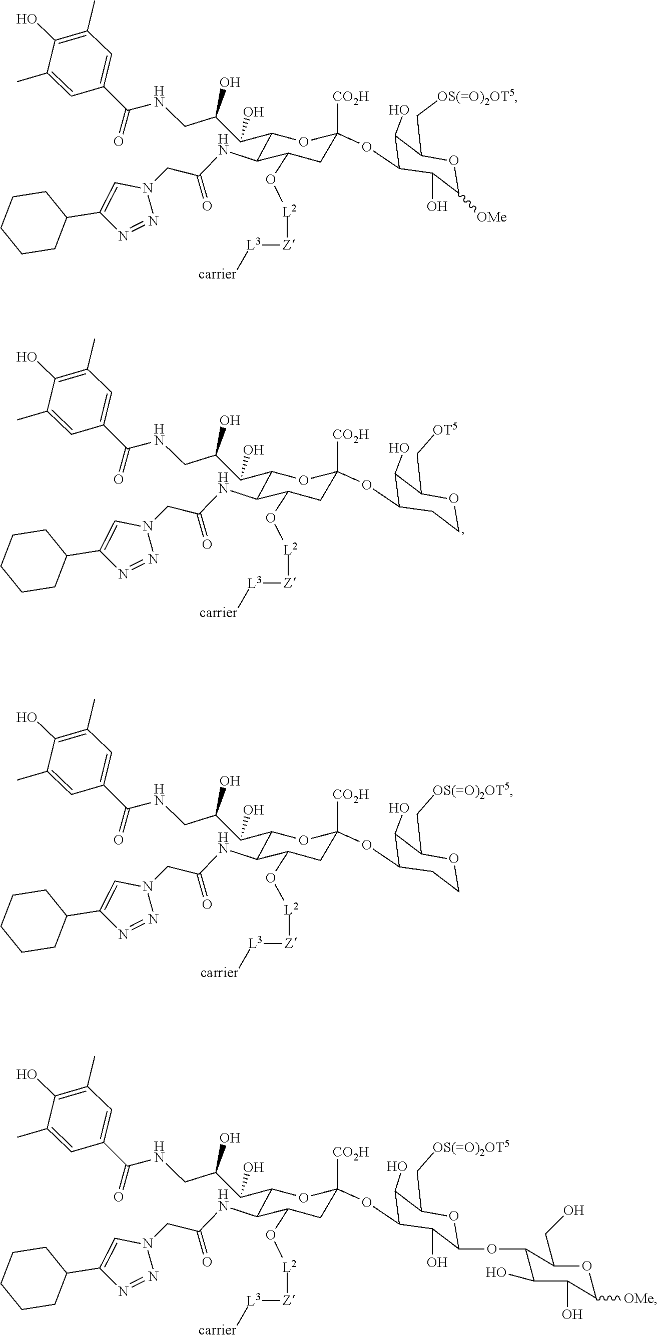

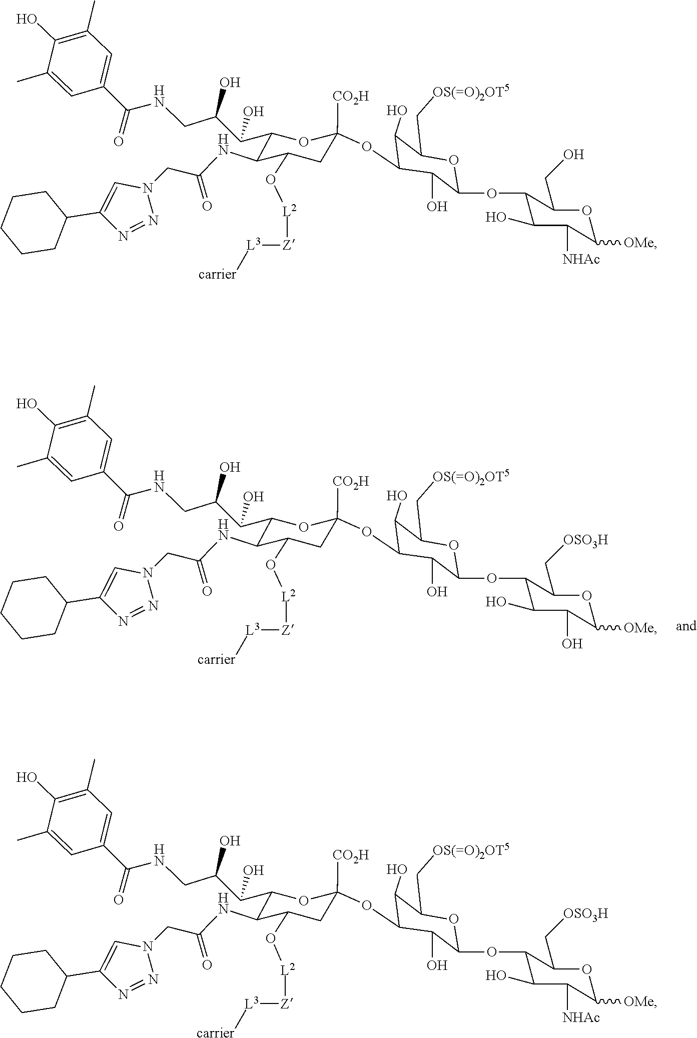

and pharmaceutically acceptable salts of any of the foregoing.

In some embodiments, the compound of Formula (II) is chosen from:

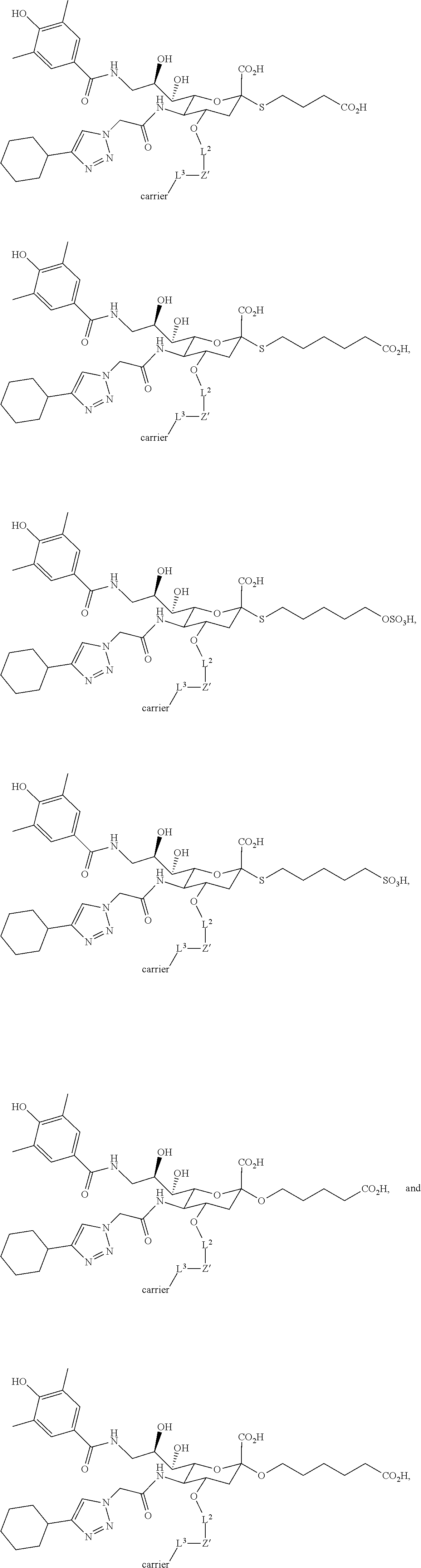

and pharmaceutically acceptable salts of any of the foregoing.

In some embodiments, the compound of Formula (II) is chosen from:

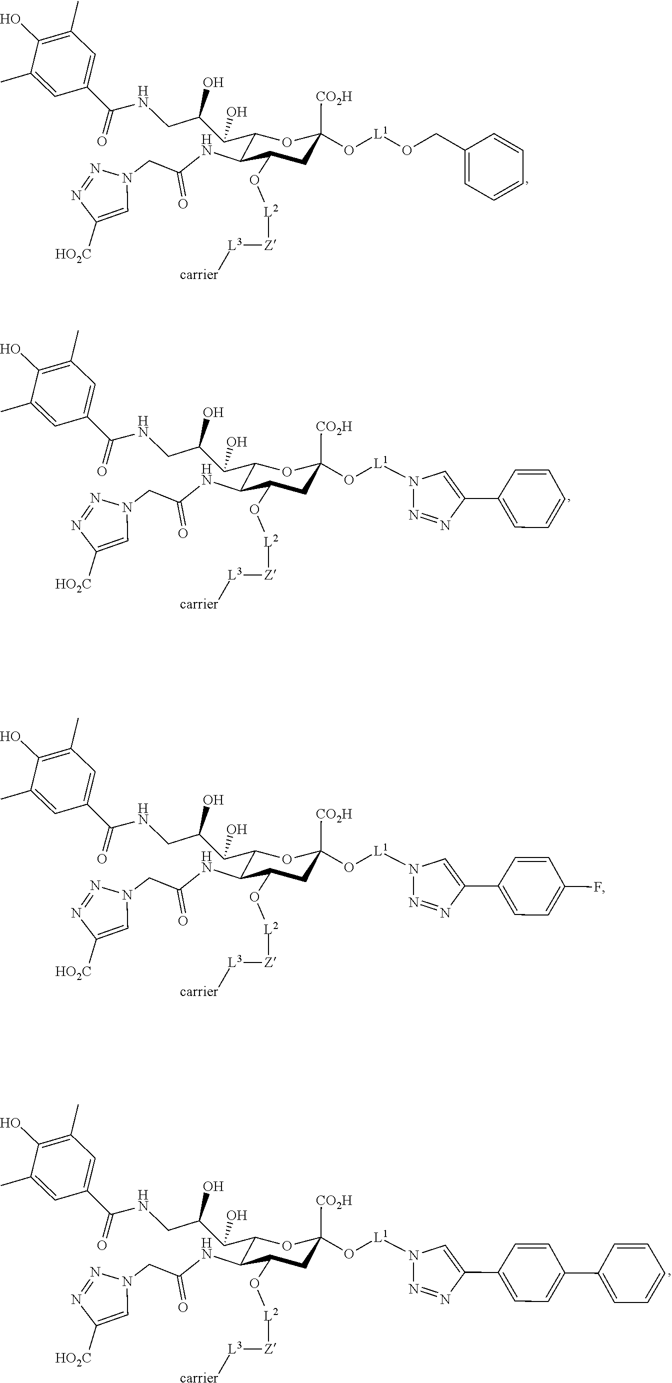

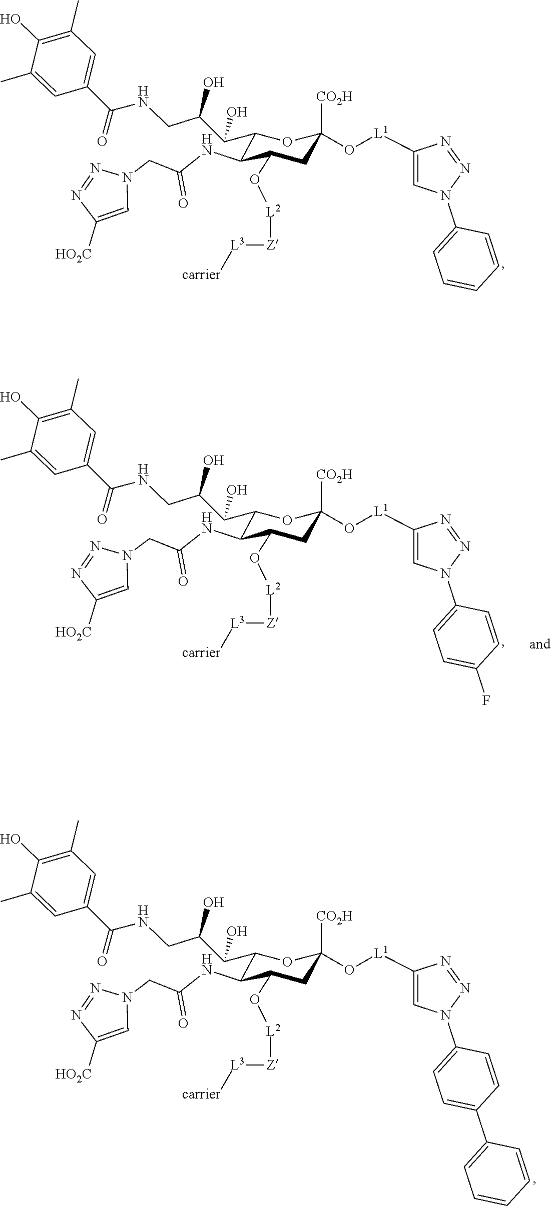

and pharmaceutically acceptable salts of any of the foregoing.

In some embodiments, the compound of Formula (II) is chosen from:

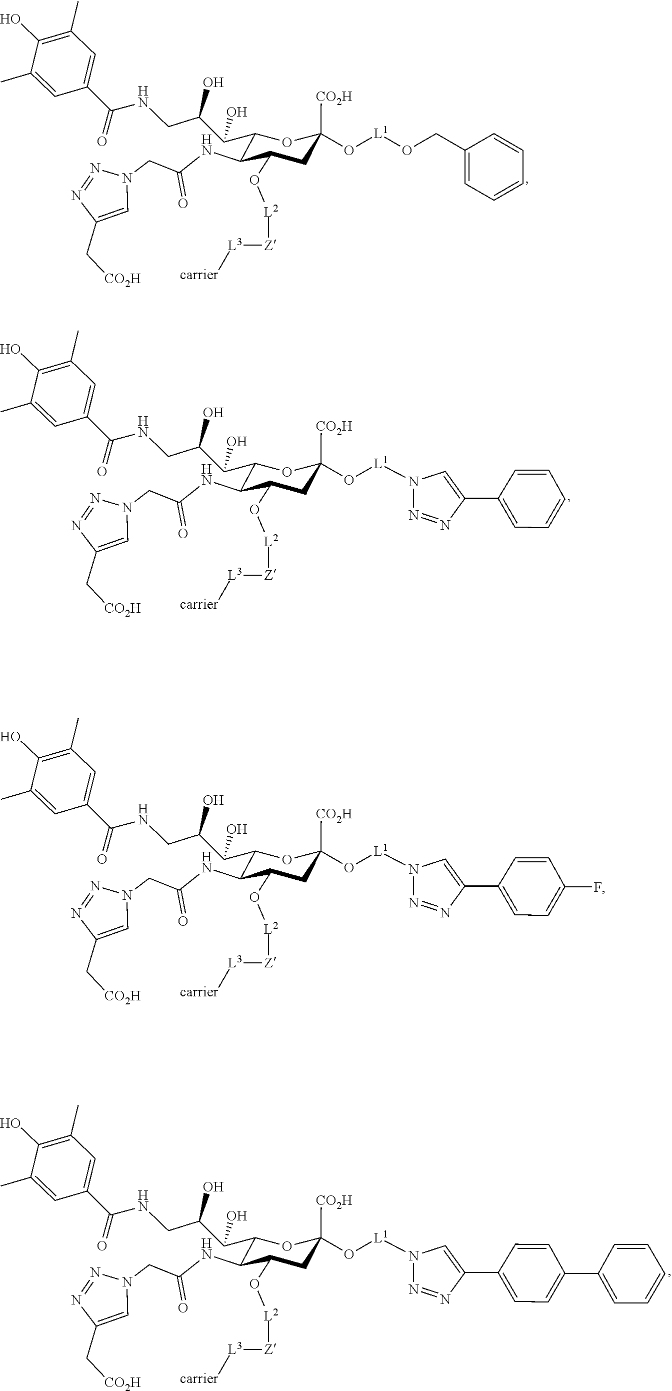

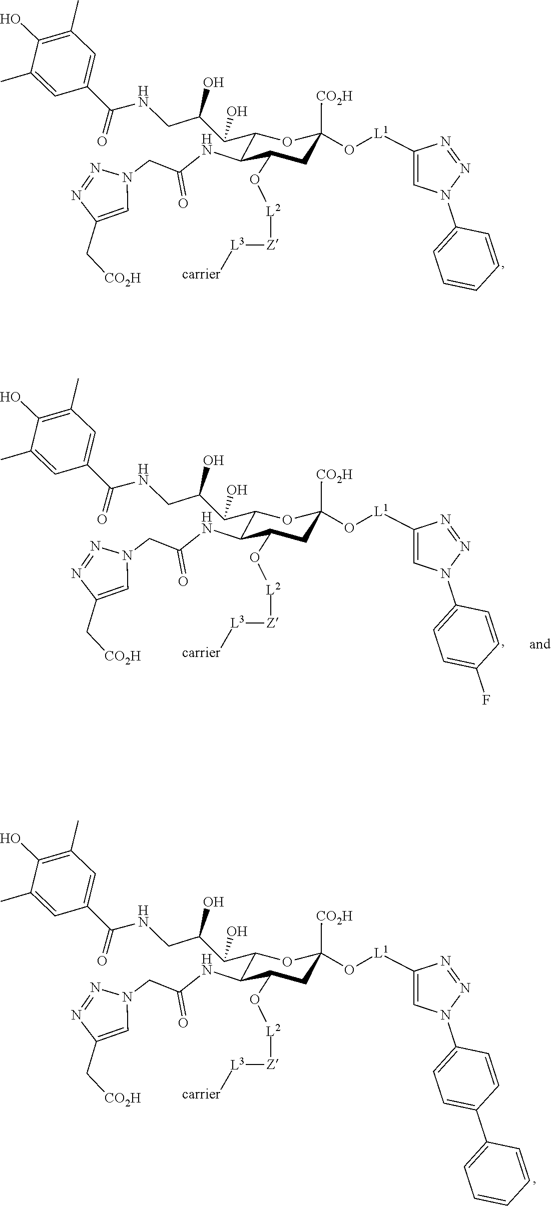

and pharmaceutically acceptable salts of any of the foregoing.

In some embodiments, the compound of Formula (II) is chosen from:

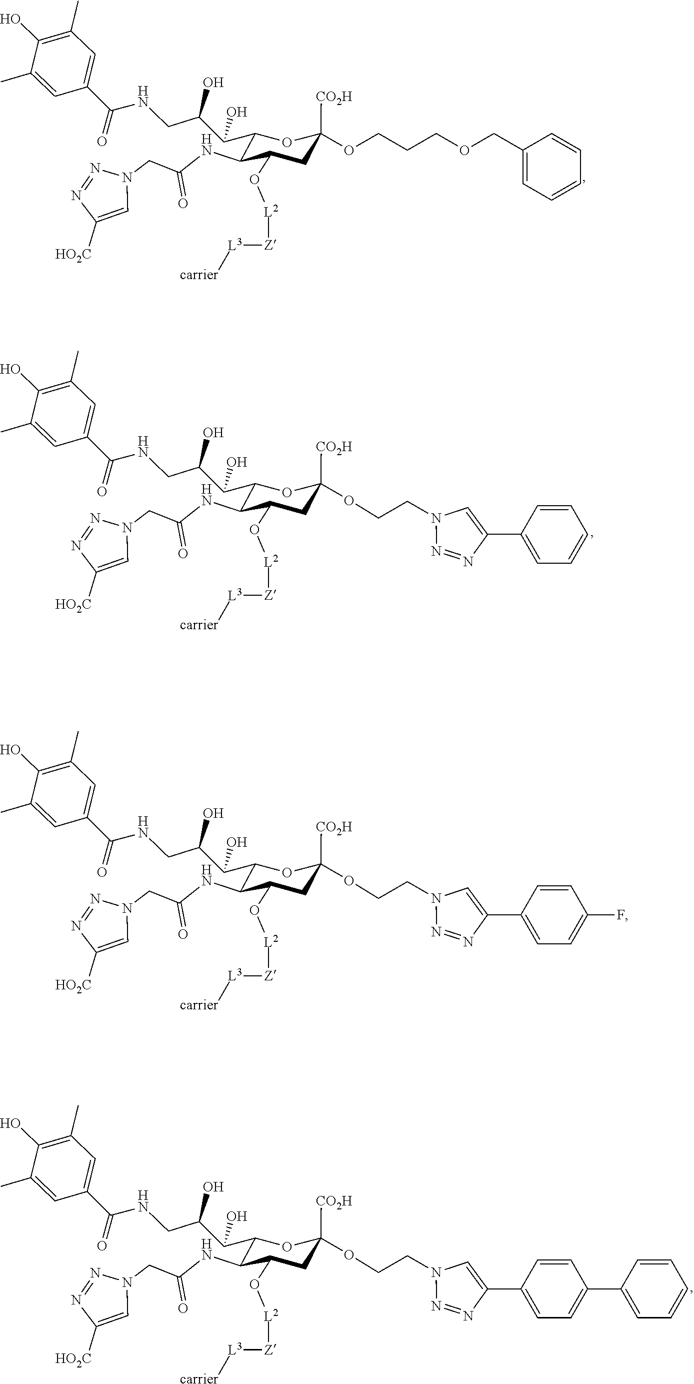

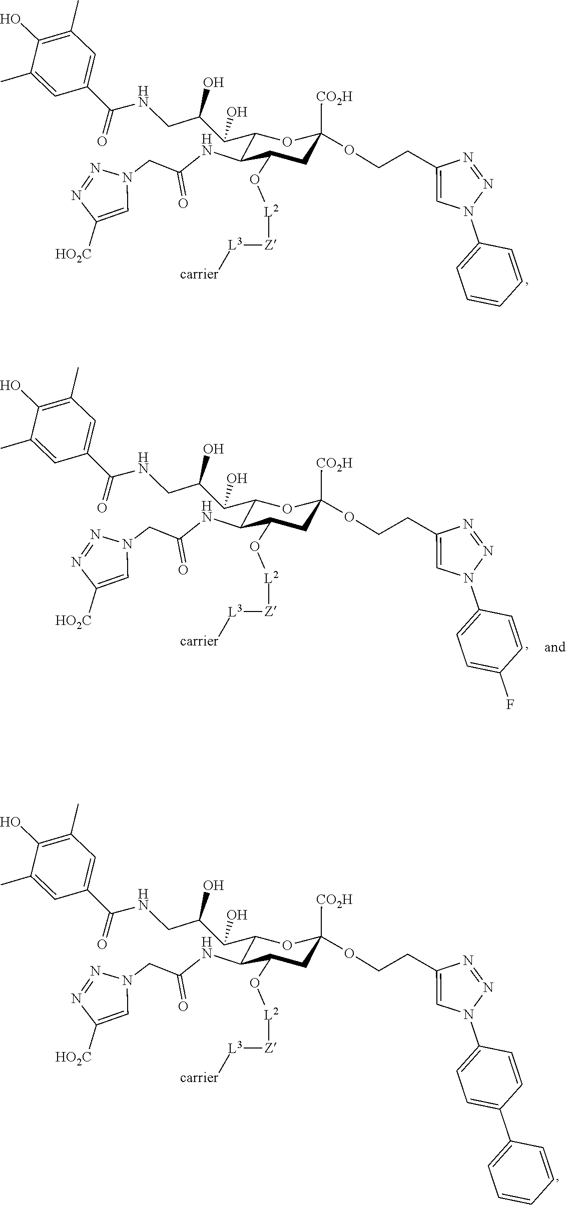

and pharmaceutically acceptable salts of any of the foregoing.

In some embodiments, the compound of Formula (II) is chosen from:

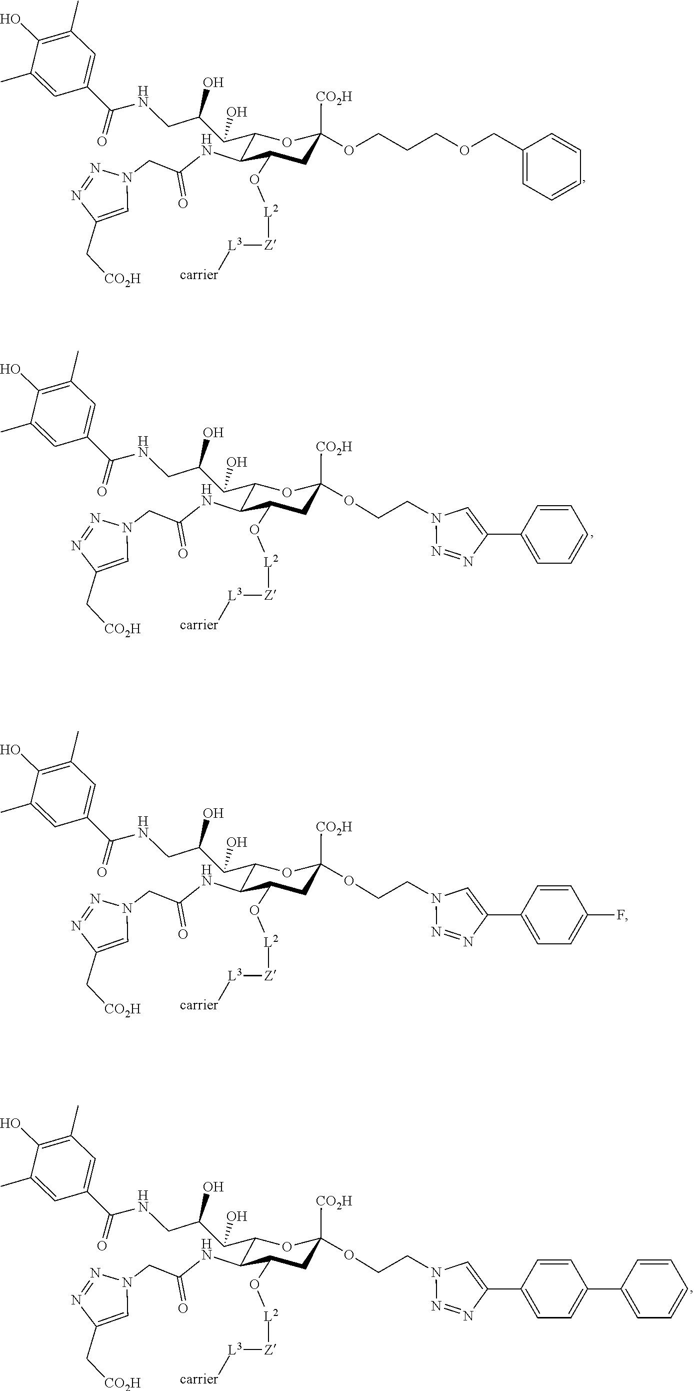

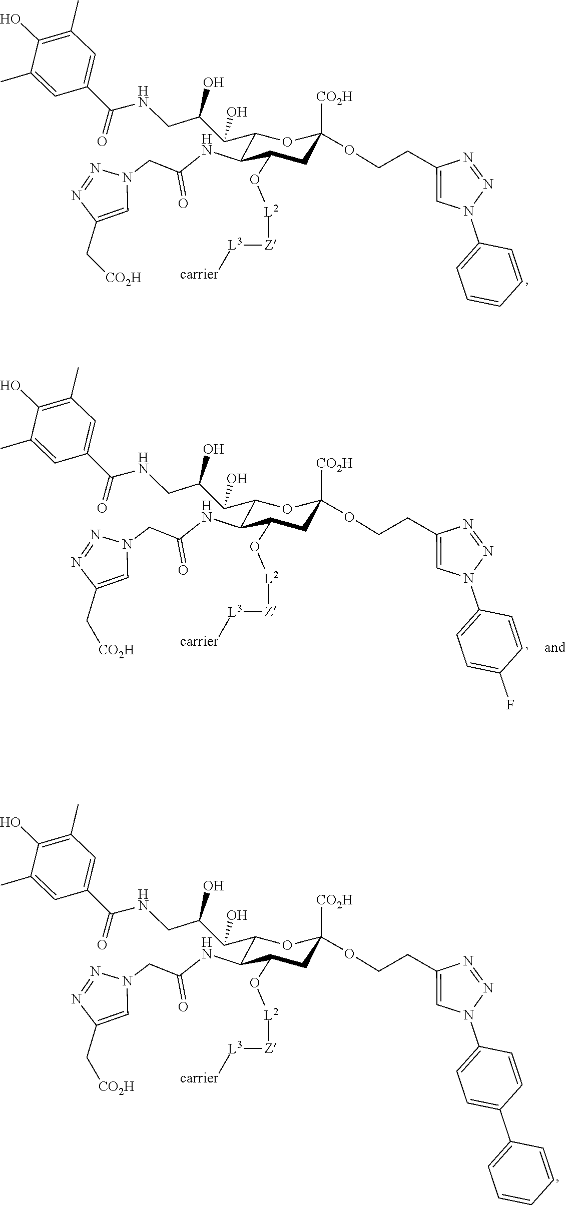

and pharmaceutically acceptable salts of any of the foregoing.

In some embodiments, the compound of Formula (II) is chosen from:

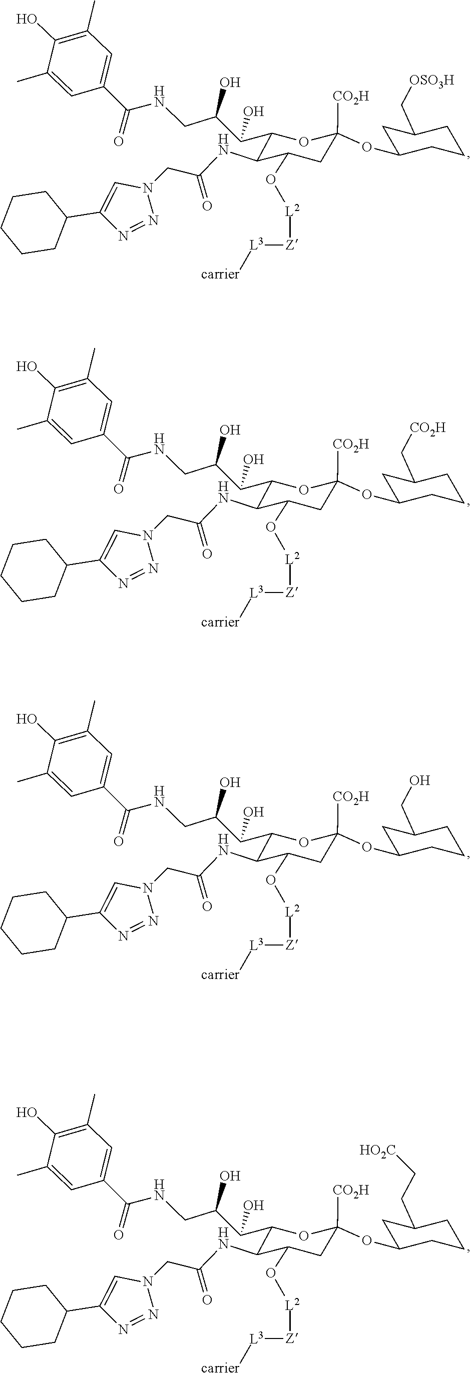

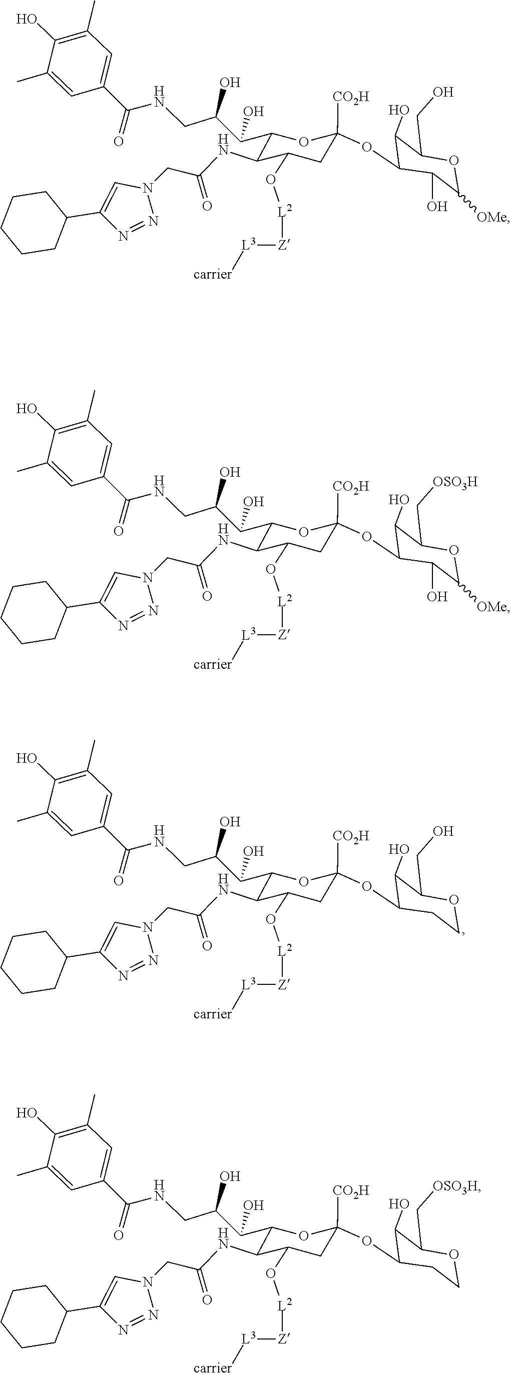

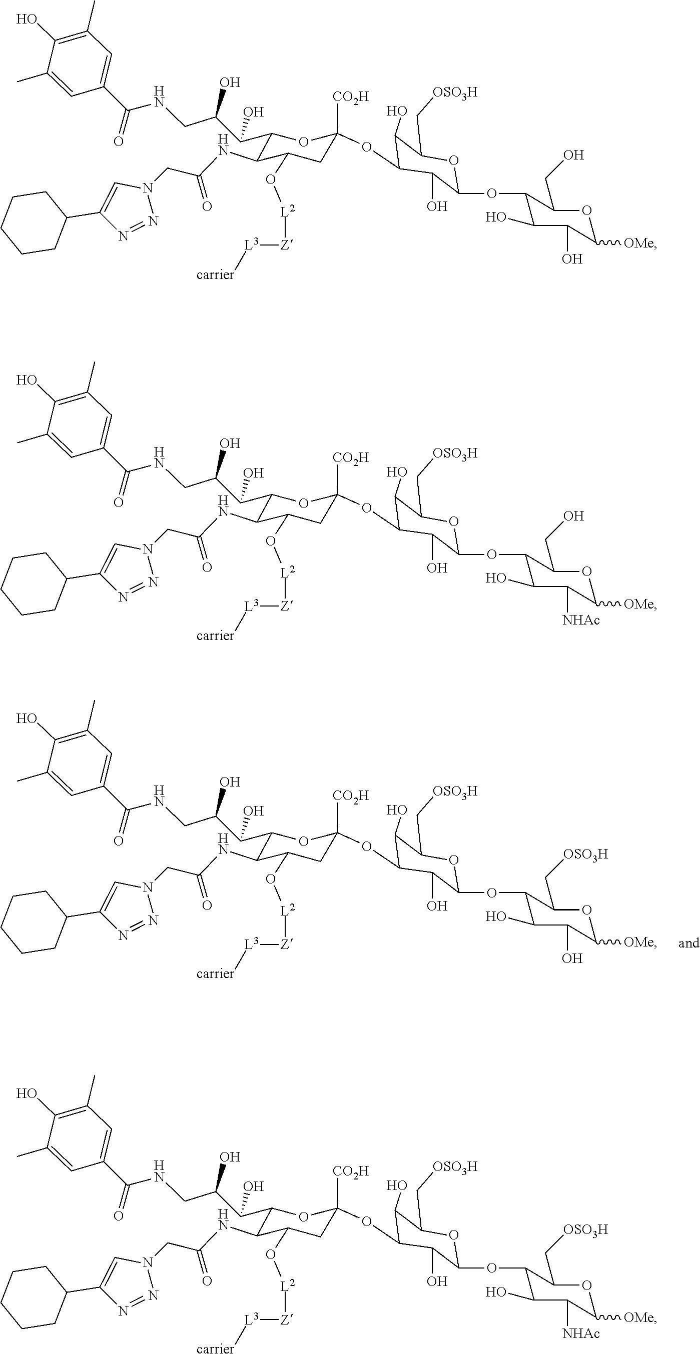

and pharmaceutically acceptable salts of any of the foregoing.

In some embodiments, at least one compound of Formula (II) or compositions comprising the same can be administered to treat a subject in need thereof or a subject who may develop a need for such treatment. In some embodiments, the at least one compound of Formula (II) or a composition comprising the same delivers at least one anti-cancer agent.

In some embodiments, a method for treating at least one disease, disorder, or condition is provided, said method comprising administering to a subject in need thereof an effective amount of at least therapeutic agent by administering the at least one compound of Formula (II) or a composition comprising the same, wherein the at least one compound of Formula (II) delivers said at least one therapeutic agent.

In some embodiments, a method for treating AML is provided, said method comprising administering to a subject in need thereof an effective amount of at least one anti-cancer agent by administering the at least one compound of Formula (II) or a composition comprising the same, wherein the at least one compound of Formula (II) delivers said at least one anti-cancer agent.

In some embodiments, a method for treatment and/or prevention of at least one disease, disorder, or condition is disclosed, the method comprising administering to a subject in need thereof an effective amount of at least one compound of Formula (II) or a composition comprising same, wherein at least one therapeutic agent is directly or indirectly linked to or associated with said at least one compound of Formula (H).

In some embodiments, a method for treatment and/or prevention of AML is disclosed, the method comprising administering to a subject in need thereof an effective amount at least one compound of Formula (II) or a composition comprising same, wherein at least one anti-cancer agent is directly or indirectly linked to or associated with said at least one compound of Formula (II).

In some embodiments, the at least one anti-cancer agent is chosen from inhibitors of phosphoinositide-3 kinase (PI3K) and inhibitors of VEGF. In some embodiments, the at least one anti-cancer agent is chosen from inhibitors of PI3K. In some embodiments, the at least one anti-cancer agent is chosen from inhibitors of VEGF. In some embodiments, the at least one anti-cancer agent is the compound named by Exelixis as “XL499.” In some embodiments, the at least one anti-cancer agent is the compound “cabo” (previously known as XL184). In some embodiments, the at least one anti-cancer agent is chosen from alkylating agents, antimetabolites, anthracyclines, plant alkaloids and topoisomerase inhibitors.

In some embodiments, the at least one compound of Formula (II) or a composition comprising the same is administered in combination with mitoxantrone. In some embodiments, the at least one compound of Formula (II) or a composition comprising the same is administered in combination with etoposide. In some embodiments, the at least one compound of Formula (II) or a composition comprising the same is administered in combination with cytarabine. In some embodiments, the at least one compound of Formula (II) or a composition comprising the same is administered in combination with at least one of mitoxantrone, etoposide, or cytarabine. In some embodiments, the at least one compound of Formula (II) or a composition comprising the same is administered in combination with daunomycin. In some embodiments, the at least one compound of Formula (II) or a composition comprising the same is administered in combination with idarubicin. In some embodiments, the at least one compound of Formula (II) or a composition comprising the same is administered in combination with MEC (mitoxantrone, etoposide, cytarabine) chemotherapy. In some embodiments, the at least one compound of Formula (II) or a composition comprising the same is administered in combination with 7+3 (cytarabine for 7 days then daunorubicin, idarubicin, or mitoxantrone for 3 days) chemotherapy.

In some embodiments, the at least one anti-cancer agent is chosen from anti-leukemic agents. In some embodiments, the anti-leukemic agents are chosen from cyclophosphamide, methotrexate, and etoposide. In some embodiments, the at least one compound of Formula (II) or a composition comprising the same is administered in combination with 6-mercaptopurine. In some embodiments, the at least one compound of Formula (II) or a composition comprising the same is administered in combination with 6-thioguanine. In some embodiments, the at least one compound of Formula (II) or a composition comprising the same is administered in combination with aminopterin. In some embodiments, the at least one compound of Formula (II) or a composition comprising the same is administered in combination with arsenic trioxide. In some embodiments, the at least one compound of Formula (II) or a composition comprising the same is administered in combination with asparaginase. In some embodiments, the at least one compound of Formula (II) or a composition comprising the same is administered in combination with cladribine. In some embodiments, the at least one compound of Formula (IT) or a composition comprising the same is administered in combination with clofarabine. In some embodiments, the at least one compound of Formula (II) or a composition comprising the same is administered in combination with cyclophosphamide. In some embodiments, the at least one compound of Formula (II) or a composition comprising the same is administered in combination with cytosine arabinoside. In some embodiments, the at least one compound of Formula (H) or a composition comprising the same is administered in combination with dasatinib. In some embodiments, the at least one compound of Formula (II) or a composition comprising the same is administered in combination with decitabine. In some embodiments, the at least one compound of Formula (II) or a composition comprising the same is administered in combination with dexamethasone. In some embodiments, the at least one compound of Formula (II) or a composition comprising the same is administered in combination with fludarabine. In some embodiments, the at least one compound of Formula (II) or a composition comprising the same is administered in combination with gemtuzumab ozogamicin. In some embodiments, the at least one compound of Formula (II) or a composition comprising the same is administered in combination with imatinib mesylate. In some embodiments, the at least one compound of Formula (II) or a composition comprising the same is administered in combination with interferon-α. In some embodiments, the at least one compound of Formula (II) or a composition comprising the same is administered in combination with interleukin-2. In some embodiments, the at least one compound of Formula (II) or a composition comprising the same is administered in combination with melphalan. In some embodiments, the at least one compound of Formula (II) or a composition comprising the same is administered in combination with methotrexate. In some embodiments, the at least one compound of Formula (H) or a composition comprising the same is administered in combination with nelarabine. In some embodiments, the at least one compound of Formula (II) or a composition comprising the same is administered in combination with nilotinib. In some embodiments, the at least one compound of Formula (II) or a composition comprising the same is administered in combination with oblimersen. In some embodiments, the at least one compound of Formula (II) or a composition comprising the same is administered in combination with pegaspargase. In some embodiments, the at least one compound of Formula (II) or a composition comprising the same is administered in combination with pentostatin. In some embodiments, the at least one compound of Formula (II) or a composition comprising the same is administered in combination with ponatinib. In some embodiments, the at least one compound of Formula (II) or a composition comprising the same is administered in combination with prednisone. In some embodiments, the at least one compound of Formula (II) or a composition comprising the same is administered in combination with rituximab. In some embodiments, the at least one compound of Formula (II) or a composition comprising the same is administered in combination with tretinoin. In some embodiments, the at least one compound of Formula (H) or a composition comprising the same is administered in combination with vincristine.

In some embodiments, the at least one compound of Formula (II) or a composition comprising the same is administered over one or more doses, with one or more intervals between doses. In some embodiments, the at least one compound of Formula (II) or a composition comprising the same is administered over 1, 2, 3, 4, 5, 6, 7, 8, 9, 10, 11, 12, 13, 14, 15, 16, 17, 18, 19, or 20 doses. In some embodiments, the at least one compound of Formula (II) or a composition comprising the same is administered at 6-hour, 12-hour, 18-hour, 24-hour, 48-hour, 72-hour, or 96-hour intervals.

In some embodiments, the subject has been diagnosed with AML as per the World Health Organization (WHO) criteria. (Arber D A et al., “The 2016 revision to the World Health Organization classification of myeloid neoplasms and acute leukemia.” Blood (2016) 127(20):2391-2405.) In some embodiments, the subject is >18 years of age with relapsed or refractory AML after 52 prior induction regiments, at least one containing anthracyclines. In some embodiments, the subject is >60 years of age with newly diagnosed AML. In some embodiments, the subject has an absolute blast count 9ABC of 5 40,000/mm. In some embodiments, the subject is medically eligible to receive MEC chemotherapy. In some embodiments, the subject is medically eligible to receive 7+3 cytarabine/idarubicin chemotherapy. In some embodiments, the subject has an Eastern Cooperative Oncology Group (ECOG) performance status of 0-2. In some embodiments, the subject has hemodynamically stable and adequate organ function. In some embodiments, the subject does not have acute promyelocytic leukemia. In some embodiments, the subject does not have acute leukemia of ambiguous lineage. In some embodiments, the subject does not have active signs or symptoms of CNS involvement by malignancy. In some embodiments, the subject has no prior G-CSF, GM-CSF, or plerixafor within 14 days of treatment with the pharmaceutical composition disclosed herein. In some embodiments, the subject has no known history or evidence of active hepatitis A, B, or C or HIV. In some embodiments, the subject does not have an uncontrolled acute life-threatening bacterial, viral, or fungal infection. In some embodiments, the subject does not have active graft versus host disease (GVHD)≥Grade 2 or extensive chronic GVHD requiring immunosuppressive therapy. In some embodiments, the subject does not have hematopoietic stem cell transplantation ≤4 months prior to the treatments disclosed herein. In some embodiments, the subject does not have clinically significant cardiovascular disease.

Whenever a term in the specification is identified as a range (e.g., C₁₋₄ alkyl) or “ranging from”, the range independently discloses and includes each element of the range. As a non-limiting example, C₁₋₄ alkyl groups includes, independently, C₁ alkyl groups, C₂ alkyl groups, C₃ alkyl groups, and C₄ alkyl groups. As another non-limiting example, “n is chosen from integers ranging from 0 to 2” includes, independently, 0, 1, and 2.

The term “at least one” refers to one or more, such as one, two, etc. For example, the term “at least one C₁₋₄ alkyl group” refers to one or more C₁₋₄ alkyl groups, such as one C₁₋₄ alkyl group, two C₁₋₄ alkyl groups, etc.

The term “1,3-dipole” includes compounds that contain a consecutive series of three atoms, a-b-c, where atom a contains a sextet of electrons in its outer shell and atom c contains an octet with at least one unshared pair of electrons in its outer shell. Because molecules that have six electrons in the outer shell of an atom are typically unstable, the a-b-c atom example is one canonical structure of a resonance hybrid, where at least one structure can be drawn. The term 1,3-dipoles includes those in which one of the canonical forms has a double bond on the sextet atom (atom a) and the other canonical form has a triple bond on that atom:

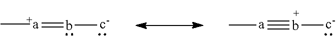

The term 1,3-dipoles also includes those in which the dipolar canonical form has a single bond on the sextet atom (atom a) and the other canonical form has a double bond on that atom:

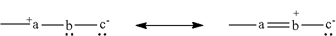

The term “1,3-dipolarophile” includes dienophiles and dienes.

The term “activated ester” means an ester derivative that has sufficient reactivity such that it can react with a nucleophile (e.g., an amino group).

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “aryl” includes hydrocarbon ring system groups comprising at least 6 carbon atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “carboxy protecting group” includes groups known in the art to protect an acidic hydrogen of a carboxyl group against undesirable reaction during synthetic procedures, e.g., to block or protect the acid functionality while the reactions involving other functional sites of the compound are carried out, and to be removable.

The terms “CD33” and “Siglec-3” are used interchangeably herein.

The term “diene” includes a molecule bearing two conjugated double bonds. The diene may also be non-conjugated, if the geometry of the molecule is constrained so as to facilitate a cycloaddition reaction (see Cookson J. Chem. Soc. 5416 (1964), which is incorporated by reference in its entirety). The atoms forming these double bonds can be carbon or a heteroatom or any combination thereof.

The term “dienophile” includes a molecule bearing an alkene group, or a double bond between a carbon and a heteroatom, or a double bond between two heteroatoms.

The term “electrophile” includes chemical moieties which can accept a pair of electrons from a nucleophile. Electrophiles include cyclic compounds such as epoxides, aziridines, episulfides, cyclic sulfates, carbonates, lactones, lactams and the like. Non-cyclic electrophiles include sulfates, sulfonates (e.g., tosylates), chlorides, bromides, iodides, and the like.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “haloalkenyl” includes alkenyl groups, as defined herein, substituted by at least one halogen, as defined herein. Non-limiting examples of haloalkenyl groups include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl wherein at least one halogen is fluoro. Unless stated otherwise specifically in the specification, a haloalkynyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be monocyclic, bicyclic, tricyclic or tetracyclic ring systems, which may include fused or bridged ring systems, and may be partially or fully saturated; any nitrogen, carbon or sulfur atom(s) in the heterocyclyl group may be optionally oxidized; any nitrogen atom in the heterocyclyl group may be optionally quaternized. Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

The term “hydroxy protecting group” includes groups known in the art to protect a hydroxyl group against undesirable reaction during synthetic procedures and to be removable. The use of hydroxy protecting groups is well known in the art for protecting groups against undesirable reactions during a synthetic procedure and many such protecting groups are known.

The term “nucleophile” includes a chemical moiety having a reactive pair of electrons. Non-limiting examples of nucleophiles include uncharged compounds such as amines, mercaptans and alcohols, and charged moieties such as alkoxides, thiolates, carbanions, and a variety of organic and inorganic anions. Illustrative anionic nucleophiles include simple anions such as azide, cyanide, thiocyanate, acetate, formate or chloroformate, and bisulfite. Organometallic reagents such as organocuprates, organozincs, organolithiums, Grignard reagents, enolates, acetylides, and the like may, under appropriate reaction conditions, be suitable nucleophiles.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

“Self-assembly” refers to the process of the formation of, for example, a carrier using components that will orient themselves in a predictable manner forming carriers predictably and reproducibly.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

This application contemplates all the isomers of the compounds disclosed herein. “Isomer” as used herein includes optical isomers (such as stereoisomers, e.g., enantiomers and diastereoisomers), geometric isomers (such as Z (zusammen) or E (entgegen) isomers), and tautomers. The present disclosure includes within its scope all the possible geometric isomers, e.g., Z and E isomers (cis and trans isomers), of the compounds as well as all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g., fractional crystallization, may be used.

The present disclosure includes within its scope all possible tautomers. Furthermore, the present disclosure includes in its scope both the individual tautomers and any mixtures thereof. Each compound disclosed herein includes within its scope all possible tautomeric forms. Furthermore, each compound disclosed herein includes within its scope both the individual tautomeric forms and any mixtures thereof. With respect to the methods, uses and compositions of the present application, reference to a compound or compounds is intended to encompass that compound in each of its possible isomeric forms and mixtures thereof. Where a compound of the present application is depicted in one tautomeric form, that depicted structure is intended to encompass all other tautomeric forms.

Biological activity of the CD33 ligands and/or CD33 ligand-bearing carriers described herein may be determined, for example, by performing at least one in vitro and/or in vivo study routinely practiced in the art.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiable cell lines, transformed cell lines, and the like.

As described herein, methods for characterizing CD33 ligands and/or CD33 ligand-bearing carriers include animal model studies.

In certain aspects, the compounds and compositions as described herein can be used to treat patients suffering from a condition associated with over expression of CD33. In some embodiments, the condition associated with over expression of CD33 is AML.

In certain aspects, the compounds and compositions as described herein can be used to treat patients suffering from cancers of the blood and complications associated therewith. Examples of cancers of the blood include AML.

Acute myelogenous leukemia (also known as acute myeloid leukemia or AML) is a cancer of white blood cells, and in particular the myeloid line. It appears that AML arises from a single progenitor cell which has undergone genetic transformation to an abnormal cell with the ability to proliferate rapidly. These abnormal immature myeloid cells accumulate in the bone marrow. This accumulation in the bone marrow interferes with the production of normal blood cells, including a reduction in red blood cells, platelets and neutrophils. Eventually the bone marrow stops working correctly.

AML is one of the most common types of leukemia among adults, and the most common acute leukemia affecting adults. In the U.S. alone, there are approximately 12,000 new cases each year. The incidence of AML is expected to increase as the population ages. In addition, in the U.S., about 11% of the cases of leukemia in childhood are AML. Chemotherapy is generally used to treat AML. Only a minority of patients are cured with current therapy.

Chemotherapy has a number of deleterious side effects. One of the side effects is myeloablative bone marrow toxicities. Bone marrow is the tissue that fills the inside of some bones. Examples of such bones are sternum, hip, femur and humerus. Bone marrow contains stem cells that develop into several types of blood cells: erythrocytes (red blood cells), leukocytes (white blood cells) and thrombocytes (platelets). Cells in the bone marrow are susceptible to the effects of chemotherapy due to their rapid rate of division. Bone marrow is prevented by chemotherapeutic agents from forming new blood cells. With time after exposure to a chemotherapeutic agent, counts of the blood cells will fall at various rates, depending upon the particular type of cell as their average life spans differ. Low white blood cell count, for example, makes an individual more susceptible to infection. Low red blood cell count, for example, causes an individual to be fatigued. Low platelet count, for example, impairs an individual's ability to make a blood clot.

As understood by a person of ordinary skill in the medical art, the terms, “treat” and “treatment,” include medical management of a disease, disorder, or condition of a subject (i.e., patient, individual) (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated with the disease, condition, or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment. Subjects in need of treatment include those who already have the disease, condition, or disorder as well as subjects prone to have or at risk of developing the disease, condition, or disorder, and those in which the disease, condition, or disorder is to be prevented (i.e., decreasing the likelihood of occurrence of the disease, disorder, or condition).

In some embodiments of the methods described herein, the subject is a human. In some embodiments of the methods described herein, the subject is a non-human animal. A subject in need of treatment as described herein may exhibit at least one symptom or sequelae of the disease, disorder, or condition described herein or may be at risk of developing the disease, disorder, or condition. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing a disease, disorder, or condition described herein can readily be determined by a person of ordinary skill in the medical and clinical arts. Determining and adjusting an appropriate dosing regimen (e.g., adjusting the amount of compound per dose and/or number of doses and frequency of dosing) can also readily be performed by a person of ordinary skill in the medical and clinical arts. One or any combination of diagnostic methods, including physical examination, assessment and monitoring of clinical symptoms, and performance of analytical tests and methods described herein, may be used for monitoring the health status of the subject.

Also provided herein are pharmaceutical compositions comprising at least one compound of Formula (II). In some embodiments, the pharmaceutical composition further comprises at least one additional pharmaceutically acceptable ingredient.

In pharmaceutical dosage forms, any one or more of the compounds of the present disclosure may be administered in the form of a pharmaceutically acceptable derivative, such as a salt, and/or it/they may also be used alone and/or in appropriate association, as well as in combination, with other pharmaceutically active compounds.

An effective amount or therapeutically effective amount refers to an amount of a compound of the present disclosure or a composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject.

An effective amount or therapeutically effective amount refers to an amount of a compound of the present disclosure or a composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject.

The minimum dose that is sufficient to provide effective therapy may be used in some embodiments. Subjects may generally be monitored for therapeutic effectiveness using assays suitable for the disease or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease or disorder may be determined according to parameters understood by a person of ordinary skill in the medical art.

Pharmaceutical compositions may be administered in any manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical composition described herein may be sterile aqueous or sterile non-aqueous solutions, suspensions or emulsions, and may additionally comprise at least one pharmaceutically acceptable excipient (i.e., a non-toxic material that does not interfere with the activity of the active ingredient). Such compositions may be in the form of a solid, liquid, or gas (aerosol). Alternatively, the compositions described herein may be formulated as a lyophilizate, or compounds described herein may be encapsulated within liposomes using technology known in the art. The pharmaceutical compositions may further comprise at least one additional pharmaceutical acceptable ingredient, which may be biologically active or inactive. Non-limiting examples of such ingredients include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in pharmaceutical compositions may be employed in the compositions described herein. Excipients for therapeutic use are well known, and are described, for example, in Remington: The Science and Practice of Pharmacy (Gennaro, 21^{st} Ed. Mack Pub. Co., Easton, PA (2005)). In general, the type of excipient is selected based on the mode of administration, as well as the chemical composition of the active ingredient(s). Pharmaceutical compositions may be formulated for the particular mode of administration. For parenteral administration, pharmaceutical compositions may further comprise water, saline, alcohols, fats, waxes, and buffers. For oral administration, pharmaceutical compositions may further comprise at least one ingredient chosen, for example, from any of the aforementioned excipients, solid excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid pharmaceutical composition may include, for example, at least one the following: a sterile diluent such as water for injection, saline solution, physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition is an injectable pharmaceutical composition, and in some embodiments, the injectable pharmaceutical composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid, or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible and may also be biodegradable. The formulation may provide a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The compositions may be formulated into pressurized acceptable propellants such as dichlorodifluoromethane, propane, nitrogen and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneräs et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug larget 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., UJrology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound or composition comprising the same.

### EXAMPLES

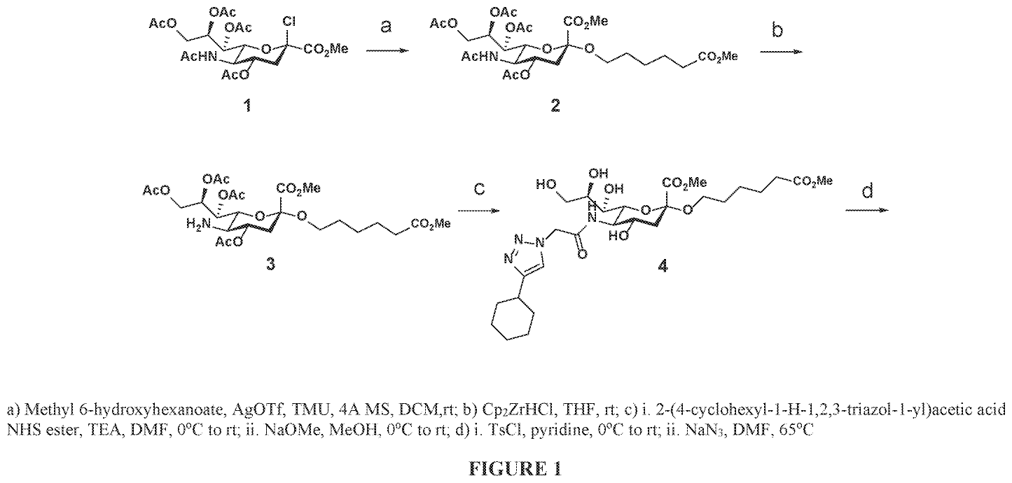

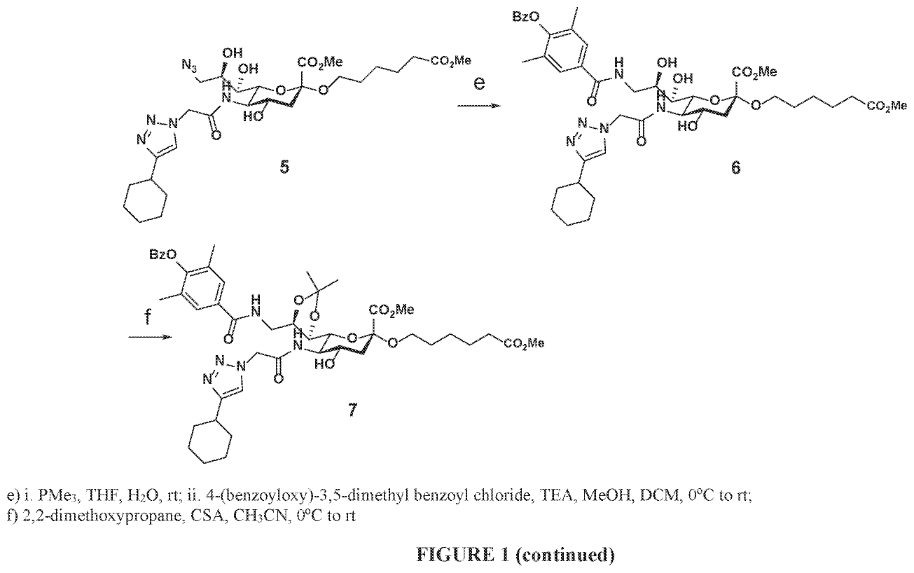

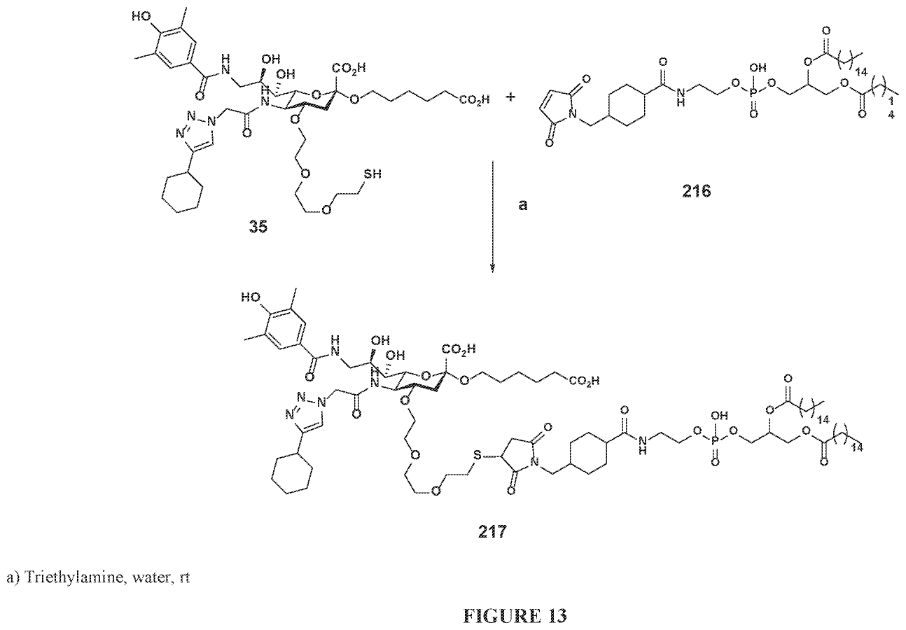

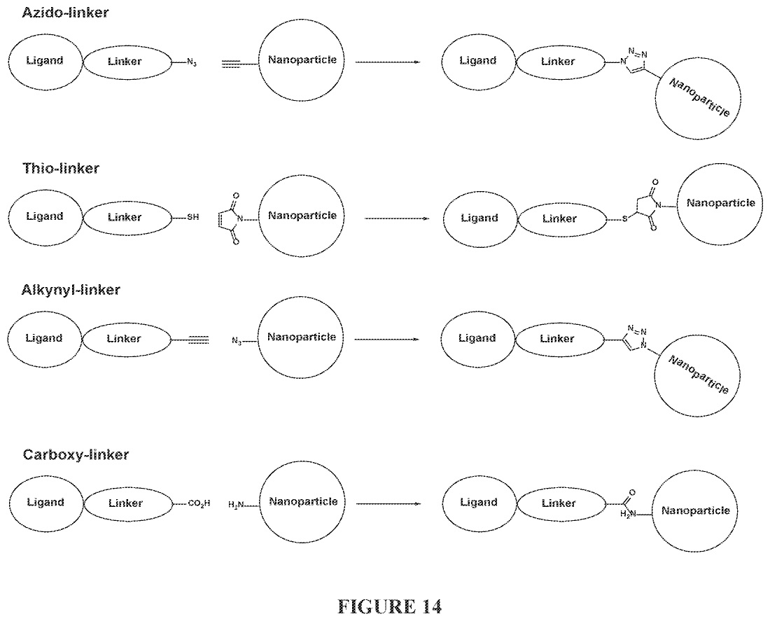

Compounds of Formula (I) may be prepared as shown in FIGS. 1-13. It is understood that one of ordinary skill in the art may be able to make these compounds by similar methods or by combining other methods known to one of ordinary skill in the art. It is also understood that one of ordinary skill in the art would be able to make other compounds of Formula (I) not specifically illustrated herein by using appropriate starting components and modifying the parameters of the synthesis as needed. In general, starting components may be obtained from sources such as Sigma Aldrich, Alfa Aesar, Maybridge, Matrix Scientific, TCI, and Fluorochem USA, etc. and/or synthesized according to sources known to those of ordinary skill in the art (see, e.g., Advanced Organic Chemistry: Reactions, Mechanisms, and Structure, 5th edition (Wiley, December 2000)) and/or prepared as described herein.

It will also be appreciated by those skilled in the art that in the processes described herein the functional groups of intermediate compounds may need to be protected by suitable protecting groups, even if not specifically described. Such functional groups include, but are not limited to, hydroxy, amino, mercapto, and carboxylic acid. Suitable protecting groups for hydroxy include, but are not limited to, trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include, but are not limited to, t-butoxycarbonyl, benzyloxycarbonyl, and the like. Suitable protecting groups for mercapto include, but are not limited to, —C(O)R″ (where R″ is alkyl, aryl or arylalkyl), p-methoxybenzyl, trityl and the like. Suitable protecting groups for carboxylic acid include, but are not limited to, alkyl, aryl, or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8: Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

### Example 1

### Prophetic Synthesis of Compound 10

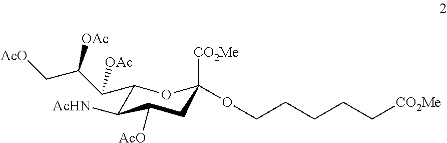

Compound 2: To a mixture of the sialyl chloride 1 and methyl 6-hydroxyhexanoate dissolved in DCM under argon is added molecular sieves and 1,1,3,3-tetramethylurea. The reaction mixture is stirred at room temperature for 30 min. Silver triflate is added and the mixture is stirred at rt until completion. The reaction mixture is filtered then concentrated. The residue is separated by flash chromatography to afford compound 2.

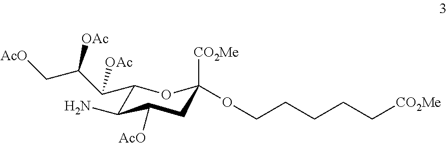

Compound 3: To a suspension of zirconocene hydrochloride (Schwarz reagent) in THF at room temperature is added compound 2. The reaction mixture is stirred at room temperature until it becomes homogenous. The reaction mixture is quenched by the addition of 0.1 N HCl, diluted with ethyl acetate and transferred to a separatory funnel. The reaction mixture is washed 2 times with water and 1 time with brine. The organic phase is dried over magnesium sulfate, filtered and concentrated. The residue is purified by flash chromatography to afford compound 3.

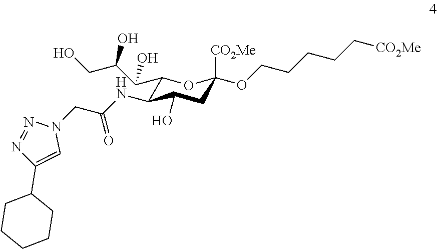

Compound 4: Compound 3 is dissolved in DMF and cooled on an ice bath. Triethylamine is added followed by 2-(4-cyclohexyl-1-H-1,2,3-triazol-1yl)acetic acid NHS ester. The reaction mixture is stirred allowing to warm to room temperature. Upon completion, the solvent is removed and the residue is dissolved in methanol. Sodium methoxide is added and the reaction mixture is stirred at room temperature until completion. The reaction mixture is quenched by the addition of acetic acid and concentrated. The residue is purified by flash chromatography to afford compound 4.

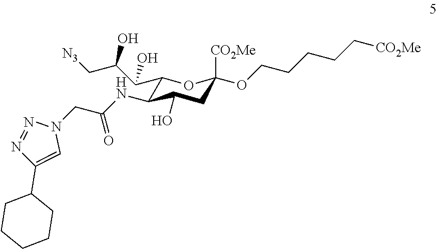

Compound 5: Compound 4 is dissolved in pyridine and cooled on an ice bath. Tosyl chloride is dissolved in pyridine and added dropwise. The reaction mixture is stirred allowing to warm to room temperature. Upon completion methanol is added. The reaction mixture is concentrated. The residue is dissolved in ethyl acetate, transferred to a separatory funnel, and washed 2 times with water and 1 time with brine. The organic phase is dried over magnesium sulfate, filtered and concentrated. The residue is dissolved in DMF. Sodium azide is added and the reaction mixture is stirred at 65° C. until completion. The solvent is removed and the residue is purified by flash chromatography to afford compound 5.

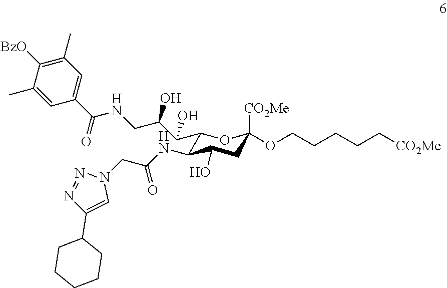

Compound 6: Compound 5 is dissolved in THF/water (1/1 v/v) at room temperature. Trimethylphosphine (1M in THF) is added. The reaction mixture is stirred at room temperature until completion. The solvent is evaporated and the residue is concentrated from methanol to remove residual water. The residue is dissolved in methanol and cooled on an ice bath. Triethylamine is added followed by dropwise addition of 4-(benzoyloxy)-3,5-dimethyl benzoyl chloride (as a 1 M solution in methylene chloride). The reaction mixture is stirred at room temperature until completion. The solvent is removed and the residue is purified by flash chromatography to afford compound 6.

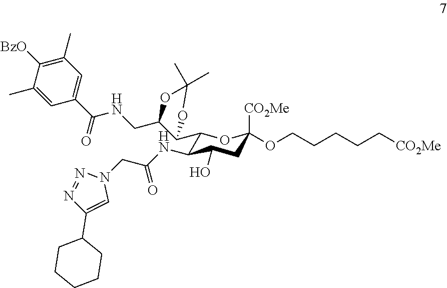

Compound 7: Compound 6 is dissolved in acetonitrile and cooled on an ice bath. Acetone dimethyl acetal is added followed by a catalytic amount of camphorsulfonic acid. The ice bath is removed and the reaction mixture is stirred at room temperature. Upon completion, the reaction mixture is quenched by the addition of triethylamine. The solvent is removed and the residue is purified by flash chromatography to afford compound 7.

The following compounds can be prepared according to FIG. 1:

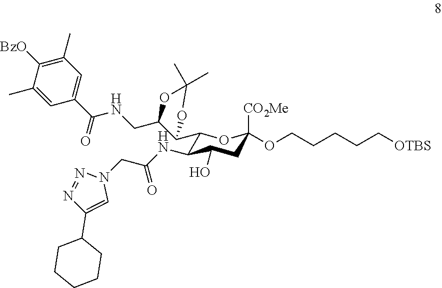

Compound 8: Compound 8 can be prepared according to FIG. 1 by using 5-[[(1,1-Dimethylethyl)dimethylsilyl]oxy]-1-pentanol in step a.

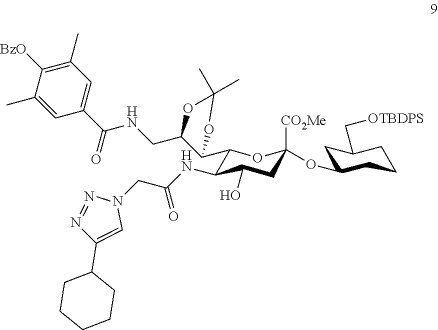

Compound 9: Compound 9 can be prepared according to FIG. 1 by using (IR,3S)-3-(tert-butyldiphenylsilyloxymethyl)cyclohexan-1-ol in step a.

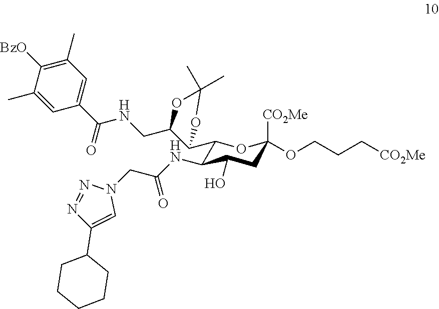

Compound 10: Compound 10 can be prepared according to FIG. 1 by using methyl 5-hydroxybutanoate in step a.

### Example 2

### Prophetic Synthesis of Compounds 12-139

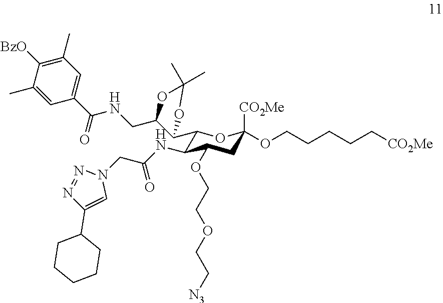

Compound 11: To a mixture of compound 10 and 2-(2-azidoethoxy)ethyl tosylate in DMF on an ice bath is added sodium hydride. The reaction mixture is stirred on the ice bath until completion. The reaction mixture is quenched by the addition of saturated ammonium chloride solution. The reaction mixture is diluted with ethyl acetate, transferred to a separatory funnel, and washed with water. The organic phase is dried over magnesium sulfate, filtered, and concentrated. The residue is purified by flash chromatography to afford compound 11.

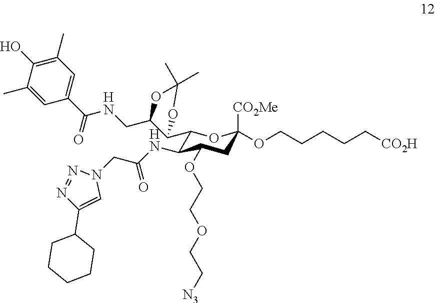

Compound 12: To a solution of compound 11 in acetonitrile on an ice bath is added 48% boron trifluoride diethyletherate. The reaction mixture is stirred on the ice bath until completion. The reaction mixture is quenched by the addition of saturated sodium bicarbonate solution. The reaction mixture is diluted with ethyl acetate, transferred to a separatory funnel, and washed with water. The organic phase is dried over magnesium sulfate, filtered, and concentrated. The residue is dissolved in methanol at room temperature and 10 N NaOH solution is added. The reaction mixture is stirred at room temperature until completion. The reaction mixture is concentrated and the residue is purified by reverse phase chromatography to afford compound 12.

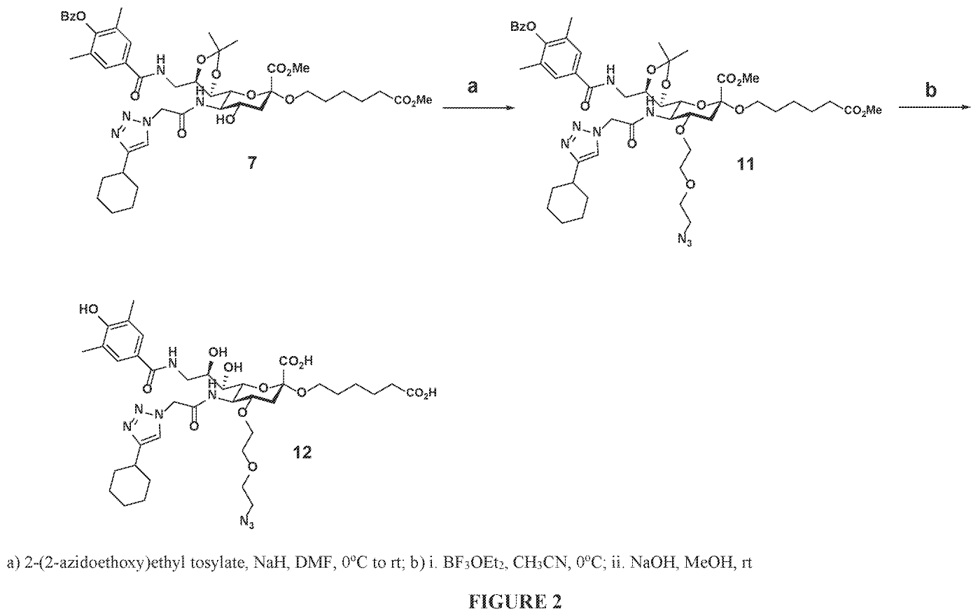

The following compounds can be prepared according to FIG. 2:

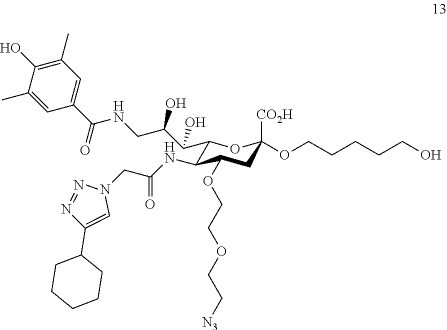

Compound 13: Compound 13 can be prepared according to FIG. 2 by using compound 8 in step a.

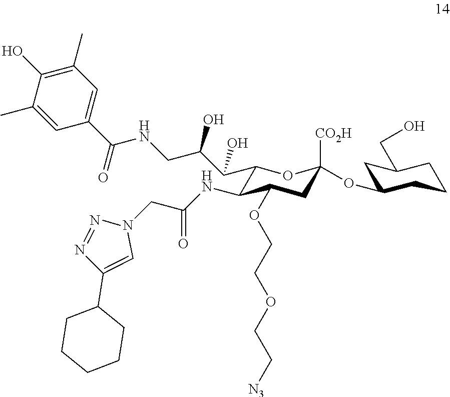

Compound 14: Compound 14 can be prepared according to FIG. 2 by using compound 9 in step a.

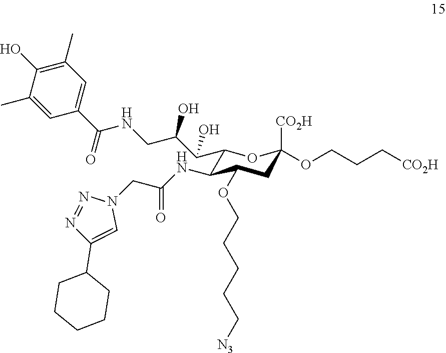

Compound 15: Compound 15 can be prepared according to FIG. 1 by using compound 10 in step a.

The following compounds can be prepared according to FIG. 2 using the appropriate alkylating agent in step b.

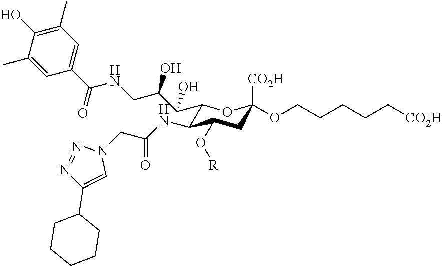

TABLE 1

| Compound | |
|---|---|
| Number | R |
| 16 | —CH₂CH₂OCH₂CH₂OCH₂CH₂—N₃ |
| 17 | —CH₂CH₂(OCH₂CH₂)₂OCH₂CH₂—N₃ |
| 18 | —CH₂CH₂(OCH₂CH₂)₃OCH₂CH₂—N₃ |
| 19 | —CH₂CH₂(OCH₂CH₂)₄OCH₂CH₂—N₃ |
| 20 | —CH₂CH₂(OCH₂CH₂)₅OCH₂CH₂—N₃ |
| 21 | —CH₂CH₂(OCH₂CH₂)₆OCH₂CH₂—N₃ |
| 22 | —CH₂CH₂(OCH₂CH₂)₇OCH₂CH₂—N₃ |
| 23 | —CH₂CH₂(OCH₂CH₂)₈OCH₂CH₂—N₃ |
| 24 | —CH₂CH₂(OCH₂CH₂)₉OCH₂CH₂—N₃ |
| 25 | —CH₂CH₂(OCH₂CH₂)₁₀OCH₂CH₂—N₃ |
| 26 | —CH₂CH₂(OCH₂CH₂)₁₁OCH₂CH₂—N₃ |
| 27 | —CH₂CH₂(OCH₂CH₂)₁₂OCH₂CH₂—N₃ |
| 28 | —CH₂CH₂(OCH₂CH₂)₁₃OCH₂CH₂—N₃ |
| 29 | —CH₂CH₂(OCH₂CH₂)₁₄OCH₂CH₂—N₃ |
| 30 | —CH₂CH₂(OCH₂CH₂)₁₅OCH₂CH₂—N₃ |
| 31 | —CH₂CH₂(OCH₂CH₂)₁₆OCH₂CH₂—N₃ |
| 32 | —CH₂CH₂(OCH₂CH₂)₂₀OCH₂CH₂—N₃ |
| 33 | —CH₂CH₂(OCH₂CH₂)₂₄OCH₂CH₂—N₃ |
| 34 | —CH₂CH₂OCH₂CH₂—SH |
| 35 | —CH₂CH₂OCHCH₂OCH₂CH₂—SH |
| 36 | —CH₂CH₂(OCH₂CH₂)₂OCH₂CH₂—SH |
| 37 | —CH₂CH₂(OCH₂CH₂)₃OCH₂CH₂—SH |
| 38 | —CH₂CH₂(OCH₂CH₂)₄OCH₂CH₂—SH |
| 39 | —CH₂CH₂(OCH₂CH₂)₅OCH₂CH₂—SH |
| 40 | —CH₂CH₂(OCH₂CH₂)₆OCH₂CH₂—SH |
| 41 | —CH₂CH₂(OCH₂CH₂)₇OCH₂CH₂—SH |
| 42 | —CH₂CH₂(OCH₂CH₂)₈OCH₂CH₂—SH |
| 43 | —CH₂CH₂(OCH₂CH₂)₉OCH₂CH₂—SH |
| 44 | —CH₂CH₂(OCH₂CH₂)₁₀OCH₂CH₂—SH |
| 45 | —CH₂CH₂(OCH₂CH₂)₁₁OCH₂CH₂—SH |
| 46 | —CH₂CH₂(OCH₂CH₂)₁₂OCH₂CH₂—SH |
| 47 | —CH₂CH₂(OCH₂CH₂)₁₃OCH₂CH₂—SH |
| 48 | —CH₂CH₂(OCH₂CH₂)₁₄OCH₂CH₂—SH |
| 49 | —CH₂CH₂(OCH₂CH₂)₁₅OCH₂CH₂—SH |
| 50 | —CH₂CH₂(OCH₂CH₂)₁₆OCH₂CH₂—SH |
| 51 | —CH₂CH₂(OCH₂CH₂)₂₀OCH₂CH₂—SH |
| 52 | —CH₂CH₂(OCH₂CH₂)₂₄OCH₂CH₂—SH |
| 53 | —CH₂CH₂OCH₂C≡CH |
| 54 | —CH₂CH₂OCH₂CH₂OCH₂C≡CH |
| 55 | —CH₂CH₂(OCH₂CH₂)₂OCH₂C≡CH |
| 56 | —CH₂CH₂(OCH₂CH₂)₃OCH₂C≡CH |
| 57 | —CH₂CH₂(OCH₂CH₂)₄OCH₂C≡CH |
| 58 | —CH₂CH₂(OCH₂CH₂)₅OCH₂C≡CH |
| 59 | —CH₂CH₂(OCH₂CH₂)₆OCH₂C≡CH |
| 60 | —CH₂CH₂(OCH₂CH₂)₇OCH₂C≡CH |
| 61 | —CH₂CH₂(OCH₂CH₂)₈OCH₂C≡CH |
| 62 | —CH₂CH₂(OCH₂CH₂)₉OCH₂C≡CH |
| 63 | —CH₂CH₂(OCH₂CH₂)₁₀OCH₂C≡CH |
| 64 | —CH₂CH₂(OCH₂CH₂)₁₁OCH₂C≡CH |
| 65 | —CH₂(OCH₂CH₂)₁₂OCH₂C≡CH |
| 66 | —CH₂CH₂(OCH₂CH₂)₁₃OCH₂C≡CH |
| 67 | —CH₂CH₂(OCH₂CH₂)₁₄OCH₂C≡CH |
| 68 | —CH₂CH₂(OCH₂CH₂)₁₅OCH₂C≡CH |
| 69 | —CH₂CH₂(OCH₂CH₂)₁₆OCH₂C≡CH |
| 70 | —CH₂CH₂(OCH₂CH₂)₂₀OCH₂C≡CH |
| 71 | —CH₂CH₂OCH₂CH₂—CO₂tBu |
| 72 | —CH₂CH₂OCH₂CH₂OCH₂CH₂—CO₂tBu |
| 73 | —CH₂CH₂(OCH₂CH₂)₂OCH₂CH₂—CO₂tBu |
| 74 | —CH₂CH₂(OCH₂CH₂)₃OCH₂CH₂—CO₂tBu |
| 75 | —CH₂CH₂(OCH₂CH₂)₄OCH₂CH₂—CO₂tBu |
| 76 | —CH₂CH₂(OCH₂CH₂)₅OCH₂CH₂—CO₂tBu |
| 77 | —CH₂CH₂(OCH₂CH₂)₆OCH₂CH₂—CO₂tBu |
| 78 | —CH₂CH₂(OCH₂CH₂)₇OCH₂CH₂—CO₂tBu |
| 79 | —CH₂CH₂(OCH₂CH₂)₈OCH₂CH₂—CO₂tBu |
| 80 | —CH₂CH₂(OCH₂CH₂)₉OCH₂CH₂—CO₂tBu |
| 81 | —CH₂CH₂(OCH₂CH₂)₁₀OCH₂CH₂—CO₂tBu |
| 82 | —CH₂CH₂(OCH₂CH₂)₁₁OCH₂CH₂—CO₂tBu |
| 83 | —CH₂CH₂(OCH₂CH₂)₁₂OCH₂CH₂—CO₂tBu |
| 84 | —CH₂CH₂(OCH₂CH₂)₁₃OCH₂CH₂—CO₂tBu |
| 85 | —CH₂CH₂(OCH₂CH₂)₁₄OCH₂CH₂—CO₂tBu |
| 86 | —CH₂CH₂(OCH₂CH₂)₁₅OCH₂CH₂—CO₂tBu |
| 87 | —CH₂CH₂(OCH₂CH₂)₁₆OCH₂CH₂—CO₂tBu |
| 88 | —CH₂CH₂(OCH₂CH₂)₂₀OCH₂CH₂—CO₂tBu |
| 89 | —CH₂CH₂—N₃ |
| 90 | —CH₂CH₂CH₂—N₃ |
| 91 | —CH₂CH₂CH₂CH₂—N₃ |
| 92 | —CH₂(CH₂CH₂)₂CH₂—N₃ |
| 93 | —CH₂(CH₂CH₂)₄CH₂—N₃ |
| 94 | —CH₂(CH₂CH₂)₆CH₂—N₃ |
| 95 | —CH₂(CH₂CH₂)₈CH₂—N₃ |
| 96 | —CH₂(CH₂CH₂)₁₀CH₂—N₃ |
| 97 | —CH₂(CH₂CH₂)₁₂CH₂—N₃ |
| 98 | —CH₂(CH₂CH₂)₁₄CH₂—N₃ |
| 99 | —CH₂(CH₂CH₂)₁₈CH₂—N₃ |
| 100 | —CH₂(CH₂CH₂)₂₂CH₂—N₃ |
| 101 | —S—CH₂CH₂—SH |
| 102 | —CH₂CH₂CH₂—SH |
| 103 | —CH₂CH₂CH₂CH₂—SH |
| 104 | —CH₂(CH₂CH₂)₂CH₂—SH |
| 105 | —CH₂(CH₂CH₂)₄CH₂—SH |
| 106 | —CH₂(CH₂CH₂)₆CH₂—SH |
| 107 | —CH₂(CH₂CH₂)₈CH₂—SH |
| 108 | —CH₂(CH₂CH₂)₁₀CH₂—SH |
| 109 | —CH₂(CH₂CH₂)₁₂CH₂—SH |
| 110 | —CH₂(CH₂CH₂)₁₄CH₂—SH |
| 111 | —CH₂(CH₂CH₂)₁₈CH₂—SH |
| 112 | —CH₂(CH₂CH₂)₂₂CH₂—SH |
| 113 | —CH₂C≡CH |
| 114 | —CH₂CH₂C≡CH |
| 115 | —CH₂CH₂CH₂C≡CH |
| 116 | —CH₂CH₂CH₂CH₂C≡CH |
| 117 | —CH₂(CH₂CH₂)₂CH₂C≡CH |
| 118 | —CH₂(CH₂CH₂)₄CH₂C≡CH |
| 119 | —CH₂(CH₂CH₂)₆CH₂C≡CH |
| 120 | —CH₂(CH₂CH₂)₈CH₂C≡CH |
| 121 | —CH₂(CH₂CH₂)₁₀CH₂C≡CH |
| 122 | —CH₂(CH₂CH₂)₁₂CH₂C≡CH |
| 123 | —CH₂(CH₂CH₂)₁₄CH₂C≡CH |
| 124 | —CH₂(CH₂CH₂)₁₈CH₂C≡CH |
| 125 | —CH₂(CH₂CH₂)₂₂CH₂C≡CH |
| 126 | —CH₂—CO₂tBu |
| 127 | —CH₂CH₂—CO₂tBu |
| 128 | —CH₂CH₂CH₂—CO₂tBu |
| 129 | —CH₂CH₂CH₂CH₂—CO₂tBu |
| 130 | —CH₂(CH₂CH₂)₂CH₂—CO₂tBu |
| 131 | —CH₂(CH₂CH₂)₄CH₂—CO₂tBu |
| 132 | —CH₂(CH₂CH₂)₆CH₂—CO₂tBu |
| 133 | —CH₂(CH₂CH₂)₈CH₂—CO₂tBu |
| 134 | —CH₂(CH₂CH₂)₁₀CH₂—CO₂tBu |
| 135 | —CH₂(CH₂CH₂)₁₂CH₂—CO₂tBu |
| 136 | —CH₂(CH₂CH₂)₁₄CH₂—CO₂tBu |
| 137 | —CH₂(CH₂CH₂)₁₈CH₂—CO₂tBu |
| 138 | —CH₂(CH₂CH₂)₂₂CH₂—CO₂tBu |
| 139 | —CH₂CH≡CH₂ |

### Example 3

### Prophetic Synthesis of Compounds 142 and 143

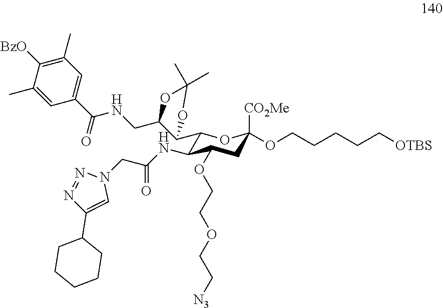

Compound 140: To a mixture of compound 8 and 2-(2-azidoethoxy)ethyl tosylate in DMF on an ice bath is added sodium hydride. The reaction mixture is stirred on the ice bath until completion. The reaction mixture is quenched by the addition of saturated ammonium chloride solution. The reaction mixture is diluted with ethyl acetate, transferred to a separatory funnel, and washed with water. The organic phase is dried over magnesium sulfate, filtered, and concentrated. The residue is purified by flash chromatography to afford compound 140.

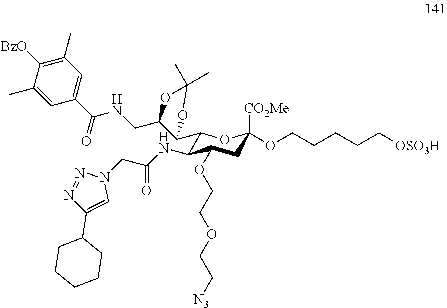

Compound 141. To a solution of compound 140 in THF is added TBAF (1.0 M in THF). The reaction mixture is stirred at room temperature until completion. The reaction mixture is quenched by the addition of saturated sodium bicarbonate solution. The reaction mixture is diluted with ethyl acetate, transferred to a separatory funnel, and washed with water. The organic phase is dried over magnesium sulfate, filtered, and concentrated. The residue is dissolved in dry DMF and SOrpyr is added under argon and the mixture is stirred at rt until completion. Solid NaHCO₃ is added to the solution, which is stirred vigorously for 10 min. Then, the suspension is filtered, and the solvents removed via co-evaporation with toluene. The residue is purified by flash chromatography to afford compound 141.

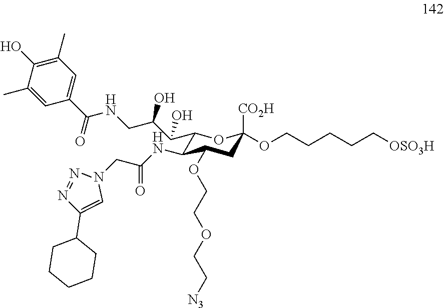

Compound 142: To a solution of compound 141 in acetonitrile on an ice bath is added 48% boron trifluoride diethyletherate. The reaction mixture is stirred on the ice bath until completion. The reaction mixture is quenched by the addition of saturated sodium bicarbonate solution. The reaction mixture is diluted with ethyl acetate, transferred to a separatory funnel, and washed with water. The organic phase is dried over magnesium sulfate, filtered, and concentrated. The residue is dissolved in methanol at room temperature and 10 N NaOH solution is added. The reaction mixture is stirred at room temperature until completion. The reaction mixture is concentrated and the residue is purified by reverse phase chromatography to afford compound 142.

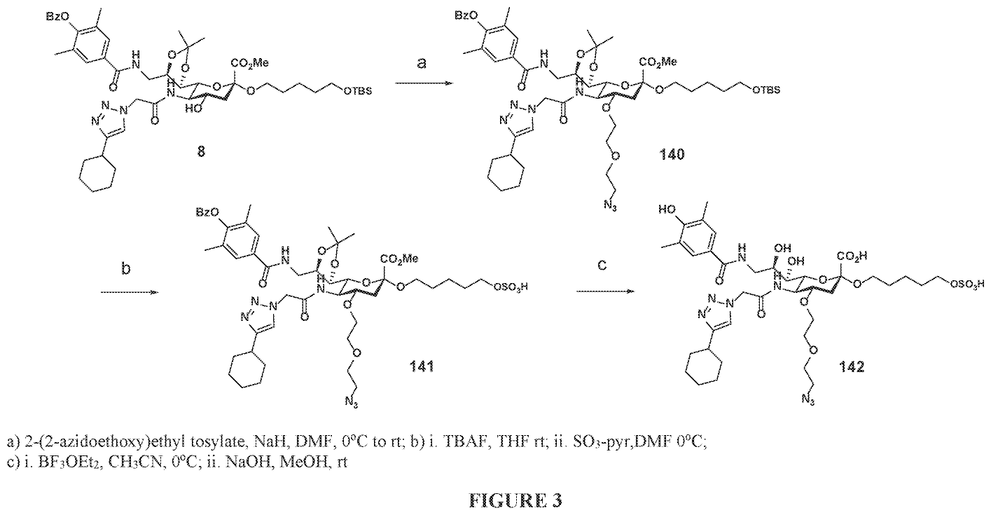

The following compounds can be prepared according to FIG. 3:

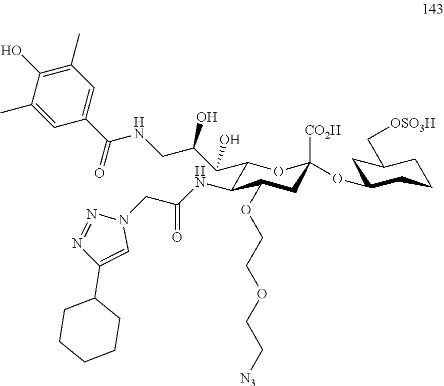

Compound 143: Compound 143 can be prepared according to FIG. 2 by using compound 9 in step a.

### Example 4

### Prophetic Synthesis of Compounds 146-150

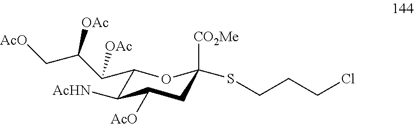

Compound 144: To a solution of sialyl chloride 1 and 3-chloropropanethiol (1.3 eq) dissolved in THF at room temperature is added 15-crown-5 (1.2 eq). The reaction mixture is cooled on an ice bath and NaH (1.5 eq) is added. The ice bath was removed and the reaction mixture was stirred at room temperature until completion. The reaction mixture is diluted with ethyl acetate, transferred to a separatory funnel and washed 3 times with water. The organic phase is concentrated and the residue is separated by flash chromatography to afford compound 144.

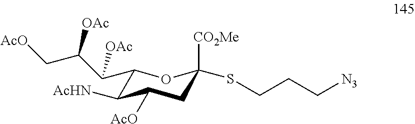

Compound 145: To a solution of compound 144 in DMF is added 15-crown-5 (1 eq) and sodium azide (10 eq). The reaction mixture is stirred at 45° C. until completion. The reaction mixture is cooled to room temperature, diluted with ethyl acetate and washed 3× with water. The organic phase is concentrated and separated by flash chromatography to afford compound 145.

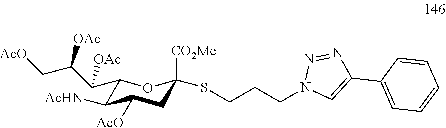

Compound 146: Compound 145, phenyl acetylene, copper sulfate (0.02 eq), and THPTA (0.02 eq) are combined in methanol at room temperature. Sodium ascorbate (1.5 eq) is added and the reaction mixture is stirred at room temperature. Upon completion, the reaction mixture is concentrated. The residue is diluted with ethyl acetate and washed 2 times with water. The organic phase is concentrated. The residue is purified by flash chromatography to afford compound 146.

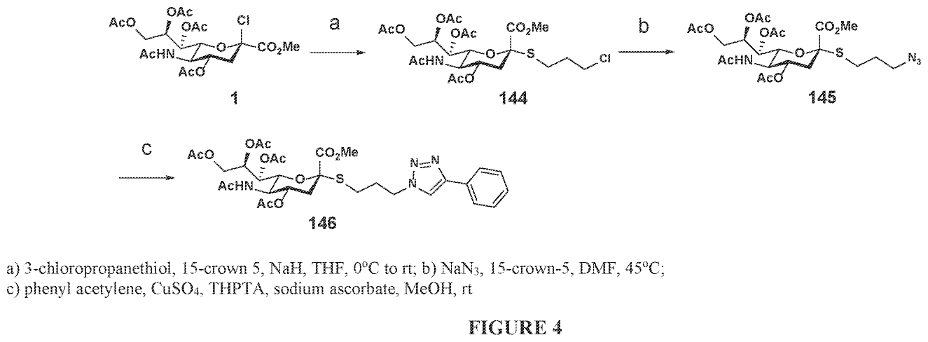

The following compounds can be prepared according to FIG. 4:

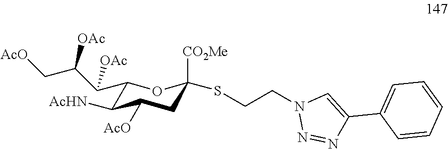

Compound 147: Compound 147 can be prepared according to FIG. 4 by using 2-chloroethanethiol in step a.

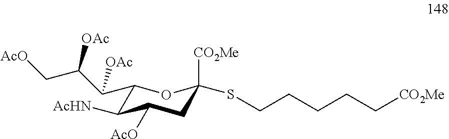

Compound 148: Compound 148 can be prepared according to FIG. 4 by using methyl 6-mercaptohexanoate in step a, and not performing steps b and c.

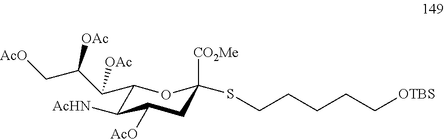

Compound 149: Compound 149 can be prepared according to FIG. 4 by using 5-tertbutyldimethylsiloxypentanethiol in step a, and not performing steps b and c.

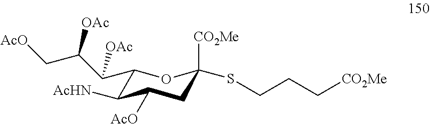

Compound 150: Compound 150 can be prepared according to FIG. 4 by using methyl 4-mercaptobutanoate in step a, and not performing steps b and c.

### Example 5

### Prophetic Synthesis of Compounds 160-168

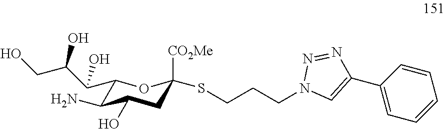

Compound 151: Compound 146 is dissolved in anhydrous methanol in a pressure tube. Methanesulfonic acid (10 eq) is added. The reaction mixture is heated at 85° C. overnight. The reaction mixture is neutralized by the addition of Dowex OH⁻ resin. The reaction mixture is filtered and concentrated to afford crude 151 which was used without further purification.

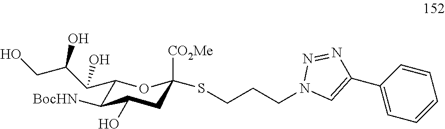

Compound 152: Compound 151 is added to a 5/1 v/v mixture of DCM/I M NaOH. The mixture is cooled on an ice bath. Di-tert-butyl dicarbonate (3 eq) is added. The reaction mixture is stirred overnight allowing to warm to room temperature. The reaction mixture is transferred to a separatory funnel and the phases separated. The organic phase is washed 2 times with water. The organic phase is concentrated and purified by flash chromatography to afford compound 152.

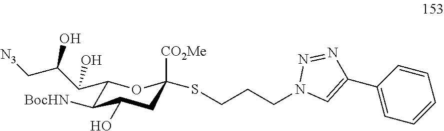

Compound 153: Compound 152 is dissolved in pyridine and cooled on an ice bath. Tosyl chloride is dissolved in pyridine and added dropwise. The reaction mixture is stirred allowing to warm to room temperature. Upon completion methanol is added. The reaction mixture is concentrated. The residue is dissolved in ethyl acetate, transferred to a separatory funnel, and washed 2 times with water and 1 time with brine. The organic phase is dried over magnesium sulfate, filtered and concentrated. The residue is dissolved in DMF. Sodium azide is added and the reaction mixture is stirred at 65° C. until completion. The solvent is removed and the residue is purified by flash chromatography to afford compound 153.

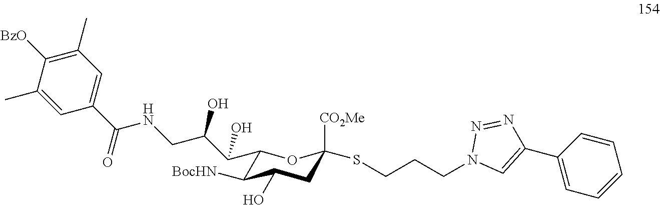

Compound 154: Compound 153 is dissolved in THF/water (1/1 v/v) at room temperature. Trimethylphosphine (1M in THF) is added. The reaction mixture is stirred at room temperature until completion. The solvent is evaporated and the residue is concentrated from methanol to remove residual water. The residue is dissolved in methanol and cooled on an ice bath. Triethylamine is added followed by dropwise addition of 4-(benzoyloxy)-3,5-dimethyl benzoyl chloride (as a 1 M solution in methylene chloride). The reaction mixture is stirred at room temperature until completion. The solvent is removed and the residue is purified by flash chromatography to afford compound 154.

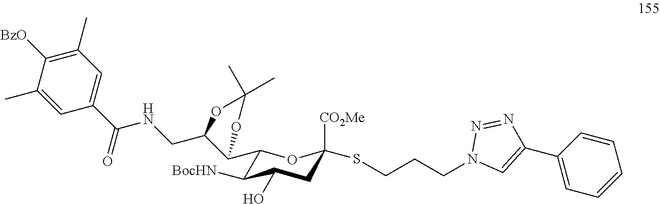

Compound 155: Compound 154 is dissolved in acetonitrile and cooled on an ice bath. Acetone dimethyl acetal is added followed by a catalytic amount of camphorsulfonic acid. The ice bath is removed and the reaction mixture is stirred at room temperature. Upon completion, the reaction mixture is quenched by the addition of triethylamine. The solvent is removed and the residue is purified by flash chromatography to afford compound 155.

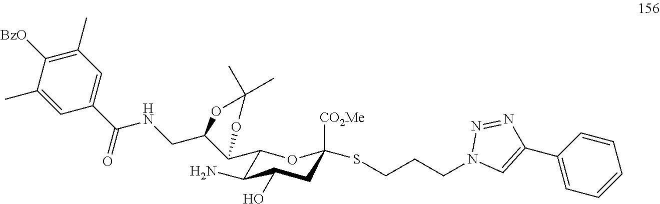

Compound 156: Compound 155 is dissolved in DCM and cooled on an ice bath. Trifluoroacetic acid (10 eq) is added and the reaction mixture is stirred on the ice bath until completion The solvent is removed and the residue is purified by flash chromatography to afford compound 156.

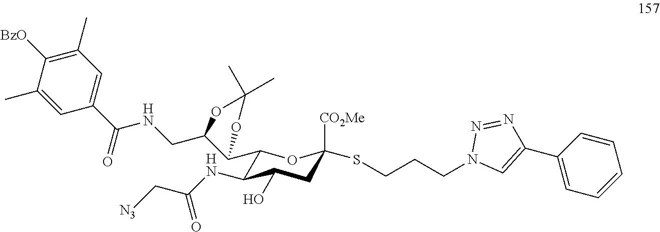

Compound 157: Compound 156 is dissolved in DMF and cooled on an ice bath. Triethylamine is added followed by 2-azidoacetic acid NHS ester. The reaction mixture is stirred allowing to warm to room temperature. Upon completion the solvent is removed and the residue is purified by flash chromatography to afford compound 157.

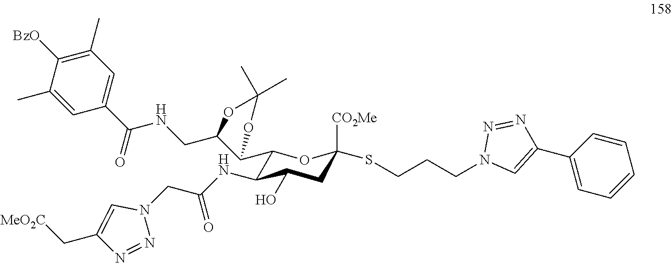

Compound 158: Compound 157, methyl 3-butynoate, copper sulfate (0.02 eq), and THPTA (0.02 eq) are combined in methanol at room temperature. The mixture is degassed by bubbling Ar through for 5 minutes. Sodium ascorbate (1.5 eq) is added and the reaction mixture is stirred at room temperature. Upon completion, the reaction mixture is concentrated. The residue is diluted with ethyl acetate and washed 2 times with water. The organic phase is concentrated and the residue is purified by flash chromatography to afford compound 158.

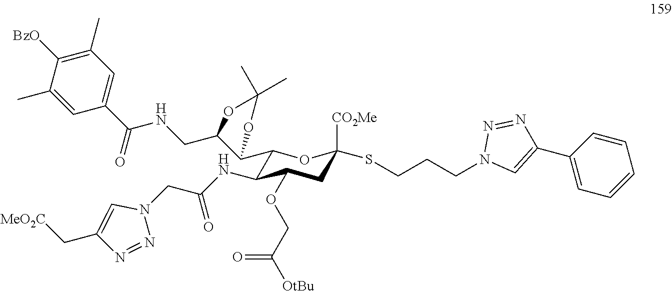

Compound 159: To a solution of compound 158 dissolved in anhydrous THF at room temperature is added tetrabutylammonium iodide (1.1 eq), tert-butyl bromoacetate (1.1 eq) and 15-crown-5 (1.1 eq). The reaction mixture is cooled on an ice bath and NaH (1.2 eq) is added. The reaction mixture is stirred on the ice bath until completion. The reaction mixture is diluted with ethyl acetate, transferred to a separatory funnel and washed with saturated ammonium chloride solution then water. The organic phase is concentrated and the residue is separated by flash chromatography to afford compound 159.

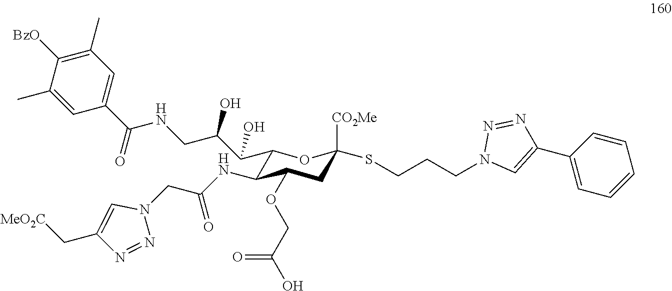

Compound 160: Compound 159 is dissolved in a 1/1 v/v mixture of trifluoroacetic acid/DCM at room temperature. The reaction mixture is stirred at 40° C. until completion. The reaction mixture is concentrated and separated by flash chromatography to afford compound 160.

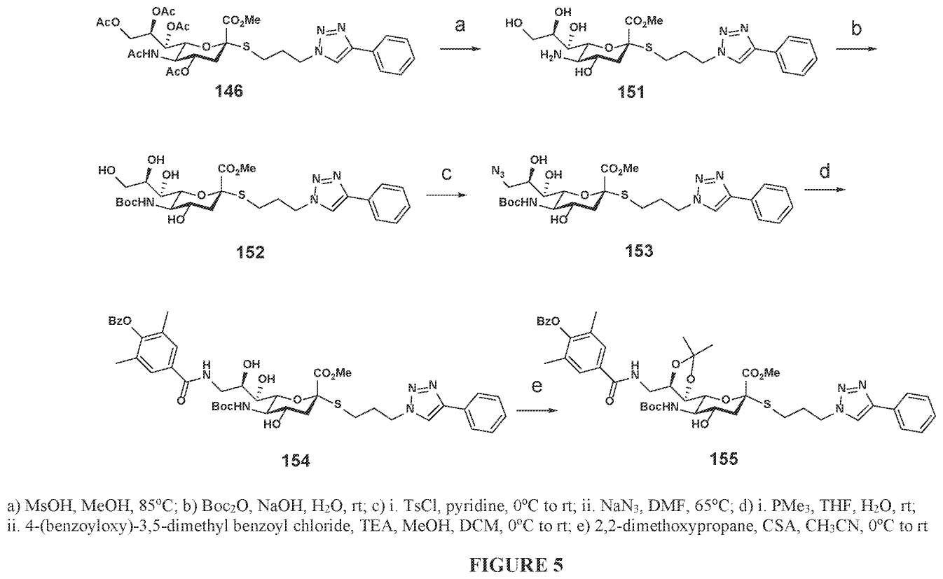

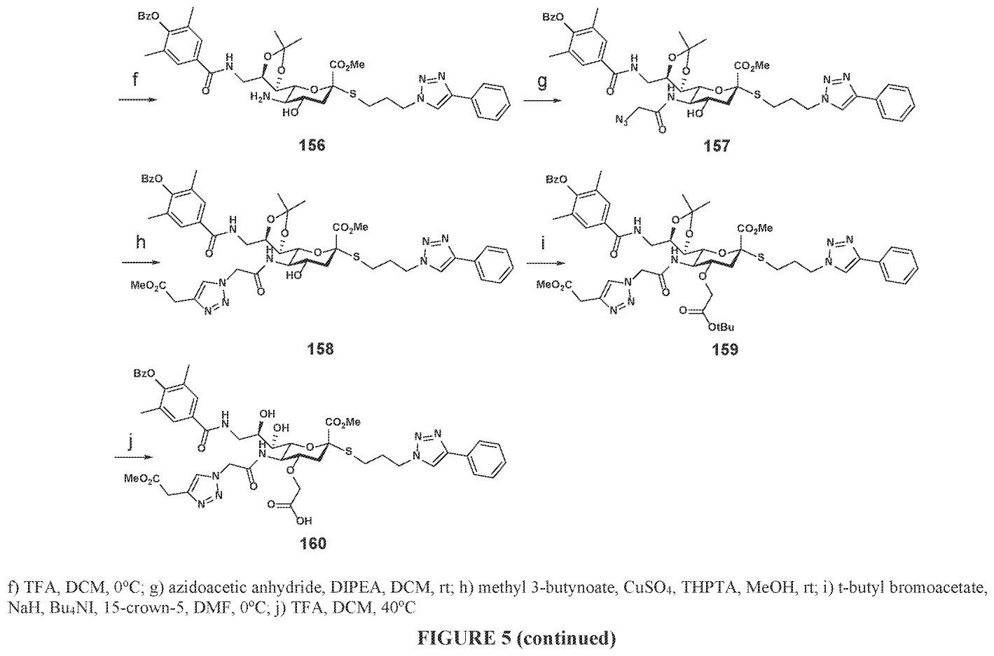

The following compounds can be prepared according to FIG. 5:

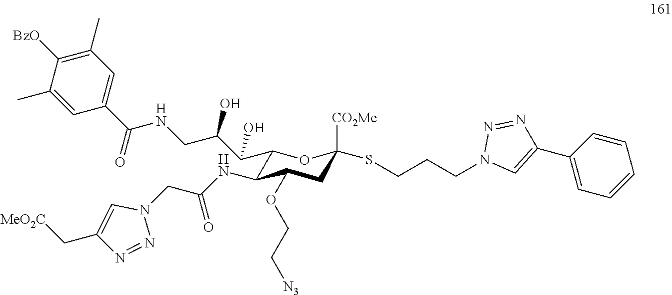

Compound 161: Compound 161 can be prepared according to FIG. 5 using 2-azidoethyl bromide in step i.

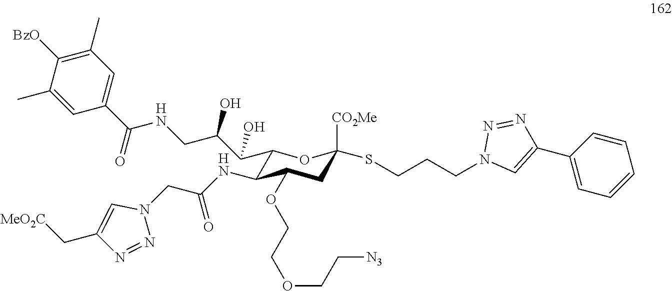

Compound 162: Compound 162 can be prepared according to FIG. 5 using 1-azido-2-(2-bromoethoxy)ethane in step i.

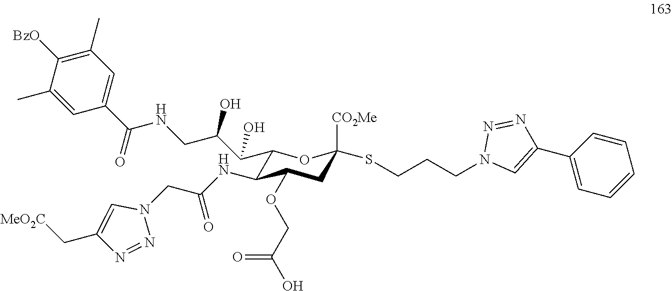

Compound 163: Compound 163 can be prepared according to FIG. 5 using methyl propioloate in step h.

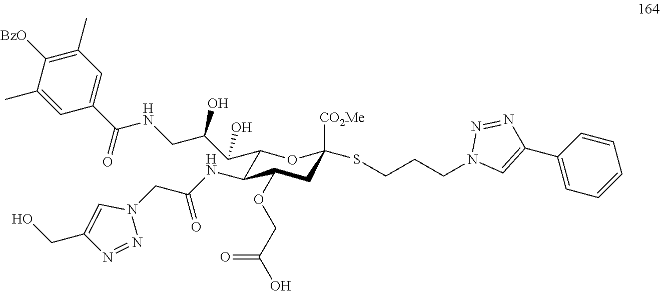

Compound 164: Compound 164 can be prepared according to FIG. 5 using propargyl trityl ether in step h.

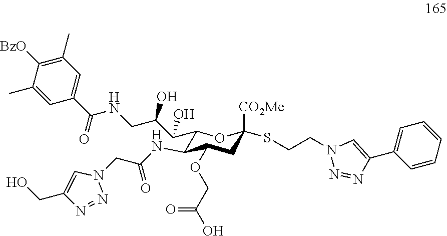

Compound 165: Compound 165 can be prepared according to FIG. 5 using compound 5 in step a.

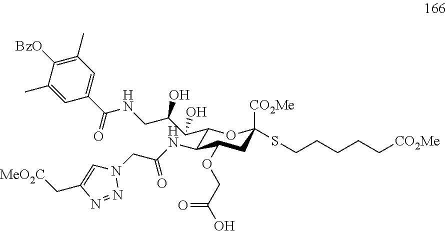

Compound 166: Compound 166 can be prepared according to FIG. 5 by using compound 148 step a.

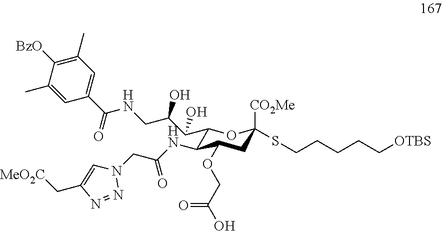

Compound 167: Compound 167 can be prepared according to FIG. 5 by using compound 149 in step a.

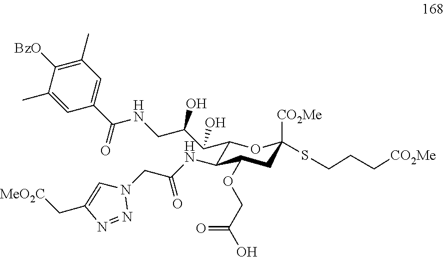

Compound 168: Compound 168 can be prepared according to FIG. 5 by using compound 150 in step a.

### Example 6

### Prophetic Syntheses of Compounds 170-176

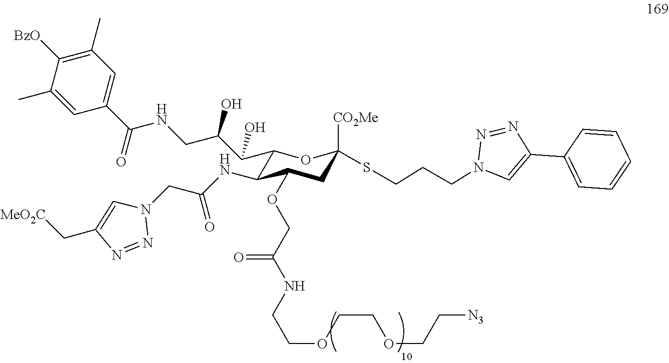

Compound 169: To a solution of compound 160 dissolved in DMF and cooled on an ice bath is added diisopropylethylamine (1.5 eq) followed by HATU (1.1 eq). The reaction mixture is stirred 10 minutes on the ice bath. Azido-PEG-11 amine (Broadpharm catalog item BP-22227, 1.3 eq) is added. The ice bath is removed and the reaction mixture is stirred at room temperature until completion. The reaction mixture is diluted with ethyl acetate, transferred to a separatory funnel, and washed 2 times with water. The organic phase is concentrated and separated by flash chromatography to afford compound 169.

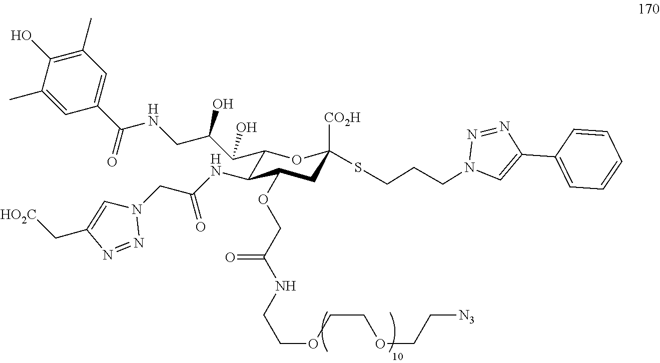

Compound 170: To a solution of compound 169 dissolved in 5/1 v/v methanol/water is added sodium methoxide (10 eq of a 1 M solution in methanol). The reaction mixture is stirred at room temperature until completion. The reaction mixture is neutralized with acetic acid then concentrated. The residue is separated by reverse phase chromatography to afford compound 170.

The following compounds can be prepared according to FIG. 6:

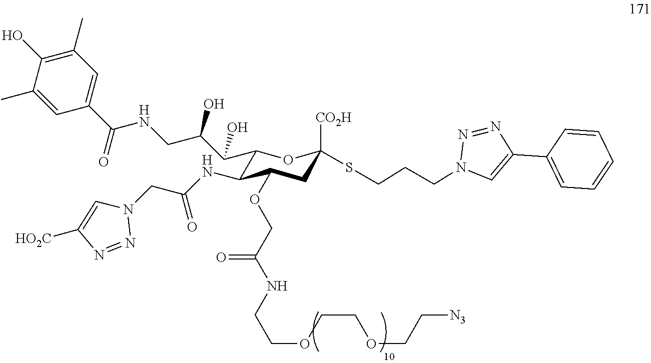

Compound 171: Compound 171 can be prepared according to FIG. 6 using compound 163 in step a.

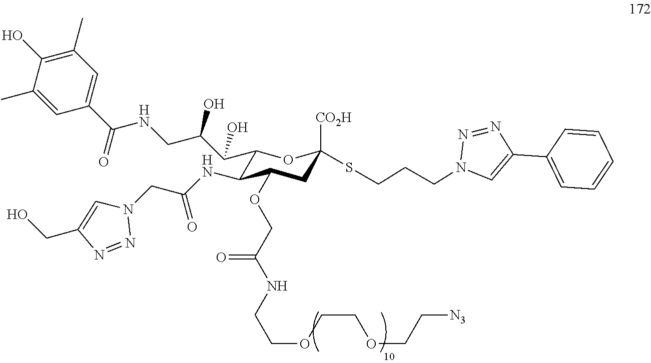

Compound 172: Compound 172 can be prepared according to FIG. 6 using compound 164 in step a.

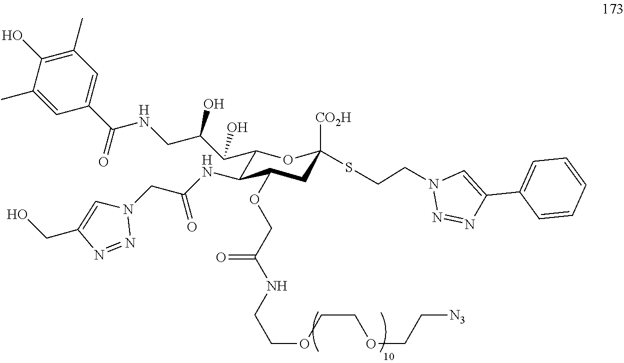

Compound 173: Compound 173 can be prepared according to FIG. 6 using compound 165 in step a.

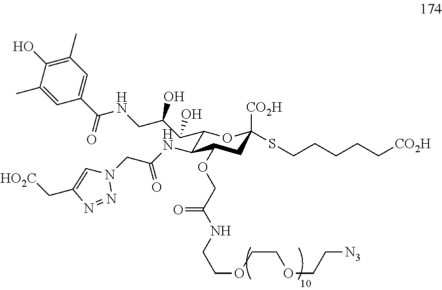

Compound 174: Compound 174 can be prepared according to FIG. 6 using compound 166 in step a.

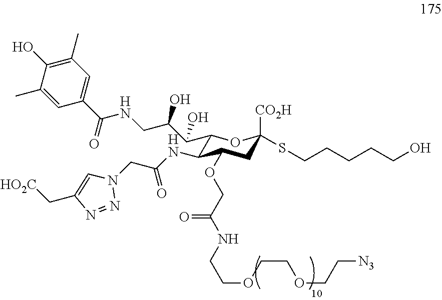

Compound 175: Compound 175 can be prepared according to FIG. 6 using compound 167 in step a.

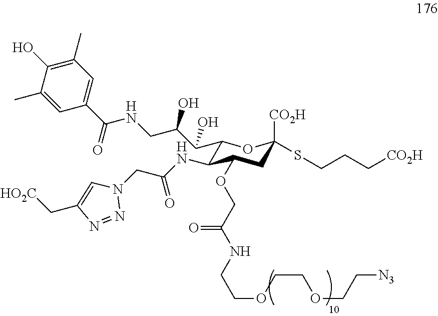

Compound 176: Compound 176 can be prepared according to FIG. 6 using compound 168 in step a.

### Example 7

### Prophetic Synthesis of Compound 177-183 and 219

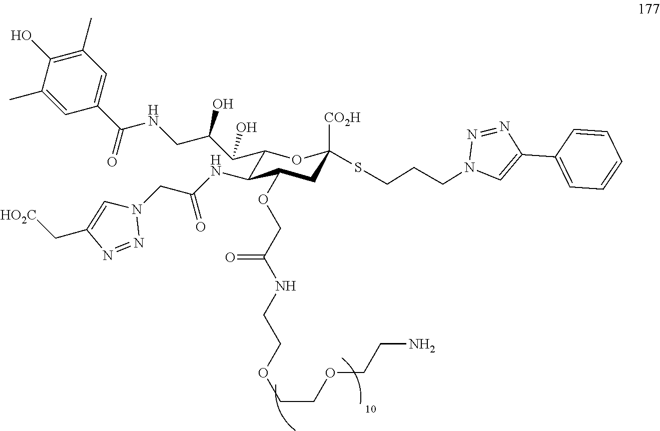

Compound 177: To a solution of compound 170 dissolved in DMF is added 1,4-dithiothreitol (3 eq) and DBU (0.1 eq). The reaction mixture is stirred at room temperature until completion. The reaction mixture is concentrated then separated by reverse phase chromatography to afford compound 177.

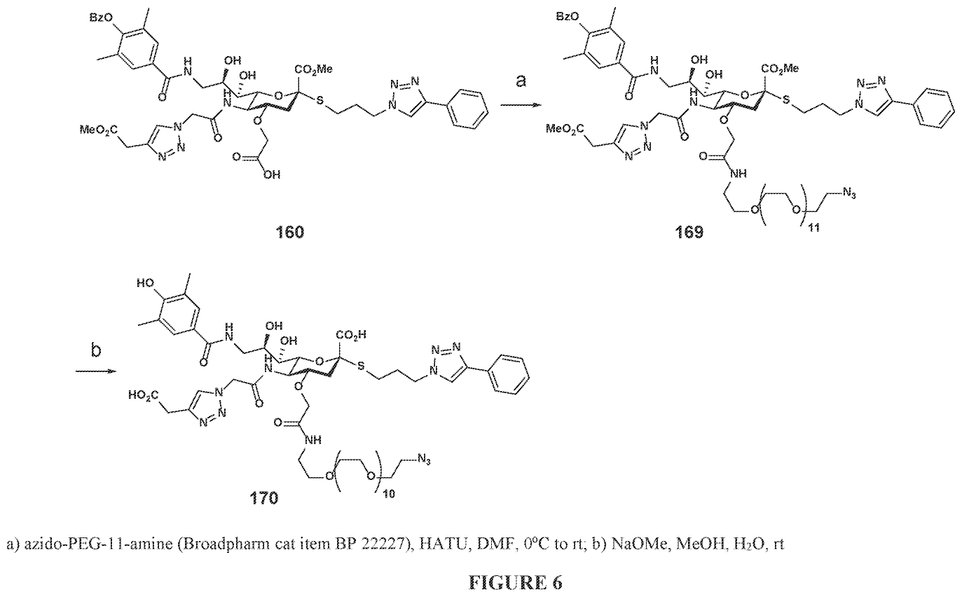

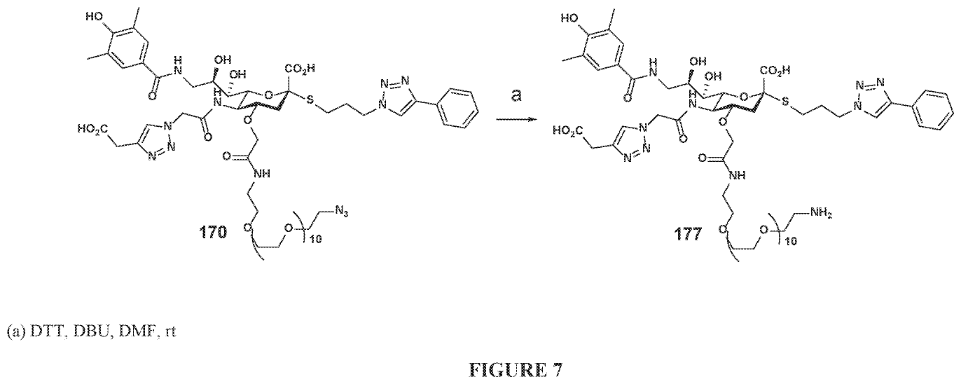

The following compounds can be prepared according to FIG. 7:

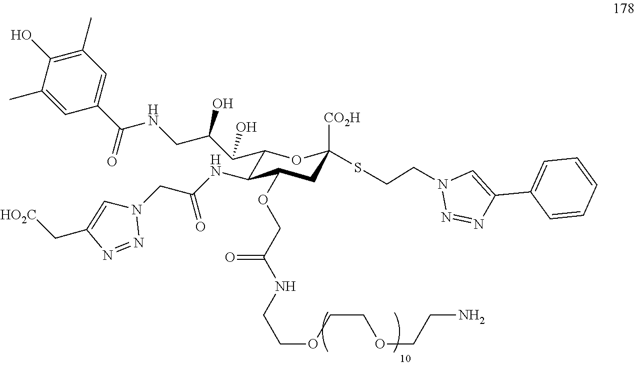

Compound 178: Compound 178 can be prepared according to FIG. 7 using compound 173 in step a.

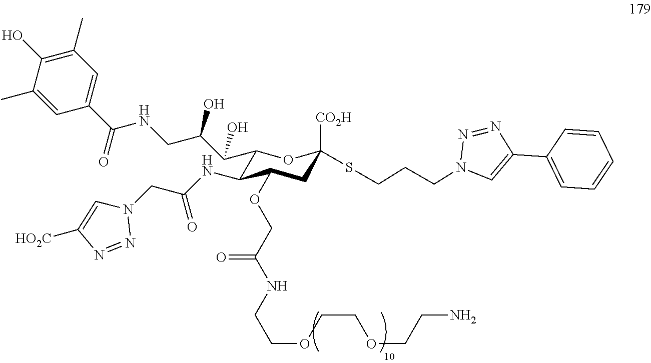

Compound 179: Compound 179 can be prepared according to FIG. 7 using compound 171 in step a.

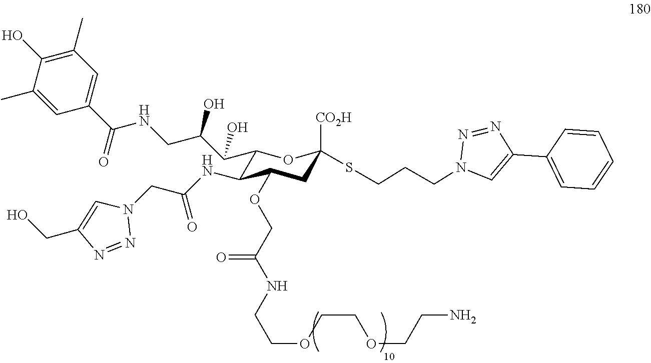

Compound 180: Compound 180 can be prepared according to FIG. 7 using compound 172 in step a.

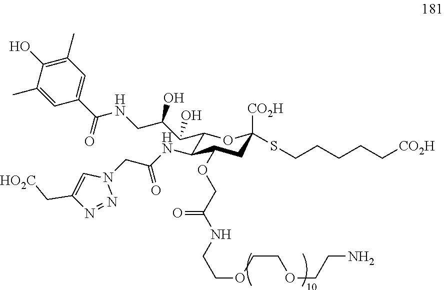

Compound 181: Compound 181 can be prepared according to FIG. 7 using compound 174 in step a.

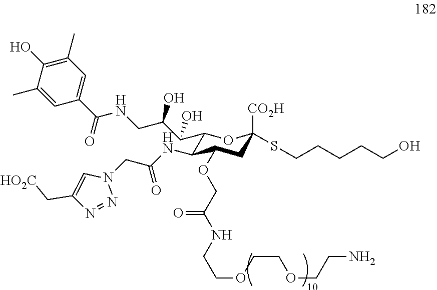

Compound 182: Compound 182 can be prepared according to FIG. 7 using compound 175 in step a.

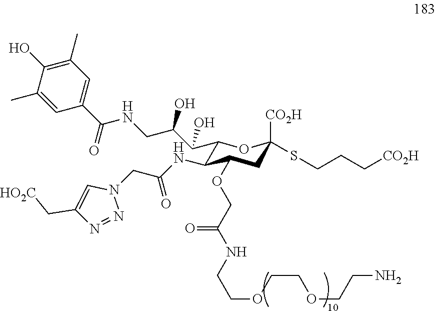

Compound 183: Compound 183 can be prepared according to FIG. 7 using compound 176 in step a.

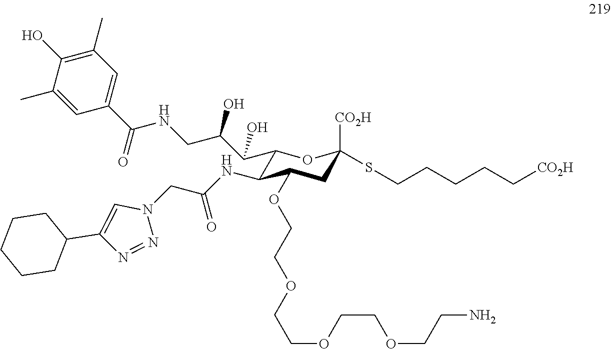

Compound 219: Compound 219 can be prepared according to FIG. 7 using compound 17 in step a.

### Example 8

### Prophetic Synthesis of Compounds 185-189

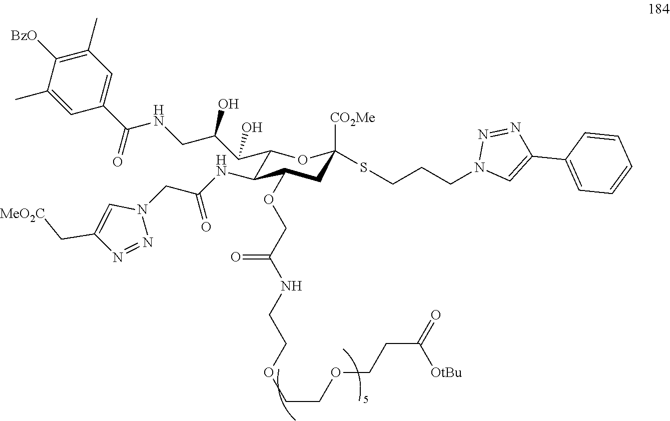

Compound 184: To a solution of compound 160 dissolved in DMF and cooled on an ice bath is added diisopropylethylamine (1.5 eq) followed by HATU (1.1 eq). The reaction mixture is stirred 10 minutes on the ice bath. Amino-PEG-6-tert-butyl ester (Broadpharm catalog item BP-20420, 1.3 eq) is added. The ice bath is removed and the reaction mixture is stirred at room temperature until completion. The reaction mixture is diluted with ethyl acetate, transferred to a separatory funnel, and washed 2 times with water. The organic phase is concentrated and separated by flash chromatography to afford compound 184.

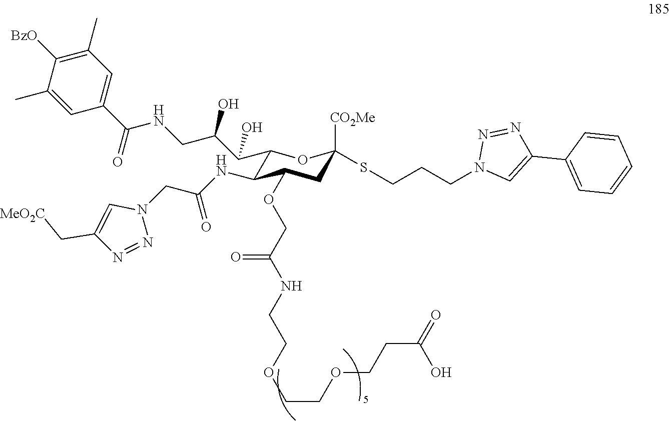

Compound 185: Compound 184 is dissolved in a 1/1 v/v mixture of trifluoroacetic acid/DCM at room temperature. The reaction mixture is stirred at 40° C. until completion. The reaction mixture is concentrated and separated by flash chromatography to afford compound 185.

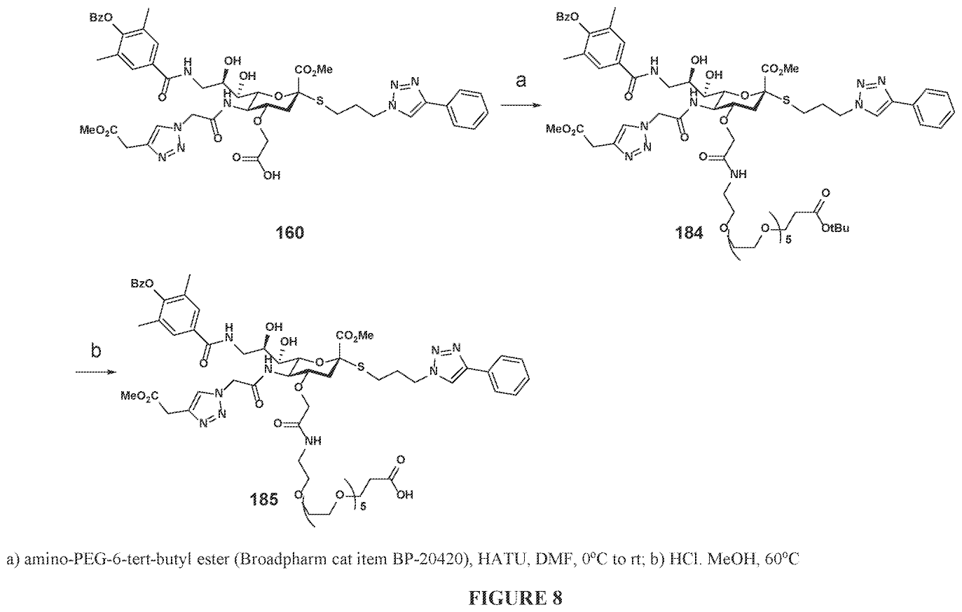

The following compounds can be prepared according to FIG. 8:

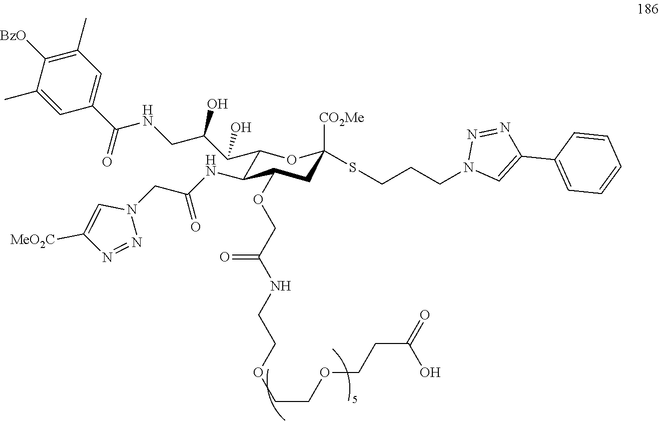

Compound 186: Compound 32 can be prepared according to FIG. 8 using compound 163 in step a.

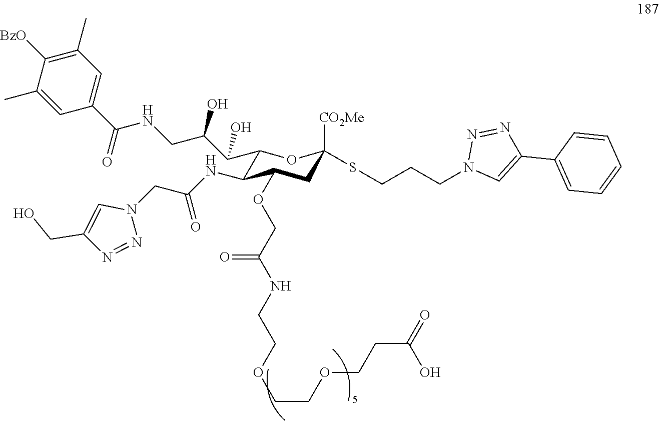

Compound 187 Compound 187 can be prepared according to FIG. 8 using compound 164 in step a.

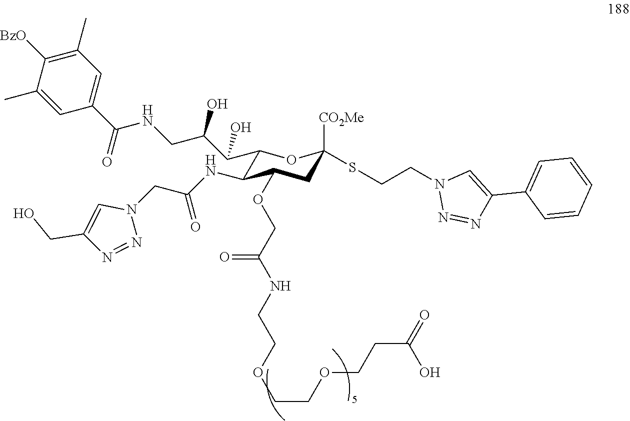

Compound 18 Compound 188 can be prepared according to FIG. 8 using compound 165 in step a.

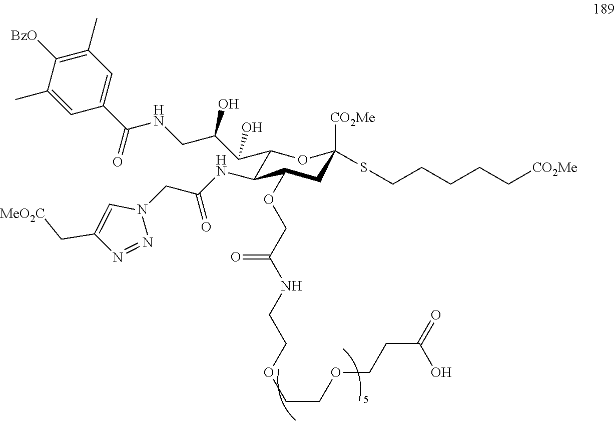

Compound 189: Compound 189 can be prepared according to FIG. 8 using compound 166 in step a.

### Example 9

### Prophetic Synthesis of Compounds 190-192

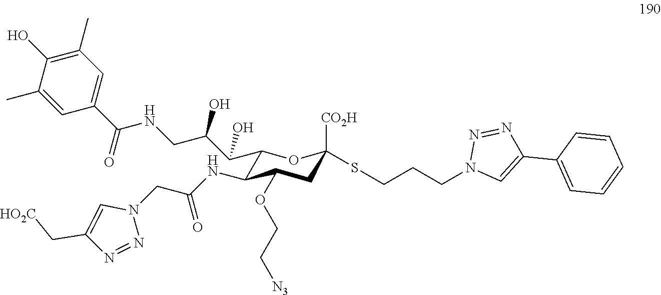

Compound 190: To a solution of compound 161 dissolved in 5/1 v/v methanol/water is added sodium methoxide (10 eq of a 1 M solution in methanol). The reaction mixture is stirred at room temperature until completion. The reaction mixture is neutralized with acetic acid then concentrated. The residue is separated by reverse phase chromatography to afford compound 190.

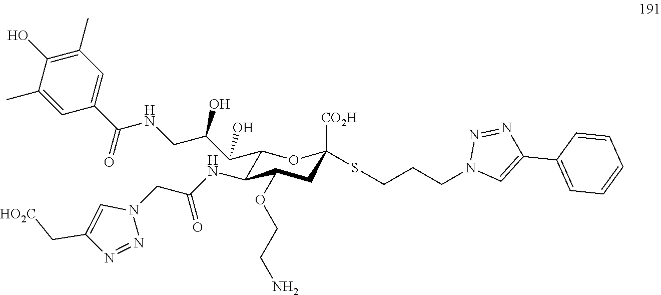

Compound 191: To a solution of compound 190 dissolved in DMF is added 1,4-dithiothreitol (3 eq) and DBU (0.1 eq). The reaction mixture is stirred at room temperature until completion. The reaction mixture is concentrated then separated by reverse phase chromatography to afford compound 191.

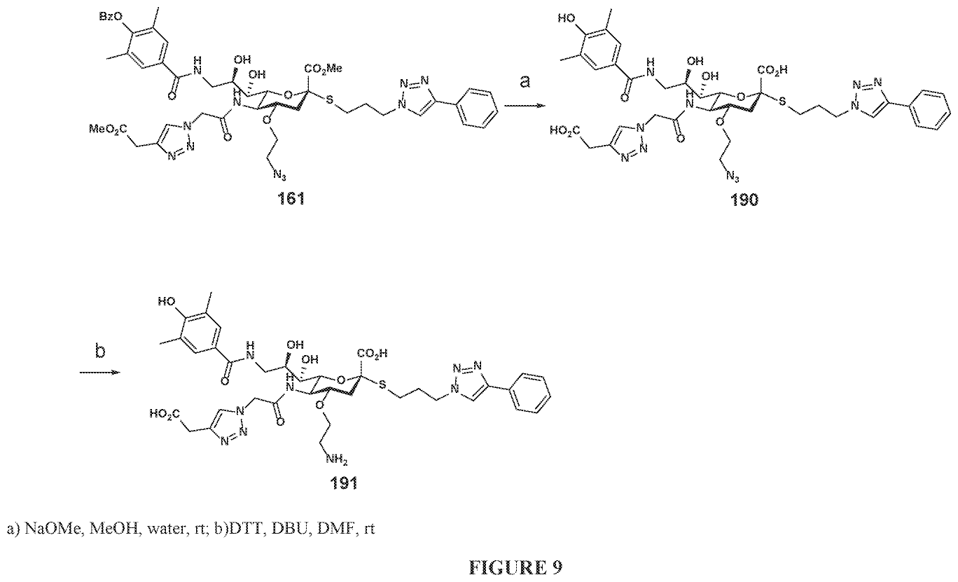

The following compounds can be prepared according to FIG. 9:

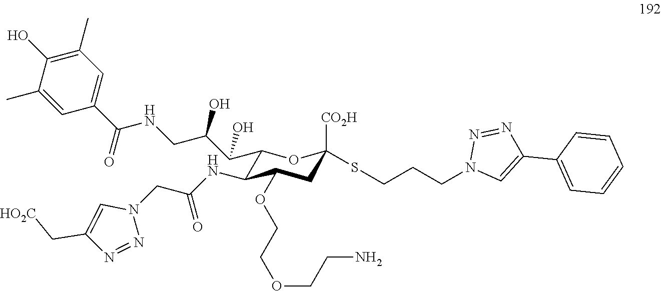

Compound 192: Compound 192 can be prepared according to FIG. 9 using compound 162 in step a.

### Example 10

### Prophetic Synthesis of Compounds 194-199 and 218

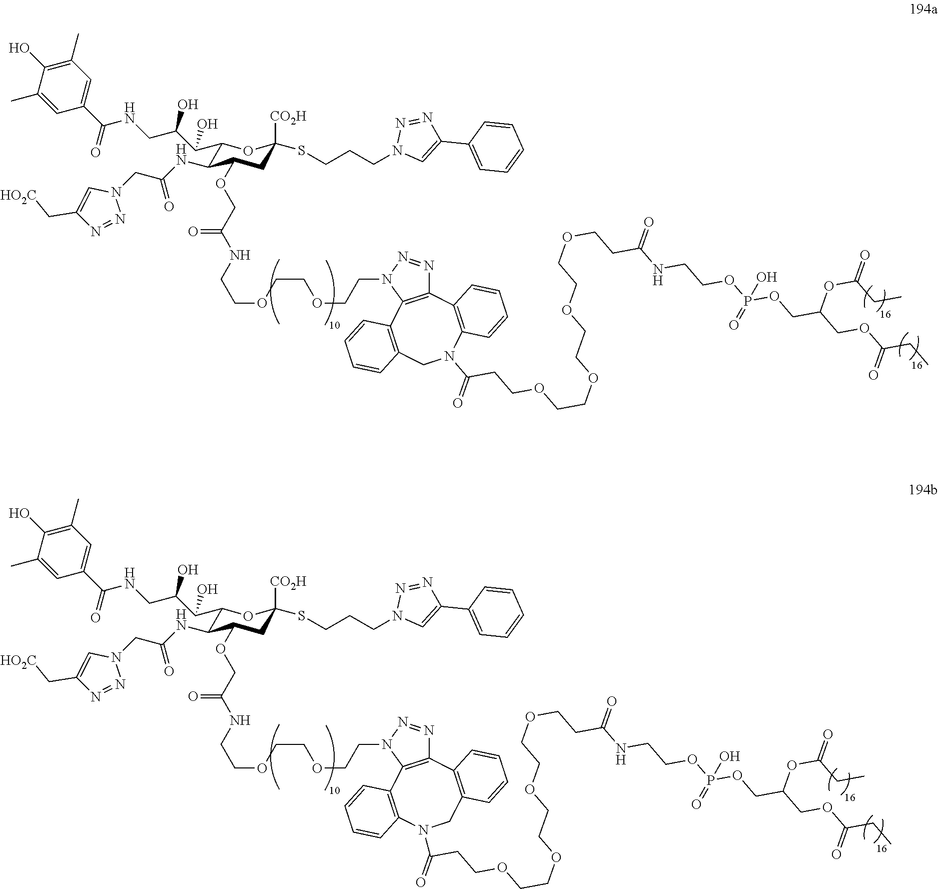

Compounds 194a and 194b: Compound 170 and DSPE PEG-4 DBCO (1.2 eq) are combined in 1/1 v/v chloroform/methanol. The reaction mixture is stirred at 50° C. until completion. The reaction mixture is concentrated and the residue is separated by HPLC to afford compounds 194a and 194b.

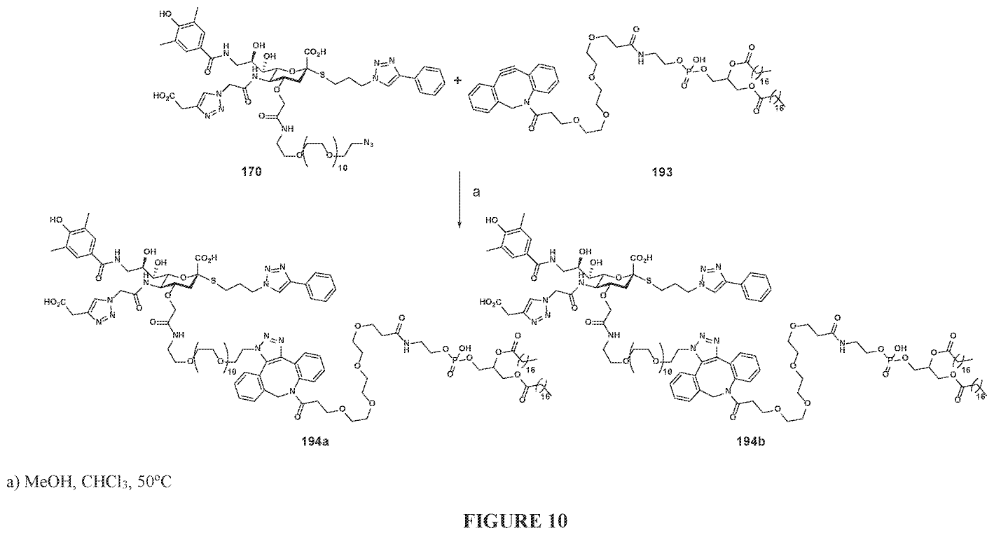

The following glycolipids can be prepared according to FIG. 10:

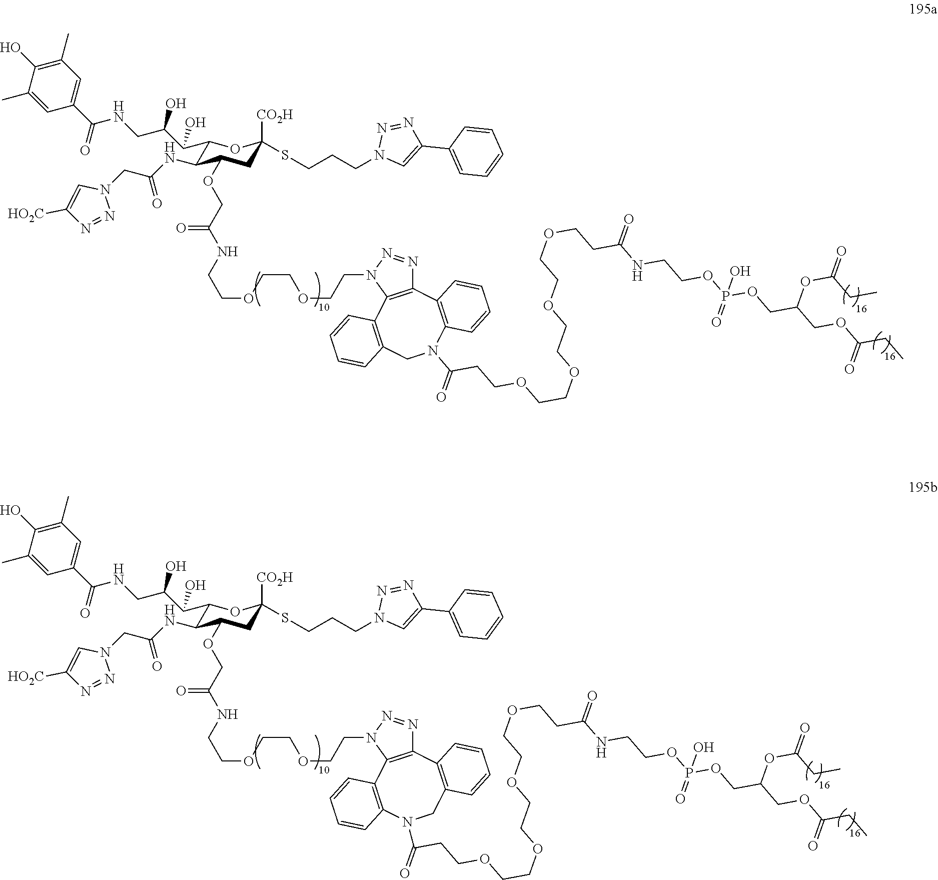

Compounds 195a and 195b: Compounds 195a and 195b can be prepared according to FIG. 10 using compound 172 in step a.

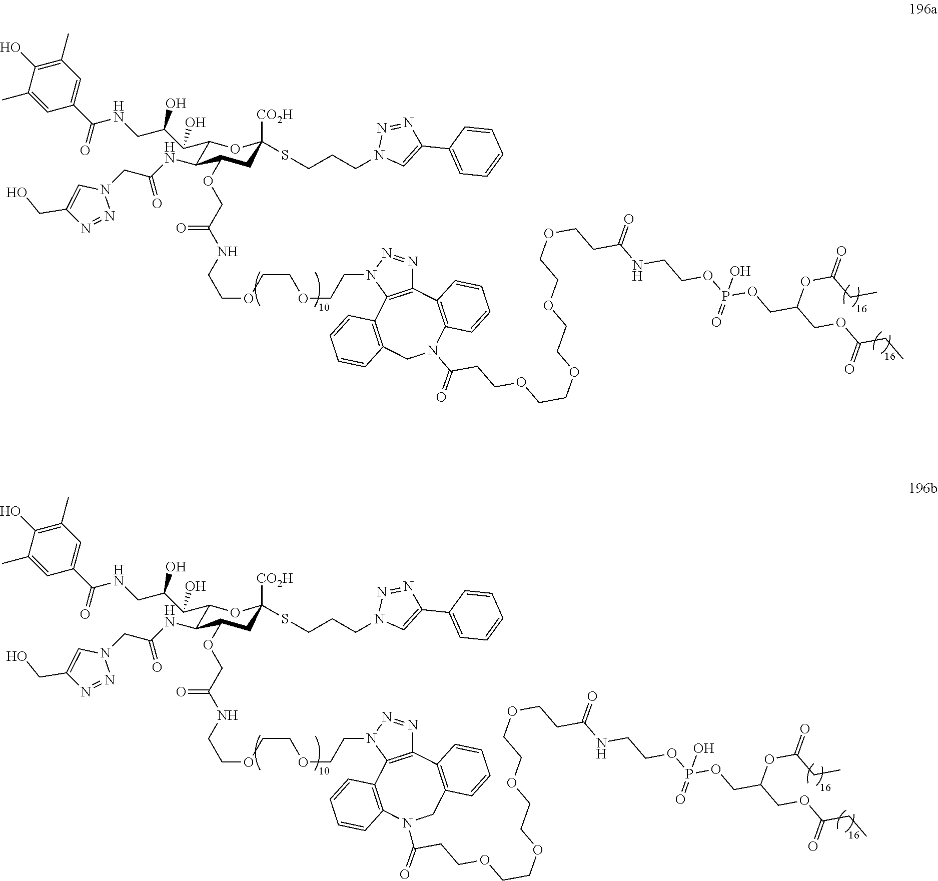

Compounds 196a and 196b: Compounds 196a and 196b can be prepared according to FIG. 10 using compound 173 in step a.

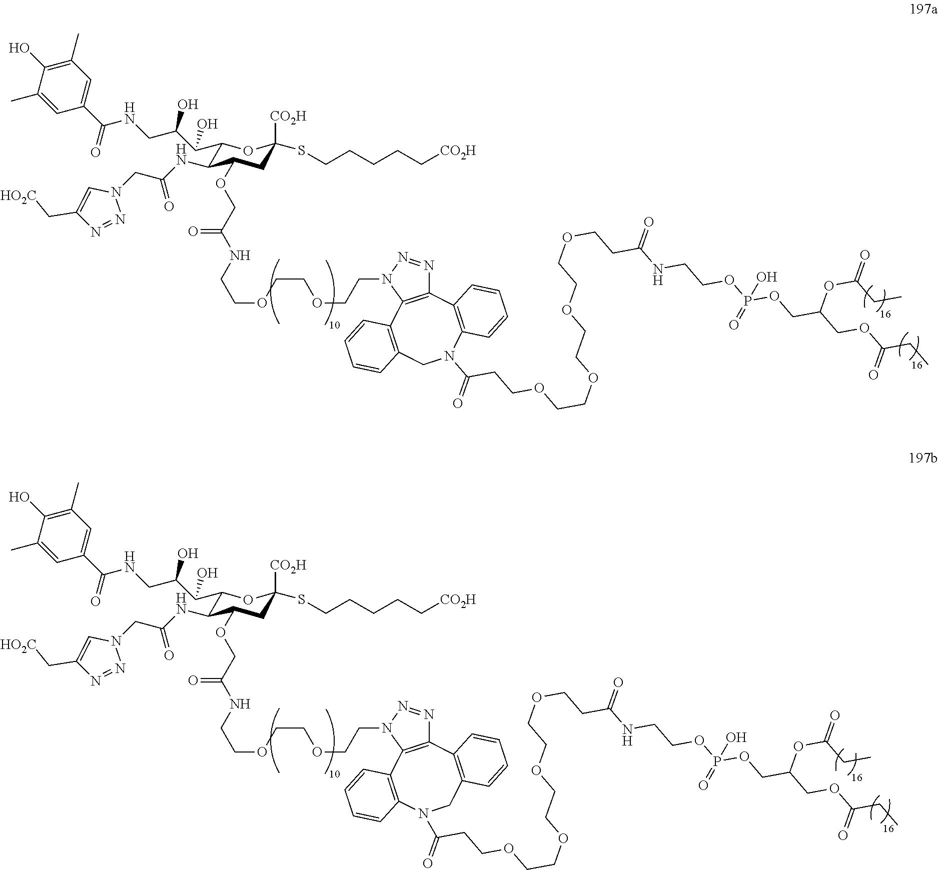

Compound 197a and 197b: Compounds 197a and 197b can be prepared according to FIG. 10 using compound 174 in step a.

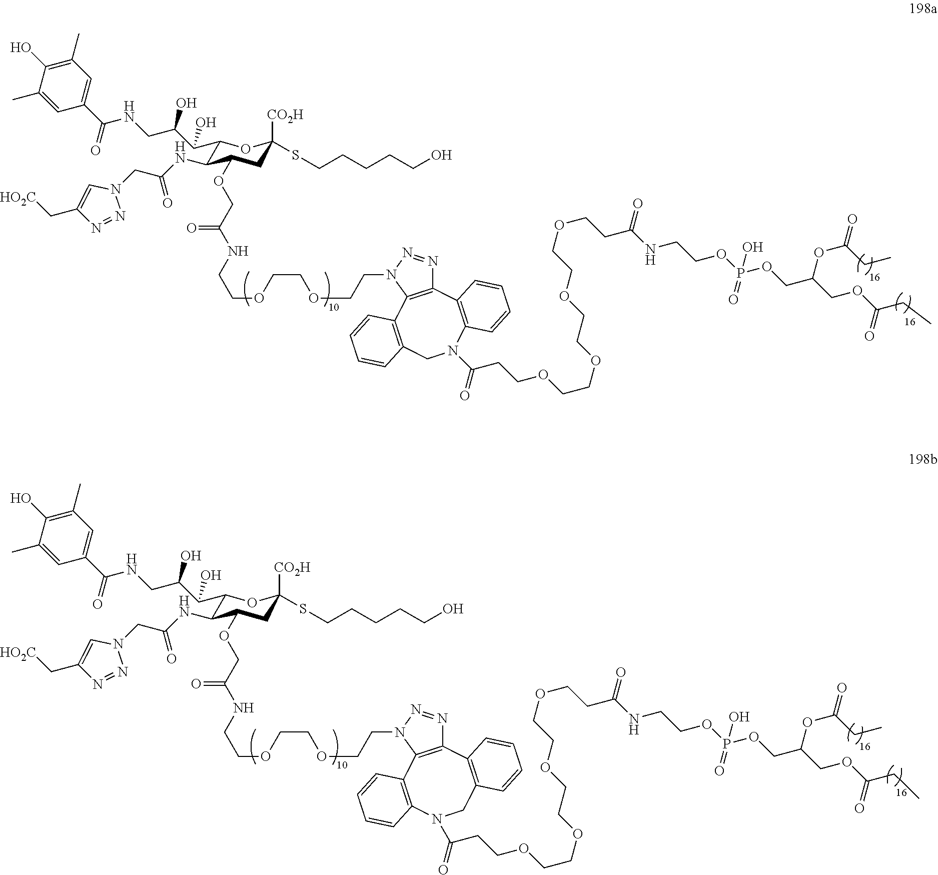

Compound 198a and 198b: Compounds 198a and 198b can be prepared according to FIG. 10 using compound 175 in step a.

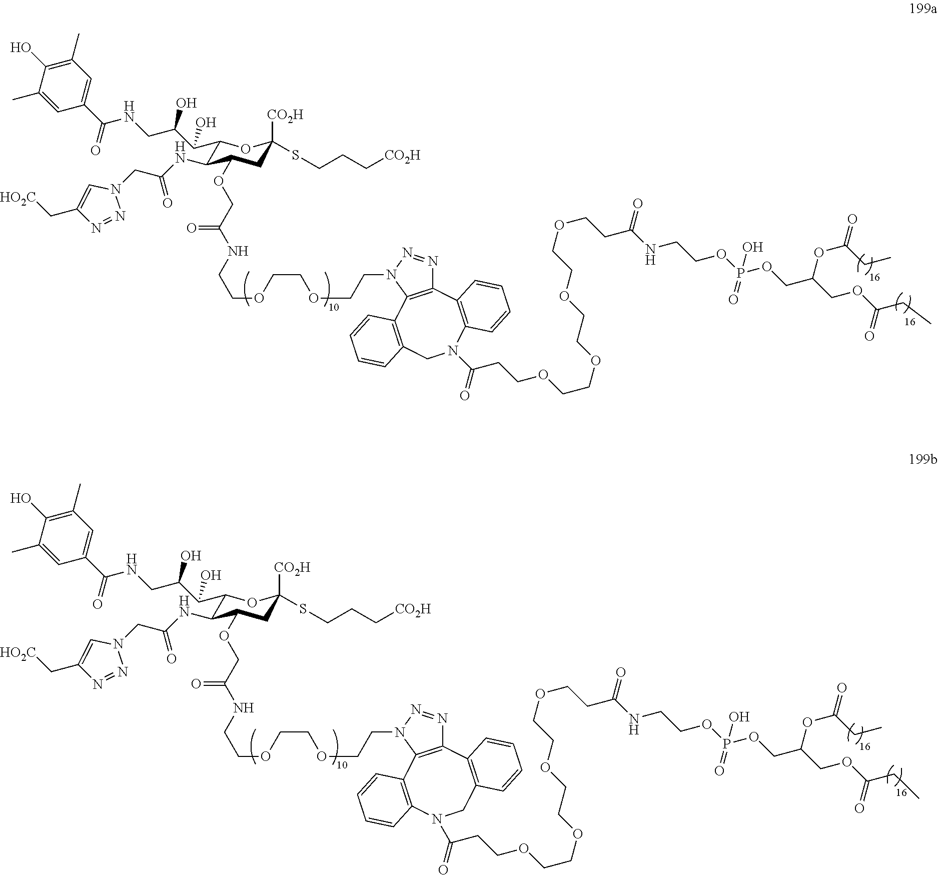

Compound 199a and 199b; Compounds 199a and 199b can be prepared according to FIG. 10 using compound 176 in step a.

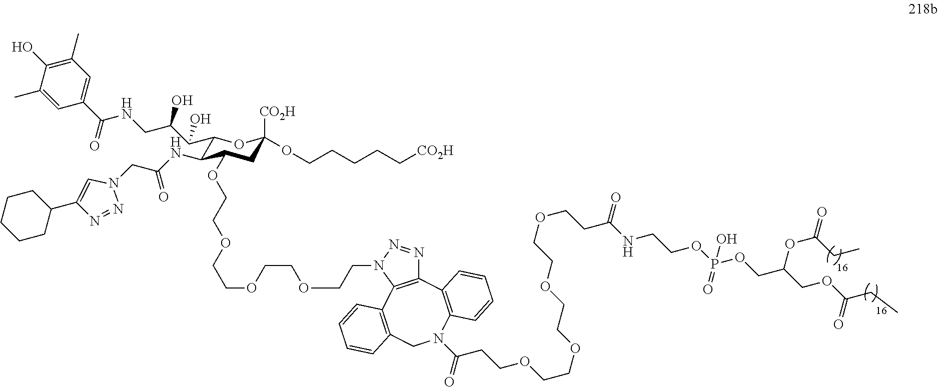

Compounds 218a and 218b: Compounds 218a and 218b can be prepared according to FIG. 10 using compound 17 in step a.

### Example 11

### Prophetic Synthesis of Compound 201-209 and 220

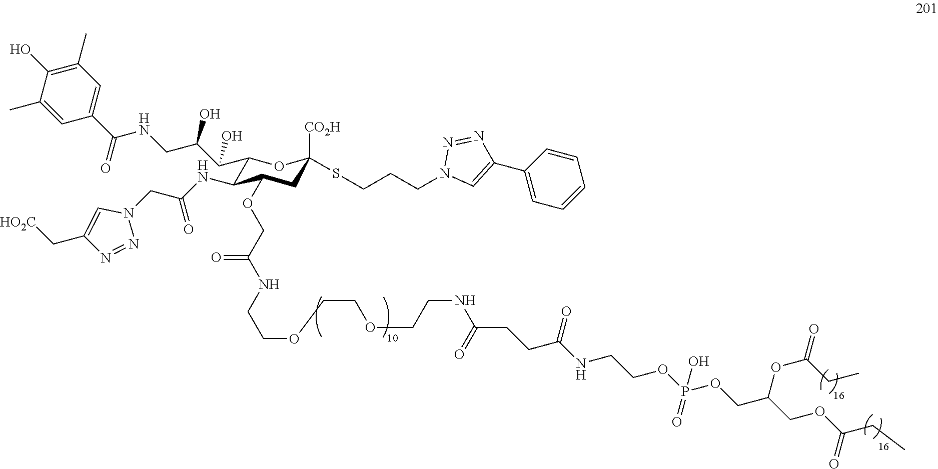

Compound 201: Compound 177 and compound 200(1.1 eq) are combined in water. The reaction mixture is stirred at room temperature until completion. The reaction mixture is separated by HPLC to afford compound 201.

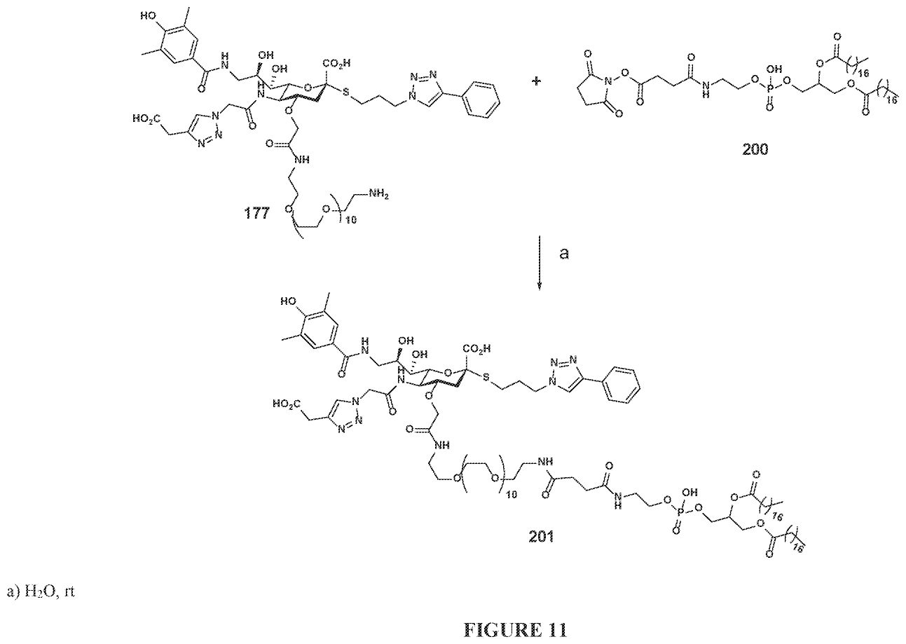

The following compounds can be prepared according to FIG. 11:

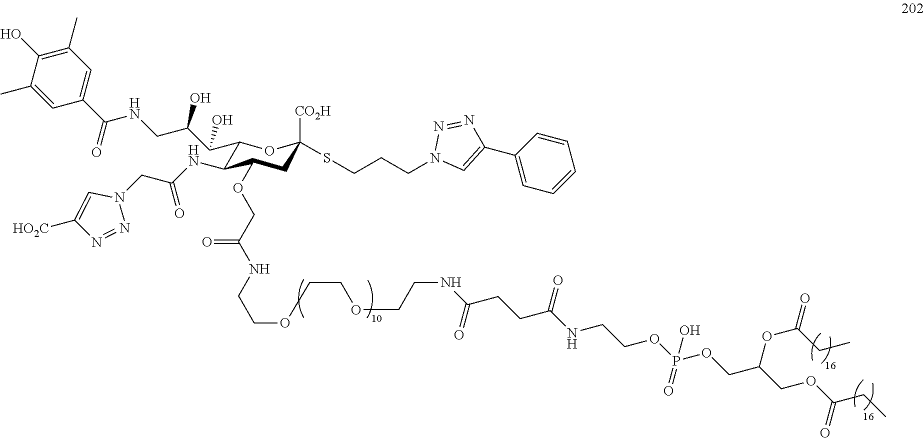

Compound 202: Compound 202 can be prepared according to FIG. 11 using compound 179 in step a.

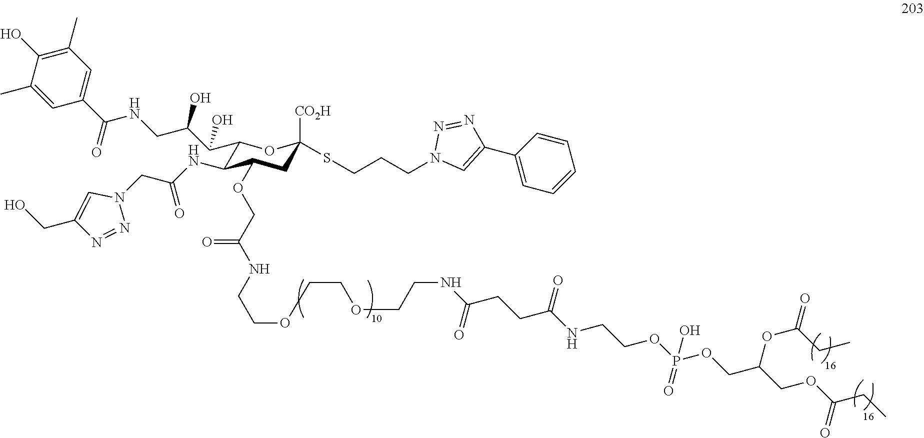

Compound 203: Compound 203 can be prepared according to FIG. 11 using compound 180 in step a.

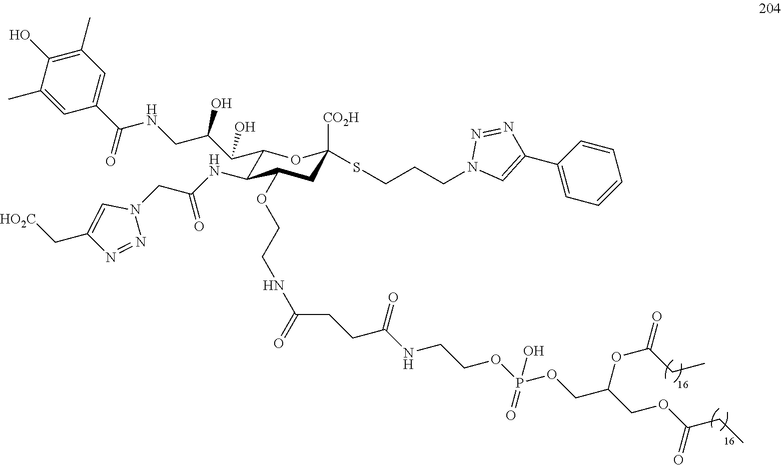

Compound 204: Compound 204 can be prepared according to FIG. 11 using compound 191 in step a.

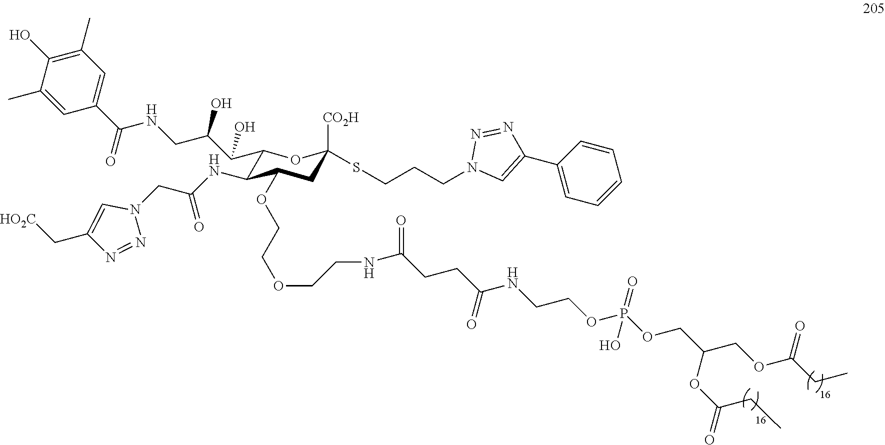

Compound 205: Compound 205 can be prepared according to FIG. 11 using compound 192 in step a.

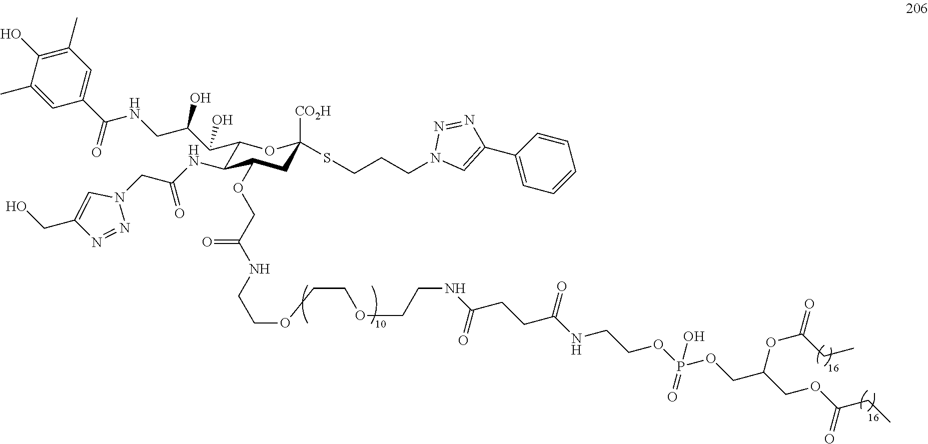

Compound 206: Compound 206 can be prepared according to FIG. 11 using compound 180 in step a.

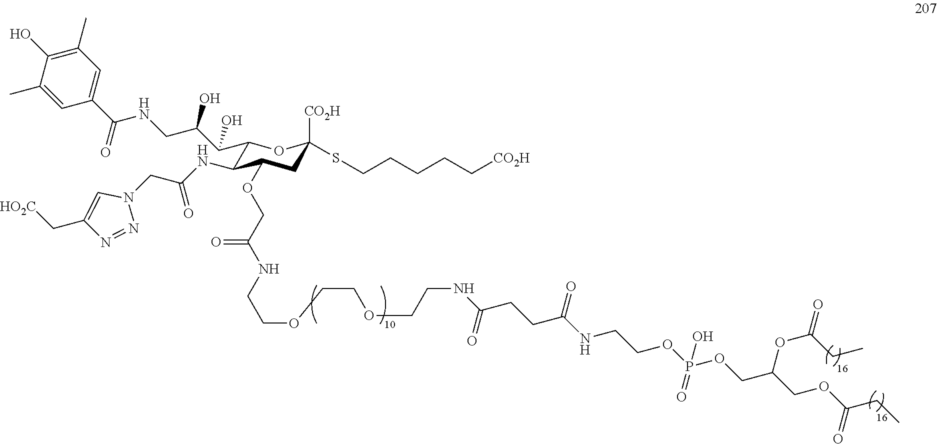

Compound 207; Compound 207 can be prepared according to FIG. 11 using compound 181 in step a.

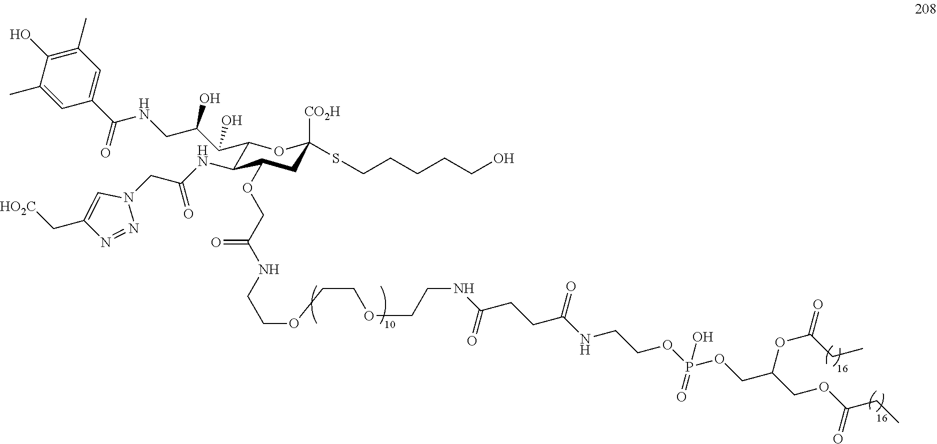

Compound 208; Compound 208 can be prepared according to FIG. 11 using compound 182 in step a.

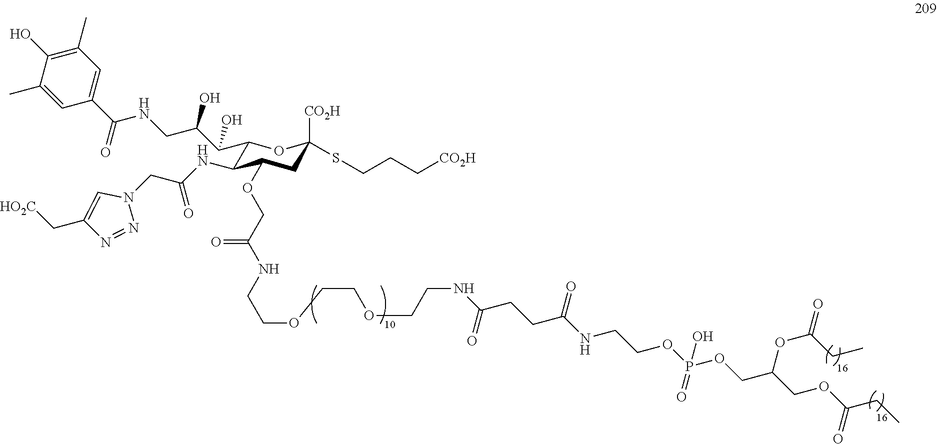

Compound 209: Compound 209 can be prepared according to FIG. 11 using compound 183 in step a.

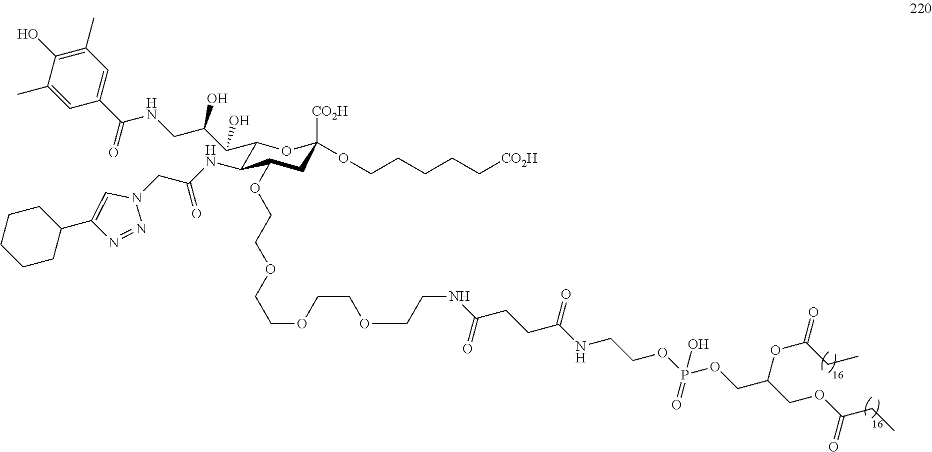

Compound 220: Compound 220 can be prepared according to FIG. 10 using compound 2019 in step a.

### Example 12

### Prophetic Synthesis of Compounds 211-215

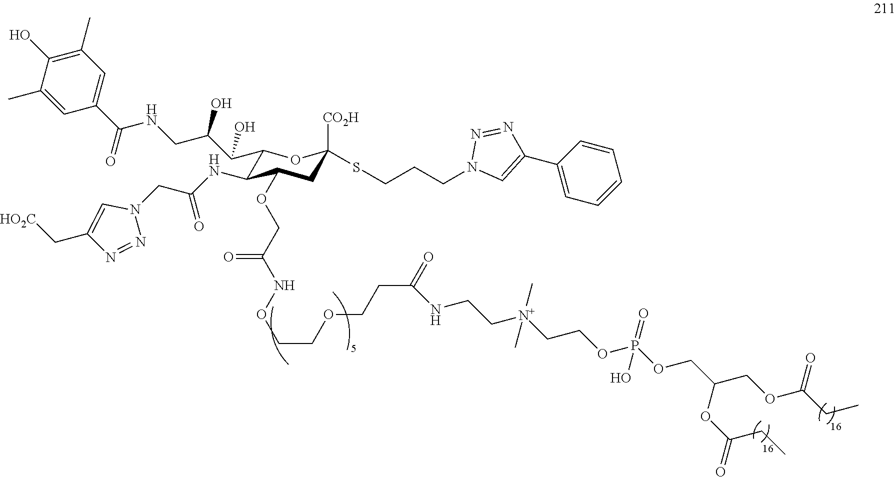

Compound 211: To a solution of compound 184 dissolved in 4/1 v/v DMF/chloroform at room temperature is added diisopropylethylamine (1.5 eq) followed by HATU (1.1 eq). The reaction mixture is stirred 10 minutes. Compound 210 (N-Aminoethyl)-1,2-distearoyl-sn-glycero-3-phosphocholine (Avanti Polar Lipids catalog item 860376, 1.3 eq) is added. The reaction mixture is stirred at room temperature until completion. The reaction mixture is concentrated. The residue is dissolved in 5/1 v/v methanol/water. Sodium methoxide (10 eq) is added and the reaction mixture is stirred at room temperature until completion. The reaction mixture is neutralized with acetic acid and concentrated. The residue is separated by HPLC to afford compound 211.

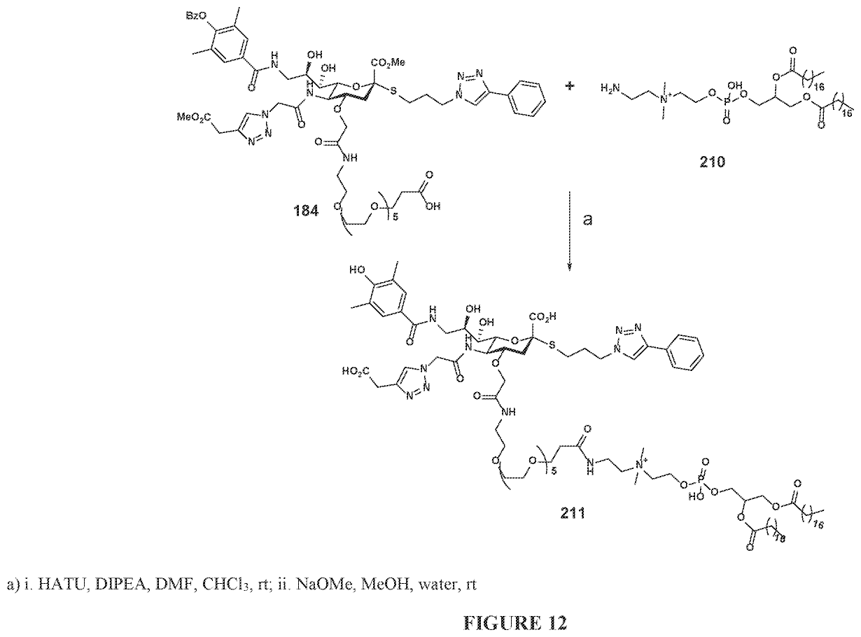

The following compounds can be prepared according to FIG. 12:

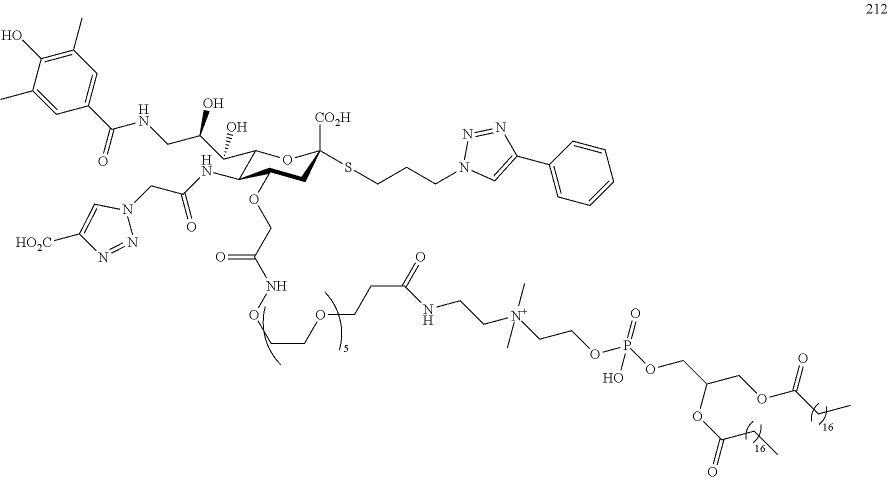

Compound 212: Compound 212 can be prepared according to FIG. 12 using compound 186 in step a.

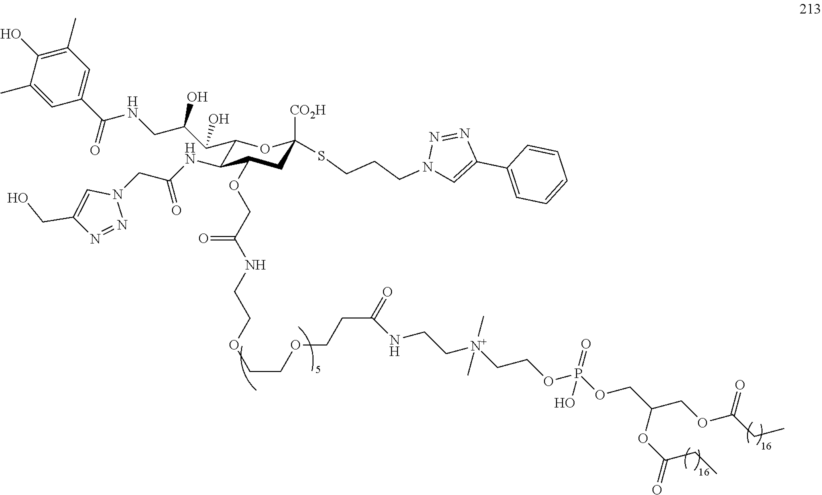

Compound 213: Compound 213 can be prepared according to FIG. 12 using compound 187 in step a.

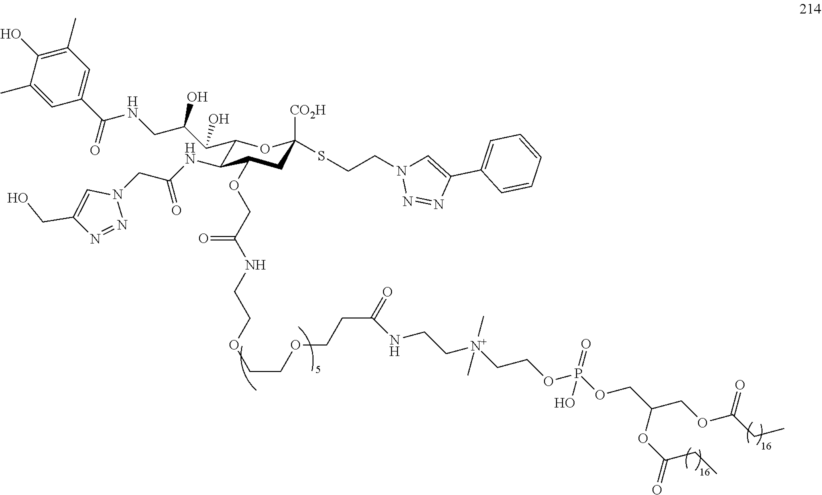

Compound 214: Compound 214 can be prepared according to FIG. 12 using compound 188 in step a.

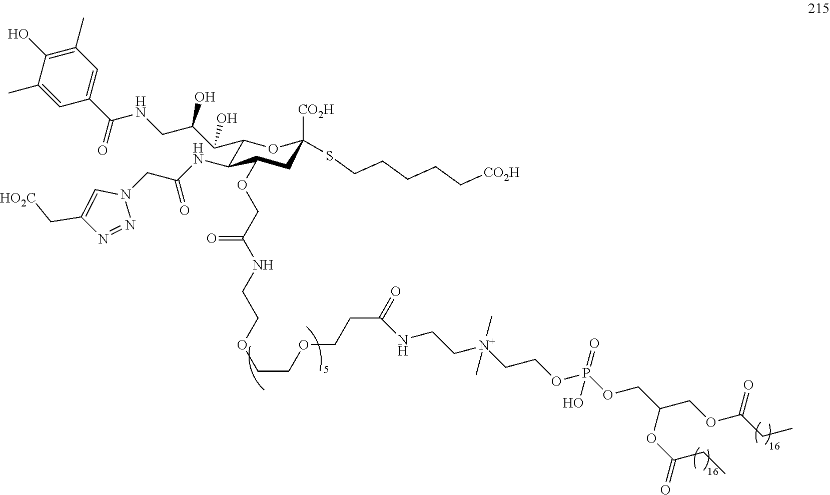

Compound 215: Compound 215 can be prepared according to FIG. 12 using compound 189 in step a.

### Example 13

### Propheitc Synthesis of Compound 217

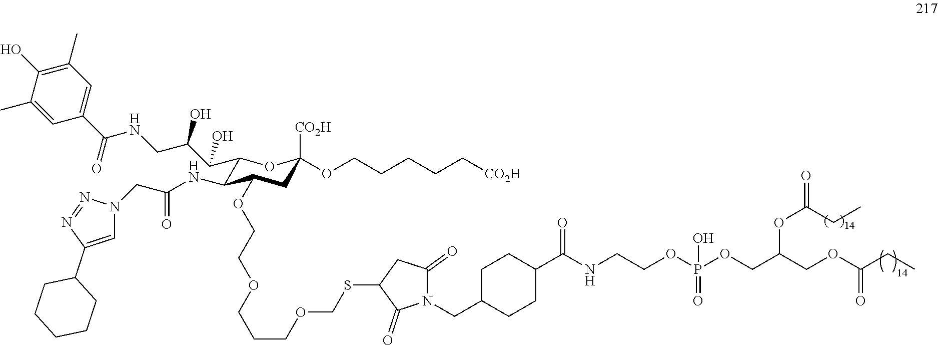

Compound 217: Compound 35 and the commercially available compound 216 are dissolved in water at room temperature. Triethylamine is added and the reaction mixture is stirred at room temperature overnight. The reaction mixture is separated by reverse phase chromatography to afford compound 217.

### Example 14

### CD33 Activity—Binding Assay

The assay to screen for and characterize glycomimetic ligands of CD33 is a competitive binding assay, which allows the determination of IC₅₀ values. CD33-Fc (BioLegend catalog item 750106) was immobilized in 96 well microtiter plates by incubation at 4° C. overnight. To reduce non-specific binding, bovine serum albumin was added to each well and incubated at room temperature for 2 hours. The plate was washed and serial dilutions of the test compounds were added to the wells in the presence of biotinylated polyacrylamide conjugated with Neu5Acα2-3Galβ1-3GalNAc-PAA-biotin (GlycoTech catalog item 01-088). After incubation at room temperature for 2 hours and washing, streptavidin/horseradish peroxidase was added to each well and incubated at room temperature for 30 minutes followed by an additional wash.

To determine the amount of glycopolymer bound to the immobilized CD33, the peroxidase substrate 3.3′0.5.5′ tetramethylbenzidine (TMB) was added. After 5 minutes, the enzyme reaction was stopped by the addition of H₃PO₄ and the absorbance of light at a wavelength of 450 nm was determined. The concentration of test compound required to inhibit binding by 50% was determined and reported as the IC₅₀ value.

1. At least one entity chosen from compounds of Formula (I): 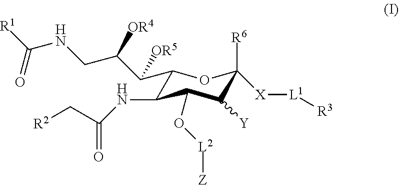
and pharmaceutically acceptable salts thereof, wherein:
R¹ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from halo, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₁₋₈ hydroxyalkyl, C₂₋₈ hydroxyalkenyl, C₂₋₈ hydroxyalkynyl, C₆₋₁₈ aryl, C₁₋₁₃ heteroaryl, —OT¹, —ST¹, —C(═O)OT¹, —C(═O) NT¹T², —NT¹T², —NT¹C(═O) T², —NT¹SO₂T², —S(═O) T¹, -SO₂T¹ and -OSO₃T¹ groups, wherein T¹ and T², which may be identical or different, are independently chosen from H, C₁₋₈ alkyl, C₁₋₈ haloalkyl, and hydroxy protecting groups, or T¹ and T² join together along with the heteroatom to which they are attached to form an optionally substituted, saturated or unsaturated, 3-10 membered ring;
R² is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from halo, C₁₋₈ alkyl, C₂₋₈ alkenyl, C₂₋₈ alkynyl, C₁₋₈ haloalkyl, C₂₋₈ haloalkenyl, C₂₋₈ haloalkynyl,
C₆₋₁₈ aryl, C₁₋₁₃ heteroaryl, -J¹-OT³, -J¹-ST³, -J¹-C(═O)OT³, -J¹-C(═O) NT³T⁴, -J¹-NT³T⁴, -J¹-NT³C(═O) T⁴, -J¹-NT³SO₂T⁴, -J¹-S(═O) T³, -J¹-SO₂T³-J¹-S(═O)₂OT³, and -J¹-P(═O) (OT³) (OT⁴) groups, wherein J¹ is chosen from a bond, divalent C₁₋₁₀ alkyl, and divalent C₁₋₁₀ haloalkyl groups and wherein T³ and T⁴, which may be identical or different, are independently chosen from H, C₁₋₈ alkyl, and C₁₋₈ haloalkyl groups, or T³ and T⁴ join together along with the heteroatom to which they are attached to form an optionally substituted, saturated or unsaturated, 3-10 membered ring;
R³ is chosen from -J²-OT⁵, -J²-C(═O)OT⁵, -J²-C(═O) NT⁵T⁶, -J²-S(═O)₂OT⁵, -J²-OS(═O)₂OT⁵, -J²-P(═O) (OT⁵) (OT⁶), C₆₋₁₈ aryloxy, C₇₋₁₉ arylalkoxy, C₆₋₁₈ aryl, and C₁₋₁₃ heteroaryl groups, wherein J² is chosen from a bond, divalent C₁₋₁₀ alkyl, and divalent C₁₋₁₀ haloalkyl groups, wherein T⁵ and T⁶, which may be identical or different, are independently chosen from H and C₁₋₈ alkyl groups, or T⁵ and T⁶ join together along with the heteroatoms to which they are attached to form an optionally substituted, saturated or unsaturated, 3-10 membered ring, and wherein the C₆₋₁₈ aryloxy, C₇₋₁₉ arylalkoxy, C₆₋₁₈ aryl, and C₁₋₁₃ heteroaryl groups are optionally substituted with one or more groups independently chosen from R⁷, halo, C₁₋₈ alkyl, C₁₋₈ haloalkyl, —OT⁷, —C(═O)OT⁷, and —C(═O) NT⁷T⁸ groups, wherein R⁷ is independently chosen from C₆₋₁₈ aryl groups which are optionally substituted with one or more groups independently chosen from halo, C₁₋₈ alkyl, C₆₋₁₈ aryl, —OT⁹, —C(═O)OT⁹, and —C(═O) NT⁹T¹⁰ groups, wherein T⁷, T⁸, T⁹, and T¹⁰, which may be identical or different, are independently chosen from H and C₁₋₈ alkyl groups, or T⁷ and T⁸ join together along with the nitrogen atom to which they are attached to form an optionally substituted, saturated or unsaturated, 3-10 membered ring and/or T⁹ and T¹⁰ join together along with the nitrogen atom to which they are attached to form an optionally substituted, saturated or unsaturated, 3-10 membered ring;
R⁴ and R⁵, which may be identical or different, are independently chosen from H and hydroxy protecting groups, or R⁴ and R⁵ join together along with the oxygen atoms to which they are attached to form an optionally substituted, saturated or unsaturated, 3-10 membered ring;
R⁶ is chosen from —C(═O)OR⁸, and —C(═O) NT¹¹T¹², wherein R⁸ is chosen from H and carboxy protecting groups and wherein T¹¹ and T¹², which may be identical or different, are independently chosen from H and C₁₋₈ alkyl groups, or T¹¹ and T¹² join together along with the nitrogen atom to which they are attached to form an optionally substituted, saturated or unsaturated, 3-10 membered ring;
L¹ and L², which may be identical or different, are independently chosen from linker groups;
X is chosen from —O—, —S—, —CH₂—, and —N(T¹³)—, wherein T¹³ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and —C(═O) T¹⁴ groups, wherein T¹⁴ is chosen from H, halo, C₁₋₈ alkyl, C₆₋₁₈ aryl, and C₁₋₁₃ heteroaryl groups;
Y is chosen from H, halo, and —OT¹⁵ groups, wherein T¹⁵ is chosen from H and C₁₋₈ alkyl groups; and
Z is chosen from lipids, nucleophiles, electrophiles, and groups capable of undergoing a cycloaddition reaction.

2. The at least one entity according to claim 1, wherein R¹ is chosen from C₆₋₁₀ aryl groups optionally substituted with one or more groups independently chosen from halo, C₁₋₈ alkyl, C₁₋₈ hydroxyalkyl, and —OT¹ groups.

3. The at least one entity according to claim 1, wherein R¹ is

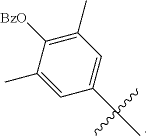

4. The at least one entity according to claim 1, wherein R¹ is

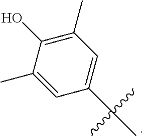 [SMILES: *c1cc(C)c(O)c(C)c1]

5. The at least one entity according to claim 1, wherein R² is chosen from triazole groups optionally substituted with one or more groups independently chosen from -J¹-OT³, -J¹-ST³, -J¹-C(═O)OT³, -J¹-C(═O) NT³T⁴, -J¹-NT³T⁴, -J¹-NT³C(═O) T⁴, -J¹-NT³SO₂T⁴, -J¹-S(═O) T³, -J¹-SO₂T³-J¹-S(═O)₂OT³, and -J¹-P(═O) (OT³) (OT⁴) groups.

6. The at least one entity according to claim 5, wherein R² is chosen from triazole groups optionally substituted with one or more groups independently chosen from -J¹-OT³ and -J¹-C(═O)OT³ groups.

7. The at least one entity according to claim 1, wherein J¹ is a bond.

8. The at least one entity according to claim 1, wherein J¹ is chosen from divalent C₁₋₁₀ alkyl groups.

9. The at least one entity according to claim 8, wherein J¹ is chosen from —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, and —(CH₂)₁₀—.

10. The at least one entity according to claim 8, wherein J¹ is chosen from

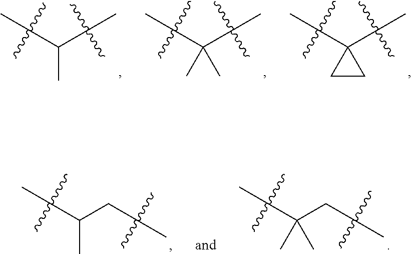

11. The at least one entity according to claim 1, wherein R² is

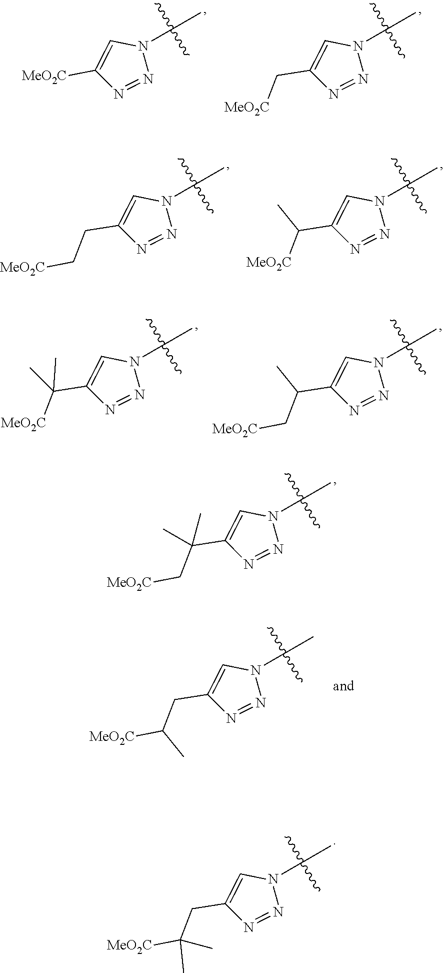

12. The at least one entity according to claim 1, wherein R² is

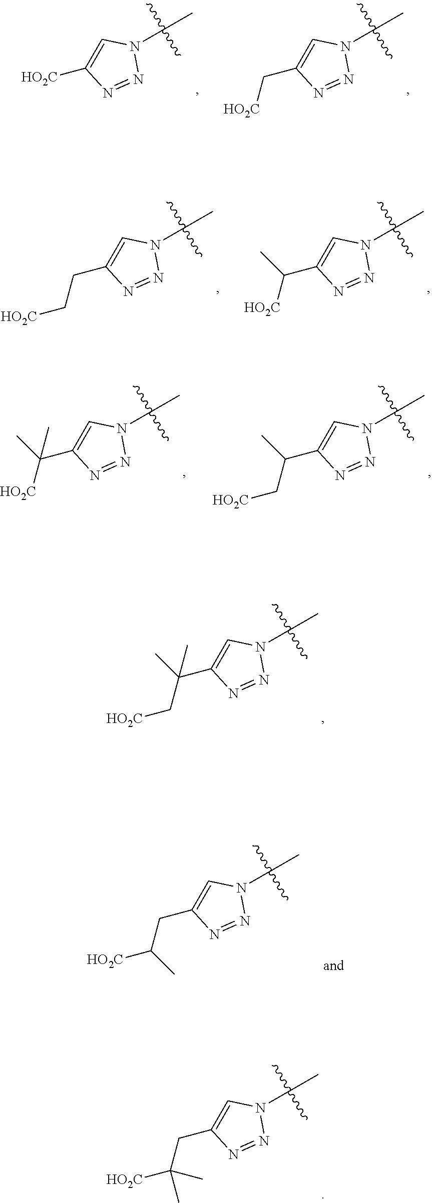

13. The at least one entity according to claim 1, wherein R² is chosen from

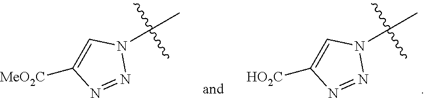 [SMILES: *n1cc(C(=O)OC)nn1 ; *n1cc(C(=O)O)nn1]

14. The at least one entity according to claim 1, wherein R² is chosen from

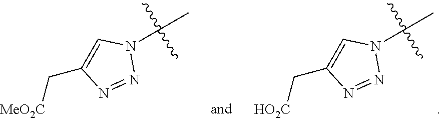 [SMILES: *n1cc(CC(=O)OC)nn1 ; *n1cc(CC(=O)O)nn1]

15. The at least one entity according to claim 1, wherein R² is

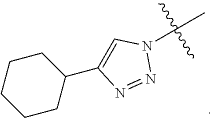 [SMILES: *n1cc(C2CCCCC2)nn1]

16. The at least one entity according to claim 1, wherein R³ is chosen from -J²-OT⁵, -J²-C(═O)OT⁵, -J²-C(═O) NT⁵T⁶, -J²-S(═O)₂OT⁵, -J²-OS(═O)₂OT⁵, and -J²-P (═O) (OT⁵) (OT⁶) groups, wherein J² is chosen from a bond, divalent C₁₋₁₀ alkyl, and divalent C₁₋₁₀ haloalkyl groups, wherein T⁵ and T⁶, which may be identical or different, are independently chosen from H and C₁₋₈ alkyl groups, or T⁵ and T⁶ join together along with the heteroatoms to which they are attached to form an optionally substituted, saturated or unsaturated, 3-10 membered ring.

17. The at least one entity according to claim 16, wherein J² is a bond.

18. The at least one entity according to claim 16, wherein J² is chosen from divalent C₁₋₁₀ alkyl groups.

19. The at least one entity according to claim 18, wherein J² is chosen from —CH₂—, —(CH₂)₂—, —(CH₂)₃—, —(CH₂)₄—, —(CH₂)₅—, —(CH₂)₆—, —(CH₂)₇—, (CH₂)₈—, —(CH₂)₉—, and —(CH₂)₁₀₋₈.

20. The at least one entity according to claim 1, wherein R³ is chosen from —OH, —CO₂H, —SO₃H, —OSO₃H, and —PO₃H₂.

21. The at least one entity according to claim 1, wherein R³ is chosen from triazole groups substituted with one or more phenyl which is optionally substituted with one or more groups independently chosen from halo and C₆₋₁₈ aryl groups.

22. The at least one entity according to claim 1, wherein R³ is chosen from C₇₋₁₉ arylalkoxy groups optionally substituted with one or more groups independently chosen from R⁷, C₁₋₈ alkyl, and C₁₋₈ haloalkyl groups.

23. The at least one entity according to claim 22, wherein R³ is —OBn optionally substituted with one or more groups independently chosen from R⁷, C₁₋₈ alkyl, and C₁₋₈ haloalkyl groups.

24. The at least one entity according to claim 1, wherein R³ is —OBn.

25. The at least one entity according to claim 1, wherein R³ is chosen from

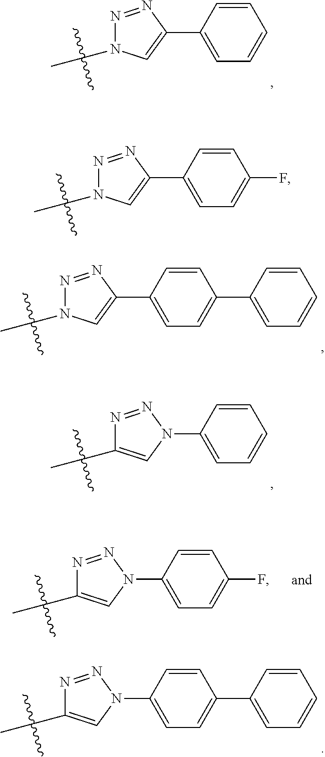 [SMILES: *n1cc(-c2ccccc2)nn1 ; *n1cc(-c2ccc(F)cc2)nn1 ; *n1cc(-c2ccc(-c3ccccc3)cc2)nn1 ; *c1cn(-c2ccccc2)nn1 ; *c1cn(-c2ccc(F)cc2)nn1 ; *c1cn(-c2ccc(-c3ccccc3)cc2)nn1]

26. The at least one entity according to claim 1, wherein at least one of R⁴ and R⁵ is H.

27. The at least one entity according to claim 1, wherein R⁴ and R⁵ are H.

28. The at least one entity according to claim 1, wherein R⁴ and R⁵ join together along with the oxygen atoms to which they are attached to form an acetonide.

29. The at least one entity according to claim 1, wherein R⁶ is chosen from —C(═O)OR⁸ groups.

30. The at least one entity according to claim 29, wherein R⁸ is H.

31. The at least one entity according to claim 29, wherein R⁸ is methyl.

32. The at least one entity according to claim 1, wherein L¹ is chosen from —(CH₂)ₘ—, —(CH₂)ₘO(CH₂)ₙ—, and (CH₂)ₘS(CH₂)ₙ— groups, wherein m and n, which may be the same or different, are independently chosen from integers ranging from 1 to 24.

33. The at least one entity according to claim 1, wherein L¹ is chosen from —(CH₂)ₘ— groups, wherein m is chosen from integers ranging from 2 to 4.

34. The at least one entity according to claim 1, wherein L¹ is chosen from

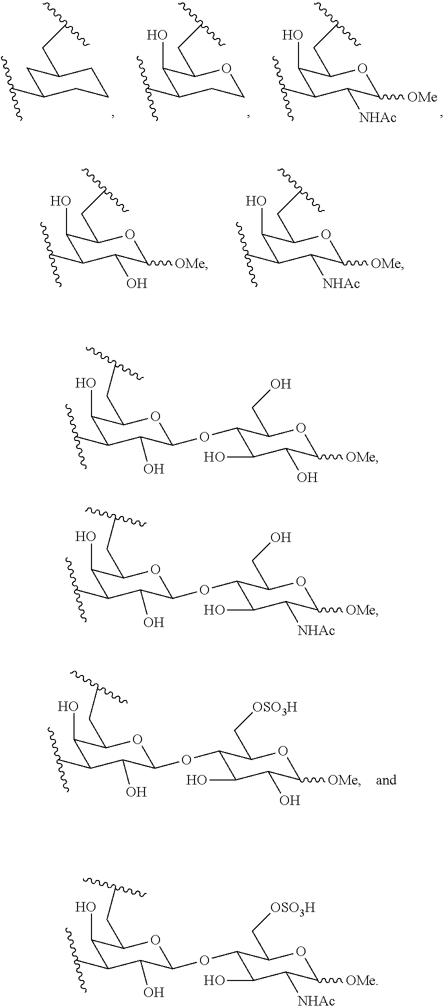

35. The at least one entity according to claim 1, wherein L² is chosen from —Q(CH₂)ₘCH₂V—, -QCH₂ (OCH₂CH₂)ₘOCH₂V—, -QCH₂CH₂ (OCH₂CH₂)ₘOCH₂V—, -QCH₂ (OCH₂CH₂)ₘOCH₂CH₂V—, and —QCH₂CH₂ (OCH₂CH₂)ₘOCH₂CH₂V— groups, wherein Q is chosen from a bond, —CH₂C(═O) NH—, and —CH₂CH₂CH₂SCH₂CH₂NH(C═O)—, wherein V is chosen from a bond, 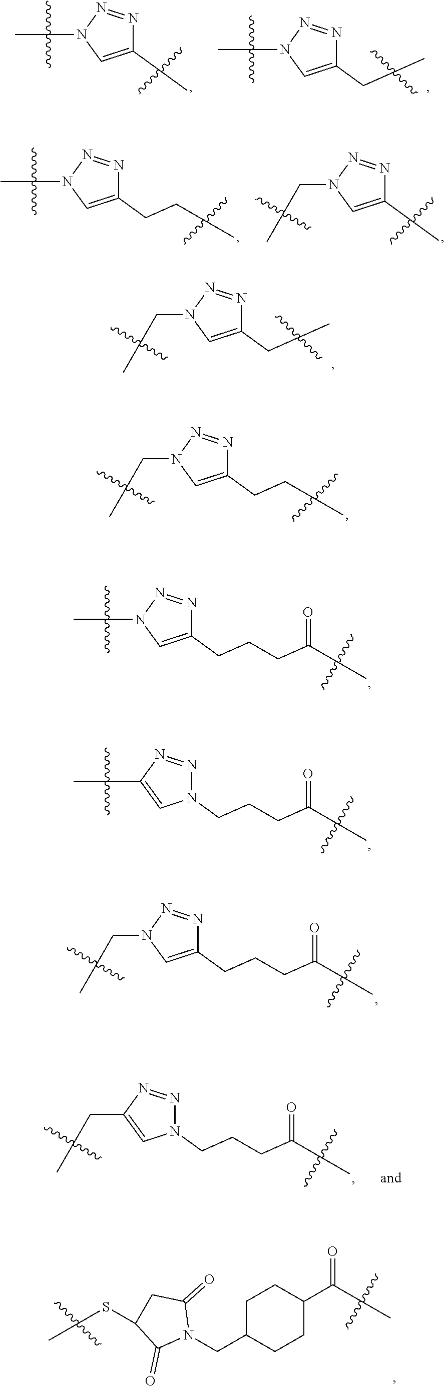
and wherein m is chosen from integers ranging from 0 to 46.

36. The at least one entity according to claim 1, wherein V is a bond.

37. The at least one entity according to claim 1, wherein Q is a bond.

38. The at least one entity according to claim 1, wherein Q is —CH₂C(═O) NH—.

39. The at least one entity according to claim 1, wherein Q is —CH₂CH₂CH₂SCH₂CH₂NH(C═O)—.

40. The at least one entity according to claim 1, wherein X is —O—.

41. The at least one entity according to claim 1, wherein X is —S—.

42. The at least one entity according to claim 1, wherein X is —CH₂₋₈.

43. The at least one entity according to claim 1, wherein Y is H.

44. The at least one entity according to claim 1, wherein Y is fluoro.

45. The at least one entity according to claim 1, wherein Z is chosen from lipids.

46. The at least one entity according to claim 45, wherein Z is chosen from phospholipids.

47. The at least one entity according to claim 1, wherein Z is chosen from —N₃, —NH₂, —SH, —OH, —Cl, —Br, —I, —CH═CH₂, —C≡CH,

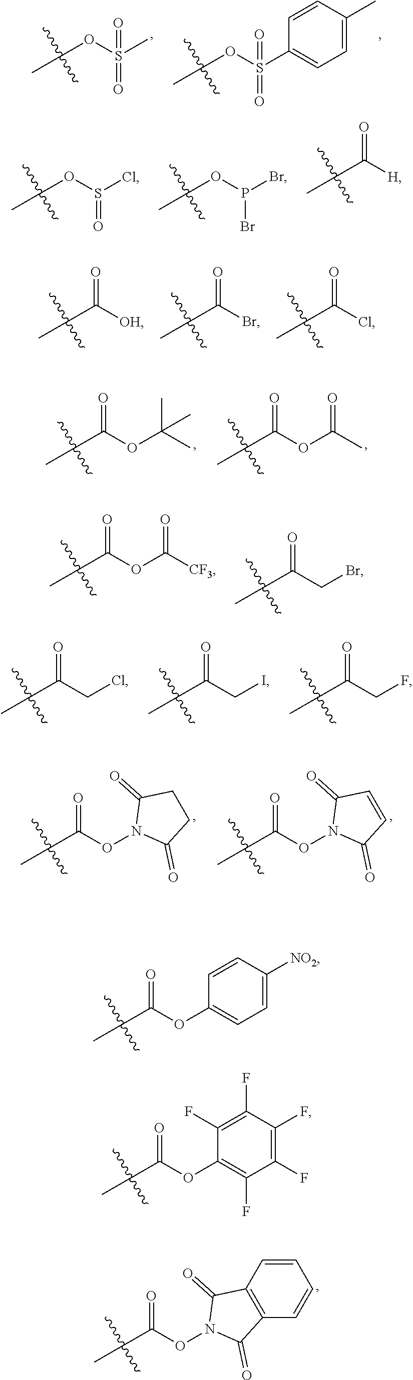

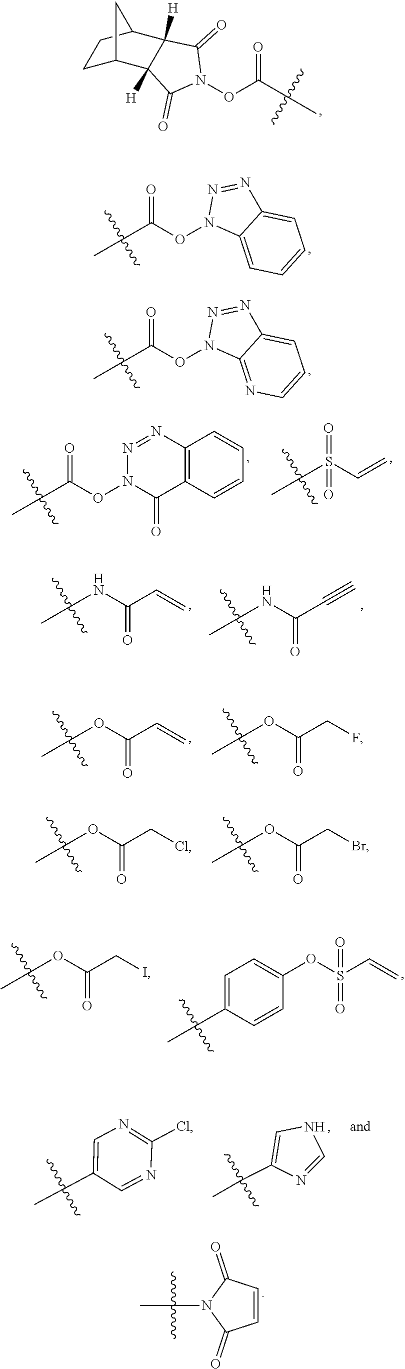

48. The at least one entity according to claim 47, wherein Z is —N₃.

49. The at least one entity according to claim 47, wherein Z is chosen from —NH₂, —SH, and —OH.

50. The at least one entity according to claim 47, wherein Z is chosen from —C(═O)H, —C(═O)OH, —C(═O)Cl, and —C(═O)O^{t}Bu.

51. The at least one entity according to claim 1, wherein Z is chosen from esters formed with t-butanol, p-nitrophenol, 2,4-dinitrophenol, trichlorophenol, 1-hydroxy-1H-benzotriazole, 1-hydroxy-6-chloro-1H-benzotriazole, and N-hydroxysuccinimide.

52. The at least one entity according to claim 1, wherein Z is chosen from —C(═O)T¹⁶ groups, wherein T¹⁶ is chosen from H, —OH, —O^{t}Bu, C₂₋₈ alkenyl, C₁₋₈ haloalkyl, C₁₋₈ alkoxy, halo, C₆₋₁₈ aryloxy, C₁₋₁₃ heteroaryloxy, and C₂₋₁₂ heterocyclyloxy groups and wherein the C₁₋₈ alkoxy, C₆₋₁₈ aryloxy, C₁₋₁₃ heteroaryloxy, and C₂₋₁₂ heterocyclyloxy groups are optionally substituted with at least one halo group.

53. The at least one entity according to claim 1, wherein Z is chosen from alkene and alkyne groups.

54. The at least one entity according to claim 1, wherein Z is —C≡CH.

55. The at least one entity according to claim 1, wherein the entity is chosen from: 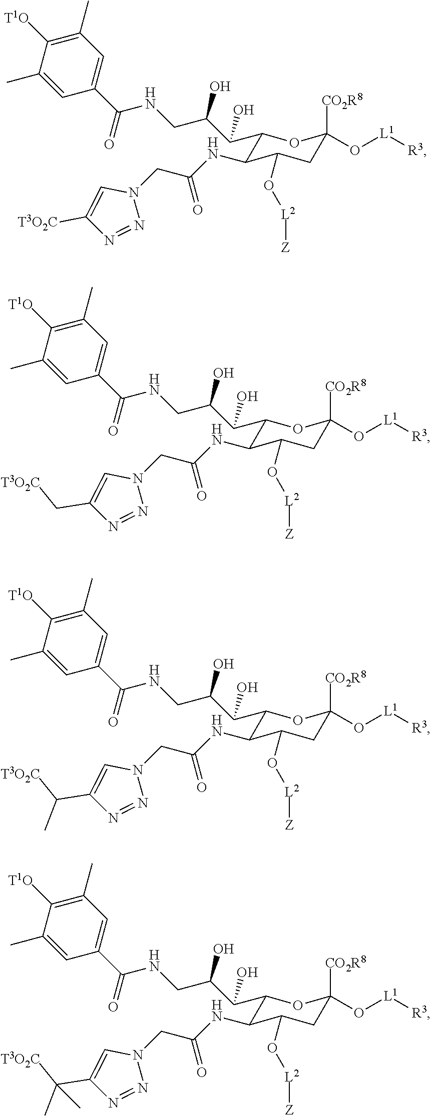 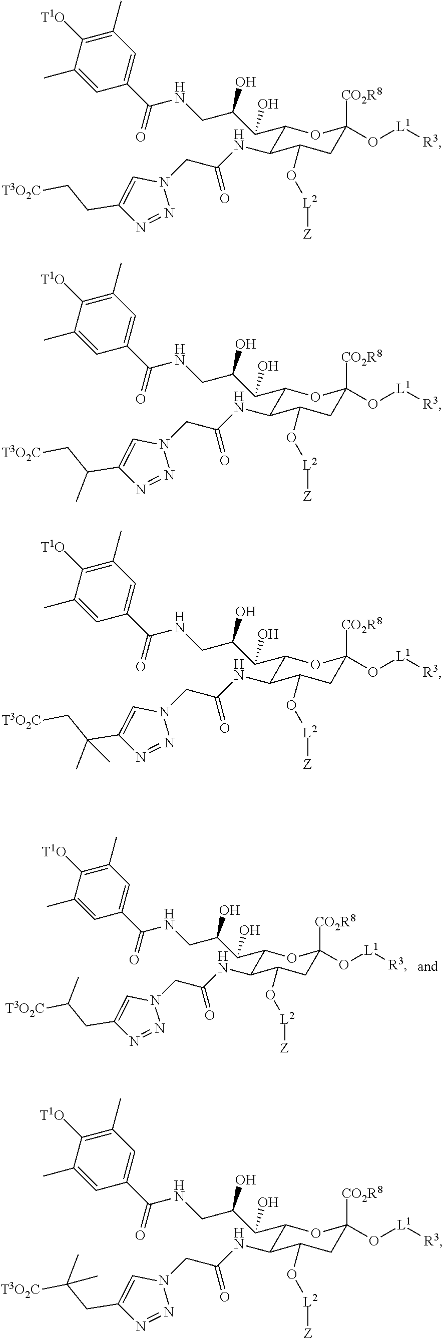
and pharmaceutically acceptable salts of any of the foregoing.

56. The at least one entity according to claim 1, wherein the entity is chosen from: 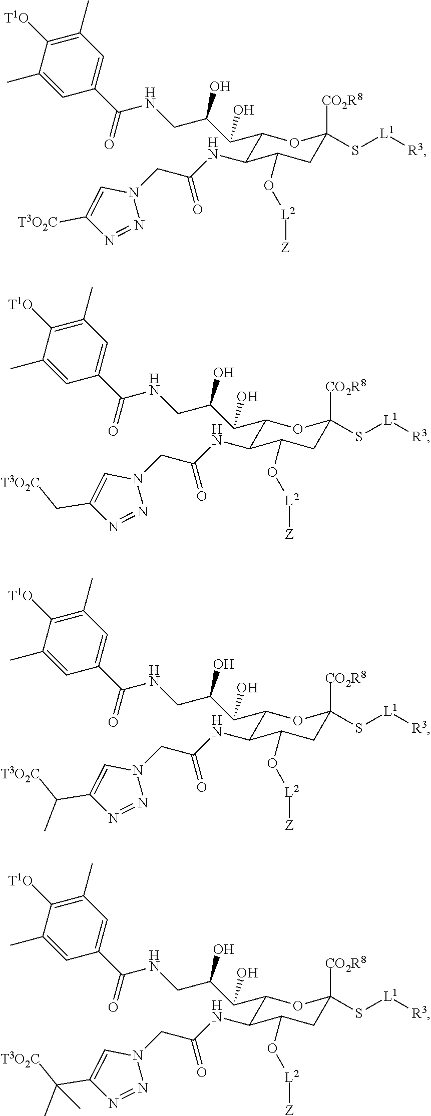 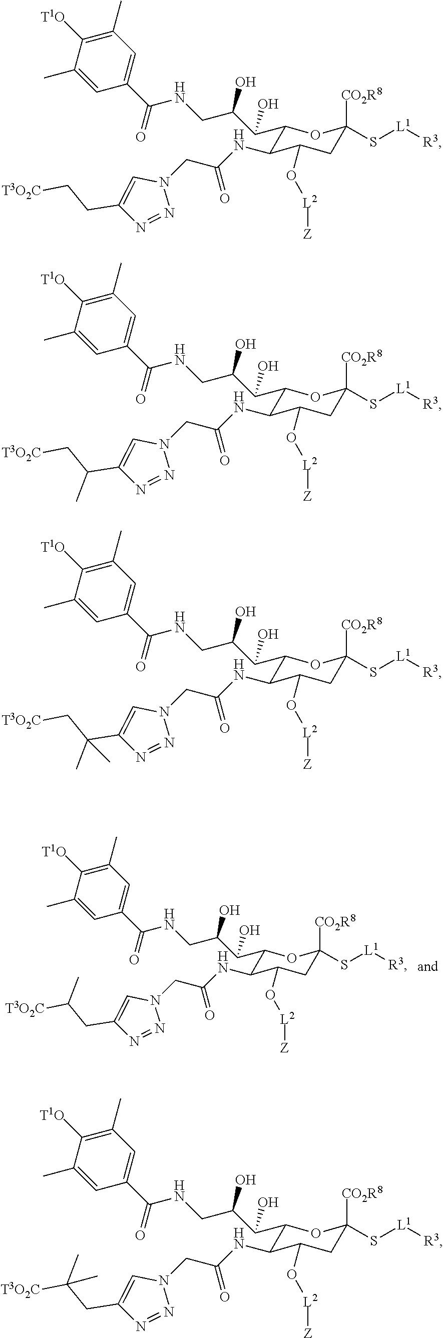
and pharmaceutically acceptable salts of any of the foregoing.

57. The at least one entity according to claim 55, wherein R³ is chosen from —OH, —CO₂H, —SO₃H, —OSO₃H, and —PO₃H₂.

58. The at least one entity according to claim 55, wherein L¹ is chosen from

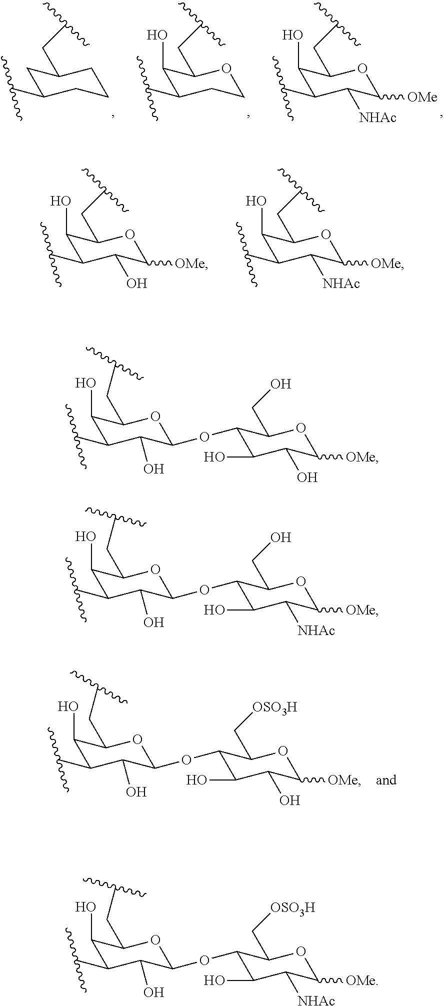

59. The at least one entity according to claim 55, wherein at least one of T¹, T³, and R⁸ is H.

60. The at least one entity according to claim 55, wherein each of T¹, T³, and R⁸ is H.

61. A process for making at least one entity chosen from compounds of Formula (II) and pharmaceutically acceptable salts thereof, wherein the process comprises reacting or associating at least one entity of claim 1 with at least one entity chosen from compounds of Formula (III): 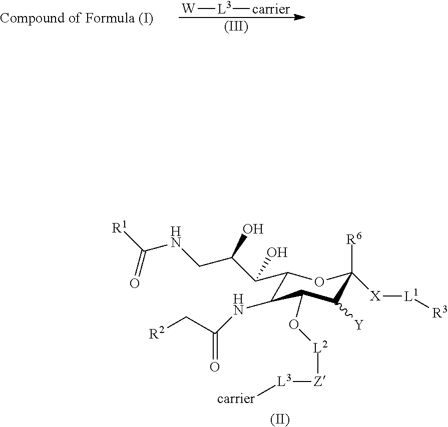
wherein:
R¹, R², R³, L¹, L², X, and Y are as defined in claim 1;
L³ is chosen from a bond and linker groups;
W is chosen from lipids, nucleophiles, electrophiles, and groups capable of undergoing a cycloaddition reaction; and
Z′ is a moiety generated by the reaction or association of the Z group of the at least entity of claim 1 with the W group of the at least one entity chosen from compounds of Formula (III).

62. The process according to claim 61, wherein the at least one entity of claim 1 is chosen from the at least one entity of claim 2.

63. The process according to claim 61, wherein L³ is a bond.

64. The process according to claim 61, wherein L³ is chosen from linker groups.

65. The process according to claim 61, wherein the carrier is chosen from particles, nanoparticles, liposomes, beads, proteins, polysaccharides, lipids, and combinations thereof.

66. The process according to claim 65, wherein the carrier is chosen from nanoparticles.

67. The process according to claim 61, wherein Z′ is chosen from a lipid-lipid non-covalent association, —NH(O=)C—, —NHCH₂—, —SH₂C—, —C(═O)HN—,

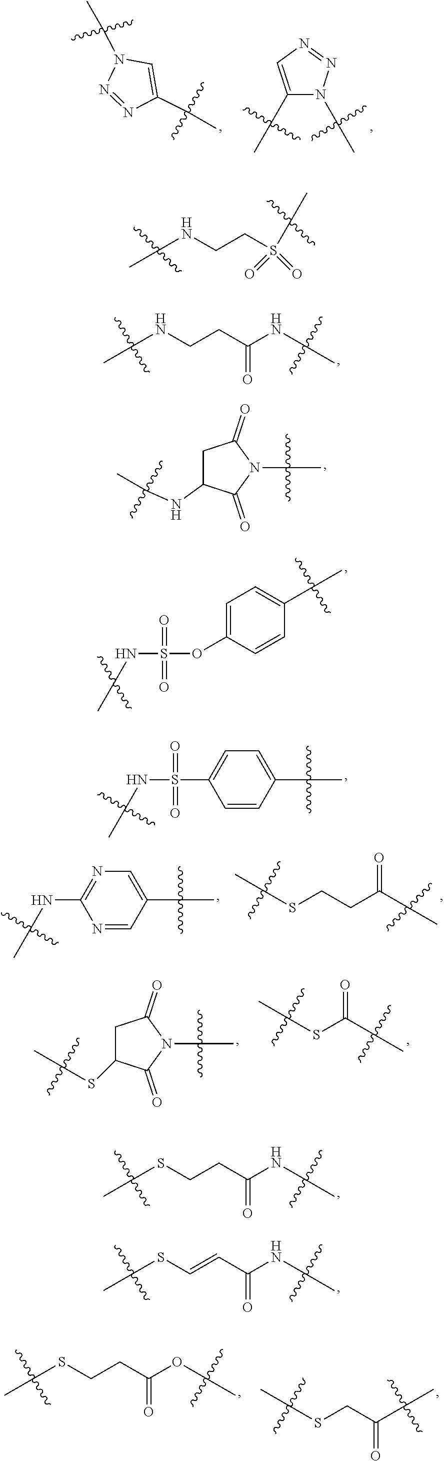

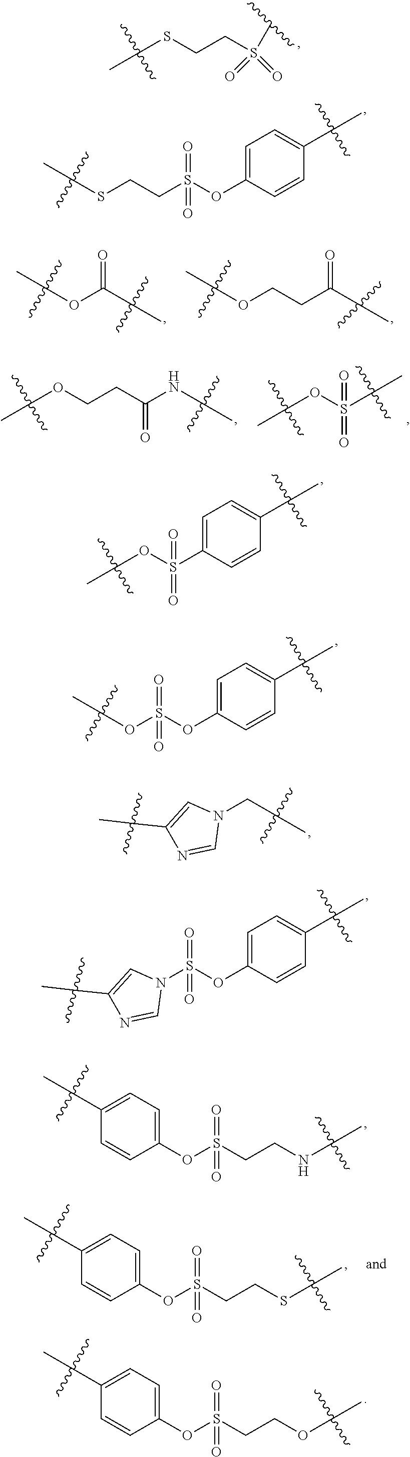

68. The process according to claim 61, wherein the at least one entity chosen from compounds of Formula (II) is chosen from: 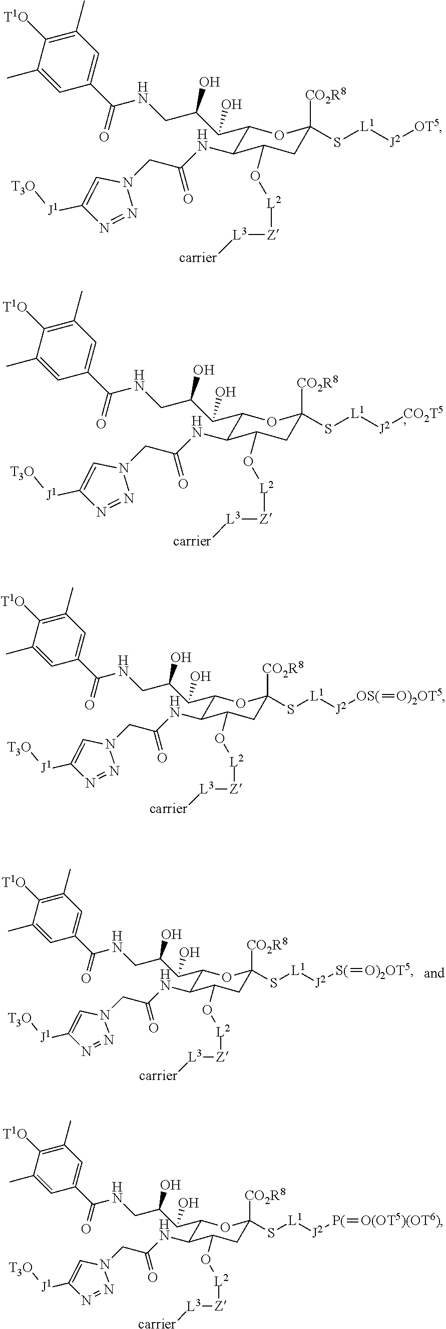
and pharmaceutically acceptable salts of any of the foregoing.

69. The process according to claim 61, wherein the at least one entity chosen from compounds of Formula (II) is chosen from: 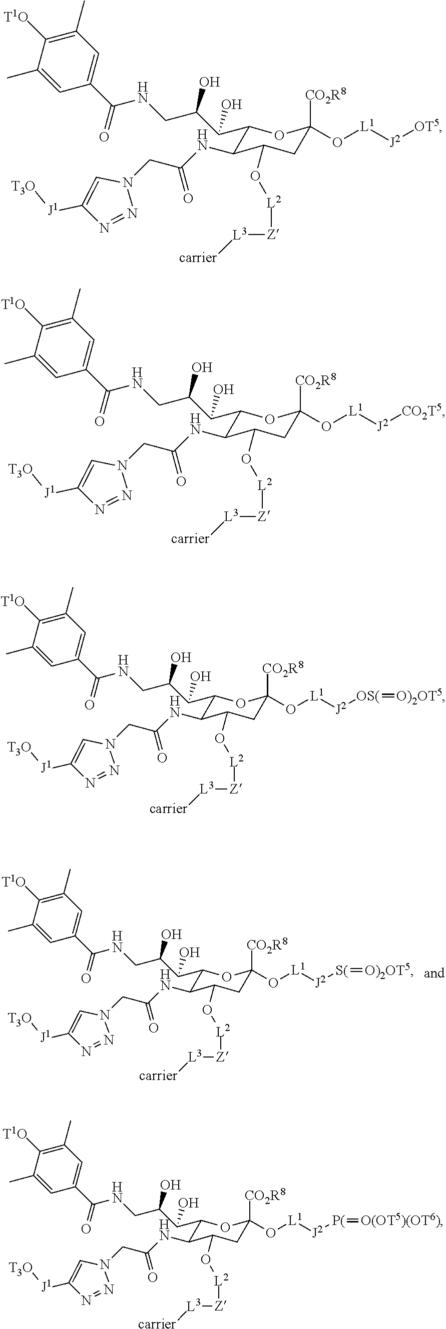
and pharmaceutically acceptable salts of any of the foregoing.

70. The process according to claim 61, wherein the at least one entity chosen from compounds of Formula (II) is chosen from: 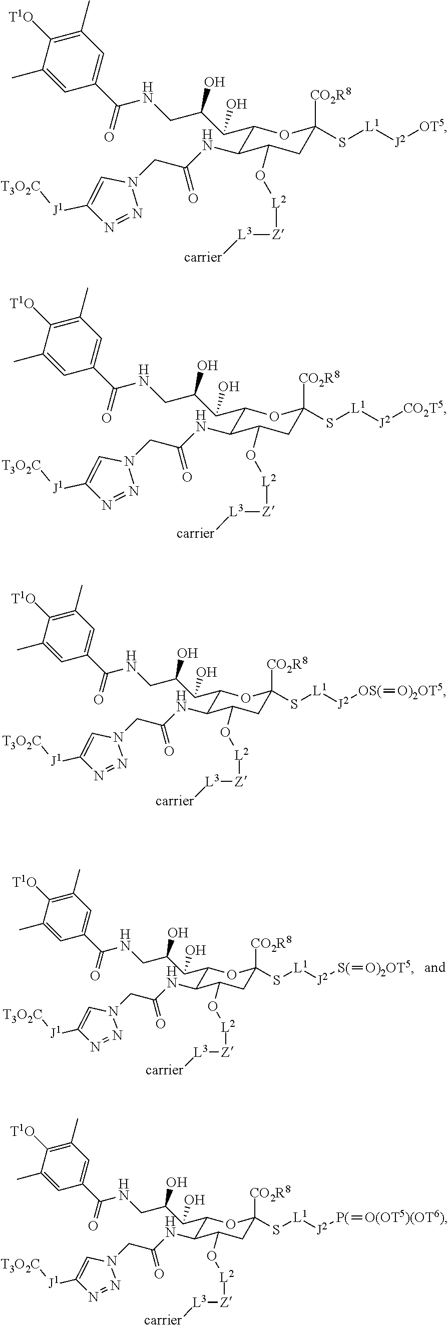
and pharmaceutically acceptable salts of any of the foregoing.

71. The process according to claim 61, wherein the at least one entity chosen from compounds of Formula (II) is chosen from: 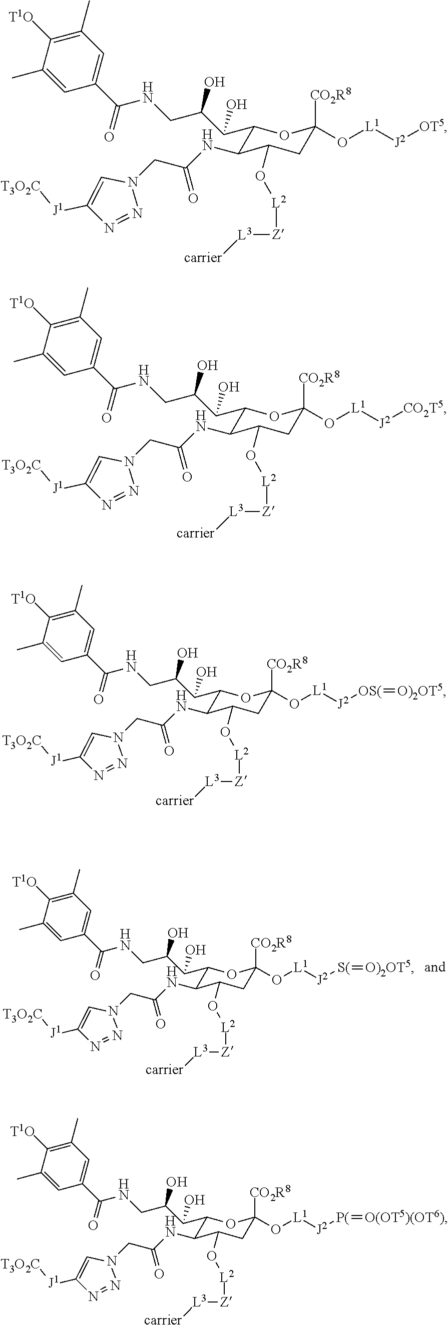
and pharmaceutically acceptable salts of any of the foregoing.

72. The process according to claim 68, wherein at least one of T¹, T³, T⁵, T⁶, and R⁸ is H.

73. The process according to claim 72, wherein T¹ is H.

74. The process according to claim 73, wherein T³ is H.

75. The process according to claim 68, wherein T⁵ and/or T⁶ is H.

76. The process according to claim 68, wherein R⁸ is H.

77. The process according to claim 68, wherein each of T¹, T³, and R⁸ is H.
